(19) United States
(12) Patent Application Publication
(10) Pub. No.: US 2025/0382273 A1
(43) Pub. Date: Dec. 18, 2025

(54) BENZOTHIAZOLE ANAOLOGUES AND USE THEREOF AS BOOSTERS OF ANTI TUBERCULOSIS DRUGS

(71) Applicants: INSTITUT NATIONAL DE LA SANTE ET DE LA RECHERCHE MEDICALE, Paris (FR); INSTITUT PASTEUR DE LILLE, Lille (FR); UNIVERSITÉ DE LILLE, Lille (FR); CENTRE NATIONAL DE LA RECHERCHE SCIENTIFIQUE, Paris (FR); CENTRE HOSPITALIER UNIVERSITAIRE DE LILLE, Lille (FR)

(72) Inventors: Kamel DJAOUT, Lille (FR); Alain BAULARD, Lille Cedex (FR); Alice BOULLOY, Lille (FR); Aurore DRENEAU, Lille (FR); Marion FLIPO, Lille Cedex (FR); Marion PRIERI, Beerse (FR); Nicolas WILLAND, Lille (FR); Léo FAÏON, Newcastle Upon Tyne (GB)

(21) Appl. No.: 18/725,989

(22) PCT Filed: Jan. 5, 2023
(86) PCT No.: PCT/EP2023/050182

§ 371 Date: Jul. 1, 2024

(30) Foreign Application Priority Data
Jan. 6, 2022 (EP) 22305007.1

(51) Int. Cl. C07D 277/82; A61K 31/428; A61K 31/429; A61K 31/4439; A61K 31/454; A61K 31/4709; A61K 31/496; A61K 31/506; A61K 31/5365; A61K 31/5377; A61P 31/06; C07D 417/04; C07D 417/10; C07D 417/12; C07D 417/14; C07D 513/04

(52) U.S. Cl. CPC C07D 277/82; A61K 31/428; A61K 31/429; A61K 31/4439; A61K 31/454; A61K 31/4709; A61K 31/496; A61K 31/506; A61K 31/5365; A61K 31/5377; A61P 31/06; C07D 417/04; C07D 417/10; C07D 417/12; C07D 417/14; C07D 513/04

(57) ABSTRACT

The present invention relates to new norbornen analogues of formula (I), wherein the norbornen moiety is in R5 and to the use of norbornen analogues as a drug, in particular for the prevention and/or treatment of a mycobacterial infection or for the prevention and/or treatment of a disease caused by infection with a mycobacterium.

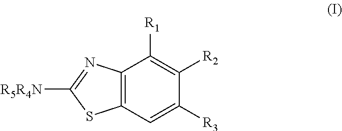

The present invention relates to new benzothiazole analogues and to the use of benzothiazole analogues as a drug, in particular for the prevention and/or treatment of a mycobacterial infection or for the prevention and/or treatment of a disease caused by infection with a mycobacterium. More particularly, said infection is a Mycobacterium tuberculosis infection.

### BACKGROUND

Tuberculosis (TB) stays the major cause of mortality worldwide from a single infectious agent. In 2019, according to the World Health Organization (WHO), it has been responsible for the death of 1.5 million persons and 10 million fall ill every year. Moreover, one third of the worldwide population would be infected by latent form of Tuberculosis and 10% would develop the disease [Global Tuberculosis Report 2019-WHO].

A wide range of treatment is still available today to treat sensitive tuberculosis but the rapid and a global emergence of multidrug resistant (MDR) and Extensive drug resistant (XDR) becomes alarming and obliges the scientific community to develop new alternative strategies.

MDR-TB is characterized by resistance to the two key first line drugs (isoniazid and rifampicin) while XDR-TB is defined as MDR-TB with additional resistance to fluoroquinolones and at least two injectable second-line drugs. Treatment duration, ranging from 6 to 9 months in the best scenario can be extended to 24 months or more in the worst-case scenario and is associated to numerous side effects leading to non-compliance from patient. Thus, treatment duration with the drug regimens complexity are part of the challenge.

In the past 10 years, most of the research in the drug discovery field has been devoted to the discovery of new active family of anti-tubercular agents. One of the common characteristics shared by old and new antituberculosis drugs is that they are prodrugs in a sense that they only become active when bioactivated in the mycobacteria. Consequently, mutations in the pathways of bioactivation are classically observed in Mycobacterium tuberculosis clinical strains that have developed resistance to prodrugs.

As a consequence, the inventors have developed molecules able to trigger alternative bioactivating pathways of the bacteria. The non-exhaustive list of potential benefits of such molecules is, i.) to enhance the potency of existing prodrugs, ii.) to improve their therapeutic index, iii.) to shorten treatment duration, iv) to reduce the emergence of resistant bacteria, v;) to re-sensitize clinical strains resistant to the prodrug.

The present invention describes the discovery of new chemical series of benzothiazole analogues here called Smart-Nim able to reprogram the bioactivation of anti-tuberculosis prodrugs of the Nitroimidazole family. These Smart-Nim molecules i.) increase the sensitivity of M. tuberculosis to nitroimidazoles, and ii.) restore the sensitivity to nitroimidazoles of M. tuberculosis clinical strains resistant to this family of antibiotics.

### DETAILED DESCRIPTION

The present invention thus relates to a compound of formula (I):

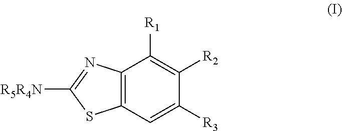

Wherein:
   R₁ is chosen from
      —H;
      —OH;
      linear or branched -(C₁-C₆)alkyl; and
      linear or branched -(C₁-C₆)alkoxy(C₂-C₆)alkynyl;
   R₂ is chosen from:
      —H;
      linear or branched -(C₁-C₆)alkyl;
      linear or branched -(C₁-C₆)alkoxy; and
      an halogen atom;
   R₃ is chosen from:
      —H;
      an halogen atom;
      linear or branched -(C₁-C₆)alkyl optionally substituted by one or more halogen atoms;
      linear or branched -(C₁-C₆)alkoxy optionally substituted by one or more halogen atoms;
      linear or branched -(C₂-C₈)alkynyl;
      linear or branched -(C₁-C₆)alkoxy-(C₂-C₆)alkynyl;
      —OR;
      —NHR;
      -(C₂-C₅) cycloalkyl;
      linear or branched -(C₂-C₆)alkynyl-(C₆-C₁₀)aryl; linear or branched -(C₁-C₆)alkyl-(C₆-C₁₀)aryl;
      -(C₆-C₁₀)aryl optionally substituted by one or more —OH, halogen atoms, linear or branched (C₁-C₆)alkyl optionally substituted by one or more halogen atoms; linear or branched (C₁-C₆)alkoxy optionally substituted by one or more halogen atoms;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;
      linear or branched -(C₂-C₆)alkynyl-(C₂-C₆) cycloalkyl;
      linear or branched -(C₆-C₁₀)aryl-(C₁-C₆)alkoxy-(C₂-C₆)alkynyl;
      —NO₂;
      linear or branched —SO₂(C₁-C₃)alkyl;
      —CN;
      —COO—(C₁-C₃)alkyl;
   R is chosen from:
      —H;
      linear or branched -(C₁-C₆)alkyl optionally substituted by one or more halogen atoms;
      -(C₆-C₁₀)aryl, said -(C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom; linear or branched -(C₁-C₆)alkoxy optionally substituted by one or more halogen atom; —CN;
   or R₂ and R₃ are linked together to form —O—(C₁-C₆)alkyl-O— group;
   R₄ is H and R₅ is:

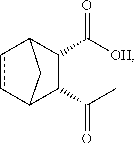

or R₄ and R₅, together with the nitrogen atom to which they are linked form:

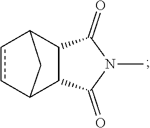

or its pharmaceutically acceptable salts or optical isomers, racemates, diastereoisomers, enantiomers or tautomers;
with the exclusion of the following compounds:
3-[(1,3-benzothiazol-2-yl)carbamoyl]bicyclo[2.2.1]hept-5-ene-2-carboxylic acid;
3-[(6-methyl-1,3-benzothiazol-2-yl)carbamoyl]bicyclo[2.2.1]hept-5-ene-2-carboxylic acid;
3-[(4-methyl-1,3-benzothiazol-2-yl)carbamoyl]bicyclo[2.2.1]hept-5-ene-2-carboxylic acid;
3-[(5,6-dimethyl-1,3-benzothiazol-2-yl)carbamoyl]bicyclo[2.2.1]hept-5-ene-2-carboxylic acid;
3-[(6-bromo-1,3-benzothiazol-2-yl)carbamoyl]bicyclo[2.2.1]hept-5-ene-2-carboxylic acid;
3-[(6-chloro-1,3-benzothiazol-2-yl)carbamoyl]bicyclo[2.2.1]hept-5-ene-2-carboxylic acid;
3-[(4,6-dimethyl-1,3-benzothiazol-2-yl)carbamoyl]bicyclo[2.2.1]hept-5-ene-2-carboxylic acid;
3-[(6-ethoxy-1,3-benzothiazol-2-yl)carbamoyl]bicyclo[2.2.1]hept-5-ene-2-carboxylic acid;
3-[(6-nitro-1,3-benzothiazol-2-yl)carbamoyl]bicyclo[2.2.1]hept-5-ene-2-carboxylic acid;
3-[(6-methylsulfonyl-1,3-benzothiazol-2-yl)carbamoyl]bicyclo[2.2.1]hept-5-ene-2-carboxylic acid;
(1R,2S,6R,7S)-4-(6-methyl-1,3-benzothiazol-2-yl)-4-azatricyclo[5.2.1.02,6]dec-8-ene-3,5-dione;
(1R,2S,6R,7S)-4-(6-ethoxy-1,3-benzothiazol-2-yl)-4-azatricyclo[5.2.1.02,6]dec-8-ene-3,5-dione;
2-[(6-ethyl-1,3-benzothiazol-2-yl)carbamoyl]bicyclo[2.2.1]hept-5-ene-3-carboxylic acid;
2-[(6-fluoro-1,3-benzothiazol-2-yl)carbamoyl]bicyclo[2.2.1]hept-5-ene-3-carboxylic acid;
2-[(5-ethoxy-1,3-benzothiazol-2-yl)carbamoyl]bicyclo[2.2.1]hept-5-ene-3-carboxylic acid;
2-(1,3-benzothiazol-2-yl)-4-ethyl-3a,4,7,7a-tetrahydroisoindole-1,3-dione;
2-(5-chloro-6-methyl-1,3-benzothiazol-2-yl)-4-ethyl-3a,4,7,7a-tetrahydroisoindole-1,3-dione;
4-ethyl-2-(4-methyl-1,3-benzothiazol-2-yl)-3a,4,7,7a-tetrahydroisoindole-1,3-dione;
2-(6-bromo-1,3-benzothiazol-2-yl)-4-ethyl-3a,4,7,7a-tetrahydroisoindole-1,3-dione;
4-ethyl-2-(6-methoxy-1,3-benzothiazol-2-yl)-3a,4,7,7a-tetrahydroisoindole-1,3-dione.

In particular, it relates to a compound of formula (I):

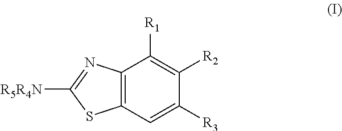

Wherein:
   R₁ is chosen from.
      —H;
      —OH;
      linear or branched -(C₁-C₆)alkyl; and
      linear or branched -(C₁-C₆)alkoxy(C₂-C₆)alkynyl;
   —R₂ is chosen from:
      —H;
      linear or branched -(C₁-C₆)alkyl;
      linear or branched -(C₁-C₆)alkoxy; and
      an halogen atom;
   R₃ is chosen from:
      —H;
      an halogen atom;
      linear or branched -(C₁-C₆)alkyl optionally substituted by one or more halogen atoms;
      linear or branched -(C₁-C₆)alkoxy optionally substituted by one or more halogen atoms;
      linear or branched -(C₂-C₅)alkynyl;
      linear or branched -(C₁-C₆)alkoxy-(C₂-C₆)alkynyl;
      —OR;
      —NHR;
      (C₂-C₈) cycloalkyl;
      linear or branched -(C₂-C₆)alkynyl-(C₆-C₁₀)aryl;
      linear or branched -(C₁-C₆)alkyl-(C₆-C₁₀)aryl;
   -(C₆-C₁₀)aryl optionally substituted by one or more —OH, halogen atoms, linear or branched (C₁-C₆)alkyl optionally substituted by one or more halogen atoms; linear or branched (C₁-C₆)alkoxy optionally substituted by one or more halogen atoms; —NRaRb;
      linear or branched -(C₂-C₆)alkynyl-(C₂-C₆) cycloalkyl
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;
      linear or branched -(C₂-C₆)alkynyl-(C₂-C₆) cycloalkyl;
      linear or branched -(C₆-C₁₀)aryl-(C₁-C₆)alkoxy-(C₂-C₆)alkynyl;
      —NO₂;
      linear or branched —SO₂(C₁-C₃)alkyl;
      —CN;
      —COO—(C₁-C₃)alkyl;
   R is chosen from:
      —H;
      linear or branched -(C₁-C₆)alkyl optionally substituted by one or more halogen atoms;
      (C₆-C₁₀)aryl, said -(C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom; linear or branched -(C₁-C₆)alkoxy optionally substituted by one or more halogen atom; —CN;
   Ra and Rb, identical or different, are chosen from:
      —H;
      linear or branched -(C₁-C₆)alkyl, wherein one of the carbon atoms of the alkyl group can be substituted by an heteroatom;
or Rₐ and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo (═O);
   Rc and Rd, identical or different, are chosen from:
      —H; and
      linear or branched -(C₁-C₆)alkyl;
   or R₂ and R₃ are linked together to form —O—(C₁-C₆)alkyl-O— group;
   R₄ is H and R₅ is:

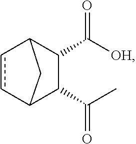

or R₄ and R₅, together with the nitrogen atom to which they are linked form:

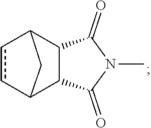

or its pharmaceutically acceptable salts or optical isomers, racemates, diastereoisomers, enantiomers or tautomers;
with the exclusion of the following compounds:
3-[(1,3-benzothiazol-2-yl)carbamoyl]bicyclo[2.2.1]hept-5-ene-2-carboxylic acid;
3-[(6-methyl-1,3-benzothiazol-2-yl)carbamoyl]bicyclo[2.2.1]hept-5-ene-2-carboxylic acid;
3-[(4-methyl-1,3-benzothiazol-2-yl)carbamoyl]bicyclo[2.2.1]hept-5-ene-2-carboxylic acid;
3-[(5,6-dimethyl-1,3-benzothiazol-2-yl)carbamoyl]bicyclo[2.2.1]hept-5-ene-2-carboxylic acid;
3-[(6-bromo-1,3-benzothiazol-2-yl)carbamoyl]bicyclo[2.2.1]hept-5-ene-2-carboxylic acid;
3-[(6-chloro-1,3-benzothiazol-2-yl)carbamoyl]bicyclo[2.2.1]hept-5-ene-2-carboxylic acid;
3-[(4,6-dimethyl-1,3-benzothiazol-2-yl)carbamoyl]bicyclo[2.2.1]hept-5-ene-2-carboxylic acid;
3-[(6-ethoxy-1,3-benzothiazol-2-yl)carbamoyl]bicyclo[2.2.1]hept-5-ene-2-carboxylic acid;
3-[(6-nitro-1,3-benzothiazol-2-yl)carbamoyl]bicyclo[2.2.1]hept-5-ene-2-carboxylic acid;
3-[(6-methylsulfonyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
2-[(6-ethyl-1,3-benzothiazol-2-yl)carbamoyl]bicyclo[2.2.1]hept-5-ene-3-carboxylic acid;
2-[(6-fluoro-1,3-benzothiazol-2-yl)carbamoyl]bicyclo[2.2.1]hept-5-ene-3-carboxylic acid;
2-[(5-ethoxy-1,3-benzothiazol-2-yl)carbamoyl]bicyclo[2.2.1]hept-5-ene-3-carboxylic acid;
(2-(benzo[d]thiazol-2-yl)-3a,4,7,7a-tetrahydro-1H-4,7-methanoisoindole-1,3 (2H)-dione
2-(5-chloro-6-methylbenzo[d]thiazol-2-yl)-3a,4,7,7a-tetrahydro-1H-4,7-methanoisoindole-1,3 (2H)-dione
2-(4-methylbenzo[d]thiazol-2-yl)-3a,4,7,7a-tetrahydro-1H-4,7-methanoisoindole-1,3 (2H)-dione
2-(6-bromobenzo[d]thiazol-2-yl)-3a,4,7,7a-tetrahydro-1H-4,7-methanoisoindole-1,3 (2H)-dione
2-(6-methoxybenzo[d]thiazol-2-yl)-3a,4,7,7a-tetrahydro-1H-4,7-methanoisoindole-1,3 (2H)-dione;3-[(6-methoxy-1,3-benzothiazol-2-yl)carbamoyl]norbornane-2-carboxylic acid;4-(6-methoxy-1,3-benzothiazol-2-yl)-4-azatricyclo[5.2.1.02,6]dec-8-ene-3,5-dione;
4-(5-chloro-6-methyl-1,3-benzothiazol-2-yl)-4-azatricyclo[5.2.1.02,6]dec-8-ene-3,5-dione;
4-(4-methyl-1,3-benzothiazol-2-yl)-4-azatricyclo[5.2.1.02,6]dec-8-ene-3,5-dione;
4-(6-bromo-1,3-benzothiazol-2-yl)-4-azatricyclo[5.2.1.02,6]dec-8-ene-3,5-dione;
4-(4-methyl-1,3-benzothiazol-2-yl)-4-azatricyclo[5.2.1.02,6]decane-3,5-dione.

In one embodiment, the (3aS,4R,7S,7aS)-[2-(2-benzothiazolyl)]-4,7-methano-1H-isoindol-1,3-(2H)-dione and/or -4-(6-ethoxy-1,3-benzothiazol-2-yl)-4-azatricyclo[5.2.1.02,6]dec-8-ene-3,5-dione are also excluded.

The invention also relates to a pharmaceutical composition comprising a compound of formula (I) as described above and a pharmaceutically acceptable excipient.

It further relates to a compound of formula (I):

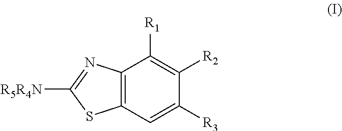

wherein:
   R₁ is chosen from
      —H;
      —OH;
      linear or branched -(C₁-C₆)alkyl; and
      linear or branched -(C₁-C₆)alcoxy(C₂-C₆)alkynyl;
   R₂ is chosen from:
      —H;
      linear or branched -(C₁-C₆)alkyl;
      linear or branched -(C₁-C₆)alkoxy; and
      an halogen atom;
   R₃ is chosen from:
      —H;
      an halogen atom;
      linear or branched -(C₁-C₆)alkyl optionally substituted by one or more halogen atoms;
      linear or branched -(C₁-C₆)alkoxy optionally substituted by one or more halogen atoms;
      linear or branched -(C₂-C₅)alkynyl;
      linear or branched -(C₁-C₆)alkoxy-(C₂-C₆)alkynyl;
      —OR;
      —NHR;
      -(C₂-C₅) cycloalkyl:
      linear or branched -(C₂-C₆)alkynyl-(C₆-C₁₀)aryl;
      linear or branched -(C₁-C₆)alkyl-(C₆-C₁₀)aryl;
      -(C₆-C₁₀)aryl optionally substituted by one or more —OH, halogen atoms, linear or branched (C₁-C₆)alkyl optionally substituted by one or more halogen atoms, linear or branched (C₁-C₆)alkoxy optionally substituted by one or more halogen atoms;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;
      linear or branched -(C₂-C₆)alkynyl-(C₂-C₆) cycloalkyl;
      linear or branched -(C₆-C₁₀)aryl-(C₁-C₆)alkoxy-(C₂-C₆)alkynyl;
      —NO₂;
      linear or branched —SO₂(C₁-C₃)alkyl;
      —CN;
      —COO—(C₁-C₃)alkyl;
   R is chosen from:
      —H;
      linear or branched -(C₁-C₆)alkyl optionally substituted by one or more halogen atoms;
      -(C₆-C₁₀)aryl, said (C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
      an heteroaryl comprising 3 to 10 members having at least one heteroatom chosen from O, N, or S; said heteroaryl being optionally substituted by one or more halogen atoms, linear or branched -(C₁-C₆)alkyl optionally substituted by one or more halogen atoms; linear or branched -(C₁-C₆)alkoxy optionally substituted by one or more halogen atom; —CN;
   or R₂ and R₃ are linked together to form —O—(C₁-C₆)alkyl-O— group;
   R₄ is H and R₅ is:

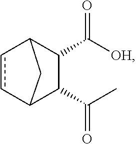

or R₄ and R₅, together with the nitrogen atom to which they are linked form:

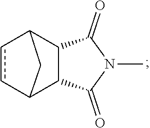

or its pharmaceutically acceptable salts or optical isomers, racemates, diastereoisomers, enantiomers or tautomers;
   for use as a drug, in particular for the treatment of a mycobacterial infection or in the treatment of a disease caused by infection with a mycobacterium, more particularly wherein the mycobacterial infection is a Mycobacterium tuberculosis infection.

In particular, it further relates to a compound of formula (I):

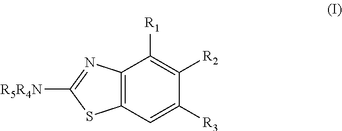

wherein:
   R₁ is chosen from
      —H;
      —OH;
      linear or branched -(C₁-C₆)alkyl; and
      linear or branched -(C₁-C₆)alcoxy(C₂-C₆)alkynyl;
   R₂ is chosen from:
      —H;
      linear or branched -(C₁-C₆)alkyl;
      linear or branched -(C₁-C₆)alkoxy; and
      an halogen atom;
   R₃ is chosen from:
      —H;
      an halogen atom;
      linear or branched -(C₁-C₆)alkyl optionally substituted by one or more halogen atoms;
      linear or branched -(C₁-C₆)alkoxy optionally substituted by one or more halogen atoms;
      linear or branched -(C₂-C₅)alkynyl;
      linear or branched -(C₁-C₆)alkoxy-(C₂-C₆)alkynyl;
      —OR;
      —NHR;
      -(C₂-C₅) cycloalkyl;
      linear or branched -(C₂-C₆)alkynyl-(C₆-C₁₀)aryl;
      linear or branched -(C₁-C₆)alkyl-(C₆-C₁₀)aryl;
      -(C₆-C₁₀)aryl optionally substituted by one or more —OH, halogen atoms, linear or branched (C₁-C₆)alkyl optionally substituted by one or more halogen atoms, linear or branched (C₁-C₆)alkoxy optionally substituted by one or more halogen atoms, —NRaRb;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;
      linear or branched -(C₂-C₆)alkynyl-(C₂-C₆) cycloalkyl;
      linear or branched -(C₆-C₁₀)aryl-(C₁-C₆)alkoxy-(C₂-C₆)alkynyl;
      —NO₂;
      linear or branched —SO₂(C₁-C₃)alkyl;
      —CN;
      —COO—(C₁-C₃)alkyl;
   R is chosen from:
      —H;
      linear or branched -(C₁-C₆)alkyl optionally substituted by one or more halogen atoms;
      -(C₆-C₁₀)aryl, said (C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
      an heteroaryl comprising 3 to 10 members having at least one heteroatom chosen from O, N, or S; said heteroaryl being optionally substituted by one or more halogen atoms, linear or branched -(C₁-C₆)alkyl optionally substituted by one or more halogen atoms; linear or branched -(C₁-C₆)alkoxy optionally substituted by one or more halogen atom; —CN;
   Ra and Rb, identical or different, are chosen from:
      —H;
      linear or branched -(C₁-C₆)alkyl, wherein one of the carbon atoms of the alkyl group can be substituted by an heteroatom;
or Ra and Rb are linked together to form an I heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo (═O);
   Rc and Rd, identical or different, are chosen from:
      —H; and
      linear or branched -(C₁-C₆)alkyl;
   or R₂ and R₃ are linked together to form —O—(C₁-C₆)alkyl-O— group;
   R₄ is H and R₅ is:

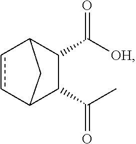

or R₄ and R₅, together with the nitrogen atom to which they are linked form:

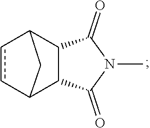

or its pharmaceutically acceptable salts or optical isomers, racemates, diastereoisomers, enantiomers or tautomers;
with the exclusion of the following compound:
3-[(6-methylsulfonyl-1,3-benzothiazol-2-yl)carbamoyl]bicyclo[2.2.1]hept-5-ene-2-carboxylic acid;
   for use as a drug, in particular for the treatment of a mycobacterial infection or in the treatment of a disease caused by infection with a mycobacterium, more particularly wherein the mycobacterial infection is a Mycobacterium tuberculosis infection.

In one embodiment, the (3aS,4R,7S,7aS)-[2-(2-benzothiazolyl)]-4,7-methano-1H-isoindol-1,3-(2H)-dione and/or the Bicyclo[2.2.1]hept-5-ene-2,3-dicarboximide-N-[2-(6-methylbenzo)thiazolyl] are also excluded.

Unless specified otherwise, the terms used hereabove or hereafter as regards to the compounds of formula (I) have the meaning ascribed to them below:
“H” refers to hydrogen.
“OH” refers to hydroxyle.
“CN” refers to carbonitrile.
“NO₂” refers to nitro group.
“oxo” refers to ═O;
“SO₂(C₁-C₃)alkyl refers to in particular SO₂CH₃.
“COO—(C₁-C₃)alkyl” refers to in particular COOCH₃ or COOC(CH₃)₃.
“halogen” refers to fluorine, chlorine, brome or iodine atom.
“alkyl” represents an aliphatic-hydrocarbon group which may be straight or branched, having 1 to 6 or 1 to 3 carbon atoms in the chain (C₁-C₆)alkyl or (C₁-C₃)alkyl, unless specified otherwise. In particular, alkyl groups have 1 to 3 carbon atoms in the chain (C₁-C₃)alkyl. Branched means that one or more alkyl groups such as methyl, ethyl or propyl are attached to a linear alkyl chain. Exemplary alkyl groups include methyl, ethyl, n-propyl, i-propyl, n-butyl, t-butyl, 2,2-dimethylbutyl, n-pentyl, n-hexyl, in particular methyl or ethyl. As mentioned, said alkyl can be substituted by one or more halogen atoms i.e. said alkyl having for example 1 to 3 carbon atoms in the chain (C₁-C₃)halogenoalkyl has one or more hydrogen atoms that has been replaced by a halogen atom such as fluorine, chlorine, bromine or iodine atom, in particular by one or more fluorine atoms. Exemplary halogenoalkyl include trifluoromethyl. In the case where it is specified that one the carbon atoms of the alkyl group can be substituted by an heteroatom, it means that one of the carbons of the chain can be replaced by an heteroatom such as O, N or S, in particular N or O.
“alkoxy” represent an alkyl group as previously defined singular bonded to oxygen. Examples of linear or branched (C₁-C₆)alkoxy includes methoxy(CH₃₀—) and ethoxy(CH₃CH₂O—). Said alkoxy can be substituted by one or more fluorine atoms such as trifluoromethoxy.
“alkynyl” refers to an aliphatic hydrocarbon group containing a carbon-carbon triple bond and which may be straight or branched having 2 to 8 or 2 to 6 carbon atoms in the chain (C₂-C₅)alkynyl or (C₂-C₆)alkynyl, unless specified otherwise. Preferred alkynyl groups have 2 to 6 carbon atoms in the chain (C₂-C₆)alkynyl. Exemplary alkynyl groups include ethynyl, propynyl, but-1-ynyl, but-2-ynyl, 3,3-dimethylbut-1-ynyl.
“cycloalkyl” refers to a saturated monocyclic or bicyclic non-aromatic hydrocarbon ring of 2 to 8 carbon atoms or 2 to 6 carbon atoms, preferably 3 to 6 carbon atoms, which can comprise one or more unsaturation. Specific examples of monocyclic cycloalkyl groups include cyclopropyl, cyclobutyl, cyclopentyl, cyclopentenyl, cyclohexyl, cyclohexenyl. Preferably, the cycloalkyl group is cyclohexyl or cyclopropyl.
“aryl” refers to an aromatic monocyclic or multicyclic hydrocarbon ring system of 6 to 10 carbon atoms, preferably of 6 carbon atoms. Exemplary aryl groups include phenyl, naphthyl, benzyl, phenanthryl, biphenyl, in particular phenyl. Said aryl or phenyl can be substituted by one or more halogen atoms such as fluorine, bromine, iodine or chlorine; (C₁-C₆)alkyl or (C₁-C₃)alkyl such as a methyl, said alkyl being optionally substituted by one to 3 halogen atoms, in particular fluorine, such as trifluoromethyl; —OH; (C₁-C₆)alkoxy or (C₁-C₃)alkoxy such as a methoxy, said alkoxy being optionally substituted by one to 3 halogen atoms, in particular fluorine, such as trifluoromethoxy.
“heteroaryl” refers to a 3 to 10, aromatic mono-, bi- or multicyclic ring wherein at least one member of the ring is a hetero atom. Hetero atoms can be O, S or N. In particular, each ring comprises from 1 to 3 hetero atoms. Examples include pyrrolyl, pyridyl, quinolyl, furan, thiophene, pyrimidinyl, pyrazolyl, pyrazinyl, indolyl, imidazolyl, in particular pyridyl, thiophene, quinolyl or pyrimidinyl. Said heteroaryl can be substituted by a halogen atom such as fluorine, bromine, iodine or chlorine, in particular fluorine or bromine; (C₁-C₆)alkyl such as a methyl, said alkyl being optionally substituted by one or more halogen atoms, in particular by 3 fluorine atoms such as trifluoromethyl; (C₁-C₆)alkoxy or (C₁-C₅)alkoxy such as a methoxy, said alkoxy being optionally substituted by one to 3 halogen atoms, in particular fluorine, such as trifluoromethoxy; CN.
“heterocycloalkyl” or “heterocycle” refers to a saturated or partially unsaturated non aromatic stable 3 to 10-membered mono, bi or multicyclic rings and wherein at least one member of the ring is a nitrogen atom. Rings can comprise more than one nitrogen atom, and can comprise other heterotaoms such as oxygen or sulfur atoms. Suitable heterocycles are for example disclosed in the Handbook of Chemistry and Physics, 76th Edition, CRC Press, Inc., 1995-1996, pages 225 to 226, the disclosure of which is hereby incorporated by reference. Examples of heterocycloalkyl include, but are not limited to piperazine, diazepane, piperidine, pyrrolidine, imidazolidine, morpholine, azetidine, diazabicyclo octanyl, diazabicycloheptanyl, azabicyclohexanyl, in particular piperazine, morpholine, pyrrolidine and piperidine. Said heterocyclalkyl is optionally substituted by one or more linear or branched -(C₁-C₆)alkyl, —COO—(C₁-C₃)alkyl, —NRcRd; oxo (═O); with Rc and Rd, identical or different, chosen from: —H and linear or branched -(C₁-C₆)alkyl. The term “substituted” generally refers to, unless specified otherwise, a substitution with one or more substituents, which may be identical or different, and which are identified herein.

The compounds of formula (I) as described herein can comprise one or more asymmetric carbon atoms. They can therefore exist in the form of enantiomers or diastereoisomers. These enantiomers and diastereoisomers, as well as their mixtures, including racemic mixtures, form part of the invention.

The compounds of formula (I) as described herein can be provided in the form of a free base or in the form of addition salts with acids or amines, which also form part of the invention.

These salts are advantageously prepared with pharmaceutically acceptable amines, but salts with other amines, useful for example for the purification or for the isolation of the compounds of formula (I) as described herein, also form part of the invention.

As used herein, the expression “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

As used herein, “pharmaceutically acceptable salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like, including mono, di or tri-salts thereof; and the salts prepared from organic acids such as acetic, propionic, succinic, tartaric, citric, methanesulfonic, benzenesulfonic, glucoronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 20th ed., Mack Publishing Company, Easton, PA, 2000, the disclosure of which is hereby incorporated by reference.

### Compounds

As mentioned, the compounds according to the invention are compounds of formula (I):

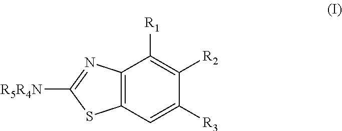

Wherein:
   R₁ is chosen from
      —H;
      —OH;
      linear or branched -(C₁-C₆)alkyl; and
      linear or branched -(C₁-C₆)alcoxy(C₂-C₆)alkynyl; and/or
   R₂ is chosen from:
      —H;
      linear or branched -(C₁-C₆)alkyl;
      linear or branched -(C₁-C₆)alkoxy; and
      an halogen atom; and/or
   R₃ is chosen from:
      —H;
      an halogen atom;
      linear or branched -(C₁-C₆)alkyl optionally substituted by one or more halogen atoms;
      linear or branched -(C₁-C₆)alkoxy optionally substituted by one or more halogen atoms;
      linear or branched -(C₂-C₅)alkynyl;
      linear or branched -(C₁-C₆)alkoxy-(C₂-C₆)alkynyl;
      —OR;
      —NHR;
      -(C₂-C₅) cycloalkyl;
      linear or branched -(C₂-C₆)alkynyl-(C₆-C₁₀)aryl;
      linear or branched -(C₁-C₆)alkyl-(C₆-C₁₀)aryl;
      -(C₆-C₁₀)aryl optionally substituted by one or more —OH, halogen atoms, linear or branched (C₁-C₆)alkyl optionally substituted by one or more halogen atoms, linear or branched (C₁-C₆)alkoxy optionally substituted by one or more halogen atoms, —NRaRb;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;
      linear or branched -(C₂-C₆)alkynyl-(C₂-C₆) cycloalkyl;
      linear or branched -(C₆-C₁₀)aryl-(C₁-C₆)alkoxy-(C₂-C₆)alkynyl;
      —NO₂;
      linear or branched —SO₂(C₁-C₃)alkyl;
      —CN;
      —COO—(C₁-C₃)alkyl;
   R is chosen from:
      —H;
      linear or branched -(C₁-C₆)alkyl optionally substituted by one or more halogen atoms;
      -(C₆-C₁₀)aryl, said -(C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom; (C₁-C₆)alkoxy optionally substituted by one or more halogen atom; —CN;
   Ra and Rb, identical or different, are chosen from:
      —H;
      linear or branched -(C₁-C₆)alkyl, wherein one of the carbon atoms of the alkyl group can be substituted by an heteroatom;
or Ra and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo;
   Rc and Rd, identical or different, are chosen from:
      —H; and
      linear or branched -(C₁-C₆)alkyl;
or R₂ and R₃ are linked together to form —O—(C₁-C₆)alkyl-O— group; and/or
   R₄ is H and R₅ is:

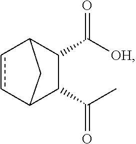

or R₄ and R₅, together with the nitrogen atom to which they are linked form:

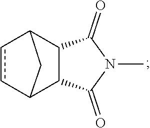

or its pharmaceutically acceptable salts or optical isomers, racemates, diastereoisomers, enantiomers or tautomers;

with the exclusion of the following compounds:
3-[(1,3-benzothiazol-2-yl)carbamoyl]bicyclo[2.2.1]hept-5-ene-2-carboxylic acid;
3-[(6-methyl-1,3-benzothiazol-2-yl)carbamoyl]bicyclo[2.2.1]hept-5-ene-2-carboxylic acid;
3-[(4-methyl-1,3-benzothiazol-2-yl)carbamoyl]bicyclo[2.2.1]hept-5-ene-2-carboxylic acid;
3-[(5,6-dimethyl-1,3-benzothiazol-2-yl)carbamoyl]bicyclo[2.2.1]hept-5-ene-2-carboxylic acid;
3-[(6-bromo-1,3-benzothiazol-2-yl)carbamoyl]bicyclo[2.2.1]hept-5-ene-2-carboxylic acid;
3-[(6-chloro-1,3-benzothiazol-2-yl)carbamoyl]bicyclo[2.2.1]hept-5-ene-2-carboxylic acid;
3-[(4,6-dimethyl-1,3-benzothiazol-2-yl)carbamoyl]bicyclo[2.2.1]hept-5-ene-2-carboxylic acid;
3-[(6-ethoxy-1,3-benzothiazol-2-yl)carbamoyl]bicyclo[2.2.1]hept-5-ene-2-carboxylic acid;
3-[(6-nitro-1,3-benzothiazol-2-yl)carbamoyl]bicyclo[2.2.1]hept-5-ene-2-carboxylic acid;
3-[(6-methylsulfonyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
2-[(6-ethyl-1,3-benzothiazol-2-yl)carbamoyl]bicyclo[2.2.1]hept-5-ene-3-carboxylic acid;
2-[(6-fluoro-1,3-benzothiazol-2-yl)carbamoyl]bicyclo[2.2.1]hept-5-ene-3-carboxylic acid;
2-[(5-ethoxy-1,3-benzothiazol-2-yl)carbamoyl]bicyclo[2.2.1]hept-5-ene-3-carboxylic acid;
(2-(benzo[d]thiazol-2-yl)-3a,4,7,7a-tetrahydro-1H-4,7-methanoisoindole-1,3 (2H)-dione
2-(5-chloro-6-methylbenzo[d]thiazol-2-yl)-3a,4,7,7a-tetrahydro-1H-4,7-methanoisoindole-1,3 (2H)-dione
2-(4-methylbenzo[d]thiazol-2-yl)-3a,4,7,7a-tetrahydro-1H-4,7-methanoisoindole-1,3 (2H)-dione
2-(6-bromobenzo[d]thiazol-2-yl)-3a,4,7,7a-tetrahydro-1H-4,7-methanoisoindole-1,3 (2H)-dione
2-(6-methoxybenzo[d]thiazol-2-yl)-3a,4,7,7a-tetrahydro-1H-4,7-methanoisoindole-1,3 (2H)-dione;
3-[(6-methoxy-1,3-benzothiazol-2-yl)carbamoyl]norbornane-2-carboxylic acid;
4-(6-methoxy-1,3-benzothiazol-2-yl)-4-azatricyclo[5.2.1.02,6]dec-8-ene-3,5-dione;
4-(5-chloro-6-methyl-1,3-benzothiazol-2-yl)-4-azatricyclo[5.2.1.02,6]dec-8-ene-3,5-dione;
4-(4-methyl-1,3-benzothiazol-2-yl)-4-azatricyclo[5.2.1.02,6]dec-8-ene-3,5-dione;
4-(6-bromo-1,3-benzothiazol-2-yl)-4-azatricyclo[5.2.1.02,6]dec-8-ene-3,5-dione;
4-(4-methyl-1,3-benzothiazol-2-yl)-4-azatricyclo[5.2.1.02,6]decane-3,5-dione.

In one embodiment, the (3aS,4R,7S,7aS)-[2-(2-benzothiazolyl)]-4,7-methano-1H-isoindol-1,3-(2H)-dione and/or 4-(6-ethoxy-1,3-benzothiazol-2-yl)-4-azatricyclo[5.2.1.02,6]dec-8-ene-3,5-dione are also excluded.

In particular, said compound is chosen from:
3-[(6-methyl-1,3-benzothiazol-2-yl)carbamoyl]norbornane-2-carboxylic acid;
(1S,2S,6R,7R)-4-(6-methyl-1,3-benzothiazol-2-yl)-4-azatricyclo[5.2.1.02,6]decane-3,5-dione;
3-[(5-methoxy-1,3-benzothiazol-2-yl)carbamoyl]bicyclo[2.2.1]hept-5-ene-2-carboxylic acid;
3-[(6-methoxy-1,3-benzothiazol-2-yl)carbamoyl]bicyclo[2.2.1]hept-5-ene-2-carboxylic acid;
3-[(5,6-dimethoxy-1,3-benzothiazol-2-yl)carbamoyl]bicyclo[2.2.1]hept-5-ene-2-carboxylic acid;
3-(6,7-dihydro-[1,4]dioxino[2,3-f][1,3]benzothiazol-2-ylcarbamoyl) bicyclo [2.2.1]hept-5-ene-2-carboxylic acid;
3-[(6-ethyl-1,3-benzothiazol-2-yl)carbamoyl]bicyclo[2.2.1]hept-5-ene-2-carboxylic acid;
3-[(6-tert-butyl-1,3-benzothiazol-2-yl)carbamoyl]bicyclo[2.2.1]hept-5-ene-2-carboxylic acid;
3-[(4,5-dimethyl-1,3-benzothiazol-2-yl)carbamoyl]bicyclo[2.2.1]hept-5-ene-2-carboxylic acid;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
3-[(6-benzyl-1,3-benzothiazol-2-yl)carbamoyl]bicyclo[2.2.1]hept-5-ene-2-carboxylic acid;
3-[[6-(4-fluorophenoxy)-1,3-benzothiazol-2-yl]carbamoyl]bicyclo[2.2.1]hept-5-ene-2-carboxylic acid;
3-[[6-[[4-(trifluoromethyl)phenoxy]-1,3-benzothiazol-2-yl]carbamoyl]bicyclo [2.2.1]hept-5-ene-2-carboxylic acid;
(1R,2S,6R,7S)-4-[6-[4-(trifluoromethyl)phenoxy]-1,3-benzothiazol-2-yl]-4-azatricyclo[5.2.1.02,6]dec-8-ene-3,5-dione;
3-[[6-(4-pyridyloxy)-1,3-benzothiazol-2-yl]carbamoyl]bicyclo[2.2.1]hept-5-ene-2-carboxylic acid;
3-[[6-[[3-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[3-(trifluoromethyl)-2-pyridyl]oxy]-1,3-benzothiazol-2-yl]-4-azatricyclo[5.2.1.02,6]dec-8-ene-3,5-dione;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[4-(trifluoromethyl)-2-pyridyl]oxy]-1,3-benzothiazol-2-yl]-4-azatricyclo[5.2.1.02,6]dec-8-ene-3,5-dione;
3-[[6-[(4-methyl-2-pyridyl)oxy]-1,3-benzothiazol-2-yl]carbamoyl]bicyclo [2.2.1]hept-5-ene-2-carboxylic acid;
2-[[2-[(1R,2S,6R,7S)-3,5-dioxo-4-azatricyclo[5.2.1.02,6]dec-8-en-4-yl]-1,3-benzothiazol-6-yl]oxy]pyridine-4-carbonitrile;
3-[[6-[[2-(trifluoromethyl)-4-pyridyl]oxy]-1,3-benzothiazol-2-yl]carbamoyl]bicyclo[2.2.1]hept-5-ene-2-carboxylic acid;
(1R,2S,6R,7S)-4-[6-[[6-(trifluoromethyl)-2-pyridyl]oxy]-1,3-benzothiazol-2-yl]-4-azatricyclo[5.2.1.02,6]dec-8-ene-3,5-dione;
3-[[6-[[5-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[5-(trifluoromethyl)-2-pyridyl]oxy]-1,3-benzothiazol-2-yl]-4-azatricyclo[5.2.1.02,6]dec-8-ene-3,5-dione;
3-[[6-[(5-bromo-2-pyridyl)oxy]-1,3-benzothiazol-2-yl]carbamoyl]bicyclo [2.2.1]hept-5-ene-2-carboxylic acid;
3-[(6-cyano-1,3-benzothiazol-2-yl)carbamoyl]bicyclo[2.2.1]hept-5-ene-2-carboxylic acid;
3-[(6-methoxycarbonyl-1,3-benzothiazol-2-yl)carbamoyl]bicyclo[2.2.1]hept-5-ene-2-carboxylic acid;
3-[(6-fluoro-1,3-benzothiazol-2-yl)carbamoyl]bicyclo[2.2.1]hept-5-ene-2-carboxylic acid;
3-[[6-(trifluoromethyl)-1,3-benzothiazol-2-yl]carbamoyl]bicyclo[2.2.1]hept-5-ene-2-carboxylic acid;
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[(5-bromo-1,3-benzothiazol-2-yl)carbamoyl]bicyclo[2.2.1]hept-5-ene-2-carboxylic acid;
3-[(6-iodo-1,3-benzothiazol-2-yl)carbamoyl]bicyclo[2.2.1]hept-5-ene-2-carboxylic acid;
3-[(6-hydroxy-1,3-benzothiazol-2-yl)carbamoyl]bicyclo[2.2.1]hept-5-ene-2-carboxylic acid;
3-[(6-phenyl-1,3-benzothiazol-2-yl)carbamoyl]-7-oxabicyclo[2.2.1]hept-5-ene-2-carboxylic acid;
3-[(6-cyclohexyl-1,3-benzothiazol-2-yl)carbamoyl]bicyclo[2.2.1]hept-5-ene-2-carboxylic acid;
3-[[6-(p-tolyl)-1,3-benzothiazol-2-yl]carbamoyl]bicyclo[2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[[6-(4-chlorophenyl)-1,3-benzothiazol-2-yl]carbamoyl]bicyclo[2.2.1]hept-5-ene-2-carboxylic acid;
3-[[6-(3,4-dichlorophenyl)-1,3-benzothiazol-2-yl]carbamoyl]bicycle [2.2.1]hept-5-ene-2-carboxylic acid;
3-[[6-[[2-(trifluoromethyl)phenyl]-1,3-benzothiazol-2-yl]carbamoyl]bicyclo [2.2.1]hept-5-ene-2-carboxylic acid;
(1R,2S,6R,7S)-4-[6-[[2-(trifluoromethyl)phenyl]-1,3-benzothiazol-2-yl]-4-azatricyclo[5.2.1.02,6]dec-8-ene-3,5-dione;
3-[[6-[3-(trifluoromethyl)phenyl]-1,3-benzothiazol-2-yl]carbamoyl]bicyclo [2.2.1]hept-5-ene-2-carboxylic acid;
3-[[6-[[4-(trifluoromethyl)phenyl]-1,3-benzothiazol-2-yl]carbamoyl]bicyclo [2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[[6-[[4-(trifluoromethoxy)phenyl]-1,3-benzothiazol-2-yl]carbamoyl]bicyclo [2.2.1]hept-5-ene-2-carboxylic acid;
(1R,2S,6R,7S)-4-[6-(4-hydroxyphenyl)-1,3-benzothiazol-2-yl]-4-azatricyclo[5.2.1.02,6]dec-8-ene-3,5-dione;
3-[[6-(4-pyridyl)-1,3-benzothiazol-2-yl]carbamoyl]bicyclo[2.2.1]hept-5-ene-2-carboxylic acid;
(1R,2S,6R,7S)-4-[6-(6-fluoro-3-pyridyl)-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo [2.2.1]hept-5-ene-2-carboxylic acid;
(1R,2S,6R,7S)-4-[6-[[6-(trifluoromethyl)-3-pyridyl]-1,3-benzothiazol-2-yl]-4-azatricyclo[5.2.1.02,6]dec-8-ene-3,5-dione;
3-[(6-pyrimidin-5-yl-1,3-benzothiazol-2-yl)carbamoyl]bicyclo[2.2.1]hept-5-ene-2-carboxylic acid
3-[[6-(6-quinolyl)-1,3-benzothiazol-2-yl]carbamoyl]bicyclo[2.2.1]hept-5-ene-2-carboxylic acid
4-[6-(3-thienyl)-1,3-benzothiazol-2-yl]-4-azatricyclo[5.2.1.02,6]dec-8-ene-3,5-dione
3-[[6-(3-furyl)-1,3-benzothiazol-2-yl]carbamoyl]bicyclo[2.2.1]hept-5-ene-2-carboxylic acid;
(1R,2S,6R,7S)-4-[6-(3-furyl)-1,3-benzothiazol-2-yl]-4-azatricyclo[5.2.1.02,6]dec-8-ene-3,5-dione;
4-[6-(4-prop-2-ynoxyphenyl)-1,3-benzothiazol-2-yl]-4-azatricyclo[5.2.1.02,69dec-8-ene-3,5-dione;
(1R,2S,6R,7S)-4-(6-ethynyl-1,3-benzothiazol-2-yl)-4-azatricyclo[5.2.1.02,6]dec-8-ene-3,5-dione;
(1R,2S,6R,7S)-4-(6-prop-2-ynoxy-1,3-benzothiazol-2-yl)-4-azatricyclo[5.2.1.02,6]dec-8-ene-3,5-dione;
(1R,2S,3R,4S)-3-[(4-hydroxy-1,3-benzothiazol-2-yl)carbamoyl]bicyclo[2.2.1]hept-5-ene-2-carboxylic acid;
3-[(4-prop-2-ynoxy-1,3-benzothiazol-2-yl)carbamoyl]bicyclo[2.2.1]hept-5-ene-2-carboxylic acid;
(1R,2S,6R,7S)-4-(4-prop-2-ynoxy-1,3-benzothiazol-2-yl)-4-azatricyclo[5.2.1.02,6]dec-8-ene-3,5-dione;
3-[[6-(2-phenylethynyl)-1,3-benzothiazol-2-yl]carbamoyl]bicyclo[2.2.1]hept-5-ene-2-carboxylic acid;
3-[[6-(3,3-dimethylbut-1-ynyl)-1,3-benzothiazol-2-yl]carbamoyl]bicyclo [2.2.1]hept-5-ene-2-carboxylic acid;
3-[[6-(2-cyclopropylethynyl)-1,3-benzothiazol-2-yl]carbamoyl]bicyclo[2.2.19hept-5-ene-2-carboxylic acid;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
(1R,2S,6R,7S)-4-[[6-[(4-methyl-2-pyridyl)oxy]-1,3-benzothiazol-2-yl]-4-azatricyclo[5.2.1.02,6]dec-8-ene-3,5-dione;
(1R,2S,6R,7S)-4-[6-[(2-chloro-4-pyridyl)oxy]-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo [2.2.1]hept-5-ene-2-carboxylic acid;
(1R,2S,6R,7S)-4-(6-anilino-1,3-benzothiazol-2-yl)-4-azatricyclo[5.2.1.02,6]dec-8-ene-3,5-dione;
4-[6-(2-pyridylamino)-1,3-benzothiazol-2-yl]-4-azatricyclo[5.2.1.02,6]dec-8-ene-3,5-dione; and
3-[[6-[(4-methyl-2-pyridyl)amino]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2 yl]carbamoyl]bicyclo[2.2.1]hept-5-ene-2-carboxylic acid;
3-[[6-[(5-fluoro-2-pyridyl)amino]-1,3-benzothiazol-2 yl]carbamoyl]bicyclo[2.2.1]hept-5-ene-2-carboxylic acid;
4-[6-[(5-fluoro-2-pyridyl)amino]-1,3-benzothiazol-2-yl]-4-azatricyclo[5.2.1.02,6]dec-8-ene-3,5-dione;
3-[[6-[(6-fluoro-2-pyridyl)amino]-1,3-benzothiazol-2-yl]carbamoyl]bicyclo[2.2.1]hept-5-ene-2-carboxylic acid;
4-[6-[(6-fluoro-2-pyridyl)amino]-1,3-benzothiazol-2-yl]-4-azatricyclo[5.2.1.02,6]dec-8-ene-3,5-dione;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethyl)-3-pyridyl]amino]-1,3-benzothiazol-2-yl]carbamoyl]bicyclo[2.2.1]hept-5-ene-2-carboxylic acid;
4-[6-[(2-methoxy-4-pyridyl)amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[6-[[5-(trifluoromethoxy)-2-pyridyl]amino]-1,3-benzothiazol-2-yl]-4-azatricyclo[5.2.1.02,6]dec-8-ene-3,5-dione;
4-[[6-[[5-(trifluoromethyl)-2-pyridyl]amino]-1,3-benzothiazol-2-yl]-4-azatricyclo[5.2.1.02,6]dec-8-ene-3,5-dione;
3-[[6-[[5-(trifluoromethyl)-2-pyridyl]amino]-1,3-benzothiazol-2-yl]carbamoyl]bicyclo[2.2.1]hept-5-ene-2-carboxylic acid;
4-[6-[[6-(trifluoromethyl)-2-pyridyl]amino]-1,3-benzothiazol-2-yl]-4-azatricyclo[5.2.1.02,6]dec-8-ene-3,5-dione;
4-[6-[(2-fluoro-4-pyridyl)amino]-1,3-benzothiazol-2-yl]-4-azatricyclo[5.2.1.02,6]dec-8-ene-3,5-dione;
3-[[6-[(2-fluoro-4-pyridyl)amino]-1,3-benzothiazol-2-yl]carbamoyl]bicyclo[2.2.1]hept-5-ene-2-carboxylic acid;
3-[[6-[4-(1-piperidyl) anilino]-1,3-benzothiazol-2-yl]carbamoyl]bicyclo[2.2.1]hept-5-ene-2-carboxylic acid;
tert-butyl 4-[4-[2-[(1R,2S,6R,7S)-3,5-dioxo-4-azatricyclo[5.2.1.02,6]dec-8-en-4-yl]-1,3-benzothiazol-6-yl]phenyl]piperazine-1-carboxylate;
4-[6-(4-morpholinophenyl)-1,3-benzothiazol-2-yl]-4-azatricyclo[5.2.1.02,6]dec-8-ene-3,5-dione;
   or its pharmaceutically acceptable salts or optical isomers, racemates, diastereoisomers, enantiomers or tautomers.

In one embodiment, said compound of formula (I) is characterized in that:
R₁ is H; and/or
R₂ is H; and/or
R₃ is chosen from:
   linear or branched -(C₂-C₆)alkynyl;
   linear or branched -(C₁-C₆)alkoxy substituted by one or more halogen atoms;
   —NHR.
   —OR;
   -(C₆-C₁₀)aryl optionally substituted by one or more linear or branched -(C₁-C₆)alkyl optionally substituted by one or more halogen atoms, linear or branched -(C₁-C₆)alkoxy optionally substituted by one or more halogen atoms, —NRaRb;
R is chosen from:
   linear or branched -(C₁-C₆)alkyl substituted by one or more halogen atoms;
   -(C₆-C₁₀)aryl optionally substituted by one or more linear or branched (C₁-C₆)alkyl optionally substituted by one or more halogen atoms;
   an heteroaryl comprising 3 to 10 members having at least one heteroatom chosen from O, N, or S; said heteroaryl being optionally substituted by one or more linear or branched -(C₁-C₆)alkyl optionally substituted by one or more halogen atoms; and
Ra and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo;
Rc and Rd, identical or different, are chosen from:
   —H; and
   linear or branched -(C₁-C₆)alkyl;

In particular, said compound of formula (I) is characterized in that:
R₁ is H; and/or
R₂ is H; and/or
R₃ is chosen from:
   linear or branched -(C₁-C₃)alkoxy substituted by one or more fluorine atoms, preferably trifluromethoxy;
   3,3-dimethylbut-1-ynyl;
   —OR;
   —NHR;
   phenyl optionally substituted by one or more trifluormethyl, methoxy trifluromethoxy and/or piperidyl; and
R is chosen from:
   linear or branched (C₁-C₃)alkyl substituted by one or more halogen atoms, preferably one or more fluor atoms;
   phenyl optionally substituted by one or more methyl and/or trifluoromethyl;
   a heteroaryl comprising 6 members and having at least one nitrogen atom as heteroatom; said heteroaryl being optionally substituted by one or more trifluoromethyl, trifluoromethoxy and/or fluorine; for example a pyridyl substituted by one or more trifluoromethyl, trifluoromethoxy and/or fluorine.

More particularly, said compound of formula (I) is chosen from:
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[[6-(3,3-dimethylbut-1-ynyl)-1,3-benzothiazol-2-yl]carbamoyl]bicyclo [2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[[6-[[4-(trifluoromethoxy)phenyl]-1,3-benzothiazol-2-yl]carbamoyl]bicyclo [2.2.1]hept-5-ene-2-carboxylic acid;
3-[[6-[[4-(trifluoromethyl)phenyl]-1,3-benzothiazol-2-yl]carbamoyl]bicyclo [2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2-yl]carbamoyl]bicyclo[2.2.1]hept-5-ene-2-carboxylic acid;
3-[[6-[(5-fluoro-2-pyridyl)amino]-1,3-benzothiazol-2-yl]carbamoyl]bicyclo[2.2.1]hept-5-ene-2-carboxylic acid;
3-[[6-[(6-fluoro-2-pyridyl)amino]-1,3-benzothiazol-2-yl]carbamoyl]bicyclo[2.2.1]hept-5-ene-2-carboxylic acid;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[[6-[[5-(trifluoromethyl)-2-pyridyl]amino]-1,3-benzothiazol-2-yl]-4-azatricyclo[5.2.1.02,6]dec-8-ene-3,5-dione;
4-[6-[[6-(trifluoromethyl)-2-pyridyl]amino]-1,3-benzothiazol-2-yl]-4-azatricyclo[5.2.1.02,6]dec-8-ene-3,5-dione;
3-[[6-[4-(1-piperidyl) anilino]-1,3-benzothiazol-2-yl]carbamoyl]bicyclo[2.2.1]hept-5-ene-2-carboxylic acid;
   or its pharmaceutically acceptable salts or optical isomers, racemates, diastereoisomers, enantiomers or tautomers.

Compounds provided herein can be formulated into pharmaceutical compositions, optionally by admixture with one or more pharmaceutically acceptable excipients.

The present invention thus also relates to a pharmaceutical composition comprising a compound of formula (I) as defined in this section, and a pharmaceutically acceptable excipient.

In particular, said pharmaceutical composition further comprises at least one other anti-mycobacterial agent.

Anti-mycobacterial agents are well known in the art. Antimycobacterial, or antituberculosis, agents include rifampin, rifabutin, isoniazid, ethambutol, streptomycin, amikacin, kanamycin, moxifloxacin, pyrazinamide, bedaquiline, linezolid, sutezolid, nitroimidazole compounds such as pretomanid and delamanid. In the context of the invention nitroimidazole compounds such as pretomanid and delamanid are particularly contemplated. Antimycobacterial agents are most commonly prescribed today in multidrug combinations.

In one embodiment, the pharmaceutical composition comprises two, three, four, five, six or seven additional anti-tuberculosis agents. For example, in the treatment of multidrug-resistant tuberculosis, it is common that combinations of four or more drugs are administered to patients. For example, in the treatment of drug-sensitive tuberculosis, it is common that combinations of three or four drugs are administered to patients.

Pretonamid (CAS number: 187235-37-6) is a compound of formula:

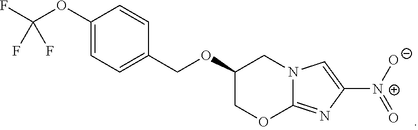 [SMILES: O=[N+]([O-])c1cn2c(n1)OC[C@@H](OCc1ccc(OC(F)(F)F)cc1)C2]

Known as an antibiotic medication used for the treatment of multi-drug-resistant tuberculosis, it is generally used together with bedaquiline and linezolid.

Delamanid (CAS number: 681492-22-8) is a compound of formula:

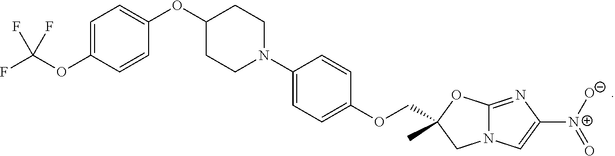 [SMILES: C[C@]1(COc2ccc(N3CCC(Oc4ccc(OC(F)(F)F)cc4)CC3)cc2)Cn2cc([N+](=O)[O-])nc2O1]

Sold under the brand name Deltyba®, it is generally used, along with other antituberculosis medications, to treat active multidrug-resistant tuberculosis.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20th ed.; Gennaro, A. R., Ed.; Lippincott Williams & Wilkins: Philadelphia, PA, 2000. Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

Examples of modes of administration include parenteral e.g. subcutaneous, intramuscular, intravenous, intradermal, as well as oral administration.

### Compounds for Use

As already mentioned, the present invention also relates to a compound of formula (I):

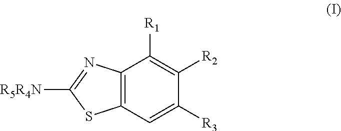

Wherein:
   R₁ is chosen from.
      —H;
      —OH;
      linear or branched -(C₁-C₆)alkyl; and
      linear or branched -(C₁-C₆)alcoxy(C₂-C₆)alkynyl; and/or
   R₂ is chosen from:
      —H;
      linear or branched -(C₁-C₆)alkyl;
      linear or branched -(C₁-C₆)alkoxy; and
      an halogen atom; and/or
   R₃ is chosen from:
      —H;
      an halogen atom;
      linear or branched -(C₁-C₆)alkyl optionally substituted by one or more halogen atoms;
      linear or branched -(C₁-C₆)alkoxy optionally substituted by one or more halogen atoms;
      linear or branched -(C₂-C₅)alkynyl;
      linear or branched -(C₁-C₆)alkoxy-(C₂-C₆)alkynyl;
      —OR;
      —NHR;
      -(C₂-C₅) cycloalkyl;
      linear or branched -(C₂-C₆)alkynyl-(C₆-C₁₀)aryl;
      linear or branched -(C₁-C₆)alkyl-(C₆-C₁₀)aryl;
      -(C₆-C₁₀)aryl optionally substituted by one or more —OH, halogen atoms, linear or branched (C₁-C₆)alkyl optionally substituted by one or more halogen atoms, linear or branched (C₁-C₆)alkoxy optionally substituted by one or more halogen atom, —NRaRb;
      an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;
      linear or branched -(C₂-C₆)alkynyl-(C₂-C₆) cycloalkyl,
      linear or branched -(C₆-C₁₀)aryl-(C₁-C₆)alkoxy-(C₂-C₆)alkynyl;
      —NO₂;
      linear or branched —SO₂(C₁-C₃)alkyl;
      —CN;
      —COO—(C₁-C₃)alkyl;
   R is chosen from:
      —H;
      linear or branched -(C₁-C₆)alkyl optionally substituted by one or more halogen atoms;
      -(C₆-C₁₀)aryl, said (C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
      an heteroaryl comprising 3 to 10 members having at least one heteroatom chosen from O, N, or S; said heteroaryl being optionally substituted by one or more halogen atoms, linear or branched -(C₁-C₆)alkyl optionally substituted by one or more halogen atoms; linear or branched -(C₁-C₆)alkoxy optionally substituted by one or more halogen atom; —CN;
   Ra and Rb, identical or different, are chosen from:
      —H;
      linear or branched -(C₁-C₆)alkyl, wherein one of the carbon atoms of the alkyl group can be substituted by an heteroatom;
or Rₐ and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo;
   Rc and Rd, identical or different, are chosen from:
      —H;
      linear or branched -(C₁-C₆)alkyl;
or R₂ and R₃ are linked together to form —O—(C₁-C₆)alkyl-O— group; and/or
R₄ is H and R₅ is:

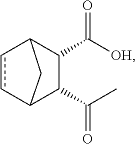

or R₄ and R₅, together with the nitrogen atom to which they are linked form:

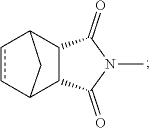

or its pharmaceutically acceptable salts or optical isomers, racemates, diastereoisomers, enantiomers or tautomers;
with the exclusion of the following compound: 3-[(6-methylsulfonyl-1,3-benzothiazol-2-yl)carbamoyl]bicyclo[2.2.1]hept-5-ene-2-carboxylic acid;
for use as a drug.

In one embodiment, the (3aS,4R,7S,7aS)-[2-(2-benzothiazolyl)]-4,7-methano-1H-isoindol-1,3-(2H)-dione and/or the Bicyclo[2.2.1]hept-5-ene-2,3-dicarboximide-N-[2-(6-methylbenzo)thiazolyl] are also excluded.

In particular, said compound of formula (I) is chosen from:
3-[(1,3-benzothiazol-2-yl)carbamoyl]bicyclo[2.2.1]hept-5-ene-2-carboxylic acid;
3-[(6-methyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
3-[(6-methyl-1,3-benzothiazol-2-yl)carbamoyl]norbornane-2-carboxylic acid;
(1S,2S,6R,7R)-4-(6-methyl-1,3-benzothiazol-2-yl) azatricyclo[5.2.1.02,6]decane-3,5-dione;
3-[(5-methoxy-1,3-benzothiazol-2-yl)carbamoyl]bicyclo[2.2.1]hept-5-ene-2-carboxylic acid;
3-[(6-methoxy-1,3-benzothiazol-2-yl)carbamoyl]bicyclo[2.2.1]hept-5-ene-2-carboxylic acid;
3-[(6-ethoxy-1,3-benzothiazol-2-yl)carbamoyl]bicyclo[2.2.1]hept-5-ene-2-carboxylic acid;
(1R,2S,6R,7S)-4-(6-ethoxy-1,3-benzothiazol-2-yl)-4-azatricyclo[5.2.1.02,6]dec-8-ene-3,5-dione;
3-[(5,6-dimethoxy-1,3-benzothiazol-2-yl)carbamoyl]bicyclo[2.2.1]hept-5-ene-2-carboxylic acid;
3-(6,7-dihydro-[1,4]dioxino[2,3-f][1,3]benzothiazol-2-ylcarbamoyl) bicyclo [2.2.1]hept-5-ene-2-carboxylic acid;
3-[(6-ethyl-1,3-benzothiazol-2-yl)carbamoyl]bicyclo[2.2.1]hept-5-ene-2-carboxylic acid;
3-[(6-tert-butyl-1,3-benzothiazol-2-yl)carbamoyl]bicyclo[2.2.1]hept-5-ene-2-carboxylic acid;
3-[(4,6-dimethyl-1,3-benzothiazol-2-yl)carbamoyl]bicyclo[2.2.1]hept-5-ene-2-carboxylic acid;
3-[(5,6-dimethyl-1,3-benzothiazol-2-yl)carbamoyl]bicyclo[2.2.1]hept-5-ene-2-carboxylic acid;
3-[(4,5-dimethyl-1,3-benzothiazol-2-yl)carbamoyl]bicyclo[2.2.1]hept-5-ene-2-carboxylic acid;
3-[(4-methyl-1,3-benzothiazol-2-yl)carbamoyl]bicyclo[2.2.1]hept-5-ene-2-carboxylic acid;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
3-[(6-benzyl-1,3-benzothiazol-2-yl)carbamoyl]bicyclo[2.2.1]hept-5-ene-2-carboxylic acid;
3-[[6-(4-fluorophenoxy)-1,3-benzothiazol-2-yl]carbamoyl]bicyclo[2.2.1]hept-5-ene-2-carboxylic acid;
3-[[6-[[4-(trifluoromethyl)phenoxy]-1,3-benzothiazol-2-yl]carbamoyl]bicyclo [2.2.1]hept-5-ene-2-carboxylic acid;
(1R,2S,6R,7S)-4-[6-[4-(trifluoromethyl)phenoxy]-1,3-benzothiazol-2-yl]-4-azatricyclo[5.2.1.02,6]dec-8-ene-3,5-dione;
3-[[6-(4-pyridyloxy)-1,3-benzothiazol-2-yl]carbamoyl]bicyclo[2.2.1]hept-5-ene-2-carboxylic acid;
3-[[6-[[3-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[3-(trifluoromethyl)-2-pyridyl]oxy]-1,3-benzothiazol-2-yl]-4-azatricyclo[5.2.1.02,6]dec-8-ene-3,5-dione;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[4-(trifluoromethyl)-2-pyridyl]oxy]-1,3-benzothiazol-2-yl]-4-azatricyclo[5.2.1.02,6]dec-8-ene-3,5-dione;
3-[[6-[(4-methyl-2-pyridyl)oxy]-1,3-benzothiazol-2-yl]carbamoyl]bicyclo [2.2.1]hept-5-ene-2-carboxylic acid;
2-[[2-[(1R,2S,6R,7S)-3,5-dioxo-4-azatricyclo[5.2.1.02,6]dec-8-en-4-yl]-1,3-benzothiazol-6-yl]oxy]pyridine-4-carbonitrile;
3-[[6-[[2-(trifluoromethyl)-4-pyridyl]oxy]-1,3-benzothiazol-2-yl]carbamoyl]bicyclo[2.2.1]hept-5-ene-2-carboxylic acid;
(1R,2S,6R,7S)-4-[6-[[6-(trifluoromethyl)-2-pyridyl]oxy]-1,3-benzothiazol-2-yl]-4-azatricyclo[5.2.1.02,6]dec-8-ene-3,5-dione;
3-[[6-[[5-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[5-(trifluoromethyl)-2-pyridyl]oxy]-1,3-benzothiazol-2-yl]-4-azatricyclo[5.2.1.02,6]dec-8-ene-3,5-dione;
3-[[6-[(5-bromo-2-pyridyl)oxy]-1,3-benzothiazol-2-yl]carbamoyl]bicyclo [2.2.1]hept-5-ene-2-carboxylic acid;
3-[(6-nitro-1,3-benzothiazol-2-yl)carbamoyl]bicyclo[2.2.1]hept-5-ene-2-carboxylic acid;
3-[(6-methylsulfonyl-1,3-benzothiazol-2-yl)carbamoyl]bicyclo[2.2.1]hept-5-ene-carboxylic acid;
3-[(6-cyano-1,3-benzothiazol-2-yl)carbamoyl]bicyclo[2.2.1]hept-5-ene-2-carboxylic acid;
3-[(6-methoxycarbonyl-1,3-benzothiazol-2-yl)carbamoyl]bicyclo[2.2.1]hept-5-ene-2-carboxylic acid;
3-[(6-fluoro-1,3-benzothiazol-2-yl)carbamoyl]bicyclo[2.2.1]hept-5-ene-2-carboxylic acid;
3-[[6-(trifluoromethyl)-1,3-benzothiazol-2-yl]carbamoyl]bicyclo[2.2.1]hept-5-ene-2-carboxylic acid;
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[(6-chloro-1,3-benzothiazol-2-yl)carbamoyl]bicyclo[2.2.1]hept-5-ene-2-carboxylic acid;
3-[(6-bromo-1,3-benzothiazol-2-yl)carbamoyl]bicyclo[2.2.1]hept-5-ene-2-carboxylic acid;
3-[(5-bromo-1,3-benzothiazol-2-yl)carbamoyl]bicyclo[2.2.1]hept-5-ene-2-carboxylic acid;
3-[(6-iodo-1,3-benzothiazol-2-yl)carbamoyl]bicyclo[2.2.1]hept-5-ene-2-carboxylic acid;
3-[(6-hydroxy-1,3-benzothiazol-2-yl)carbamoyl]bicyclo[2.2.1]hept-5-ene-2-carboxylic acid;
3-[(6-phenyl-1,3-benzothiazol-2-yl)carbamoyl]-7-oxabicyclo[2.2.1]hept-5-ene-2-carboxylic acid;
3-[(6-cyclohexyl-1,3-benzothiazol-2-yl)carbamoyl]bicyclo[2.2.1]hept-5-ene-2-carboxylic acid;
3-[[6-(p-tolyl)-1,3-benzothiazol-2-yl]carbamoyl]bicyclo[2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[[6-(4-chlorophenyl)-1,3-benzothiazol-2-yl]carbamoyl]bicyclo[2.2.1]hept-5-ene-2-carboxylic acid;
3-[[6-(3,4-dichlorophenyl)-1,3-benzothiazol-2-yl]carbamoyl]bicycle [2.2.1]hept-5-ene-2-carboxylic acid;
3-[[6-[[2-(trifluoromethyl)phenyl]-1,3-benzothiazol-2-yl]carbamoyl]bicyclo [2.2.1]hept-5-ene-2-carboxylic acid;
(1R,2S,6R,7S)-4-[6-[[2-(trifluoromethyl)phenyl]-1,3-benzothiazol-2-yl]-4-azatricyclo[5.2.1.02,6]dec-8-ene-3,5-dione;
3-[[6-[3-(trifluoromethyl)phenyl]-1,3-benzothiazol-2-yl]carbamoyl]bicyclo [2.2.1]hept-5-ene-2-carboxylic acid;
3-[[6-[[4-(trifluoromethyl)phenyl]-1,3-benzothiazol-2-yl]carbamoyl]bicyclo [2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[[6-[[4-(trifluoromethoxy)phenyl]-1,3-benzothiazol-2-yl]carbamoyl]bicyclo [2.2.1]hept-5-ene-2-carboxylic acid;
(1R,2S,6R,7S)-4-[6-(4-hydroxyphenyl)-1,3-benzothiazol-2-yl]-4-azatricyclo[5.2.1.02,6]dec-8-ene-3,5-dione;
3-[[6-(4-pyridyl)-1,3-benzothiazol-2-yl]carbamoyl]bicyclo[2.2.1]hept-5-ene-2-carboxylic acid;
(1R,2S,6R,7S)-4-[6-(6-fluoro-3-pyridyl)-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo [2.2.1]hept-5-ene-2-carboxylic acid;
(1R,2S,6R,7S)-4-[6-[[6-(trifluoromethyl)-3-pyridyl]-1,3-benzothiazol-2-yl]-4-azatricyclo[5.2.1.02,6]dec-8-ene-3,5-dione;
3-[(6-pyrimidin-5-yl-1,3-benzothiazol-2-yl)carbamoyl]bicyclo[2.2.1]hept-5-ene-2-carboxylic acid;
3-[[6-(6-quinolyl)-1,3-benzothiazol-2-yl]carbamoyl]bicyclo[2.2.1]hept-5-ene-2-carboxylic acid;
4-[6-(3-thienyl)-1,3-benzothiazol-2-yl]-4-azatricyclo[5.2.1.02,6]dec-8-ene-3,5-dione;
3-[[6-(3-furyl)-1,3-benzothiazol-2-yl]carbamoyl]bicyclo[2.2.1]hept-5-ene-2-carboxylic acid;
(1R,2S,6R,7S)-4-[6-(3-furyl)-1,3-benzothiazol-2-yl]-4-azatricyclo[5.2.1.02,6]dec-8-ene-3,5-dione;
4-[6-(4-prop-2-ynoxyphenyl)-1,3-benzothiazol-2-yl]-4-azatricyclo[5.2.1.02,69dec-8-ene-3,5-dione;
(1R,2S,6R,7S)-4-(6-ethynyl-1,3-benzothiazol-2-yl)-4-azatricyclo[5.2.1.02,6]dec-8-ene-3,5-dione;
(1R,2S,6R,7S)-4-(6-prop-2-ynoxy-1,3-benzothiazol-2-yl)-4-azatricyclo[5.2.1.02,6]dec-8-ene-3,5-dione;
(1R,2S,3R,4S)-3-[(4-hydroxy-1,3-benzothiazol-2-yl)carbamoyl]bicyclo[2.2.1]hept-5-ene-2-carboxylic acid;
3-[(4-prop-2-ynoxy-1,3-benzothiazol-2-yl)carbamoyl]bicyclo[2.2.1]hept-5-ene-2-carboxylic acid;
(1R,2S,6R,7S)-4-(4-prop-2-ynoxy-1,3-benzothiazol-2-yl)-4-azatricyclo[5.2.1.02,6]dec-8-ene-3,5-dione;
3-[[6-(2-phenylethynyl)-1,3-benzothiazol-2-yl]carbamoyl]bicyclo[2.2.1]hept-5-ene-2-carboxylic acid;
3-[[6-(3,3-dimethylbut-1-ynyl)-1,3-benzothiazol-2-yl]carbamoyl]bicyclo [2.2.1]hept-5-ene-2-carboxylic acid;
3-[[6-(2-cyclopropylethynyl)-1,3-benzothiazol-2-yl]carbamoyl]bicyclo[2.2.19hept-5-ene-2-carboxylic acid;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
(1R,2S,6R,7S)-4-[[6-[(4-methyl-2-pyridyl)oxy]-1,3-benzothiazol-2-yl]-4-azatricyclo[5.2.1.02,6]dec-8-ene-3,5-dione;
(1R,2S,6R,7S)-4-[6-[(2-chloro-4-pyridyl)oxy]-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo [2.2.1]hept-5-ene-2-carboxylic acid;
(1R,2S,6R,7S)-4-(6-anilino-1,3-benzothiazol-2-yl)-4-azatricyclo[5.2.1.02,6]dec-8-ene-3,5-dione;
4-[6-(2-pyridylamino)-1,3-benzothiazol-2-yl]-4-azatricyclo[5.2.1.02,6]dec-8-ene-3,5-dione; and
3-[[6-[(4-methyl-2-pyridyl)amino]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2 yl]carbamoyl]bicyclo[2.2.1]hept-5-ene-2-carboxylic acid;
3-[[6-[(5-fluoro-2-pyridyl)amino]-1,3-benzothiazol-2 yl]carbamoyl]bicyclo[2.2.1]hept-5-ene-2-carboxylic acid;
4-[6-[(5-fluoro-2-pyridyl)amino]-1,3-benzothiazol-2-yl]-4-azatricyclo[5.2.1.02,6]dec-8-ene-3,5-dione;
3-[[6-[(6-fluoro-2-pyridyl)amino]-1,3-benzothiazol-2-yl]carbamoyl]bicyclo[2.2.1]hept-5-ene-2-carboxylic acid;
4-[6-[(6-fluoro-2-pyridyl)amino]-1,3-benzothiazol-2-yl]-4-azatricyclo[5.2.1.02,6]dec-8-ene-3,5-dione;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethyl)-3-pyridyl]amino]-1,3-benzothiazol-2-yl]carbamoyl]bicyclo[2.2.1]hept-5-ene-2-carboxylic acid;
4-[6-[(2-methoxy-4-pyridyl)amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[6-[[5-(trifluoromethoxy)-2-pyridyl]amino]-1,3-benzothiazol-2-yl]-4-azatricyclo[5.2.1.02,6]dec-8-ene-3,5-dione;
4-[[6-[[5-(trifluoromethyl)-2-pyridyl]amino]-1,3-benzothiazol-2-yl]-4-azatricyclo[5.2.1.02,6]dec-8-ene-3,5-dione;
3-[[6-[[5-(trifluoromethyl)-2-pyridyl]amino]-1,3-benzothiazol-2-yl]carbamoyl]bicyclo[2.2.1]hept-5-ene-2-carboxylic acid; 4-[6-[[6-(trifluoromethyl)-2-pyridyl]amino]-1,3-benzothiazol-2-yl]-4-azatricyclo[5.2.1.02,6]dec-8-ene-3,5-dione;
4-[6-[(2-fluoro-4-pyridyl)amino]-1,3-benzothiazol-2-yl]-4-azatricyclo[5.2.1.02,6]dec-8-ene-3,5-dione;
3-[[6-[(2-fluoro-4-pyridyl)amino]-1,3-benzothiazol-2-yl]carbamoyl]bicyclo[2.2.1]hept-5-ene-2-carboxylic acid;
3-[[6-[4-(1-piperidyl) anilino]-1,3-benzothiazol-2-yl]carbamoyl]bicyclo[2.2.1]hept-5-ene-2-carboxylic acid;
tert-butyl 4-[4-[2-[(1R,2S,6R,7S)-3,5-dioxo-4-azatricyclo[5.2.1.02,6]dec-8-en-4-yl]-1,3-benzothiazol-6-yl]phenyl]piperazine-1-carboxylate;
4-[6-(4-morpholinophenyl)-1,3-benzothiazol-2-yl]-4-azatricyclo[5.2.1.02,6]dec-8-ene-3,5-dione;
   or its pharmaceutically acceptable salts or optical isomers, racemates, diastereoisomers, enantiomers or tautomers.

More particularly, said compound of formula (I) is characterized in that:
R₁ is H; and/or
R₂ is H; and/or
R₃ is chosen from:
   linear or branched -(C₁-C₆)alkyl optionally substituted by one or more halogen atoms;
   linear or branched -(C₁-C₆)alkoxy optionally substituted by one or more halogen atoms;
   linear or branched (C₂-C₆)alkynyl;
   —OR;
   —NHR;
   -(C₆-C₁₀)aryl optionally substituted by one or more linear or branched (C₁-C₆)alkyl optionally substituted by one or more halogen atoms, linear or branched (C₁-C₆)alkoxy optionally substituted by one or more halogen atoms, NRaRb;
R is chosen from:
   linear or branched -(C₁-C₆)alkyl optionally substituted by one or more halogen atoms;
   -(C₆-C₁₀)aryl optionally substituted by one or more linear or branched -(C₁-C₆)alkyl optionally substituted by one or more halogen atoms;
   an heteroaryl comprising 3 to 10 members having at least one heteroatom chosen from O, N, or S; said heteroaryl being optionally substituted by one or more linear or branched -(C₁-C₆)alkyl optionally substituted by one or more halogen atoms; and
Ra and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo;
Rc and Rd, identical or different, are chosen from:
   —H; and
   linear or branched -(C₁-C₆)alkyl.

Still particularly, said compound of formula (I) is characterized in that:
R₁ is H; and/or
R₂ is H; and/or
R₃ is chosen from:
   linear or branched -(C₁-C₃)alkyl, preferably methyl;
   linear or branched -(C₁-C₃)alkoxy, preferably ethoxy;
   linear or branched -(C₁-C₃)alkoxy substituted by fluorine atoms, preferably trifluromethoxy;
   3,3-dimethylbut-1-ynyl;
   —OR;
   —NHR;
   phenyl optionally substituted by one or more trifluoromethyl, methoxy, trifluoromethoxy and/or piperidyl; and
R is chosen from:
   linear or branched -(C₁-C₃)alkyl optionally substituted by one or more halogen atoms, preferably one or more fluor atoms;
   phenyl optionally substituted by one or more methyl and/or, trifluormethyl;
   a heteroaryl comprising 6 members and having at least one nitrogen atom as heteroatom; said heteroaryl being optionally substituted by one or more trifluoromethyl, trifluoromethoxy and/or fluorine; for example a pyridyl substituted by one or more trifluoromethyl, trifluoromethoxy and/or fluorine.

In one embodiment, said compound of formula (I) is chosen from:
3-[(6-ethoxy-1,3-benzothiazol-2-yl)carbamoyl]bicyclo[2.2.1]hept-5-ene-2-carboxylic acid;
(1R,2S,6R,7S)-4-(6-ethoxy-1,3-benzothiazol-2-yl)-4-azatricyclo[5.2.1.02,6]dec-8-ene-3,5-dione;
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[[6-(3,3-dimethylbut-1-ynyl)-1,3-benzothiazol-2-yl]carbamoyl]bicyclo [2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[[6-[[4-(trifluoromethoxy)phenyl]-1,3-benzothiazol-2-yl]carbamoyl]bicyclo [2.2.1]hept-5-ene-2-carboxylic acid;
3-[[6-[[4-(trifluoromethyl)phenyl]-1,3-benzothiazol-2-yl]carbamoyl]bicyclo [2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[(6-methyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2-yl]carbamoyl]bicyclo[2.2.1]hept-5-ene-2-carboxylic acid;
3-[[6-[(5-fluoro-2-pyridyl)amino]-1,3-benzothiazol-2-yl]carbamoyl]bicyclo[2.2.1]hept-5-ene-2-carboxylic acid;
3-[[6-[(6-fluoro-2-pyridyl)amino]-1,3-benzothiazol-2-yl]carbamoyl]bicyclo[2.2.1]hept-5-ene-2-carboxylic acid;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[[6-[[5-(trifluoromethyl)-2-pyridyl]amino]-1,3-benzothiazol-2-yl]-4-azatricyclo[5.2.1.02,6]dec-8-ene-3,5-dione;
4-[6-[[6-(trifluoromethyl)-2-pyridyl]amino]-1,3-benzothiazol-2-yl]-4-azatricyclo[5.2.1.02,6]dec-8-ene-3,5-dione;
3-[[6-[4-(1-piperidyl) anilino]-1,3-benzothiazol-2-yl]carbamoyl]bicyclo[2.2.1]hept-5-ene-2-carboxylic acid;
   or its pharmaceutically acceptable salts or optical isomers, racemates, diastereoisomers, enantiomers or tautomers.

As already mentioned, said compound can be used to prevent and/or treat a mycobacterial infection.

Mycobacterial infections are well known in the art. A mycobacterial infection is one caused by infection with a mycobacterium.

The mycobacterium may be a member of one of the following groups of mycobacterium: Mycobacterium tuberculosis complex (MTC), Mycobacterium avium comp/ex (MAC), Mycobacterium gordonae clade, Mycobacterium kansasii clade, Mycobacterium chelonae clade, Mycobacterium fortuitum clade, Mycobacterium parafortuitum clade or Mycobacterium vaccae clade. The mycobacterium may also be Mycobacterium ulcerans or Mycobacterium leprae.

In particular, the mycobacterium is a member of the Mycobacterium tuberculosis complex (MTC). Members of Mycobacterium tuberculosis complex (MTC) include Mycobacterium tuberculosis, Mycobacterium africanum, Mycobacterium bovis, Mycobacterium bovis BCG, Mycobacterium canetti, Mycobacterium caprae, Mycobacterium microti and Mycobacterium pinnipedii.

These mycobacteria are causative agents of human and animal tuberculosis. Mycobacterium tuberculosis is the major cause of human tuberculosis.

Still particularly, the infection is a Mycobacterium tuberculosis infection. In other words, the mycobacterial infection is caused by infection with Mycobacterium tuberculosis.

In one embodiment, the Mycobacterium tuberculosis is multidrug-resistant. In another embodiment the Mycobacterium tuberculosis is resistant to nitroimidazole compounds such as pretomanid and/or delamanid.

These two drugs have been defined in the previous section.

In another aspect, the invention relates to a compound of Formula (I) as described herein, or a pharmaceutically acceptable salt thereof, for use in the treatment of a disease caused by infection with a mycobacterium.

In particular, the mycobacterium is selected from those hereinbefore described.

For example, the mycobacterial infection may be caused by infection with a mycobacterium selected from the following: Mycobacterium tuberculosis, Mycobacterium africanum, Mycobacterium bovis, Mycobacterium bovis BCG, Mycobacterium canetti, Mycobacterium caprae, Mycobacterium microti, Mycobacterium pinnipedii, Mycobacterium avium, Mycobacterium avium paratuberclosis, Mycobacterium avium silaticum, Mycobacterium avium hominissuis, Mycobacterium columbiense, Mycobacterium indicus pranii, Mycobacterium asiaticum, Mycobacterium gordonae, Mycobacterium gastri, Mycobacterium kansasii, Mycobacterium abscessus, Mycobacterium bolletii, Mycobacterium chlonae, include Mycobacterium boenickei, Mycobacterium brisbanense, Mycobacterium cosmeticum, Mycobacterium fortuitum, Mycobacterium fortuitum subspecies acetamidolyticum, Mycobacterium houstonense, Mycobacterium mageritense, Mycobacterium neworleansense, Mycobacterium peregrinum, Mycobacterium porcinum, Mycobacterium senegalense, Mycobacterium septicum, Mycobacterium austroafricanum, Mycobacterium diernhoferi, Mycobacterium frederiksbergense, Mycobacterium hodleri, Mycobacterium neoaurum, Mycobacterium parafortuitum, Mycobacterium ulcerans and Mycobacterium leprae.

Diseases caused by infection with a mycobacterium include, but are not limited to, tuberculosis (e.g. from Mycobacterium tuberculosis), leprosy (e.g. from Mycobacterium leprae), Johne's disease (e.g. from Mycobacterium avium subspecies paratuberculosis), Buruli or Bairnsdale ulcer (e.g. from Mycobacterium ulcerans), Crohn's disease (e.g. from Mycobacterium avium subspecies paratuberculosis), pulmonary disease or pulmonary infection, pneumonia, bursa, synovial, tendon sheaths, localized abscess, lymphadenitis, skin and soft tissue infections, Lady Windermere syndrome (e.g. from Mycobacterium avium complex (MAC)), MAC lung disease, disseminated Mycobacterium avium complex (DMAC), disseminated Mycobacterium avium intracellulare complex (DMAIC), hot-tub lung (e.g. from Mycobacterium avium complex), MAC mastitis, MAC pyomyositis, or granuloma disease.

In particular, in the context of the invention, the disease is tuberculosis.

In one embodiment, the invention thus also relates to a method of treatment of a mycobacterial infection in a subject in need thereof, said treatment comprising administering to said subject a therapeutically effective amount of a compound of Formula (I) as described in this section, or pharmaceutically acceptable salt thereof.

As described herein, a mycobacterial infection is one caused by infection with a mycobacterium. The mycobacterium is as hereinbefore described.

In one embodiment, the invention relates to a method of treatment of a Mycobacterium tuberculosis infection.

In another embodiment, the invention relates to a method of treatment of a disease caused by infection with a mycobacterium in a subject in need thereof, said treatment comprising administering to said subject a therapeutically effective amount of a compound of Formula (I), or a pharmaceutically acceptable salt thereof.

In particular in the context of the invention, the disease is tuberculosis.

In one embodiment, said compound is used in combination with at least one other anti-mycobacterial agent.

Anti-mycobacterial agents are well known in the art. Antimycobacterial, or antituberculosis, agents include rifampin, rifabutin, isoniazid, ethambutol, streptomycin, amikacin, kanamycin, moxifloxacin, pyrazinamide, bedaquiline, linezolid, sutezolid, nitroimidazole compounds such as pretomanid and delamanid. In the context of the invention nitroimidazole compounds such as pretomanid and delamanid are particularly contemplated.

Antimycobacterial agents are most commonly prescribed today in multidrug combinations.

In one embodiment, the combination comprises two, three, four, five, six or seven additional anti-tuberculosis agents. For example, in the treatment of multidrug-resistant tuberculosis, it is common that combinations of four or more drugs are administered to patients. For example, in the treatment of drug-sensitive tuberculosis, it is common that combinations of three or four drugs are administered to patients.

In particular the combination comprises pretonamid and/or delamanid.

Pretonamid (CAS number: 187235-37-6) is a compound of formula:

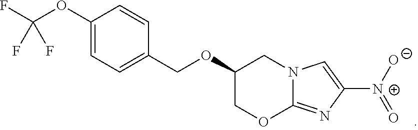 [SMILES: O=[N+]([O-])c1cn2c(n1)OC[C@@H](OCc1ccc(OC(F)(F)F)cc1)C2]

Known as an antibiotic medication used for the treatment of multi-drug-resistant tuberculosis, it is generally used together with bedaquiline and linezolid.

Delamanid (CAS number: 681492-22-8) is a compound of formula:

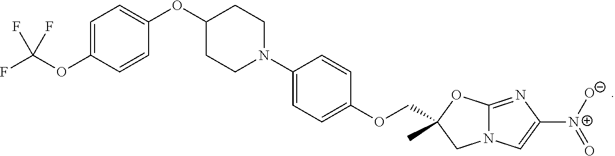 [SMILES: C[C@]1(COc2ccc(N3CCC(Oc4ccc(OC(F)(F)F)cc4)CC3)cc2)Cn2cc([N+](=O)[O-])nc2O1]

Sold under the brand name Deltyba®, it is generally used, along with other antituberculosis medications, to treat active multidrug-resistant tuberculosis.

In another aspect, the invention also relates to a combination of (a) a compound of formula (|) as defined in this section and (b) at least one other anti-mycobacterial agent.

The anti-mycobacterial agent is as defined herein.

The terms “treat”, “treating”, “treated” or “treatment”, as used in the context of the invention, refer to therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The terms “prevent”, “prevention”, “preventing” or “prevented”, as used in the context of the present invention, refer to the prevention of the onset, recurrence or spread of a disease or disorder or infection, or of one or more symptoms thereof. In certain embodiments, the terms refer to the treatment with or administration of a compound provided herein prior to the onset of symptoms, particularly to patients at risk of disease or disorder or infection provided herein. The terms encompass the inhibition or reduction of a symptom of the particular disease or disorder or infection. Subjects with familial history of a disease or disorder or infection in particular are candidates for preventive regimens in certain embodiments. In addition, subjects who have a history of recurring symptoms are also potential candidates for the prevention. In this regard, the term “prevention” may be interchangeably used with the term “prophylactic treatment”.

In particular, the subject in need of a treatment against mycobacterial infection or a disease caused by infection with a mycobacterium is a subject afflicted with such disease or infection.

In the context of the present invention, the identification of the subjects who are in need of treatment of herein-described diseases and conditions is conducted as above mentioned and is well within the ability and knowledge of the man skilled in the art. A clinician skilled in the art can readily identify, by the above-mentioned technics, those subjects who are in need of such treatment. In particular, the prevention and/or treatment as described herein relates to subjects who have been already administered or simultaneously received at least one anti-mycobacterial agent, more particularly at least one antituberculosis agent, and preferably pretomanid and/or delamanid. In said context, a compound of formula (I) as described herein can act as a booster of the anti-mycobacterial agent.

By “booster” is meant that the compound of formula (I) according to the invention will increase the potency of the anti-mycobacterial agent.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the particular compound administered; the mode of administration; the bioavailability characteristic of the preparation administered; the dose regimen selected; the use of concomitant medication; and other relevant circumstances.

As used herein, an «effective amount” refers to an amount which is effective in reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment and chronic use.

The term “patient” or “subject” refers to a warm-blooded animal such as a mammal, in particular a human, male or female, unless otherwise specified, which is afflicted with, or has the potential to be afflicted with one or more diseases and conditions described herein.

In particular, the compounds of the combination according to the invention are administered separately, sequentially or simultaneously. More particularly, when combinations are contemplated, the compound of formula (I) as described herein can be administered simultaneously with at least one anti-mycobacterial agent, or at least one anti-mycobacterial agent can be administered first.

The amount of the compound according to the invention, which is required to achieve the desired biological effect, will vary depending upon a number of factors, including the dosage of the drug to be administered, the chemical characteristics (e.g. hydrophobicity) of the compounds employed, the potency of the compounds, the type of resistance, the state of resistance in the patient, and the route of administration.

Compounds provided herein can be formulated into pharmaceutical compositions, optionally by admixture with one or more pharmaceutically acceptable excipients.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20th ed.; Gennaro, A. R., Ed.; Lippincott Williams & Wilkins: Philadelphia, PA, 2000. Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

Examples of modes of administration include parenteral e.g. subcutaneous, intramuscular, intravenous, intradermal, as well as oral administration.

### Process of Preparation

The present invention is also concerned with the process of preparation of the compounds of formula (I) as described herein.

The compounds and process of the present invention may be prepared in a number of ways well known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art.

It will be appreciated that the compounds of the present invention may contain one or more asymmetrically substituted carbon atoms, and may be isolated in optically active or racemic forms. Thus, all chiral, diastereomeric, racemic forms, isomeric forms of a structure are intended, unless the specific stereochemistry or isomeric form is specifically indicated. It is well known in the art how to prepare and isolate such optically active forms.

For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxyl, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, 4th ed. (2007), John Wiley & Sons Inc., 1999; J. F. W. McOmie in Protective Groups in Organic Chemistry, Plenum Press, 1973.

The compound thus prepared may be recovered from the reaction mixture by conventional means. For example, the compounds may be recovered by distilling off the solvent from the reaction mixture or, if necessary, after distilling off the solvent from the reaction mixture, pouring the residue into water followed by extraction with a water-immiscible organic solvent and distilling off the solvent from the extract. Additionally, the product can, if desired, be further purified by various well-known techniques, such as recrystallization, reprecipitation or the various chromatography techniques, notably column chromatography or preparative thin layer chromatography.

The reactions can be carried out by the skilled person by applying or adapting the methods illustrated in the examples hereinafter.

In particular, compounds of formula (I) can be prepared according to protocols mentioned in the experimental part below.

Further, the process of the invention may also comprise the additional step of isolating the compound of formula (I). This can be done by the skilled person by any of the known conventional means, such as the recovery methods described above.

Generally, the starting products are commercially available mainly from Aldrich or Acros or other typical chemicals supplier or may be obtained by applying or adapting any known methods or those described in the examples.

In the context of the present invention, it should be understood that “a compound for use for the prevention and/or treatment of” is equivalent to “the use of a compound for the prevention and/or treatment of” and to “the use of a compound for the manufacture of a medicament for the prevention and/or treatment of”.

The invention will be further illustrated by the following figure and examples.

### EXAMPLES

### Part A: Synthesis of the Compounds According to the Invention

The compounds of the invention can be prepared by a process (a) to (c);
(a) As depicted in Scheme 1, by reaction of a suitable anhydride with a substituted 2-aminobenzothiazole in the presence or absence of a base.
(b) As depicted in Scheme 2, by cyclisation using a dehydrating agent such as T3P.
(c) As depicted in Scheme 3, by alkylation using a halogenated alkyl derivative in the presence of a base.

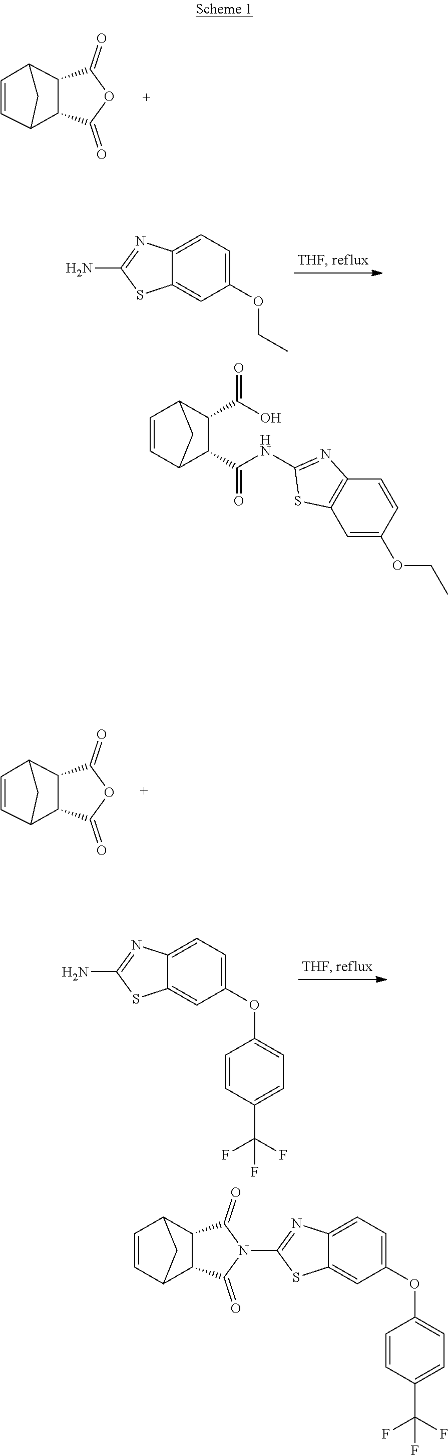

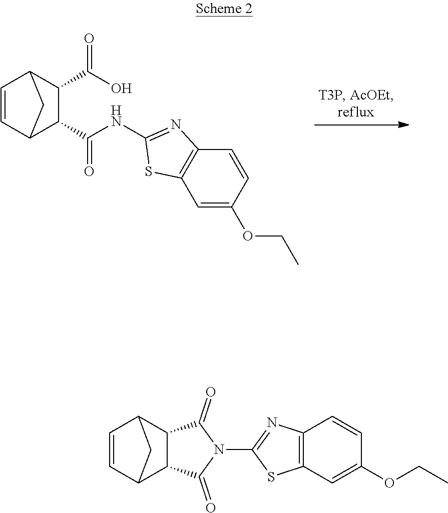 [SMILES: CCOc1ccc2nc(NC(=O)[C@@H]3C4C=CC(C4)[C@@H]3C(=O)O)sc2c1 ; CCOc1ccc2nc(N3C(=O)[C@@H]4C5C=CC(C5)[C@@H]4C3=O)sc2c1]

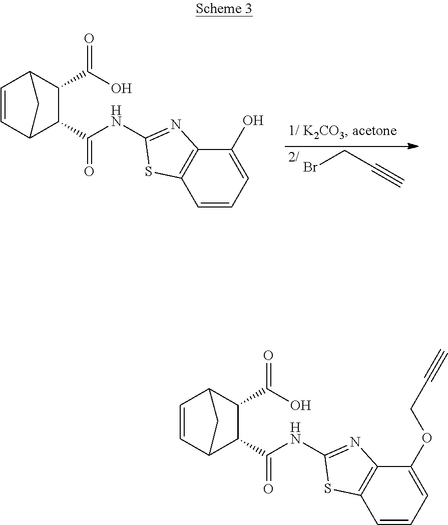

The synthesis of 2-aminobenzothiazole derivatives can be carried out by a process (d) to (h):
(d) As depicted in Scheme 3, by a Hugerschoff type reaction where an aniline is reacted with ammonium thiocyanate in the presence of bromine and acetic acid.

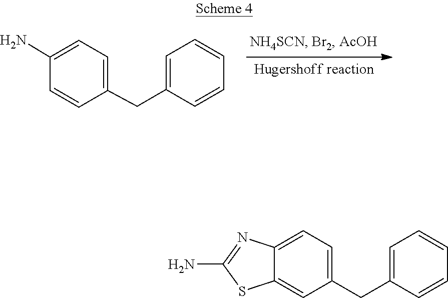 [SMILES: Nc1ccc(Cc2ccccc2)cc1 ; Nc1nc2ccc(Cc3ccccc3)cc2s1]

(e) As depicted in Scheme 5, by reaction of a hydroxy substituted 2-aminobenzothiazol with a halogenated pyridine in the presence of a base.

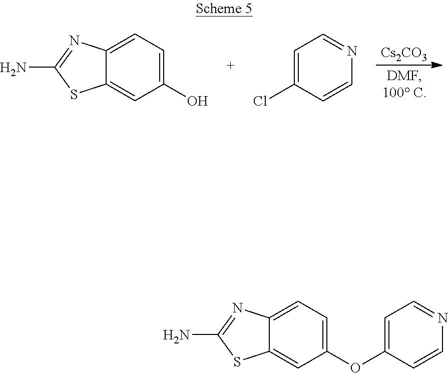

(f) As depicted in Scheme 6, by reaction of a 2-aminobenzothiazol substituted by a halogen atom with a terminal alkyne in the presence of palladium, copper iodine and a base, in a sonogashira type reaction.

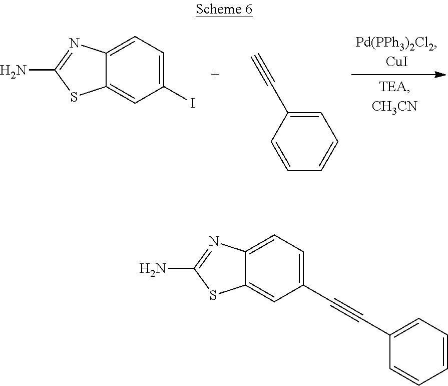

(g) As depicted in Scheme 7, by reaction of a 2-aminobenzothiazol substituted by a halogen atom with a boronic acid in the presence of palladium and a base, in a suzuki type reaction.

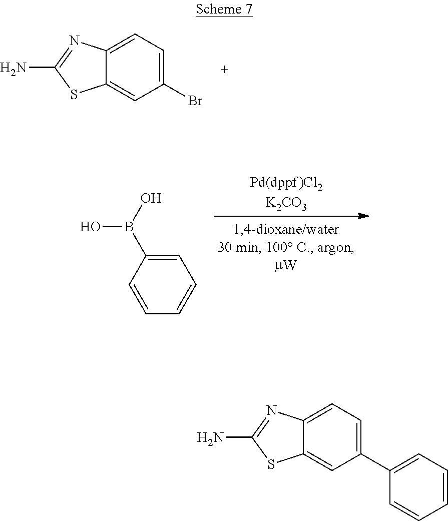 [SMILES: Nc1nc2ccc(Br)cc2s1 ; OB(O)c1ccccc1 ; Nc1nc2ccc(-c3ccccc3)cc2s1]

(h) As depicted in Scheme 8, by reaction of a Boc-protected 2-aminobenzothiazol substituted by a halogen atom with an aminopyridine in the presence of palladium, a phosphine and a base, in a Buchwald type reaction.

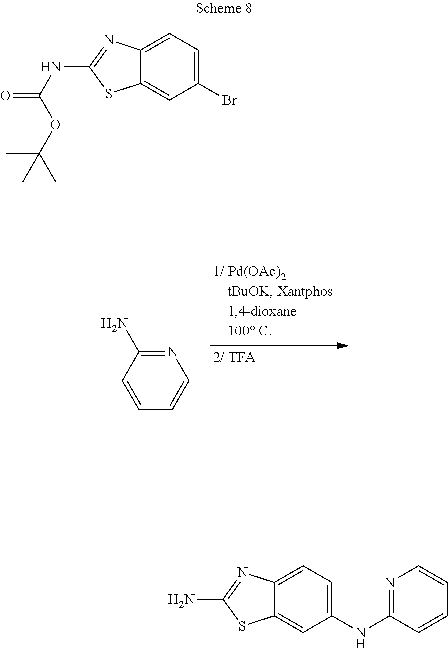 [SMILES: CC(C)(C)OC(=O)Nc1nc2ccc(Br)cc2s1 ; Nc1ccccn1 ; Nc1nc2ccc(Nc3ccccn3)cc2s1]

Method X: HPLC-MS analysis was performed on LC-MS Waters Alliance Micromass ZQ 2000 system equipped with a Waters 2747 sample manager, a Waters 2695 separations module, a Waters 2996 photodiode array detector (210-400 nm at a 1.2 nm resolution) and a Waters Micromass ZQ2000 detector (scan 100-800). XBridge C18 column (3.5 μm particle size, dimensions 50 mm×4.6 mm) was used for HPLC analysis. The injection volume was 20 μL. For a 5 min analysis, the elution was done at pH 3.8 from 100% H₂O/0.1% ammonium formate to 2% H₂O/98% CH₃CN/0.1% ammonium formate (or HCOOH) over 3.5 min. A flow rate at 2 mL/min was used. For a 30 min analysis, the elution was done at pH 3.8 from 100% H₂O/0.1% ammonium formate (or HCOOH) to 2% H₂O/98% CH₃CN/0.1% ammonium formate over 7 min. A flow rate at 1 mL/min was used. Purity (%) was determined by reversed phase HPLC (30 min analysis), using UV detection (215 nm). HRMS analyses were performed on a LCT Premier XE Micromass, using a C18 X-Bridge 3.5 μm particle size column, dimensions 50 mm*4.6 mm. A gradient starting from 98% H₂O 5 mM ammonium formate pH=3.8 and reaching 100% CH₃CN 5 mM ammonium formate pH=3.8 within 3 min at a flow rate of 1 mL/min was used.

The invention will now be illustrated by the following non-limiting examples.

### Example 1:3-[(6-methyl-1,3-benzothiazol-2-yl)carbamoyl]bicyclo[2.2.1]hept-5-ene-2-carboxylic acid

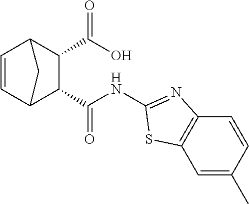 [SMILES: Cc1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (1 g, 6.1 mmol) in THF (4 mL) was added 6-methyl-1,3-benzothiazol-2-amine (1 g, 6.1 mmol). The mixture was heated under reflux and after 16 h, a precipitate was observed. The reaction was cooled to room temperature and the precipitate was removed by filtration and washed with THF to give the title compound as a white powder (1.5 g, 77%).

¹H NMR (300 MHZ, DMSO-d6) δ=12.15 (s, 1H), 11.8 (s, 1H), 7.69 (br s, 1H), 7.57 (d, J=8.0 Hz, 1H), 7.20 (dd, J=8.5, 1.6 Hz, 1H), 6.14 (br s, 2H), 3.45 (dd, J=10.4, 3.3 Hz, 1H), 3.35 (dd, J=9.9 Hz, 3.2 Hz, 1H), 3.11 (s, 1H), 3.06 (s 1H), 2.38 (s, 3H), 1.32(dd, J=17.2, 8.2 Hz, 2H).

LC-MS: method X, RT=3.92 min (10 min run), MS (ESI) m/z: 329 (M+H)⁺; 327 (M−H RT=2.47 min (5 min run), MS (ESI) m/z: 329 (M+H)⁺; 327 (M−H)⁻

HRMS (ES+) calculated for C17H17N2O3S [M+H]⁺: 329.0960 found 329.0963

Purification of the two enantiomers of example 1 was achieved by SFC (Supercritical Fluid Chromatography). The sample was solubilised at a concentration of 8 mg/ml in a 50/50 DCM/MeOH mixture and filtered prior to injection. 100 μL were injected on an IA column with a flow rate of 3 mL/min and with 10% of methanol as co-solvent. Fractions were collected in acetonitrile and then lyophilised to afford both pure enantiomers example 1a and 1b.

### Example 2: (1R,2S,6R,7S)-4-(6-methyl-1,3-benzothiazol-2-yl)-4-azatricyclo[5.2.1.02,6]dec-8-ene-3,5-dione

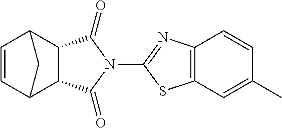 [SMILES: Cc1ccc2nc(N3C(=O)[C@@H]4C5C=CC(C5)[C@@H]4C3=O)sc2c1]

To a solution of 3-[(6-methyl-1,3-benzothiazol-2-yl)carbamoyl]bicyclo[2.2.19hept-5-ene-2-carboxylic acid (compound of example 1, 3.9 g, 12 mmol) in EtOAc (20 mL) was added T3P (50% in AcOEt, 28.22 mL, 95 mmol). The solution was stirred for 4 h under reflux. The organic layer was washed twice with water, dried, filtrated and evaporated under reduced pressure to give the title compound as a white powder (3.1 g, 84% yield).

¹H NMR (300 MHZ, DMSO-d6) δ=7.89 (s, 1H), 7.88 (d, J=8.0 Hz, 1H), 7.36 (ddd, J=8.4, 1.7, 0.6 Hz, 1H), 6.22(t, J=1.8 Hz, 2H), 3.62(dd, J=3.0, 1.6 Hz, 2H), 3.43-3.36 (m, 2H), 2.45 (s, 3H), 1.66-1.58 (m, 2H).

LC-MS: method X, RT=2.82 min, MS (ESI) m/z: 311 (M+H)⁺

HRMS (ES+) calculated for C17H15N2O2S [M+H]⁺: 311.0854 found 311.0850

### Example 3:3-[(6-methyl-1,3-benzothiazol-2-yl)carbamoyl]norbornane-2-carboxylic acid

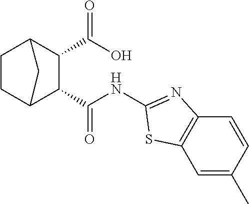 [SMILES: Cc1ccc2nc(NC(=O)[C@@H]3C4CCC(C4)[C@@H]3C(=O)O)sc2c1]

To a solution of 3-[(6-methyl-1,3-benzothiazol-2-yl)carbamoyl]bicyclo[2.2.1]hept-5-ene-2-carboxylic acid (100 mg, 0.305 mmol) in MeOH (1 mL) at 0° C. was added a solution of hydrazine (923 μL, 12.2 mmol) and hydrogene peroxyde (4933 μL, 0.152 mmol). The mixture was stirred at 0° C. for 20 minutes. Then, the reaction was acidified with ammonium formiate to pH 4. The precipitate was filtered and washed with DCM. The filtrate was evaporated under reduce pressure to give the title compound as a white powder (5.9 mg, 6%).

¹H NMR (300 MHZ, DMSO-d6) δ=12.17 (s, 1H), 11.9 (s, 1H), 7.73 (s, 1H), 7.59 (d, J=8.2 Hz, 1H), 7.22(dd, J=8.2, 1.1 Hz, 1H), 3.20 (dd, J=11.7, 4.3 Hz, 1H), 2.98 (dd, J=11.7, 2.9 Hz, 1H), 2.58 (s, 1H,), 2.40 (s, 4H), 1.88-1.80 (m, 1H), 1.61 (m, 1H), 1.47 (d, J=9.7 Hz, 1H), 1.34 (m, 3H).

LC-MS: method X, RT=2.56 min, MS (ESI) m/z: 331 (M+H)⁺; 329 (M−H)⁻

HRMS (ES+) calculated for C17H19N2O3S [M+H]⁺: 331.1116 found 331.1106

### EXAMPLE 4: (1S,2S,6R,7R)-4-(6-methyl-1,3-benzothiazol-2-yl)-4-azatricyclo[5.2.1.02,6]decane-3,5-dione

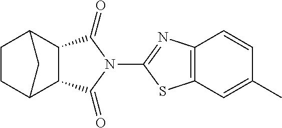 [SMILES: Cc1ccc2nc(N3C(=O)[C@@H]4C5CCC(C5)[C@@H]4C3=O)sc2c1]

### Step 1: (1S,2S,6R,7R)-4-oxatricyclo[5.2.1.02,6]decane-3,5-dione

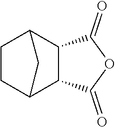 [SMILES: O=C1OC(=O)[C@H]2C3CCC(C3)[C@@H]12]

To a solution of (2S,6R)-4-oxatricyclo[5.2.1.02,6]dec-8-ene-3,5-dione (50 mg, 0.305 mmol) in MeOH (4 mL) was added ammonium formiate (57.6 mg, 0.313 mmol) and Pd/C (32.4 mg, 0.003 mmol). The reaction was heated under reflux overnight. Then, the reaction was cooled to room temperature and filtered over a pad of celite. The filtrate was evaporated and subjected to the next step without further purification (yellow oil, 40.0 mg).

LC-MS: method X, RT=2.05 min, MS (ESI) m/z: 167 (M+H)⁺

### Step 2: (1S,2S,6R,7R)-4-(6-methyl-1,3-benzothiazol-2-yl)-4-azatricyclo[5.2.1.02,6]decane-3,5-dione

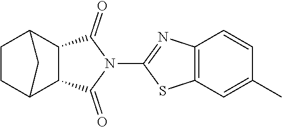 [SMILES: Cc1ccc2nc(N3C(=O)[C@@H]4C5CCC(C5)[C@@H]4C3=O)sc2c1]

To a solution of (2S,6R)-4-oxatricyclo[5.2.1.02,6]decane-3,5-dione (40 mg, 0.241 mmol) in THF (0.2 mL) was added dropwise a solution of 6-methyl-1,3-benzothiazol-2-amine (43.5 mg, 0.265 mmol) in THF (2.3 mL). After 48 h, the reaction mixture was heated under reflux for further 32 h. Then, the reaction was cooled to room temperature and evaporated under reduce pressure. The crude was purified by reverse column chromatography (C18, from 20/80 ACN-ammonium formiate/water-ammonium formiate to 100% water-ammonium formiate over 40 minutes). Collected fractions were lyophilized and dried to afford the title compound as white solid (7.5 mg, 8% over two steps).

¹H NMR (300 MHZ, DMSO-d6) δ=7.92(m, J=8.2, 1.2 Hz, 2H), 7.38 (dd, J=8.2, 1.2 Hz, 1H), 3.37 (m, 2H), 2.71 (s, 2H), 2.47 (s, 3H), 1.70 (d, J=9.6 Hz, 1H), 1.61-1.55 (m, 3H), 1.29-1.23 (m, 2H).

LC-MS: method X, RT=2.84 min, MS (ESI) m/z: 313 (M+H)⁺

HRMS (ES+) calculated for C17H17N2O2S [M+H]⁺: 313.1011 found 313.1012

### Example 5:3-[(5-methoxy-1,3-benzothiazol-2-yl) carbamoyl]bicyclo[2.2.1]hept-5-ene-2-carboxylic acid

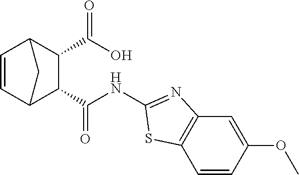 [SMILES: COc1ccc2sc(NC(=O)[C@@H]3C4C=CC(C4)[C@@H]3C(=O)O)nc2c1]

To a solution of (1R,2S,6R,7S)-4-oxatricyclo[5.2.1.02,6]dec-8-ene-3,5-dione (100 mg, 0.61 mmol) in THF (10 mL) was added 5-methoxy-1,3-benzothiazol-2-amine (110 mg, 0.61 mmol) and the mixture was stirred under reflux overnight. As the reaction was not complete, DIEA was added (105.4 μl, 0.61 mmol) and the reaction was stirred under reflux. After 7 h, the solvent was evaporated under reduce pressure. The crude was solubilized in a cooled aqueous solution of K₂CO₃/KOH, and then filtered. The aqueous phase was acidified to pH 1 by the addition of a solution of 1 M HCl to afford white-milk precipitate. The precipitate was recrystallized from AcOEt/EtOH to afford the title compound as white solid (16.4 mg, 8%).

¹H NMR (300 MHZ, DMSO-d6) δ=12.21 (brs, 1H), 7.77 (d, J=8.7 Hz, 1H), 7.23 (d, J=1.7 Hz, 1H), 6.89 (dd, J=8.6, 1.9 Hz, 1H), 6.15 (s, 2H), 3.80 (s, 3H), 3.48-3.38 (m, 2H), 3.09 (d, J=13.4 Hz, 2H), 1.33 (q, J=8.3 Hz, 2H).

LC-MS: method X, RT=8.30 min (30 min run), MS (ESI) m/z: 345 (M+H)⁺; 343 (M−H)⁻

HRMS (ES+) calculated for C17H17N2O4S [M+H]⁺: 345.0909 found 345.0912.

### Example 6:3-[(6-methoxy-1,3-benzothiazol-2-yl)carbamoyl]bicyclo[2.2.1]hept-5-ene-2-carboxylic acid

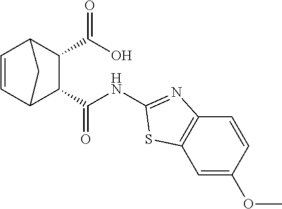 [SMILES: COc1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (20 mg, 0.122 mmol) in THF (0.1 mL) was added 6-methoxy-1,3-benzothiazol-2-amine (24.2 mg, 0.134 mmol) and the reaction mixture was heated under reflux. After 3 h, the solvent was evaporated under reduce pressure. The crude was purified by column chromatography (from 100 DCM to 70/30 DCM/DCM-MeOH (90/10)) to the afford the title compound as white solid (4.6 mg, 11%).

¹H NMR (300 MHZ, DMSO-d6) δ=12.11 (s, 1H), 11.87 (s, 1H), 7.58 (d, J=8.9 Hz, 1H), 7.51 (d, J=2.5 Hz, 1H), 7.01-6.97 (dd, J=8.9, 2.5 Hz, 1H), 6.16 (t, J=1.3 Hz, 2H), 3.79 (s, 3H), 3.48-43 (dd, J=10.2, 3.3 Hz, 1H), 3.38-33 (dd, J=10.2, 3.30 Hz, 1H), 3.12(s, 2H), 3.07 (s, 2H), 1.37-29 (q, J=16.9, 8.2 Hz, 2H).

LC-MS: method X, RT=8.15 min (30 min run), MS (ESI) m/z: 345 (M+H)⁺; 343 (M−H)⁻

HRMS (ES+) calculated for C17H17N2O4S [M+H]⁺: 345.0909 found 345.0910.

### Example 7:3-[(6-ethoxy-1,3-benzothiazol-2-yl)carbamoyl]bicyclo[2.2.1]hept-5-ene-2-carboxylic acid

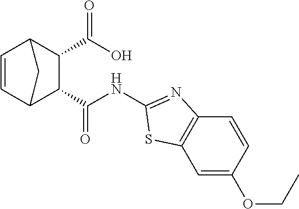 [SMILES: CCOc1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (115 mg, 0.7 mmol) in THF (2 mL) was added 6-ethoxy-1,3-benzothiazol-2-amine (136 mg, 0.7 mmol) and the mixture was stirred under reflux. After 4 h, the observed precipitate was filtered, washed with THF and dried to give the title compound as white solid (81 mg, 32%).

¹H NMR (300 MHZ, DMSO-d6) δ=12.10 (s, 1H), 11.85 (s, 1H), 7.57 (d, J=8.7 Hz, 1H), 7.49 (d, J=2.6 Hz, 1H), 6.99 (dd, J=8.7, 2.5 Hz, 1H), 6.16 (br s, 2H), 4.05 (q, J=6.9 Hz, 2H), 3.46 (dd, J=10.2, 3.2 Hz, 1H,), 3.35 (dd, J=10.2, 3.2 Hz, 1H), 3.12(s, 1H), 3.07 (s, 1H), 1.40-11.27 (m, 5H).

LC-MS: method X, RT=4.34 min (10 min run), MS (ESI) m/z: 359 (M+H)⁺; 357 (M−H)⁻

HRMS (ES+) calculated for C18H19N2O4S [M+H]⁺: 359.1066 found 359.1073.

### Example 8: (1R,2S,6R,7S)-4-(6-ethoxy-1,3-benzothiazol-2-yl)-4-azatricyclo[5.2.1.02,6]dec-8-ene-3,5-dione

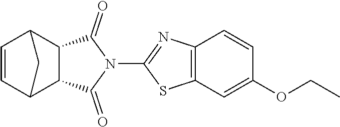 [SMILES: CCOc1ccc2nc(N3C(=O)[C@@H]4C5C=CC(C5)[C@@H]4C3=O)sc2c1]

To a solution of 3-[(6-ethoxy-1,3-benzothiazol-2-yl)carbamoyl]bicyclo[2.2.19hept-5-ene-2-carboxylic acid (164 mg, 0.458 mmol) in AcOEt (2 mL) was added T3P (50% in AcOEt, 545 μL, 0.915 mmol). The reaction was stirred at room temperature for 5 h. The observed precipitate was filtered. AcOEt was added to the filtrate and was washed with water twice. The organic layer was dried, filtered and concentrated under reduced pressure. The crude was purified by column chromatography (from 100% cyclohexane to 50/50 cyclohexnae/AcOEt) to afford the title compound (55 mg, 35%).

¹H NMR (300 MHZ, DMSO-d6) δ=7.87 (d, J=8.9 Hz, 1H), 7.66 (d, J=2.5 Hz, 1H), 7.11 (dd, J=8.6, 2.4 Hz, 1H), 6.21 (t, J=1.8 Hz, 2H), 4.09 (q, J=7.0 Hz, 2H), 3.60 (dd, J=2.7, 1.5 Hz, 2H), 3.37 (s, 2H), 1.62(br s, 2H), 1.35 (t, J=7.0 Hz, 3H).

LC-MS: method X, RT=4.58 min (10 min run), MS (ESI) m/z: 341 (M+H)⁺

HRMS (ES+) calculated for C18H17N2O3S [M+H]⁺: 341.0960 found 341.0963

### Example 9:3-[(5,6-dimethoxy-1,3-benzothiazol-2-yl)carbamoyl]bicyclo[2.2.1]hept-5-ene-2-carboxylic acid

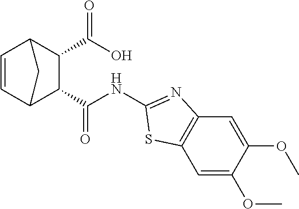 [SMILES: COc1cc2nc(NC(=O)[C@@H]3C4C=CC(C4)[C@@H]3C(=O)O)sc2cc1OC]

To a solution of (1R,2S,6R,7S)-4-oxatricyclo[5.2.1.02,6]dec-8-ene-3,5-dione (78.1 mg, 0.476 mmol) in THF (3 mL) was added 5,6-dimethoxy-1,3-benzothiazol-2-amine (100 mg, 0.476 mmol) and the mixture was stirred under reflux. After 6 h, the observed precipitate was filtered, washed with THF and dried to give the title compound as light brown solid (85 mg, 48%).

¹H NMR (300 MHZ, DMSO-d6) δ=12.07 (s, 1H), 11.81 (s, 1H), 7.48 (s, 1H), 7.25 (s, 1H), 6.15 (br s, 2H), 3.81 (s, 3H), 3.79 (s, 3H), 3.47 (dd, J=9.8, 2.6 Hz, 1H), 3.35 (dd, J=9.8, 2.8 Hz, 1H), 3.11 (s, 1H), 3.06 (s, 1H), 1.43-1.25 (m, 2H).

LC-MS: method X, RT=3.38 min (10 min run), MS (ESI) m/z: 375 (M+H)⁺; 373 (M−H)⁻

HRMS (ES+) calculated for C18H19N2O5S [M+H]⁺: 375.1015 found 375.1022

### Example 10:3-(6,7-dihydro-[1,4]dioxino[2,3-f][1,3]benzothiazol-2-ylcarbamoyl) bicyclo[2.2.1]hept-5-ene-2-carboxylic acid

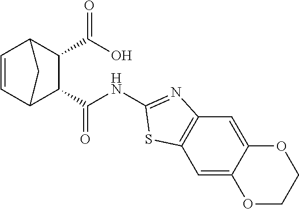 [SMILES: O=C(Nc1nc2cc3c(cc2s1)OCCO3)[C@@H]1C2C=CC(C2)[C@@H]1C(=O)O]

To a solution of 6,7-dihydro-[1,4]dioxino[2,3-f][1,3]benzothiazol-2-amine (146 mg, 0.7 mmol) in THF (3 mL) cooled to 0° C. was added NaH (9.7 mg, 0.7 mmol). Then, the solution was added dropwise to a solution of (1R,2S,6R,7S)-4-oxatricyclo[5.2.1.02,6]dec-8-ene-3,5-dione (115 mg, 0.7 mmol) in THF (3 mL). The reaction mixture was heated under reflux. After 4 h, the reaction was cooled to room temperature and THF was added. The observed precipitate was filtered, washed with THF and dried to give the title compound as white solid (84 mg, 32%).

¹H NMR (300 MHZ, DMSO-d6) δ=12.10 (s, 1H), 11.83 (s, 1H), 7.38 (s, 1H), 7.14 (s, 1H), 6.13 (m, 2H), 4.25 (s, 4H), 3.44 (dd, J=10.0, 2.7 Hz, 1H), 3.33 (m, 1H), 3.10 (s, 1H), 3.06 (s, 1H), 1.31 (dd, J=16.7, 8.1 Hz, 2H).

LC-MS: method X, RT=3.58 min (10 min run), MS (ESI) m/z: 373 (M+H)⁺; 371 (M−H)⁻

HRMS (ES+) calculated for C18H17N2O5S [M+H]⁺: 373.0858 found 373.0866

### Example 11:3-[(6-ethyl-1,3-benzothiazol-2-yl)carbamoyl]bicyclo[2.2.1]hept-5-ene-2-carboxylic acid

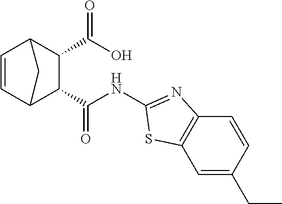 [SMILES: CCc1ccc2nc(NC(=O)[C@@H]3C4C=CC(C4)[C@@H]3C(=O)O)sc2c1]

To a solution of 6-ethyl-1,3-benzothiazol-2-amine (125 mg, 0.7 mmol) in THF (3 mL) cooled to 0° C. was added NaH (9.6 mg, 0.7 mmol). Then, the solution was added dropwise to a solution of (1R,2S,6R,7S)-4-oxatricyclo[5.2.1.02,6]dec-8-ene-3,5-dione (115 mg, 0.7 mmol) in THF (3 mL). The reaction mixture was heated under reflux. After 4 h, the reaction was cooled to room temperature and THF was added. The observed precipitate was filtered, washed with THF and dried. ACN was added and the precipitate was filtered, concentrated and dried to give the title compound as white solid (120 mg, 50%).

¹H NMR (300 MHZ, DMSO-d6) δ=12.22(s, 1H), 11.91 (s, 1H), 7.73 (d, J=1.2 Hz, 1H), 7.59 (d, J=8.2 Hz, 1H), 7.24 (dd, J=8.2, 1.6 Hz, 1H), 6.14 (s, 2H), 3.46 (dd, J=10.2, 3.5 Hz, 1H), 3.35 (dd, J=10.2, 3.3 Hz, 1H), 3.11 (s, 1H), 3.06 (s, 1H), 2.69 (q, J=7.6 Hz, 2H), 1.32(q, J=8.4 Hz, 2H), 1.21 (t, J=7.6 Hz, 3H).

LC-MS: method X, RT=4.62 min (10 min run), MS (ESI) m/z: 343 (M+H)⁺; 341 (M−H)⁻

HRMS (ES+) calculated for C18H19N2O3S [M+H]⁺: 343.1116 found 343.1106

### Example 12:3-[(6-tert-butyl-1,3-benzothiazol-2-yl)carbamoyl]bicyclo[2.2.1]hept-5-ene-2-carboxylic acid

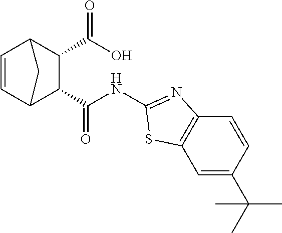 [SMILES: CC(C)(C)c1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (115 mg, 0.7 mmol) in THF (3 mL) was added 6-tert-butyl-1,3-benzothiazol-2-amine (144 mg, 0.7 mmol) and the mixture was stirred under reflux. After 3 h, the observed precipitate was filtered, washed with ACN and dried to give the title compound as white solid (79 mg, 30%).

¹H NMR (300 MHZ, DMSO-d6) δ=12.15 (brs, 1H), 11.91 (brs, 1H), 7.91 (s, 1H), 7.61 (d, J=8.3 Hz, 1H), 7.46 (d, J=7.6 Hz, 1H), 6.16 (s, 2H), 3.46 (d, J=10.3 Hz, 1H), 3.34 (d, J=10.2 Hz, 1H), 3.13 (s, 1H), 3.08 (s, 1H), 1.33 (br s, 11H).

LC-MS: method X, RT=5.10 min (10 min run), MS (ESI) m/z: 371 (M+H)⁺; 369 (M−H)⁻

### Example 13:3-[(4,6-dimethyl-1,3-benzothiazol-2-yl)carbamoyl]bicyclo[2.2.1]hept-5-ene-2-carboxylic acid

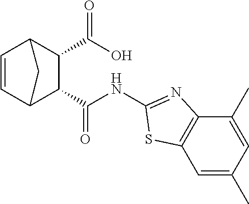 [SMILES: Cc1cc(C)c2nc(NC(=O)[C@@H]3C4C=CC(C4)[C@@H]3C(=O)O)sc2c1]

To a solution of 4,6-dimethyl-1,3-benzothiazol-2-amine (125 mg, 0.7 mmol) in THF (3 mL) cooled to 0° C. was added NaH (26.8 mg, 0.7 mmol). Then, the solution was added dropwise to a solution of (1R,2S,6R,7S)-4-oxatricyclo[5.2.1.02,6]dec-8-ene-3,5-dione (115 mg, 0.7 mmol) in THF (3 mL). The reaction mixture was heated under reflux overnight. Then, the reaction was cooled to room temperature and evaporated under reduce pressure. ACN was added and the observed precipitate was filtered, concentrated and dried to give the title compound as white solid (72 mg, 30%).

¹H NMR (300 MHZ, DMSO-d6) δ=12.28 (brs, 1H), 11.95 (brs, 1H), 7.51 (s, 1H), 7.03 (s, 1H), 6.14 (s, 2H), 3.45 (dd, J=10.8, 3.2 Hz, 1H), 3.34 (dd, J=10.8, 3.2 Hz, 1H), 3.10 (s, 1H), 3.06 (s, 1H), 2.5 (s, 3H), 2.34 (s, 3H), 1.30 (m, 2H)

LC-MS: method X, RT=4.68 min (10 min run), MS (ESI) m/z: 343 (M+H)⁺; 341 (M−H)⁻

HRMS (ES+) calculated for C18H19N2O3S [M+H]⁺: 343.1116 found 343.1107

### Example 14:3-[(5,6-dimethyl-1,3-benzothiazol-2-yl)carbamoyl]bicyclo[2.2.1]hept-5-ene-2-carboxylic acid

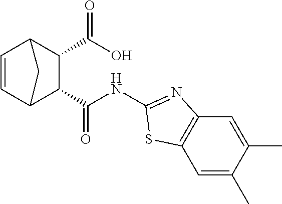 [SMILES: Cc1cc2nc(NC(=O)[C@@H]3C4C=CC(C4)[C@@H]3C(=O)O)sc2cc1C]

Commercially available. Bought from Vitas-M Laboratory.

### Example 15:3-[(4,5-dimethyl-1,3-benzothiazol-2-yl)carbamoyl]bicyclo[2.2.1]hept-5-ene-2-carboxylic acid

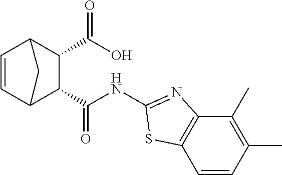 [SMILES: Cc1ccc2sc(NC(=O)[C@@H]3C4C=CC(C4)[C@@H]3C(=O)O)nc2c1C]

To a solution of 4,5-dimethyl-1,3-benzothiazol-2-amine (125 mg, 0.7 mmol) in THF (3 mL) cooled to 0° C. was added NaH (9.66 mg, 0.42 mmol). Then, the solution was added dropwise to a solution of (1R,2S,6R,7S)-4-oxatricyclo[5.2.1.02,6]dec-8-ene-3,5-dione (115 mg, 0.7 mmol) in THF (3 mL). The reaction mixture was heated under reflux. After 4 h, THF was added and the observed precipitate was filtered, washed with ACN and THF and dried to give the title compound as white solid (103 mg, 43%).

¹H NMR (300 MHz, DMSO-d6) δ=12.25 (brs, 1H), 11.91 (brs, 1H), 7.60 (d, J=7.9 Hz, 1H), 7.08 (d, J=8.3 Hz, 1H), 6.15 (t, J=1.2 Hz, 2H), 3.47 (dd, J=10.0, 3.3 Hz, 1H), 3.36 (dd, J=10.0, 3.4 Hz, 1H), 3.12(s, 1H), 3.08 (s, 1H), 2.49 (s, 3H), 2.33 (s, 3H), 1.33 (m, 2H).

LC-MS: method X, RT=4.72 min (10 min run), MS (ESI) m/z: 343 (M+H)⁺; 341 (M−H)⁻ HRMS (ES+) calculated for C18H19N2O3S [M+H]⁺: 343.1116 found 343.1102

### Example 16:3-[(4-methyl-1,3-benzothiazol-2-yl)carbamoyl]bicyclo[2.2.1]hept-5-ene-2-carboxylic acid

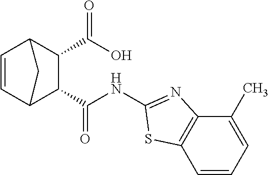 [SMILES: Cc1cccc2sc(NC(=O)[C@@H]3C4C=CC(C4)[C@@H]3C(=O)O)nc12]

To a solution of (1R,2S,6R,7S)-4-oxatricyclo[5.2.1.02,6]dec-8-ene-3,5-dione (115 mg, 0.7 mmol) in THF (3 mL) was added 4-methyl-1,3-benzothiazol-2-amine (115 mg, 0.7 mmol) and the mixture was stirred under reflux. After 3 h, the observed precipitate was filtered, washed with THF and ACN and dried to give the title compound as white solid (117 mg, 51%).

¹H NMR (300 MHZ, DMSO-d6) δ=12.32(brs, 1H) 11.85 (brs, 1H), 7.73 (d, J=7.3 Hz, 1H), 7.22(d, J=6.9 Hz, 1H), 7.16 (t, J=7.5 Hz, 1H), 6.22-6.09 (m, 2H), 3.47 (dd, J=10.2, 3.1 Hz, 1H), 3.37 (dd, J=10.1, 3.1 Hz, 1H), 3.12(s, 1H), 3.08 (s, 1H), 2.55 (s, 3H) 1.41-1.25 (m, 2H).

LC-MS: method X, RT=4.00 min (10 min run), MS (ESI) m/z: 329 (M+H)⁺; 327 (M−H)⁻

HRMS (ES+) calculated for C17H17N2O3S [M+H]⁺: 329.0960 found 329.0966

### Example 17:3-[(6-phenoxy-1,3-benzothiazol-2-yl) carbamoyl]bicyclo[2.2.1]hept-5-ene-2-carboxylic acid

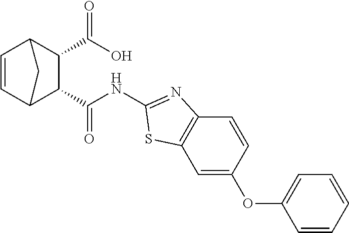 [SMILES: O=C(Nc1nc2ccc(Oc3ccccc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (696 mg, 4.2 mmol) in THF (5 mL) was added 6-phenoxy-1,3-benzothiazol-2-amine (1.0 g, 4.1 mmol). The mixture was stirred under reflux overnight. Then, the observed precipitate was filtered, washed with ACN and THF and dried to afford the title compound as white solid (1.39 g, 83%).

¹H NMR (300 MHZ, DMSO-d6) δ=12.24 (s, 1H), 12.00 (s, 1H), 7.71 (d, J=8.7 Hz, 1H), 7.64 (d, J=2.4 Hz, 1H), 7.42-7.33 (m, 2H), 7.71-7.07 (m, 2H), 7.04-6.98 (m, 2H), 6.16 (s, 2H), 3.47 (dd, J=10.0, 3.0 Hz, 1H), 3.38 (dd, J=10.0, 3.1 Hz, 1H), 3.13 (s, 1H), 3.09 (s, 1H), 1.37 (d, J=8.3 Hz), 1.31 (dt, J=8.9, 2.3 Hz, 1H).

LC-MS: method X, RT=4.62 min (10 min run), MS (ESI) m/z: 407 (M+H)⁺; 405 (M−H)⁻

HRMS (ES+) calculated for C22H19N2O4S [M+H]⁺: 407.1066 found 407.1064

### Example 18: [2-hydroxy-1,1-bis(hydroxymethyl)ethyl]ammonium: (1R,2S,3R,4S)-3-[(6-phenoxy-1,3-benzothiazol-2-yl) carbamoyl]bicyclo[2.2.1]hept-5-ene-2-carboxylate

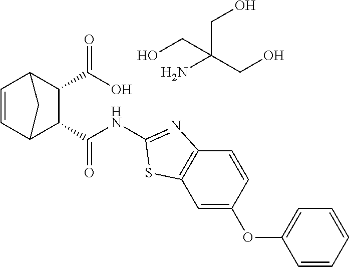 [SMILES: O=C(Nc1nc2ccc(Oc3ccccc3)cc2s1)[C@@H]1C2C=CC(C2)[C@@H]1C(=O)O ; NC(CO)(CO)CO]

To a solution of (1R,2S,3R,4S)-3-[(6-phenoxy-1,3-benzothiazol-2-yl)carbamoyl]bicyclo[2.2.1]hept-5-ene-2-carboxylic acid (5.3 g, 13.0 mmol) in a mixture MeOH/water (32/8 mL) was added 2-amino-2-(hydroxymethyl) propane-1,3-diol (4.6 g, 38 mmol) and the mixture was heated at 70° C. for 15 min. A thick suspension was observed and DMSO was added till complete solubilisation. Then, the mixture was filtrated and cooled to room temperature. Over the process, a precipitate was formed, filtrated, washed with water and dried to afford the title compound as white crystal (2.3 g, 33%). The process was repeated to afford supplementary 1.55 g of compound.

¹H NMR (300 MHZ, DMSO-d6) δ=7.68 (d, J=8.7 Hz, 1H), 7.60 (d, J=2.4 Hz, 1H), 7.42-7.33 (m, 2H), 7.15-7.06 (m, 2H), 7.00 (dd, J=8.2, 1.1 Hz, 2H), 6.17 (dd, J=5.5, 2.9 Hz, 1H), 6.13 (dd, J=5.4, 2.8 Hz, 1H), 3.39 (dd, J=10.0, 3.0 Hz, 1H), 3.34 (s, 6H), 3.30 (dd, J=10.0, 3.3 Hz, 1H), 3.18 (s, 3H), 3.13 (s, 1H), 3.08 (s, 1H), 1.33 (d, J=8.4 Hz, 1H), 1.26 (dt, J=8.4, 1.7 Hz, 1H).

LC-MS: method X, RT=4.62 min (10 min run), MS (ESI) m/z: 407 (M+H)⁺; 405 (M−H)⁻

### Example 19: (1R,2S,6R,7S)-4-(6-phenoxy-1,3-benzothiazol-2-yl)-4-azatricyclo[5.2.1.02,6]dec-8-ene-3,5-dione

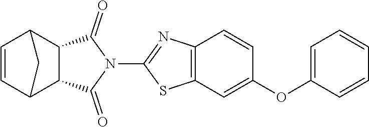 [SMILES: O=C1[C@@H]2C3C=CC(C3)[C@@H]2C(=O)N1c1nc2ccc(Oc3ccccc3)cc2s1]

To a solution of 3-[(6-phenoxy-1,3-benzothiazol-2-yl)carbamoyl]bicyclo[2.2.1]hept-5-ene-2-carboxylic acid (108 mg, 0.27 mmol) in AcOEt (1 mL) was added T3P (50% in AcOEt, 316 μL, 0.53 mmol) and the mixture was stirred at room temperature. After 5 h, the observed precipitate was filtered. EtOAc was added to the filtrate and washed twice with water. The organic layer was dried, filtered and concentrated under reduce pressure. The crude was purified by column chromatography (from 100% cyclohexane to 50/50 cyclohexane/AcOEt) to afford the title compound as white solid (64 mg, 62%).

¹H NMR (300 MHZ, DMSO-d6) δ=8.00 (d, J=9.0 Hz, 1H), 7.77 (d, J=2.4 Hz, 1H), 7.44-7.36 (m, 2H), 7.23 (dd, J=8.9 Hz, J=2.5 Hz, 1H), 7.16 (dt, J=7.5, 1.0 Hz, 1H), 7.08-7.02 (m, 2H), 6.21 (t, J=1.8 Hz, 2H), 3.62(dd, J=2.9, 1.6 Hz, 2H), 3.40-3.35 (m, 2H) 1.66-1.58 (m, 2H).

LC-MS: method X, RT=5.18 min (10 min run), MS (ESI) m/z: 389 (M+H)⁺

HRMS (ES+) calculated for C22H17N2O3S [M+H]⁺: 389.0960 found 389.0959

### Example 20:3-[(6-benzyl-1,3-benzothiazol-2-yl)carbamoyl]bicyclo[2.2.1]hept-5-ene-2-carboxylic acid

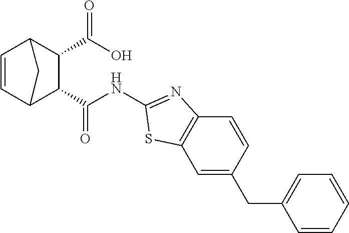 [SMILES: O=C(Nc1nc2ccc(Cc3ccccc3)cc2s1)[C@@H]1C2C=CC(C2)[C@@H]1C(=O)O]

### Step 1:6-benzyl-1,3-benzothiazol-2-amine

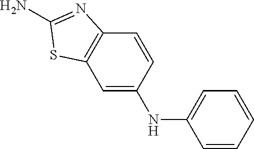 [SMILES: Nc1nc2ccc(Nc3ccccc3)cc2s1]

To a solution of 4-benzylaniline (397 mg, 2.17 mmol) in acetic acid (4 mL) was added ammonium thiocyanate (330 mg, 4.33 mmol). The mixture was cooled to 5° C. and a solution of bromine (0.111 mL, 2.17 mmol) in 2 mL of acetic acid was added. The mixture was stirred at room temperature. After 4 h, the reaction was filtered and washed with ethyl acetate. The obtained precipitate was neutralised with a saturated solution of Na₂CO₃. The aqueous phase was extracted with ethyl acetate 3 times. The combined organic layers were washed with sat. sol. of Na₂CO₃ and brine and then dried, filtered and concentrated under reduce pressure. The crude was purified by column chromatography (from 100% cyclohexane to 50/50 cyclohexane/ethyl acetate) to give the title compound as white solid (37.2 mg, 7%).

¹H NMR (300 MHZ, CDCl₃) δ=7.47 (d, J=8.3 Hz, 1H), 7.38 (s, 1H), 7.32-7.27 (m, 2H), 7.24-7.18 (m, 4H), 4.03 (s, 2H).

LC-MS: method X, RT=2.73 min (5 min run), MS (ESI) m/z: 241 (M+H)⁺; 239 (M−H)⁻

### Step 2:3-[(6-benzyl-1,3-benzothiazol-2-yl)carbamoyl]bicyclo[2.2.1]hept-5-ene-2-carboxylic acid

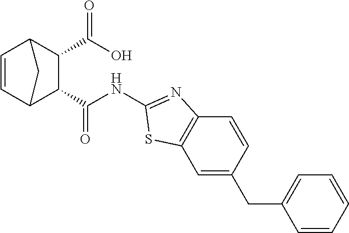 [SMILES: O=C(Nc1nc2ccc(Cc3ccccc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (26 mg, 0.144 mmol) in THF (4 mL) was added 6-benzyl-1,3-benzothiazol-2-amine (34.7 mg, 0.144 mmol). The mixture was stirred under reflux. After 24 h, the observed precipitate was filtered, washed with THF and dried to afford the title compound as white solid (21.7 mg, 37%).

¹H NMR (300 MHZ, DMSO-d6) δ=12.2(s, 1H), 11.84 (s, 1H), 7.77 (s, 1H), 7.60 (d, J=8.1 Hz, 1H), 7.34-7.12(m, 6H), 6.14 (s, 2H), 4.02(s, 2H), 3.50-3.34 (m, 2H), 3.11 (s, 1H), 3.06 (s, 1H), 1.32(m, 2H).

LC-MS: method X, RT=12.13 min (30 min run), MS (ESI) m/z: 405 (M+H)⁺; 403 (M−H)⁻

HRMS (ES+) calculated for C23H2ON2O3S [M+H]⁺: 405.1195 found 405.1287

### Example 21:3-[[6-(4-fluorophenoxy)-1,3-benzothiazol-2-yl]carbamoyl]bicyclo[2.2.1]hept-5-ene-2-carboxylic acid

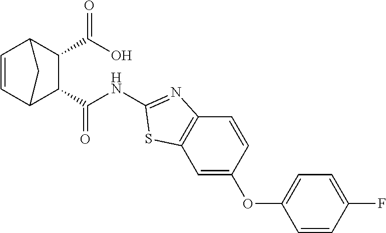 [SMILES: O=C(Nc1nc2ccc(Oc3ccc(F)cc3)cc2s1)[C@@H]1C2C=CC(C2)[C@@H]1C(=O)O]

### Step 1:6-4-fluorophenoxy)-1,3-benzothiazol-2-amine

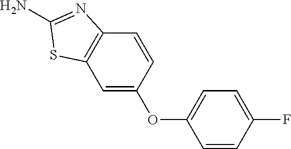 [SMILES: Nc1nc2ccc(Oc3ccc(F)cc3)cc2s1]

To a solution of 4-(4-fluorophenoxy) aniline (438 mg, 2.16 mmol) in acetic acid (0.5M) was added NH₄SCN (820 mg, 10.8 mmol), and the reaction was stirred for 90 minutes. Then, the mixture was cooled to 0° C. and a solution of bromine (0.166 mL, 3.23 mmol) in acetic acid (1M) was added. The mixture was allowed to warm to room temperature and stirred for 16 h. Then, the reaction was quenched by the addition of a saturated solution of Na₂CO₃. The reaction was made alkaline till pH 8. Ethyl acetate was added and the aqueous phase was extracted 3 times with ethyl acetate. The combined organic phases were dried, filtered and concentrated under reduce pressure. The crude was purified by column chromatography (from 100% cyclohexane to 20/80 cyclohexane/AcOEt) to afford the title compound as a slightly yellow solid (297 mg, 53% yield).

¹H NMR (300 MHZ, CDCl₃) δ=7.49 (d, J=8.7 Hz, 1H), 7.21 (d, J=2.4 Hz, 1H), 7.07-6.91 (m, 5H), 5.38 (brs, 2H).

### Step 2:3-[[6-(4-fluorophenoxy)-1,3-benzothiazol-2-yl]carbamoyl]bicyclo[2.2.1]hept-5-ene-2-carboxylic acid

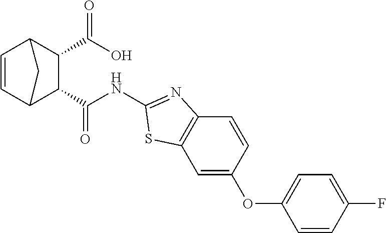 [SMILES: O=C(Nc1nc2ccc(Oc3ccc(F)cc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (63.1 mg, 0.384 mmol) in THF (3 mL) was added 6-(4-fluorophenoxy)-1,3-benzothiazol-2-amine (100 mg, 0.384 mmol) and the mixture was stirref under reflux. After 24 h, the observed precipitate was filtered and washed with THF and ACN, and dried to afford the title compound as white solid (83.8 mg, 50%).

¹H NMR (300 MHZ, DMSO-d6) δ=12.22(s, 1H), 11.84 (s, 1H), 7.68 (d, J=8.8 Hz, 1H), 7.60 (d, J=2.5 Hz, 1H), 7.26-7.17 (m, 2H), 7.12-7.02(m, 3H), 6.15 (s, 2H), 3.49-3.42(dd, J=10.2, 3.0 Hz, 1H), 3.39-3.33 (dd, J=10.2, 3.0 Hz, 1H), 3.12(s, 1H), 3.07 (s, 1H), 1.39-1.27 (m, 2H).

LC-MS: method X, RT=12.07 min (30 min run), MS (ESI) m/z: 425 (M+H)⁺; 423 (M−H)⁻

HRMS (ES+) calculated for C22H18N2O4SF [M+H]⁺: 425.0971 found 425.0974

### Example 22:3-[[6-[4-(trifluoromethyl)phenoxy]-1,3-benzothiazol-2-yl]carbamoyl]bicyclo[2.2.1]hept-5-ene-2-carboxylic acid

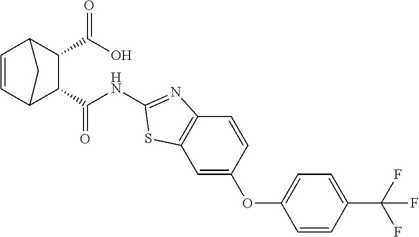 [SMILES: O=C(Nc1nc2ccc(Oc3ccc(C(F)(F)F)cc3)cc2s1)[C@@H]1C2C=CC(C2)[C@@H]1C(=O)O]

### Step 1:6-[4-(trifluoromethyl)phenoxy]-1,3-benzothiazol-2-amine

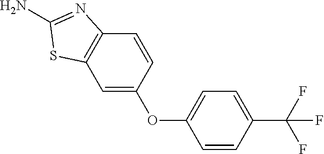 [SMILES: Nc1nc2ccc(Oc3ccc(C(F)(F)F)cc3)cc2s1]

To a solution of 4-[4-trifluoromethyl)phenoxy]aniline (246 mg, 0.971 mmol) in acetic acid (0.5 M) was added NH₄SCN (370 mg, 4.86 mmol). The mixture was cooled to 0° C. and a solution of Br₂(0.05 mL, 1.07 mmol) in acetic acid (1M) was added. The reaction was allowed to warm to room temperature and stirred overnight. After 16 h, the reaction was quenched by the addition of a saturated solution of Na₂CO₃. The mixture was made alkaline till pH 10. Ethyl acetate was added and the aqueous phase was extracted with ethyl acetate twice.

The organic phase was dried, filtered and concentrated under reduce pressure. The crude was purified by column chromatography (from 100% cyclohexane to 80/20 AcOEt/cyclohexane) to give the title compound as yellow oil (198 mg, 66%).

¹H NMR (300 MHz, DMSO-d6) δ=7.70 (d, J=9.0 Hz, 2H), 7.54 (d, J=2.4 Hz, 1H), 7.50 (s, 2H), 7.37 (d, J=8.6 Hz, 1H), 7.09 (d, J=8.7 Hz, 2H), 7.00 (dd, J=8.6, 2.5 Hz, 1H).

LC-MS: method X, RT=2.97 min (5 min run), MS (ESI) m/z: 311 (M+H)⁺; 309 (M−H)⁻

### 3-[[6-[4-(trifluoromethyl)phenoxy]-1,3-benzothiazol-2-yl]carbamoyl]bicyclo[2.2.1]hept-5-ene-2-carboxylic acid

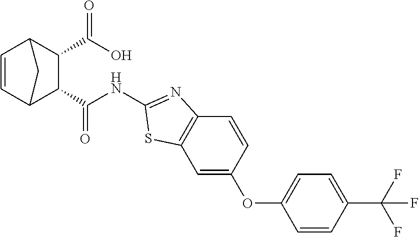 [SMILES: O=C(Nc1nc2ccc(Oc3ccc(C(F)(F)F)cc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (48.1 mg, 0.293 mmol) in THF (3 mL) was added 6-[4-(trifluoromethyl)phenoxy]-1,3-benzothiazol-2-amine (91 mg, 0.293 mmol) and the mixture was stirref under reflux. After 3 h, the observed precipitate was filtered and washed with THF and ACN, and dried to afford the title compound as white solid (13 mg, 9%).

¹H NMR (300 MHZ, DMSO-d6) δ=12.30 (s, 1H), 11.84 (s, 1H), 7.82-7.75 (m, 2H), 7.72(d, J=8.6 Hz, 2H), 7.22(d, J=8.5 Hz, 1H), 7.14 (d, J=8.6 Hz, 2H), 6.16 (s, 2H), 3.47-3.42 (m, 2H), 3.14 (s, 1H), 3.09 (s, 1H), 1.34 (m, 2H).

LC-MS: method X, RT=13.40 min (30 min run), MS (ESI) m/z: 475 (M+H)⁺; 473 (M−H)⁻

HRMS (ES+) calculated for C23H18N2O4SF3 [M+H]⁺: 475.0939 found 475.0914

### Example 23: (1R,2S,6R,7S)-4-[6-[4-(trifluoromethyl)phenoxy]-1,3-benzothiazol-2-yl]-4-azatricyclo[5.2.1.02,6]dec-8-ene-3,5-dione

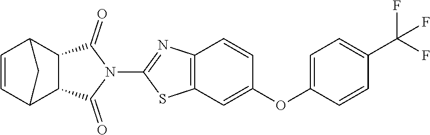 [SMILES: O=C1[C@@H]2C3C=CC(C3)[C@@H]2C(=O)N1c1nc2ccc(Oc3ccc(C(F)(F)F)cc3)cc2s1]

To a solution of (1R,2S,6R,7S)-4-oxatricyclo[5.2.1.02,6]dec-8-ene-3,5-dione (52.9 mg, 0.322 mmol) in THF (3 mL) was added 6-[4-(trifluoromethyl)phenoxy]-1,3-benzothiazol-2-amine (compound of step of example 21, 100 mg, 0.322 mmol) and the mixture was stirred under reflux. After 20 h, the observed precipitate was filtered and washed with THF and ACN, and dried to afford the title compound as white solid (91 mg, 62%).

¹H NMR (300 MHZ, DMSO-d6) δ=8.07 (d, J=8.9 Hz, 1H), 7.95 (d, J=2.5 Hz, 1H), 7.75 (d, J=8.7 Hz, 2H), 7.35 (dd, J=8.9, 2.5 Hz, 1H), 7.20 (d, J=8.7 Hz, 2H), 6.23 (t, J=1.9 Hz, 2H), 3.64 (dd, J=2.93, 1.6 Hz, 2H), 3.40 (m, 2H), 1.64 (m, 2H).

LC-MS: method X, RT=14.93 min (30 min run), MS (ESI) m/z: 457 (M+H)⁺

HRMS (ES+) calculated for C23H16N2O3SF3 [M+H]⁺: 457.0834 found 457.0835

### Example 24:3-[[6-(4-pyridyloxy)-1,3-benzothiazol-2-yl]carbamoyl]bicyclo[2.2.1]hept-5-ene-2-carboxylic acid

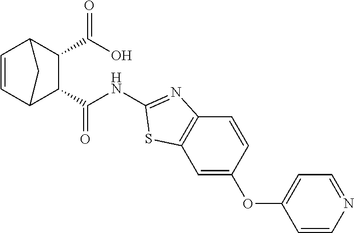 [SMILES: O=C(Nc1nc2ccc(Oc3ccncc3)cc2s1)[C@@H]1C2C=CC(C2)[C@@H]1C(=O)O]

### Step 1:6-(4-pyridyloxy)-1,3-benzothiazol-2-amine

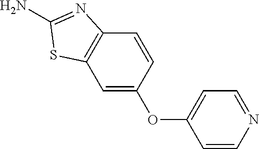 [SMILES: Nc1nc2ccc(Oc3ccncc3)cc2s1]

To a solution of 2-amino-1,3-benzothiazol-6-ol (200 mg, 1.20 mmol) in DMF (5 mL) was added Cs₂CO₃ (1.180 g, 1.81 mmol) and 4-chloropyridine (205 mg, 1.81 mmol). The reaction mixture was stirred at 100° C. After 40 h, the reaction was cooled to room temperature. Water was added and the aqueous phase was extracted 3 times with ethyl acetate. The combined organic phase was washed with brine and then dried, filtered and concentrated under reduce pressure. The crude was purified by column chromatography (from 100% cyclohexane to 100% ethyl acetate) to afford the title compound as white solid (96 mg, 33%).

¹H NMR (300 MHZ, DMSO-d6) δ=8.42(m, 2H), 7.56 (d, J=2.4 Hz, 1H), 7.51 (s, 2H), 7.37 (d, J=8.6 Hz, 1H), 7.03-6.99 (dd, J=8.6, 2.5 Hz, 1H), 6.88 (m, 2H).

LC-MS: method X, RT=1.75 min (5 min run), MS (ESI) m/z: 244 (M+H)⁺; 242(M−H)⁻

### Step 2:3-[[6-(4-pyridyloxy)-1,3-benzothiazol-2-yl]carbamoyl]bicyclo[2.2.1]hept-5-ene-2-carboxylic acid

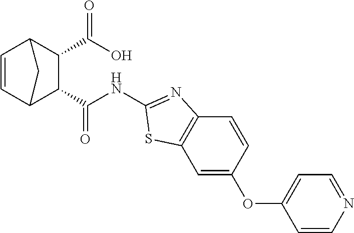 [SMILES: O=C(Nc1nc2ccc(Oc3ccncc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (58 mg, 0.353 mmol) in THF (4 mL) was added 6-(4-pyridyloxy)-1,3-benzothiazol-2-amine (86 mg, 0.353 mmol). The mixture was heated under reflux. After 5 h, the solvent was evaporated under reduce pressure. The solid was washed with THE ad ACN, filtered and dried to afford the title compound as white solid (51.9 mg, 36%).

¹H NMR (300 MHZ, DMSO-d6) δ=12.31 (brs, 1H), 11.89 (brs, 1H), 8.44 (s, 2H), 7.83 (d, J=2.4 Hz, 1H), 7.76 (d, J=8.7 Hz, 1H), 7.24-7.20 (dd, J=8.7, 2.5 Hz, 1H), 6.92(d, J=5.9 Hz, 2H), 6.16 (m, 2H), 3.49-3.44 (dd, J=10.0, 3.0 Hz, 1H), 3.40-3.35 (m, 1H), 3.13 (s, 1H), 3.08 (s, 1H), 1.38-1.29 (m, 2H).

LC-MS: method X, RT=2.10 min (5 min run), MS (ESI) m/z: 408 (M+H)⁺; 406 (M−H)⁻

HRMS (ES+) calculated for C21H17N304S [M+H]⁺: 408.1018 found 408.1034

### Examples 25 and 26:3-[[6-[[3-(trifluoromethyl)-2-pyridyl]oxy]-1,3-benzothiazol-2-yl]carbamoyl]bicyclo[2.2.1]hept-5-ene-2-carboxylic acid and (1R,2S,6R,7S)-4-6-[[3-(trifluoromethyl)-2-pyridyl]oxy]-1,3-benzothiazol-2-yl]-4-azatricyclo[5.2.1.02,6]dec-8-ene-3,5-dione

### Step 1:6-[[3-(trifluoromethyl)-2-pyridyl]oxy]-1,3-benzothiazol-2-amine

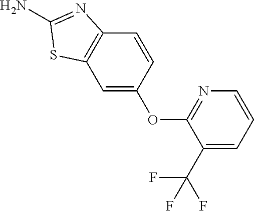 [SMILES: Nc1nc2ccc(Oc3ncccc3C(F)(F)F)cc2s1]

To a solution of 2-amino-1,3-benzothiazol-6-ol (500 mg, 3.01 mmol) in DMF (7 mL) was added Cs₂CO₃ (2.94 g, 9.03 mmol) and 2-chloro-3-(trifluoromethyl)pyridine (819 mg, 4.51 mmol). The reaction mixture was stirred at 100° C. After 48 h, the reaction was cooled to room temperature. Water was added and the aqueous phase was extracted 3 times with ethyl acetate. The combined organic phase was washed with brine and then dried, filtered and concentrated under reduce pressure. The crude was purified by column chromatography (from 100% cyclohexane to 100% ethyl acetate) to afford the title compound as brown oil (595 mg, 64%).

¹H NMR (300 MHZ, DMSO-d6) δ=8.28 (m, 1H), 7.98 (m, 1H), 7.55 (d, J=8.7 Hz, 1H), 7.42 (d, J=2.3 Hz, 1H), 7.15-7.11 (dd, J=8.7, 2.4 Hz, 1H), 7.07 (m, 1H), 6.12(brs, 2H).

LC-MS: method X, RT=2.60 min (5 min run), MS (ESI) m/z: 312(M+H)⁺; 310 (M−H)⁻ Step 2:

To a solution of (1R,2S,6R,7S)-4-oxatricyclo[5.2.1.02,6]dec-8-ene-3,5-dione (160 mg, 0.98 mmol) in THF (4 mL) was added 6-[[3-(trifluoromethyl)-2-pyridyl]oxy]-1,3-benzothiazol-2-amine (303 mg, 0.98 mmol). The mixture was heated under reflux. After 24 h, the solvent was evaporated under reduce pressure and the crude was purified by column chromatography (from 100% cyclohexane to 90/10 AcOEt/MeOH) to afford both title compound as white solid (Example 25, 16 mg, 3.5%; Example 26, 58 mg, 13%).

### Example 25 3-[[6-[[3-(trifluoromethyl)-2-pyridyl]oxy]-1,3-benzothiazol-2-yl]carbamoyl]bicyclo[2.2.1]hept-5-ene-2-carboxylic acid

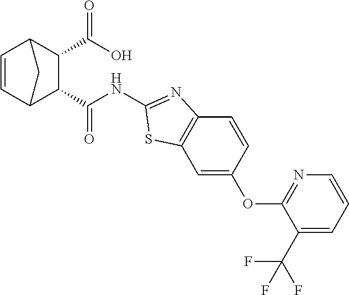 [SMILES: O=C(Nc1nc2ccc(Oc3ncccc3C(F)(F)F)cc2s1)[C@@H]1C2C=CC(C2)[C@@H]1C(=O)O]

¹H NMR (300 MHZ, DMSO-d6) δ=12.30 (brs, 1H), 11.88 (brs, 1H), 8.37 (d, J=4.0 Hz, 1H), 8.26 (d, J=7.7 Hz, 1H), 7.81 (d, J=2.4 Hz, 1H), 7.73 (d, J=8.7 Hz, 1H), 7.31 (dd, J=7.5, 5.2 Hz, 1H), 7.20 (dd, J=8.7, 2.5 Hz, 1H), 6.16 (m, 2H), 3.49-3.45 (dd, J=10.0, 3.0 Hz, 1H), 3.40-3.36 (m, 1H), 3.13 (s, 1H), 3.08 (s, 1H), 1.38-1.29 (m, 2H).

LC-MS: method X, RT=2.78 min (5 min run), MS (ESI) m/z: 476 (M+H)⁺; 474 (M−H)⁻

HRMS (ES+) calculated for C22H17F3N3O4S [M+H]⁺: 476.0892 found 476.0895

### Example 26

### (1R,2S,6R,7S)-4-[6-[[3-(trifluoromethyl)-2-pyridyl]oxy]-1,3-benzothiazol-2-yl]-4-azatricyclo[5.2.1.02,6]dec-8-ene-3,5-dione

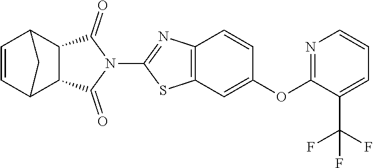 [SMILES: O=C1[C@@H]2C3C=CC(C3)[C@@H]2C(=O)N1c1nc2ccc(Oc3ncccc3C(F)(F)F)cc2s1]

¹H NMR (300 MHZ, DMSO-d6) δ=8.27 (dd, J=4.9, 1.7 Hz, 1H), 8.12(d, J=8.9 Hz, 1H), 8.01 (dd, J=7.5, 1.7 Hz, 1H), 7.69 (d, J=2.3 Hz, 1H), 7.32-7.28 (dd, J=8.9, 2.4 Hz, 1H), 7.14-7.09 (dd, J=7.6, 5.0, 1H), 6.28 (s, 2H), 3.58-3.53 (m, 4H), 1.82(d, J=8.9 Hz, 1H), 1.64 (d, J=8.9 Hz, 1H).

LC-MS: method X, RT=3.08 min (5 min run), MS (ESI) m/z: 458 (M+H)⁺

HRMS (ES+) calculated for C22H15F3N303S [M+H]⁺: 458.0786 found 458.0780

### Example 27:3-[[6-[[4-(trifluoromethyl)-2-pyridyl]oxyl-1,3-benzothiazol-2-yl]carbamoyl]bicyclo[2.2.1]hept-5-ene-2-carboxylic acid

### Step 1:6-[[4-(trifluoromethyl)-2-pyridyl]oxy]-1,3-benzothiazol-2-amine

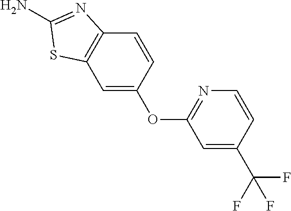 [SMILES: Nc1nc2ccc(Oc3cc(C(F)(F)F)ccn3)cc2s1]

To a solution of 2-amino-1,3-benzothiazol-6-ol (2 g, 12.0 mmol) in DMF (15 mL) was added 2-chloro-4-(trifluoromethyl)pyridine (1550 μL, 12.0 mmol) and Cs₂CO₃ (11.8 g, 36.1 mmol). The mixture was heated at 100° C. for 20 h. Then, the reaction was cooled to room temperature. Water was added and the aqueous phase was extracted 3 times with ethyl acetate. The combined organic phases were dried, filtered and concentrated under reduce pressure. The crude was purified by column chromatography (from 100% cylohexane to 100% AcOEt). The collected fractions were concentrated and the observed precipitate was washed with cyclohexane to afford the title compound as white solid (933 mg, 25%).

¹H NMR (300 MHZ, DMSO-d6) δ=8.38 (d, J=5.2 Hz, 1H), 7.55 (d, J=2.2 Hz, 1H), 7.48-7.44 (m, 3H), 7.40 (m, 1H), 7.34 (d, J=8.8 Hz, 1H), 7.04-7.00 (dd, J=8.6, 2.5 Hz, 1H).

LC-MS: method X, RT=2.68 min (5 min run), MS (ESI) m/z: 312(M+H)⁺; 310 (M−H)⁻

### Step 2:3-[[6-[[4-(trifluoromethyl)-2-pyridyl]oxy]-1,3-benzothiazol-2-yl]carbamoyl]bicyclo[2.2.1]hept-5-ene-2-carboxylic acid

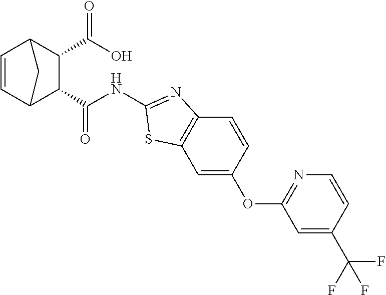 [SMILES: O=C(Nc1nc2ccc(Oc3cc(C(F)(F)F)ccn3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (38 mg, 0.231 mmol) in THF (2 mL) was added 6-[[4-(trifluoromethyl)-2-pyridyl]oxy]-1,3-benzothiazol-2-amine (72.1 mg, 0.231 mmol). The mixture was heated under reflux. After 40 h, the solvent was evaporated and the crude was purified by column chromatography (from 100% cyclohexane to 90/10 AcOEt/methanol) to afford the title compound as white solid (67.5 mg, 61%).

¹H NMR (300 MHZ, DMSO-d6) δ=12.34 (brs, 1H), 11.87 (brs, 1H), 8.40 (d, J=5.0 Hz, 1H), 7.82(d, J=2.4 Hz, 1H), 7.74 (d, J=8.9 Hz, 1H), 7.51-7.47 (m, 2H), 7.26-7.22(dd, J=8.7, 2.4 Hz, 1H), 6.17 (m, 2H), 3.50-3.45 (dd, J=10.0, 3.2 Hz, 1H), 3.41-3.18 (m, 1H), 3.14 (s, 1H), 3.09 (s, 1H), 1.39-1.30 (m, 2H).

LC-MS: method X, RT=12.03 min (30 min run), MS (ESI) m/z: 476 (M+H)⁺; 474 (M−H)⁻

HRMS (ES+) calculated for C22H17N304SF3 [M+H]⁺: 476.0892 found 476.0892

### Example 28: (1R,2S,6R,7S)-4-[6-[[4-(trifluoromethyl)-2-pyridyl]oxy]-1,3-benzothiazol-2-yl]-4-azatricyclo[5.2.1.02,6]dec-8-ene-3,5-dione

### Step 1:6-[[4-(trifluoromethyl)-2-pyridyl]oxy]-1,3-benzothiazol-2-amine

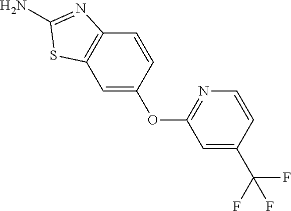 [SMILES: Nc1nc2ccc(Oc3cc(C(F)(F)F)ccn3)cc2s1]

To a solution of 2-amino-1,3-benzothiazol-6-ol (2 g, 12.0 mmol) in DMF (15 mL) was added 2-chloro-4-(trifluoromethyl)pyridine (1550 μL, 12.0 mmol) and Cs₂CO₃ (11.8 g, 36.1 mmol). The mixture was heated at 100° C. for 20 h. Then, the reaction was cooled to room temperature. Water was added and the aqueous phase was extracted 3 times with ethyl acetate. The combined organic phases were dried, filtered and concentrated under reduce pressure. The crude was purified by column chromatography (from 100% cylohexane to 100% AcOEt). The collected fractions were concentrated and the observed precipitate was washed with cyclohexane to afford the title compound as white solid (933 mg, 25%).

¹H NMR (300 MHZ, DMSO-d6) δ=8.38 (d, J=5.2 Hz, 1H), 7.55 (d, J=2.2 Hz, 1H), 7.48-7.44 (m, 3H), 7.40 (m, 1H), 7.34 (d, J=8.8 Hz, 1H), 7.04-7.00 (dd, J=8.6, 2.5 Hz, 1H).

LC-MS: method X, RT=2.68 min (5 min run), MS (ESI) m/z: 312(M+H)⁺; 310 (M−H)⁻

### Step 2:(1R,2S,6R,7S)-4-[6-[[4-(trifluoromethyl)-2-pyridyl]oxy]-1,3-benzothiazol-2-yl]-4-azatricyclo[5.2.1.02,6]dec-8-ene-3,5-dione

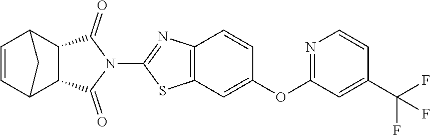 [SMILES: O=C1[C@@H]2C3C=CC(C3)[C@@H]2C(=O)N1c1nc2ccc(Oc3cc(C(F)(F)F)ccn3)cc2s1]

To a solution of (1R,2S,6R,7S)-4-oxatricyclo[5.2.1.02,6]dec-8-ene-3,5-dione (492 mg, 3.0 mmol) in THF (4 mL) was added 6-[[4-(trifluoromethyl)-2-pyridyl]oxy]-1,3-benzothiazol-2-amine (933 mg, 3.0 mmol). The mixture was heated under reflux. After 40h. the solvent was evaporated and the crude was purified by column chromatography (from 100% cyclohexane to 90/10 AcOEt/methanol) to afford the title compound as white solid (311.2 mg, 23%).

¹H NMR (300 MHZ, CDCl₃) δ=8.32(d, J=5.2 Hz, 1H), 8.12(d, J=8.8 Hz, 1H), 7.67 (d, J=2.3 Hz, 1H), 7.31-7.27 (dd, J=8.8, 2.3 Hz, 1H), 7.23 (d, J=5.2, 1H), 7.21 (s, 1H), 6.28 (s, 2H), 3.58 (s, 2H), 3.56 (d, 2H), 1.83 (d, J=9.0 Hz, 1H), 1.66 (d, J=8.8 Hz, 1H).

LC-MS: method X, RT=13.67 min (30 min run), MS (ESI) m/z: 458 (M+H)⁺

HRMS (ES+) calculated for C22H15F3N303S [M+H]⁺: 458.0786 found 458.0775

### Example 29 and 30: 3-[[6-[(4-methyl-2-pyridyl)oxy]-1,3-benzothiazol-2-yl]carbamoyl]bicyclo[2.2.1]hept-5-ene-2-carboxylic acid/(1R,2S,6R,7S)-4-[6-1 (4-methyl-2-pyridyl)oxy]-1,3-benzothiazol-2-yl]-4-azatricyclo[5.2.1.02,6]dec-8-ene-3,5-dione

### Step 1:6-[(4-methyl-2-pyridyl)oxy]-1,3-benzothiazol-2-amine

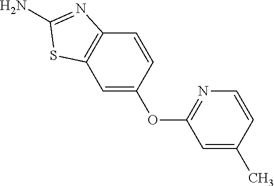 [SMILES: Cc1ccnc(Oc2ccc3nc(N)sc3c2)c1]

To a solution of 2-amino-1,3-benzothiazol-6-ol (300 mg, 1.81 mmol) in DMF (5 mL) was added 2-chloro-4-methyl-pyridine (202 L, 1.81 mmol) and Cs₂CO₃ (1.76 g, 5.42 mmol). The mixture was heated at 100° C. After 15 days, the reaction was cooled to room temperature. Water was added and the aqueous phase was extracted 3 times with ethyl acetate. The combined organic phases were dried, filtered and concentrated under reduce pressure. The crude was purified by column chromatography (from 100% cylohexane to 100% AcOEt) to afford the title compound with as white solid (93.3 mg, 20%).

¹H NMR (300 MHZ, DMSO-d6) δ=7.98-7.96 (dd, J=5.2, 0.4 Hz, 1H), 7.45-7.44 (dd, J=2.5, 0.3 Hz, 1H), 7.42(brs, 2H), 7.33-7.30 (dd, J=8.6, 0.3 Hz, 1H), 6.95-6.91 (m, 2H), 6.78 (q, J=0.7 Hz, 1H), 2.30 (s, 3H).

LC-MS: method X, RT=2.30 min (5 min run), MS (ESI) m/z: 258 (M+H)⁺

### Step 2:3-[[6-[(4-methyl-2-pyridyl)oxy]-1,3-benzothiazol-2-yl]carbamoyl]bicyclo[2.2.1]hept-5-ene-2-carboxylic acid

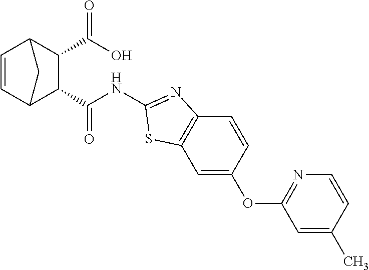 [SMILES: Cc1ccnc(Oc2ccc3nc(NC(=O)[C@@H]4C5C=CC(C5)[C@@H]4C(=O)O)sc3c2)c1]

To a solution of (1R,2S,6R,7S)-4-oxatricyclo[5.2.1.02,6]dec-8-ene-3,5-dione (60 mg, 0.37 mmol) in THF (2 mL) was added 6-[(4-methyl-2-pyridyl)oxy]-1,3-benzothiazol-2-amine (94 mg, 0.37 mmol). The mixture was heated under reflux. After 20 h, the observed precipitate was filtrated, washed with THF and ACN, and dried to afford the title acid compound as white solid (Example 29, 36.8 mg, 24%).

The filtrate was evaporated and the crude was purified by column chromatography (from 100% cyclohexane to 90/10 AcOEt/methanol) to afford the title compound as white solid (Example 30, 19.3 mg, 13%).

### Example 29

¹H NMR (300 MHZ, DMSO-d6) δ=12.24 (brs, 1H), 11.86 (brs, 1H), 7.98 (d, J=5.1 Hz, 1H), 7.70 (s, 1H), 7.69 (d, J=10.4 Hz, 1H), 7.14 (dd, J=8.7, 2.5 Hz, 1H), 6.95 (d, J=5.1 Hz, 1H), 6.85 (s, 1H), 6.16 (m, 2H), 3.49-3.44 (dd, J=10.0, 3.1 Hz, 1H), 3.39-3.34 (m, 1H), 3.13 (s, 1H), 3.08 (s, 1H), 2.07 (s, 3H), 1.37 (d, J=8.2 Hz, 1H), 1.30 (d, J=8.3 Hz, 1H).

LC-MS: method X, RT=2.54 min (5 min run), MS (ESI) m/z: 422(M+H)⁺; 420 (M−H)⁻

HRMS (ES+) calculated for C22H2ON304S [M+H]⁺: 422.1175 found 422.1177

### Example 30

### (1R,2S,6R,7S)-4-[[6-[(4-methyl-2-pyridyl)oxy]-1,3-benzothiazol-2-yl]-4-azatricyclo [5.2.1.02,6]dec-8-ene-3,5-dione

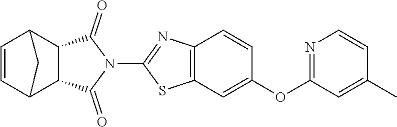 [SMILES: Cc1ccnc(Oc2ccc3nc(N4C(=O)[C@@H]5C6C=CC(C6)[C@@H]5C4=O)sc3c2)c1]

¹H NMR (300 MHZ, CDCl₃) δ=8.08 (d, J=8.9 Hz, 1H), 8.06 (d, J=5.2 Hz, 1H), 7.26 (dd, J=8.9, 2.3 Hz, 1H), 6.86 (d, J=5.2 Hz, 1H), 6.74 (s, 1H), 6.28 (brs, 2H), 3.57-3.54 (m, 4H), 2.36 (s, 3H), 1.82(d, J=8.9 Hz, 1H), 1.64 (d, J=8.9 Hz, 1H).

LC-MS: method X, RT=2.87 min (5 min run), MS (ESI) m/z: 404 (M+H)⁺

HRMS (ES+) calculated for C22H18N303S [M+H]⁺: 404.1069 found 404.1046

### Example 31:2-[[2-[(1R,2S,6R,7S)-3,5-dioxo-4-azatricyclo[5.2.1.02,6]dec-8-en-4-yl]-1,3-benzothiazol-6-yl]oxy]pyridine-4-carbonitrile

### Step 1:2-[(2-amino-1,3-benzothiazol-6-yl)oxy]pyridine-4-carbonitrile

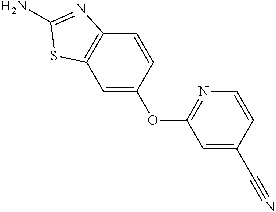 [SMILES: N#Cc1ccnc(Oc2ccc3nc(N)sc3c2)c1]

To a solution of 2-amino-1,3-benzothiazol-6-ol (400 mg, 2.41 mmol) in DMF (5 mL) was added 2-chloropyridine-4-carbonitrile (500 mg, 3.61 mmol) and Cs₂CO₃ (2.35 g, 7.22 mmol). The mixture was heated at 100° C. After 48 h, the reaction was cooled to room temperature. Water was added and the aqueous phase was extracted 3 times with ethyl acetate. The combined organic phases were dried, filtered and concentrated under reduce pressure. The crude was purified by column chromatography (from 100% cylohexane to 100% AcOEt) to afford the title compound with as white solid (45 mg, 7%).

LC-MS: method X, RT=2.23 min (5 min run), MS (ESI) m/z: 269 (M+H)⁺; 267 (M−H)⁻

### Step 2:2-[[2-[(1R,2S,6R,7S)-3,5-dioxo-4-azatricyclo[5.2.1.02,6]dec-8-en-4-yl]-1,3-benzothiazol-6-yl]oxy]pyridine-4-carbonitrile

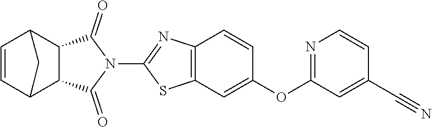 [SMILES: N#Cc1ccnc(Oc2ccc3nc(N4C(=O)[C@@H]5C6C=CC(C6)[C@@H]5C4=O)sc3c2)c1]

To a solution of (1R,2S,6R,7S)-4-oxatricyclo[5.2.1.02,6]dec-8-ene-3,5-dione (28 mg, 0.17 mmol) in THF (2 mL) was added 2-[(2-amino-1,3-benzothiazol-6-yl)oxy]pyridine-4-carbonitrile (45.8 mg, 0.17 mmol). The mixture was heated under reflux. After 36 h, the solvent was evaporated under reduce pressure. The crude was purified by column chromatography (from 100% cycloheaxne to 90/10 AcOEt, MeOH) to afford the title compound as white solid (5.2 mg, 7.4%).

¹H NMR (300 MHZ, DMSO-d6) δ=8.30 (d, J=5.8 Hz, 1H), 8.13 (d, J=8.9 Hz, 1H), 7.66 (d, J=2.3 Hz, 1H), 7.27 (dd, J=6.7, 2.1 Hz, 1H), 7.26-7.221 (m, 2H), 6.27 (t, J=1.6 Hz, 2H), 3.61-3.53 (m, 4H), 1.82(dt, J=8.9, 1.4 Hz, 1H), 1.66 (d, J=8.8 Hz, 1H).

LC-MS: method X, RT=2.80 min (5 min run), MS (ESI) m/z: 415 (M+H)⁺

HRMS (ES+) calculated for C22H15N403S [M+H]⁺: 415.0865 found 415.0865

### Example 32:3-[[6-[[2-(trifluoromethyl)-4-pyridyl]oxy]-1,3-benzothiazol-2-yl]carbamoyl]bicyclo[2.2.1]hept-5-ene-2-carboxylic acid

### Step 1:6-[[2-(trifluoromethyl)-4-pyridyl]oxy]-1,3-benzothiazol-2-amine

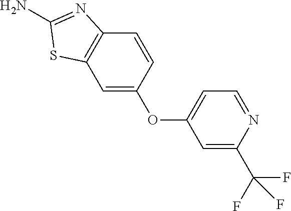 [SMILES: Nc1nc2ccc(Oc3ccnc(C(F)(F)F)c3)cc2s1]

To a solution of 2-amino-1,3-benzothiazol-6-ol (500 mg, 3.01 mmol) in DMF (7 mL) was added 4-chloro-2-(trifluoromethyl)pyridine (546 mg, 3.01 mmol) and Cs₂CO₃ (2.94 g, 9.03 mmol). The mixture was heated at 100° C. for 24 h. Then, the reaction was cooled to room temperature. Water was added and the aqueous phase was extracted 3 times with ethyl acetate. The combined organic phases were dried, filtered and concentrated under reduce pressure. The crude was purified by column chromatography (from 100% cyclohexane to 100% AcOEt) to afford the title compound as white solid (129 mg, 14%).

¹H NMR (300 MHZ, DMSO-d6) δ=8.60 (d, J=5.7 Hz, 1H), 7.64 (d, J=2.5 Hz, 1H), 7.57 (brs, 2H), 7.42-7.39 (m, 2H), 7.13 (dd, J=5.7, 2.5 Hz, 1H), 7.08 (dd, J=8.6, 2.5 Hz, 1H).

LC-MS: method X, RT=2.54 min (5 min run), MS (ESI) m/z: 312(M+H)⁺; 310 (M−H)⁻

### Step 2:3-[[6-[[2-(trifluoromethyl)-4-pyridyl]oxy]-1,3-benzothiazol-2-yl]carbamoyl]bicyclo[2.2.1]hept-5-ene-2-carboxylic acid

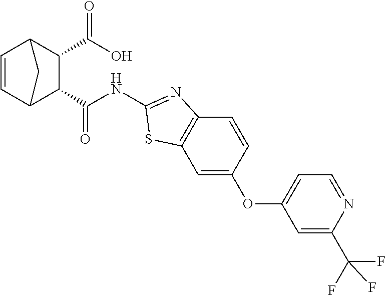 [SMILES: O=C(Nc1nc2ccc(Oc3ccnc(C(F)(F)F)c3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (68 mg, 0.41 mmol) in THF (2 mL) was added 6-[[2-(trifluoromethyl)-4-pyridyl]oxy]-1,3-benzothiazol-2-amine (129 mg, 0.41 mmol). The mixture was heated under reflux. After 24 h, the solvent was evaporated and the crude was purified by column chromatography (from 100% cyclohexane to 90/10 AcOEt/methanol) to afford the title acid compound as white solid (76.6 mg, 39%).:

¹H NMR (300 MHZ, DMSO-d6) δ=12.37 (brs, 1H), 11.99 (brs, 1H), 8.61 (d, J=5.7 Hz, 1H), 7.92(d, J=2.4 Hz, 1H), 7.79 (d, J=8.7 Hz, 1H), 7.44 (d, J=2.3 Hz, 1H), 7.29 (dd, J=8.7, 2.5 Hz, 1H), 7.16 (dd, J=5.7, 2.4 Hz, 1H), 6.16 (m, 2H), 3.46 (dd, J=9.9, 3.2 Hz, 1H), 3.38 (m, 1H), 3.13 (s, 1H), 3.08 (s, 1H), 1.37 (d, J=8.2 Hz, 1H), 1.31 (d, J=8.2 Hz, 1H).

LC-MS: method X, RT=11.48 min (30 min run), MS (ESI) m/z: 476 (M+H)⁺; 474 (M−H)⁻

HRMS (ES+) calculated for C22H17F3N3O4S [M+H]⁺: 476.0892 found 476.0884

### Example 33: (1R,2S,6R,7S)-4-[6-[(2-chloro-4-pyridyl)oxy]-1,3-benzothiazol-2-yl]-4-azatricyclo[5.2.1.02,6]dec-8-ene-3,5-dione

### Step 1:6-[(2-chloro-4-pyridyl)oxy]-1,3-benzothiazol-2-amine

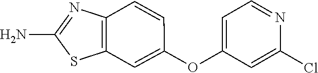 [SMILES: Nc1nc2ccc(Oc3ccnc(Cl)c3)cc2s1]

To a solution of 2-amino-1,3-benzothiazol-6-ol (500 mg, 3.01 mmol) in DMF (5 mL) was added 4-bromo-2-chloro-pyridine (868 mg, 4.51 mmol) and Cs2CO3 (2.94 g, 9.03 mmol). The mixture was heated at 100° C. for 24 h. Then, the reaction was cooled to room temperature. Water was added and the aqueous phase was extracted 3 times with ethyl acetate. The combined organic phases were dried, filtered and concentrated under reduce pressure. The crude was purified by column chromatography (from 100% cyclohexane to 100% AcOEt) to afford the title compound as white solid (206 mg, 25% yield).

¹H NMR (300 MHZ, DMSO-d6) δ=8.26 (d, J=5.7 Hz, 1H), 7.60 (d, J=2.5 Hz, 1H), 7.54 (brs, 2H), 7.38 (d, J=8.6 Hz, 1H), 7.05 (dd, J=8.6, 2.5 Hz, 1H), 6.96 (d, J=2.0 Hz, 1H), 6.92(dd, J=5.7, 2.3 Hz, 1H).

LC-MS: method X, RT=2.33 min (5 min run), MS (ESI) m/z: 278 (M+H)⁺; 276 (M−H)⁻

### Step 2: (1R,2S,6R,7S)-4-[6-[(2-chloro-4-pyridyl)oxy]-1,3-benzothiazol-2-yl]-4-azatricyclo [5.2.1.02,6]dec-8-ene-3,5-dione

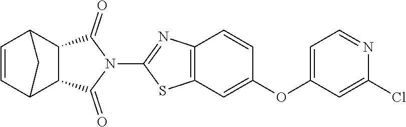 [SMILES: O=C1[C@@H]2C3C=CC(C3)[C@@H]2C(=O)N1c1nc2ccc(Oc3ccnc(Cl)c3)cc2s1]

To a solution of (1R,2S,6R,7S)-4-oxatricyclo[5.2.1.02,6]dec-8-ene-3,5-dione (121 mg, 0.74 mmol) in THF (3 mL) was added 6-[(2-chloro-4-pyridyl)oxy]-1,3-benzothiazol-2-amine (206 mg, 0.74 mmol). The reaction was heated under reflux. After 16 h, the observed precipitate was filtered, washed with THF and ACN and dried to afford the title compound as white solid (182 mg, 58%).

¹H NMR (300 MHZ, CDCl₃): 8.25 (d, J=5.6 Hz, 1H), 8.14 (d, J=8.8 Hz, 1H), 7.59 (d, J=2.3 Hz, 1H), 7.23 (dd, J=8.8, 2.3 Hz, 1H), 6.86-6.78 (m, 2H), 6.28 (s, 2H), 3.59 (s, 2H), 3.57 (s, 2H), 1.84 (d, J=8.9 Hz, 1H), 1.67 (d, J=8.9 Hz, 1H).

LC-MS: method X, RT=2.90 min (5 min run), MS (ESI) m/z: 424 (M+H)⁺

HRMS (ES+) calculated for C21H15ClN303S [M+H]⁺: 424.0523 found 424.0526

### Example 34: (1R,2S,6R,7S)-4-[6-[[6-(trifluoromethyl)-2-pyridyl]oxy]-1,3-benzothiazol-2-yl]-4-azatricyclo[5.2.1.02,6]dec-8-ene-3,5-dione

### Step 1:6-[[6-(trifluoromethyl)-2-pyridyl]oxy]-1,3-benzothiazol-2-amine

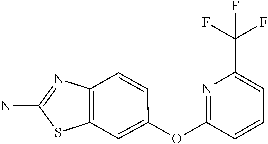 [SMILES: Nc1nc2ccc(Oc3cccc(C(F)(F)F)n3)cc2s1]

To a solution of 2-amino-1,3-benzothiazol-6-ol (500 mg, 3.01 mmol) in DMF (7 mL) was added 2-chloro-6-(trifluoromethyl)pyridine (819 mg, 4.51 mmol) and Cs₂CO₃ (2.94 g, 9.03 mmol). The reaction was stirred at 100° C. After 24 h, water and ethyl acetate were added and the aqueous phase was extracted 3 times with ethyl acetate. The combined organic phases were dried, filtered and concentrated under reduce pressure. The crude was purified by column chromatography (from 80/20 cyclo/AcOEt to 100% AcOEt) to afford the title compound as yellow oil (427 mg, 45.6%).

¹H NMR (300 MHZ, CDCl₃) δ=7.82(t, J=11.1 Hz, 1H), 7.52(d, J=8.6 Hz, 1H), 7.42(d, J=2.1 Hz, 1H), 7.38 (d, J=7.2 Hz, 1H), 7.10 (dd, J=8.6, 2.5 Hz, 1H), 7.00 (d, J=8.4 Hz, 1H), 6.03 (brs, 2H).

LC-MS: method X, RT=2.63 min (5 min run), MS (ESI) m/z: 312(M+H)⁺; 310 (M−H)⁻

### Step 2: (1R,2S,6R,7S)-4-[6-[[6-(trifluoromethyl)-2-pyridyl]oxy]-1,3-benzothiazol-2-yl]-4-azatricyclo[5.2.1.02,6]dec-8-ene-3,5-dione

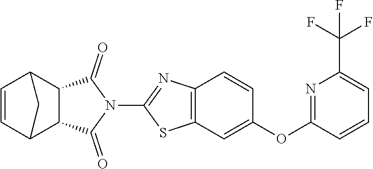 [SMILES: O=C1[C@@H]2C3C=CC(C3)[C@@H]2C(=O)N1c1nc2ccc(Oc3cccc(C(F)(F)F)n3)cc2s1]

To a solution of (1R,2S,6R,7S)-4-oxatricyclo[5.2.1.02,6]dec-8-ene-3,5-dione (140 mg, 0.85 mmol) in THF (2 mL) was added 6-[[6-(trifluoromethyl)-2-pyridyl]oxy]-1,3-benzothiazol-2-amine (265 mg, 0.85 mmol). The mixture was heated under reflux. After 24 h, the solvent was evaporated and the crude was purified by column chromatography (from 100% cyclohexane to 90/10 AcOEt/methanol) to afford the title compound as white solid (309 mg, 80%).

¹H NMR (300 MHZ, CDCl₃) δ=8.10 (d, J=8.9 Hz, 1H), 7.89-7.83 (m, 1H), 7.71 (d, J=2.2 Hz, 1H), 7.41 (d, J=7.3 Hz, 1H), 7.31 (dd, J=8.9, 2.4 Hz, 1H), 7.09 (d, J=8.4 Hz, 1H), 6.29 (t, J=1.6 Hz, 2H), 3.62-3.52(m, 4H), 1.86-1.81 (dt, J=8.9, 1.5 Hz, 1H), 1.67-1.64 (m, 1H).

LC-MS: method X, RT=13.43 min (30 min run), MS (ESI) m/z: 458 (M+H)⁺

HRMS (ES+) calculated for C22H15F3N303S [M+H]⁺: 458.0786 found 458.0773

### Example 35:3-[[6-[[5-(trifluoromethyl)-2-pyridyl]oxy]-1,3-benzothiazol-2-yl]carbamoyl]bicyclo[2.2.1]hept-5-ene-2-carboxylic acid

### Step 1:6-[[5-(trifluoromethyl)-2-pyridyl]oxy]-1,3-benzothiazol-2-amine

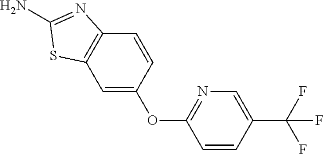 [SMILES: Nc1nc2ccc(Oc3ccc(C(F)(F)F)cn3)cc2s1]

To a solution of 2-amino-1,3-benzothiazol-6-ol (500 mg, 3.01 mmol) in DMF (7 mL) was added 2-chloro-5-(trifluoromethyl)pyridine (819 mg, 4.51 mmol) and Cs₂CO₃ (2.94 g, 9.03 mmol). The reaction was stirred at 100° C. After 24 h, water and ethyl acetate were added and the aqueous phase was extracted 3 times with ethyl acetate. The combined organic phases were dried, filtered and concentrated under reduce pressure. The crude was purified by column chromatography (from 80/20 cyclo/AcOEt to 100% AcOEt) to afford the title compound as orange oil (364.5 mg, 39%).

¹H NMR (300 MHZ, CDCl₃) δ=8.43 (s, 1H), 7.90 (dd, J=8.7, 2.5 Hz, 1H), 7.56 (d, J=8.7 Hz, 1H), 7.40 (d, J=2.4 Hz, 1H), 7.10 (dd, J=8.7, 2.4 Hz, 1H), 7.02(d, J=8.7 Hz, 1H), 6.00 (brs, 2H).

LC-MS: method X, RT=2.66 min (5 min run), MS (ESI) m/z: 312(M+H)⁺; 310 (M−H)⁻

### Step 2:3-[[6-[[5-(trifluoromethyl)-2-pyridyl]oxy]-1,3-benzothiazol-2-yl]carbamoyl]bicyclo[2.2.1]hept-5-ene-2-carboxylic acid

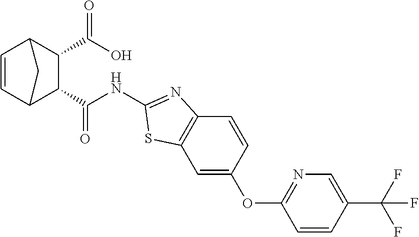 [SMILES: O=C(Nc1nc2ccc(Oc3ccc(C(F)(F)F)cn3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (148 mg, 0.90 mmol) in THF (2 mL) was added 6-[[5-(trifluoromethyl)-2-pyridyl]oxy]-1,3-benzothiazol-2-amine (281 mg, 0.90 mmol). The mixture was heated under reflux. After 16 h, the observed precipitate was filtered, washed with THF and can and dried to afford the title acid compound as white solid (155 mg, 36%). The filtrate was evaporated under reduce pressure and purified by column chromatography (from 100% cyclohexane to 90/10 AcOEt/methanol) to afford the title succinimide compound as white solid (146.5 mg, 36%).

¹H NMR (300 MHZ, DMSO-d6) δ=12.29 (brs, 1H), 11.88 (brs, 1H), 8.56 (m, 1H), 8.23 (dd, J=9.1, 2.3 Hz, 1H), 7.86 (d, J=2.3 Hz, 1H), 7.74 (d, J=8.8 Hz, 1H), 7.25 (d, J=8.7 Hz, 1H), 7.24 (dd, J=8.7, 2.5 Hz, 1H), 6.16 (m, 2H), 3.47 (dd, J=10.0, 3.1 Hz, 1H), 3.38 (m, 1H), 3.13 (s, 1H), 3.08 (s, 1H), 1.34 (m, 2H).

LC-MS: method X, RT=12.02 min (30 min run), MS (ESI) m/z: 476 (M+H)⁺; 474 (M−H)⁻

HRMS (ES+) calculated for C22H17F3N304S [M+H]⁺: 476.0892 found 476.0877

### Example 36: (1R,2S,6R,7S)-4-[6-[[5-(trifluoromethyl)-2-pyridyl]oxy]-1,3-benzothiazol-2-yl]-4-azatricyclo[5.2.1.02,6]dec-8-ene-3,5-dione

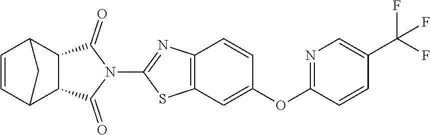 [SMILES: O=C1[C@@H]2C3C=CC(C3)[C@@H]2C(=O)N1c1nc2ccc(Oc3ccc(C(F)(F)F)cn3)cc2s1]

To a solution of (1R,2S,6R,7S)-4-oxatricyclo[5.2.1.02,6]dec-8-ene-3,5-dione (148 mg, 0.90 mmol) in THF (2 mL) was added 6-[[5-(trifluoromethyl)-2-pyridyl]oxy]-1,3-benzothiazol-2-amine (compound of step 1 of example 31, 281 mg, 0.90 mmol). The mixture was heated under reflux. After 16 h. the observed precipitate was filtered, washed with THF and ACN and dried to afford the title acid compound as white solid (155 mg, 36%). The filtrate was evaporated under reduce pressure and purified by column chromatography (from 100% cyclohexane to 90/10 AcOEt/methanol) to afford the title succinimide compound as white solid (146.5 mg, 36%).

¹H NMR (300 MHZ, CDCl₃) δ=8.44-8.41 (m, 1H), 8.12(d, J=8.9 Hz, 1H), 7.95-7.91 (dd, J=8.6, 2.6 Hz, 1H), 7.66 (d, J=2.2 Hz, 1H), 7.30-7.26 (dd, J=8.9, 2.4 Hz, 1H), 7.05 (d, J=8.6 Hz, 1H), 6.28 (t, J=1.5 Hz, 2H), 3.61-3.53 (m, 4H), 1.82(d, J=8.9 Hz, 1H), 1.65 (d, J=8.9 Hz, 1H).

LC-MS: method X, RT=3.14 min (5 min run), MS (ESI) m/z: 458 (M+H)⁺

HRMS (ES+) calculated for C22H15F3N303S [M+H]⁺: 458.0786 found 458.0773

### Example 37:3-[[6-[(5-bromo-2-pyridyl)oxy]-1,3-benzothiazol-2-yl]carbamoyl]bicyclo [2.2.1]hept-5-ene-2-carboxylic acid

### Step 1:6-[(5-bromo-2-pyridyl)oxy]-1,3-benzothiazol-2-amine

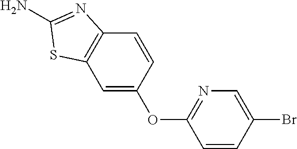 [SMILES: Nc1nc2ccc(Oc3ccc(Br)cn3)cc2s1]

To a solution of 2-amino-1,3-benzothiazol-6-ol (200 mg, 1.20 mmol) in DMF (5 mL) was added 5-bromo-2-chloro-pyridine (232 mg, 1.20 mmol) and Cs₂CO₃ (1.18 g, 3.61 mmol). The reaction was stirred at 100° C. After 48 h, water and ethyl acetate were added and the aqueous phase was extracted 3 times with ethyl acetate. The combined organic phases were dried, filtered and concentrated under reduce pressure. The crude was purified by column chromatography (from 80/20 cyclo/AcOEt to 100% AcOEt) to afford the title compound as white solid (33.5, 9%).

¹H NMR (300 MHZ, CDCl₃) δ=8.20 (dd, J=2.5, 0.5 Hz, 1H), 7.75 (dd, J=8.7, 2.5 Hz, 1H), 7.50 (d, J=8.7 Hz, 1H), 7.35 (d, J=2.4 Hz, 1H), 7.07-7.03 (dd, J=8.7, 2.4 Hz, 1H), 6.83 (dd, J=8.7, 0.5 Hz, 1H).

LC-MS: method X, RT=2.55 min (5 min run), MS (ESI) m/z: 324 (M+2H)⁺; 320 (M−2H)

### Step 2:3-[[6-[(5-bromo-2-pyridyl)oxy]-1,3-benzothiazol-2-yl]carbamoyl]bicyclo[2.2.1]hept-5-ene-2-carboxylic acid

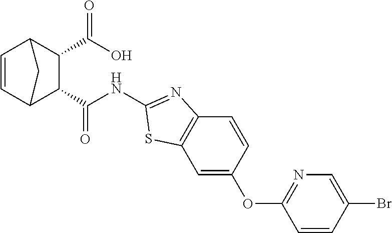 [SMILES: O=C(Nc1nc2ccc(Oc3ccc(Br)cn3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (14 mg, 0.09 mmol) in THF (2 mL) was added 6-[(5-bromo-2-pyridyl)oxy]-1,3-benzothiazol-2-amine (27.5 mg, 0.09 mmol). The mixture was heated under reflux. After 48 h. the observed precipitate was filtered, washed with THF and ACN and dried to afford the title acid compound as white solid (7.8 mg, 20%).

¹H NMR (300 MHZ, DMSO-d6) δ=12.26 (brs, 1H), 11.88 (brs, 1H), 8.26 (dd, J=2.6, 0.6 Hz, 1H), 8.05 (dd, J=8.7, 2.6 Hz, 1H), 7.76 (d, J=2.4 Hz, 1H), 7.73-7.70 (d, J=8.7 Hz, 1H), 7.19 (dd, J=8.7, 2.5 Hz, 1H), 7.07-7.04 (dd, J=8.7, 0.6 Hz, 1H), 6.16 (m, 2H), 3.47 (dd, J=10.0, 3.2 Hz, 1H), 3.38 (dd, J=10.0, 3.2 Hz, 1H), 3.13 (s, 1H), 3.08 (s, 1H), 1.34 (m, 2H).

LC-MS: method X, RT=11.42 min (30 min run), MS (ESI) m/z: 488 (M+2H)⁺; 484 (M−2H)⁻

HRMS (ES+) calculated for C21H15BrN3O3S [M+H]⁺: 486.0123 found 486.0126

### Example 38:3-[(6-nitro-1,3-benzothiazol-2-yl)carbamoyl]bicyclo[2.2.1]hept-5-ene-2-carboxylic acid

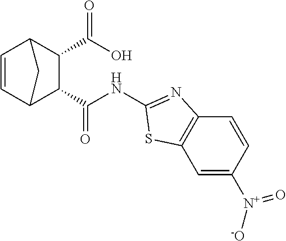

To a solution of (1R,2S,6R,7S)-4-oxatricyclo[5.2.1.02,6]dec-8-ene-3,5-dione (115 mg, 0.7 mmol) in THF (1.5 mL) was added 6-nitro-1,3-benzothiazol-2-amine (115 g, 0.7 mmol). The mixture was stirred under reflux overnight. Then, the observed precipitate was filtrated, washed with ACN and THF and dried to afford the title compound as beige solid (31 mg, 12%).

¹H NMR (300 MHZ, DMSO-d6) δ=12.67 (brs, 1H), 11.94 (brs, 1H), 9.01 (d, J=2.3 Hz, 1H), 8.26 (dd, J=9.0, 2.4 Hz, 1H), 7.84 (d, J=9.0 Hz, 1H), 6.21-6.10 (m, 2H), 3.49 (dd, J=10.0, 2.8 Hz, 1H), 3.42(dd, J=10.0, 2.8 Hz, 1H), 3.15 (s, 1H), 3.10 (s, 1H), 1.36 (m, 2H).

LC-MS: method X, RT=3.88 min (10 min run), MS (ESI) m/z: 360 (M+H)⁺; 358 (M−H)⁻

HRMS (ES+) calculated for C16H14N305S [M+H]⁺: 360.0654 found 360.0656

### Example 39:3 [(6-methylsulfonyl-1,3-benzothiazol-2-yl)carbamoyl]bicyclo[2.2.1]hept-5-ene-2-carboxylic acid

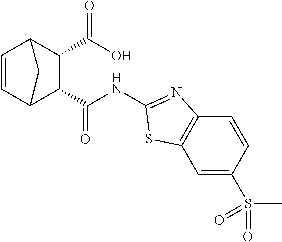 [SMILES: CS(=O)(=O)c1ccc2nc(NC(=O)[C@@H]3C4C=CC(C4)[C@@H]3C(=O)O)sc2c1]

Commercially available. Bought from Vitas-M Laboratory.

### Example 40:3-[(6-cyano-1,3-benzothiazol-2-yl)carbamoyl]bicyclo[2.2.1]hept-5-ene-2-carboxylic acid

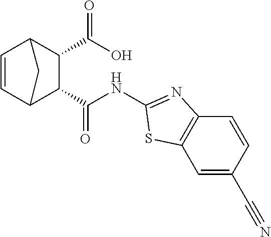 [SMILES: N#Cc1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (115 mg, 0.7 mmol) in THF (1.5 mL) was added 2-amino-1,3-benzothiazole-5-carbonitrile (123 mg, 0.7 mmol). The mixture was stirred under reflux overnight. Then, the observed precipitate was filtrated, washed with ACN and THF and dried to afford the title compound as yellow solid (40 mg, 17%).

¹H NMR (300 MHZ, DMSO-d6) δ=12.58 (s, 1), 11.82(s, 1H), 8.53 (s, 1H), 7.84-7.79 (m, 2H), 6.24-6.01 (m, 2H), 3.48 (dd, J=10.0, 3.4 Hz, 1H), 3.41 (dd, J=10.0, 3.4 Hz, 1H), 3.14 (s, 1H), 3.10 (s, 1H), 1.34 (q, J=8.6 Hz, 2H).

LC-MS: method X, RT=4.14 min (10 min run), MS (ESI) m/z: 340 (M+H)⁺; 338 (M−H)⁻

HRMS (ES+) calculated for C17H14N303S [M+H]⁺: 340.0756 found 340.0746.

### Example 41:3-[(6-methoxycarbonyl-1,3-benzothiazol-2-yl)carbamoyl]bicyclo[2.2.1]hept-5-ene-2-carboxylic acid

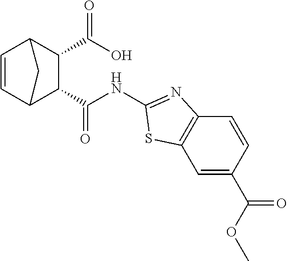 [SMILES: COC(=O)c1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (115 mg, 0.7 mmol) in THF (1.5 mL) was added methyl 2-amino-1,3-benzothiazole-6-carboxylate (146 mg, 0.7 mmol). The mixture was stirred under reflux overnight. Then, the observed precipitate was filtrated, washed with ACN and THF and dried to afford the title compound as beige solid (162 mg, 62%).

¹H NMR (300 MHz, DMSO-d6) δ=12.48 (s, 1), 11.87 (s 1H), 8.59 (d, J=1.5 Hz, 1H), 7.98 (dd, J=8.5, 2.0 Hz, 1H), 8.45 (d, J=8.5 Hz, 1H), 6.22-6.10 (m, 2H), 3.86 (s, 3H), 3.47 (dd, J=10.2, 3.2 Hz, 1H), 3.38 (dd, J=10.3, 3.2 Hz, 1H), 3.13 (s, 1H), 3.08 (s, 1H), 1.41-1.26 (m, 2H).

LC-MS: method X, RT=3.58 min (10 min run), MS (ESI) m/z: 373 (M+H)⁺; 371 (M−H)⁻

HRMS (ES+) calculated for C18H17N2O5S [M+H]⁺: 373.0858 found 373.0859

### Example 42:3-[(6-fluoro-1,3-benzothiazol-2-yl)carbamoyl]bicyclo[2.2.1]hept-5-ene-2-carboxylic acid

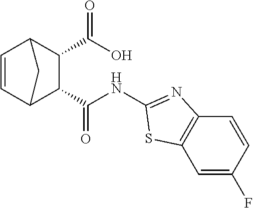 [SMILES: O=C(Nc1nc2ccc(F)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (115 mg, 0.7 mmol) in THF (1.5 mL) was added 5-fluoro-1,3-benzothiazol-2-amine (118 mg, 0.7 mmol).

The mixture was stirred under reflux. After 6 h, the mixture was evaporated under reduce pressure and solubilized in ACN. The observed precipitate was filtrated, washed with ACN and THF and dried to afford the title compound as brown solid (87 mg, 37%).

¹H NMR (300 MHZ, DMSO-d6) δ=12.28 (brs, 1H), 11.87 (brs, 1H), 7.84 (dd, J=8.2, 2.7 Hz, 1H), 7.70 (dd, J=8.8, 4.8 Hz, 1H), 7.25 (td, J=9.2, 2.7 Hz, 1H), 6.19-6.12(m, 2H), 3.46 (dd, J=10.2, 3.2 Hz, 1H), 3.38 (dd, J=10.1, 3.3 Hz, 1H), 3.13 (s, 1H), 3.08 (s, 1H), 1.33 (dd, J=17.2, 8.2 Hz, 2H).

LC-MS: method X, RT=3.77 min (5 min run), MS (ESI) m/z: 333 (M+H)⁺; 331 (M−H)⁻

HRMS (ES+) calculated for C16H14N2O3SF [M+H]⁺: 333.0709 found 333.0729

### Example 43:3-[[6-(trifluoromethyl)-1,3-benzothiazol-2-yl]carbamoyl]bicyclo[2.2.1]hept-5-ene-2-carboxylic acid

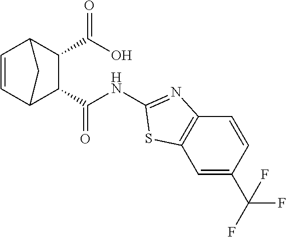 [SMILES: O=C(Nc1nc2ccc(C(F)(F)F)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (115 mg, 0.7 mmol) in THF (1.5 mL) was added 6-(trifluoromethyl)-1,3-benzothiazol-2-amine (153 mg, 0.7 mmol). The mixture was stirred under reflux overnight. The observed precipitate was filtrated, washed with ACN and THF and dried to afford the title compound as white solid (127 mg, 48%).

¹H NMR (300 MHZ, DMSO-d6) δ=12.51 (s, 1H), 11.92(s, 1H), 8.44 (s, 1H), 7.8 (d, J=8.5 Hz, 1H), 7.71 (dd, J=8.5, 1.5 Hz, 1H), 6.20 (dd, J=5.6, 2.9 Hz, 1H), 6.14 (dd, J=5.7, 2.9 Hz, 1H), 3.5 (dd, J=10.2, 3.2 Hz, 1H), 3.40 (dd, J=10.2, 3.2 Hz, 1H), 3.14 (s, 1H), 3.09 (s, 1H), 1.41-1.28 (m, 2H).

LC-MS: method X, RT=4.84 min (10 min run), MS (ESI) m/z: 383 (M+H)⁺; 381 (M−H)⁻

HRMS (ES+) calculated for C17H14N2O3F3S [M+H]⁺: 383.0677 found 383.0657

### Example 44:3-[[6-(trifluoromethoxy)-1,3-benzothiazol-2-yl]carbamoyl]bicyclo[2.2.1]hept-5-ene-2-carboxylic acid

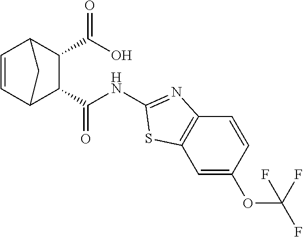 [SMILES: O=C(Nc1nc2ccc(OC(F)(F)F)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (115 mg, 0.7 mmol) in THF (1.5 mL) was added 6-(trifluoromethoxy)-1,3-benzothiazol-2-amine (164 mg, 0.7 mmol). The mixture was stirred under reflux. After 4 h, the observed precipitate was filtrated, washed with ACN and THF and dried to afford the title compound as white solid (140 mg, 50%).

¹H NMR (300 MHZ, DMSO-d6) δ=12.38 (s, 1H), 11.89 (s, 1H), 8.07 (d, J=2.2 Hz, 1H), 7.77 (d, J=8.6 Hz, 1H), 7.38 (ddd, J=8.7, 2.3, 0.9 Hz, 1H), 6.22-6.13 (m, 2H), 3.47 (dd, J=10.2, 3.4 Hz, 1H), 3.39 (dd, J=10.2, 3.3 Hz, 1H), 3.13 (s, 1H), 3.09 (s, 1H), 1.41-1.28 (m, 2H).

LC-MS: method X, RT=4.90 min (10 min run), MS (ESI) m/z: 399 (M+H)⁺; 397 (M−H)⁻

HRMS (ES+) calculated for C17H14N2O4F3S [M+H]⁺: 399.0626 found 399.0630.

### Example 45: (1R,2S,6R,7S)-4-[6-(trifluoromethoxy)-1,3-benzothiazol-2-yl]-4-azatricyclo[5.2.1.02,6]dec-8-ene-3,5-dione

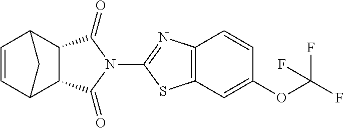 [SMILES: O=C1[C@@H]2C3C=CC(C3)[C@@H]2C(=O)N1c1nc2ccc(OC(F)(F)F)cc2s1]

To a solution of 3-[[6-(trifluoromethoxy)-1,3-benzothiazol-2-yl]carbamoyl]bicyclo[2.2.1]hept-5-ene-2-carboxylic acid (140 mg, 0.35 mmol) in AcOEt (2 mL) was added T3P (50% in AcOEt, 416 μL, 0.70 mmol) and the mixture was stirred at room temperature. After 5 h, EtOAc was added and the organic phase was washed with water twice. The organic layer was dried, filtered and concentrated under reduce pressure. The crude was purified by column chromatography (from 100% cyclohexane to 50/50 cyclohexane/AcOEt) to afford the title compound as white solid (95 mg, 71%).

¹H NMR (300 MHZ, DMSO-d6) δ=8.26 (dd, J=2.2, 0.8 Hz, 1H), 8.09 (d, J=8.9 Hz, 1H), 7.52(ddd, J=8.9, 2.2, 0.8 Hz, 1H), 6.21 (t, J=1.8 Hz, 2H), 3.64 (dd, J=2.8, 1.8 Hz, 2H), 3.42-3.36 (m, 2H), 1.69-1.54 (m, 2H).

LC-MS: method X, RT=5.07 min (10 min run), MS (ESI) m/z: 381 (M+H)⁺

### Example 46:3-[(6-chloro-1,3-benzothiazol-2-yl)carbamoyl]bicyclo[2.2.1]hept-5-ene-2-carboxylic acid

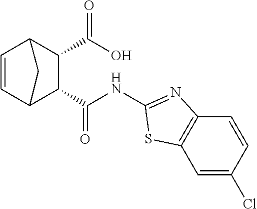 [SMILES: O=C(Nc1nc2ccc(Cl)cc2s1)[C@@H]1C2C=CC(C2)[C@@H]1C(=O)O]

Commercially available. Bought from Vitas-M Laboratory.

### Example 47:3-[(6-bromo-1,3-benzothiazol-2-yl)carbamoyl]bicyclo[2.2.1]hept-5-ene-2-carboxylic acid

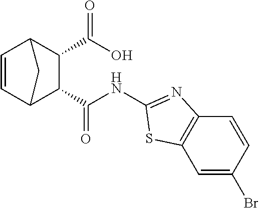 [SMILES: O=C(Nc1nc2ccc(Br)cc2s1)[C@@H]1C2C=CC(C2)[C@@H]1C(=O)O]

Commercially available. Bought from Vitas-M Laboratory.

### Example 48:3-[(5-bromo-1,3-benzothiazol-2-yl)carbamoyl]bicyclo[2.2.1]hept-5-ene-2-carboxylic acid

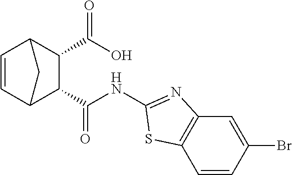 [SMILES: O=C(Nc1nc2cc(Br)ccc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (115 mg, 0.7 mmol) in THF (1.5 mL) was added 5-bromo-1,3-benzothiazol-2-amine (160 mg, 0.7 mmol). The mixture was stirred under reflux. After 3 h, the observed precipitate was filtrated, washed with ACN and THF and dried to afford the title compound as white solid (120 mg, 44%).

¹H NMR (300 MHZ, DMSO-d6) δ=12.34 (brs, 1H), 11.88 (brs, 1H), 8.20 (s, 1H), 8.61 (d, J=8.6 Hz, 1H), 7.53 (d, J=8.5 Hz, 1H), 6.15 (br s, 2H), 3.46 (dd, J=10.0, 2.9 Hz, 1H), 3.37 (dd, J=10.0, 2.9 Hz, 1H), 3.12(s, 1H), 3.07 (s, 1H), 1.33 (dd, J=17.1, 8.2 Hz, 2H).

LC-MS: method X, RT=4.20 min (10 min run), MS (ESI) m/z: 395 (M+H)⁺; 393 (M−H)⁻

HRMS (ES+) calculated for C16H14N2O3SBr [M+H]⁺: 392.9909 found 392.9906

### Example 49:3-[(6-iodo-1,3-benzothiazol-2-yl)carbamoyl]bicyclo[2.2.1]hept-5-ene-2-carboxylic acid

### Step 1:3-[(6-iodo-1,3-benzothiazol-2-yl)carbamoyl]bicyclo[2.2.1]hept-5-ene-2-carboxylic acid

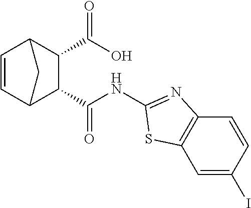 [SMILES: O=C(Nc1nc2ccc(I)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (101 mg, 0.6 mmol) in THF (3 mL) was added 6-iodo-1,3-benzothiazol-2-amine (170 mg, 0.6 mmol). The mixture was stirred under reflux. After 50 h, the observed precipitate was filtrated, washed with THF and dried to afford the title compound as grey solid (197 mg, 73%).

¹H NMR (300 MHZ, DMSO-d6) δ=12.33 (brs, 1H), 11.97 (brs, 1H), 8.33 (d, J=1.6 Hz, 1H), 7.69 (dd, J=8.4 Hz, J=1.7 Hz, 1H), 7.50 (d, J=8.4 Hz, 1H), 6.20-6.11 (m, 2H), 3.47 (dd, J=10.1 Hz, J=3.1 Hz, 1H), 3.37 (dd, J=10.1 Hz, J=3.1 Hz, 1H), 3.12(s, 1H), 3.08 (s, 1H), 1.34 (q, J=8.5 Hz, 2H).

LC-MS: method X, RT=2.70 min (5 min run), MS (ESI) m/z: 441 (M+H)⁺; 439 (M−H)⁻

HRMS (ES+) calculated for C16H14N2O3SI [M+H]⁺: 440.9770 found 440.9785

### Example 50:3-[(6-hydroxy-1,3-benzothiazol-2-yl)carbamoyl]bicyclo[2.2.1]hept-5-ene-2-carboxylic acid

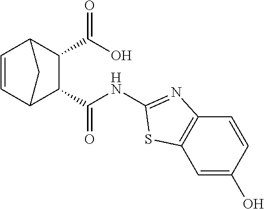 [SMILES: O=C(Nc1nc2ccc(O)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (150 mg, 0.91 mmol) in THF (1 mL) was added 2-amino-1,3-benzothiazol-6-ol (152 mg, 0.91 mmol). The mixture was stirred under reflux overnight. Then, the observed precipitate was filtrated, washed with ACN and THF and dried to afford the title compound as grey solid (85 mg, 28%).

¹H NMR (300 MHZ, DMSO-d6) δ=12.03 (s, 1H), 11.84 (s, 1H), 9.47 (s, 1H), 7.50 (d, J=8.5 Hz, 1H), 7.22(d, J=2.2 Hz, 1H), 6.86 (dd, J=8.6, 2.3 Hz, 1H), 6.22-6.09 (m, 2H), 3.52-3.27 (m, 2H), 3.12(s, 1H), 3.07 (s, 1H), 1.36 (d, J=8.0 Hz, 1H), 1.30 (d, J=8.1 Hz, 1H)

LC-MS: method X, RT=1.98 min (5 min run), MS (ESI) m/z: 331 (M+H)⁺; 329 (M−H)⁻

HRMS (ES+) calculated for C16H15N2O4S [M+H]⁺: 331.0753 found 331.0773

### Example 51:3-[(6-phenyl-1,3-benzothiazol-2-yl)carbamoyl]-7-oxabicyclo[2.2.1]hept-5-ene-2-carboxylic acid

### Step 1:6-phenyl-1,3-benzothiazol-2-amine

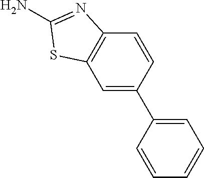 [SMILES: Nc1nc2ccc(-c3ccccc3)cc2s1]

In a sealed tube were introduced phenylboronic acid (274 mg, 2.25 mmool), 6-bromo-1,3-benzothiazol-2-amine (344 mg, 1.5 mmol), Pd(dppf)Cl₂(220 mg, 0.3 mmol), and K₂CO₃ (138 mg, 1 mmol). The mixture was purged with argon for 15 min, prior to the addition of a mixture of Dioxane/water (2 mL, 3:1). The solution was heated at 100° C. under microwave irradiation for 30 min. The reaction mixture was filtrated through cotton and the filtrate was evaporated under reduce pressure. The crude was purified by reverse flash chromatography (C18 from 90/10 water/MeOH to 100% MeOH). Collected fractions were evaporated and resolubilized in ACN. The observed precipitate was filtrated and dried to afford the title compound as beige solid (213 mg, 62%).

¹H NMR (300 MHZ, DMSO-d6) δ=7.97 (d, J=1.7 Hz, 1H), 7.64 (d, J=7.6 Hz, 2H), 7.52 (m, 3H), 7.45-7.35 (m, 3H), 7.3 (t, J=7.6 Hz, 1H).

LC-MS: method X, RT=3.00 min (5 min run), MS (ESI) m/z: 227 (M+H)⁺

### Step 2:3-[(6-phenyl-1,3-benzothiazol-2-yl)carbamoyl]-7-oxabicyclo[2.2.1]hept-5-ene-2-carboxylic acid

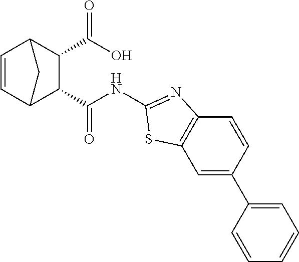 [SMILES: O=C(Nc1nc2ccc(-c3ccccc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (116 mg, 0.7 mmol) in THF (1 mL) was added 6-phenyl-1,3-benzothiazol-2-amine (158 mg, 0.7 mmol). The mixture was stirred under reflux. After 4 h, the observed precipitate was filtrated, washed with ACN and THF and dried to afford the title compound as white solid (184 mg, 67%).

¹H NMR (300 MHZ, DMSO-d6) δ=12.30 (brs, 1H), 11.88 (brs, 1H), 8.25 (s, 1H), 7.72(br s, 4H), 7.47 (t, J=8.3 Hz, 2H), 7.35 (t, J=6.8 Hz, 1H), 6.18 (brs, 2H), 3.49 (d, J=9.3 Hz, 1H), 3.38 (d, J=9.4 Hz, 1H), 3.14 (s, 1H), 3.09 (s, 1H), 1.34 (m, 2H).

LC-MS: method X, RT=2.82 min (5 min run), MS (ESI) m/z: 391 (M+H)⁺; 389 (M−H)⁻

HRMS (ES+) calculated for C22H19N2O3S [M+H]⁺: 391.1116 found 391.1076

### Example 52:3-[(6-cyclohexyl-1,3-benzothiazol-2-yl)carbamoyl]bicyclo[2.2.1]hept-5-ene-2-carboxylic acid

### Step 1:6-cyclohexyl-1,3-benzothiazol-2-amine

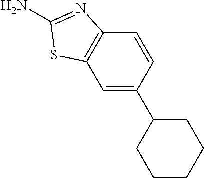 [SMILES: Nc1nc2ccc(C3CCCCC3)cc2s1]

To a solution of 4-cyclohexylaniline (162 mg, 0.92 mmol) in AcOH (0.5 M) was added ammonium thiocyanate (352 mg, 4.62 mmol). The reaction was stirred at room temperature for 90 minutes. Then the mixture was cooled to 0° C., and a solution of bromine (52.1 μL, 1.02 mmol) in AcOH (1 M) was added. The reaction was stirred for 1 h at 0° C. and then allowed to warm to room temperature and stirred overnight. After 16 h, the reaction was poored into cold water and made alkaline (untill pH 10) by the addition of a saturated solution of Na₂CO₃. Ethyl acetate was added and the aqueous phase was extracted 3 times with ethyl acetate. The combined organic phases were dried, filtered and concentrated under reduce pressure. The crude was purified by column chromatography (from 100% cyclohexane to 50/50 cyclohexane/AcOEt) to afford the title compound as white solid (154 mg, 72%).

¹H NMR (300 MHZ, DMSO-d6) δ=7.49 (d, J=1.6 Hz, 1H), 7.32(s, 2H), 7.23 (d, J=8.2 Hz, 1H), 7.06 (dd, J=8.3, 1.8 Hz, 1H), 1.79-1.71 (m, 5H), 1.39-1.22(m, 5H).

LC-MS: method X, RT=3.07 min (5 min run), MS (ESI) m/z: 233 (M+H)⁺

### Step 2: 3-[(6-cyclohexyl-1,3-benzothiazol-2-yl)carbamoyl]bicyclo[2.2.1]hept-5-ene-2-carboxylic acid

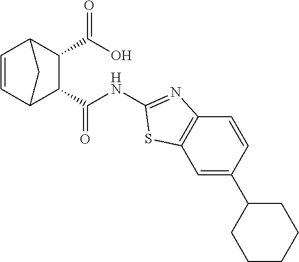 [SMILES: O=C(Nc1nc2ccc(C3CCCCC3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (98.9 mg, 0.603 mmol) in THF (3 mL) was added 6-cyclohexyl-1,3-benzothiazol-2-amine (140 mg, 0.603 mmol). The mixture was stirred under reflux overnight. Then, the observed precipitate was filtrated, washed with ACN and THF and dried to afford the title compound as white solid (74 mg, 31%).

¹H NMR (300 MHZ, DMSO-d6) δ=12.16 (s, 1H), 11.85 (s, 1H), 7.76 (d, J=1.6 Hz, 1H), 7.60 (d, J=8.3 Hz, 1H), 7.27 (dd, J=8.4, 1.8 Hz, 1H), 6.17 (t, J=1.6 Hz, 2H), 3.47 (dd, J=10.1, 3.1 Hz, 1H), 3.37 (dd, J=10.2, 3.3 Hz, 1H), 3.12(s, 1H), 3.09 (s, 1H), 2.59 (m, 1H), 1.82-1.73 (m, 5H), 1.46-1.34 (m, 7H).

LC-MS: method X, RT=13.35 min (30 min run), MS (ESI) m/z: 397 (M+H)⁺; 395 (M−H)⁻

HRMS (ES+) calculated for C22H25N2O3S [M+H]⁺: 397.1586 found 397.1595

### Example 53:3-[[6-(p-tolyl)-1,3-benzothiazol-2-yl]carbamoyl]bicyclo[2.2.1]hept-5-ene-2-carboxylic acid

### Step 1:6-(p-tolyl)-1,3-benzothiazol-2-amine

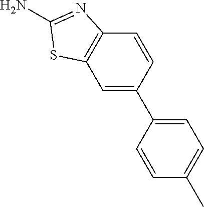 [SMILES: Cc1ccc(-c2ccc3nc(N)sc3c2)cc1]

In a sealed tube were introduced p-tolylboronic acid (102 mg, 0.75 mmool), 6-bromo-1,3-benzothiazol-2-amine (115 mg, 0.5 mmol), Pd(dppf)Cl₂(73.2 mg, 0.1 mmol), and K₂CO₃ (138 mg, 1 mmol). The mixture was purged with argon for 15 min, prior to the addition of a mixture of Dioxane/water (4 mL, 3:1). The solution was heated at 100° C. under microwave irradiation for 30 min. The reaction mixture was filtrated through cotton and the filtrate was evaporated under reduce pressure. The crude was dissolved in EtOAc, and washed twice with water. The organic layer was dried, filtered and concentrated under reduce pressure.

The crude was resolubilized in ACN. The observed precipitate was filtrated and dried to afford the title compound as beige solid (32 mg, 27%).

¹H NMR (300 MHZ, DMSO-d6) δ=7.93 (d, J=1.7 Hz, 1H), 7.54 (d, J=8.2 Hz, 2H), 7.51-7.45 (m, 3H), 7.36 (d, J=8.3 Hz, 1H), 7.23 (d, J=8.2 Hz, 2H), 2.32(s, 3H).

LC-MS: method X, RT=2.88 min (5 min run), MS (ESI) m/z: 241 (M+H)⁺; 239 (M−H)⁻

### Step 2:3-[[6-(p-tolyl)-1,3-benzothiazol-2-yl]carbamoyl]bicyclo[2.2.1]hept-5-ene-2-carboxylic acid

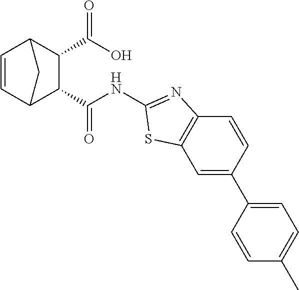 [SMILES: Cc1ccc(-c2ccc3nc(NC(=O)[C@@H]4C5C=CC(C5)[C@@H]4C(=O)O)sc3c2)cc1]

To a solution of (1R,2S,6R,7S)-4-oxatricyclo[5.2.1.02,6]dec-8-ene-3,5-dione (20.5 mg, 0.125 mmol) in THF (3 mL) was added 6-(p-tolyl)-1,3-benzothiazol-2-amine (30 mg, 0.125 mmol). The mixture was stirred under reflux. After 3 h, the observed precipitate was filtrated, washed with ACN and THF and dried to afford the title compound as beige solid (21 mg, 42%).

¹H NMR (300 MHZ, DMSO-d6) δ=12.29 (s, 1H), 11.90 (s, 1H), 8.22(d, J=1.3 Hz, 1H), 7.75 (d, J=8.5 Hz, 1H), 7.69 (dd, J=8.4, 1.7 Hz, 1H), 7.62(d, J=8.1 Hz, 2H), 7.27 (d, J=8.1 Hz, 2H), 6.17 (brs, 2H), 3.49 (dd, J=10.2, 3.2 Hz, 1H), 3.38 (dd, J=10.1, 3.1 Hz, 1H), 3.14 (s, 1H), 3.09 (s, 1H), 2.35 (s, 3H), 1.35 (q, J=8.2 Hz, 2H).

LC-MS: method X, RT=3.00 min (5 min run), MS (ESI) m/z: 405 (M+H)⁺; 403 (M−H)⁻

HRMS (ES+) calculated for C23H21N2O3S [M+H]⁺: 405.1273 found 405.1273

### Example 54:3-[[6-(4-methoxyphenyl)-1,3-benzothiazol-2-yl]carbamoyl]bicyclo[2.2.19hept-5-ene-2-carboxylic acid

### Step 1:6-(4-methoxyphenyl)-1,3-benzothiazol-2-amine

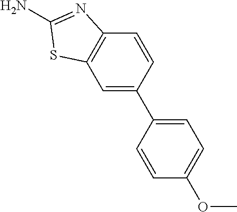 [SMILES: COc1ccc(-c2ccc3nc(N)sc3c2)cc1]

In a sealed tube were introduced (4-methoxyphenyl) boronic acid (160 mg, 1.05 mmool), 6-bromo-1,3-benzothiazol-2-amine (160 mg, 0.7 mmol), Pd(dppf)Cl₂(51.2 mg, 0.07 mmol), and K₂CO₃ (193 mg, 1.4 mmol). The mixture was purged with argon for 15 min, prior to the addition of a mixture of Dioxane/water (4 mL, 3:1). The solution was heated at 100° C. under microwave irradiation for 30 min. The reaction mixture was filtrated through cotton and a pad of celite and the filtrate was evaporated under reduce pressure. The crude was dissolved in EtOAc, and washed twice with water. The organic layer was dried, filtered and concentrated under reduce pressure. The crude was resolubilized in ACN. The observed precipitate was filtrated and dried to afford the title compound as light brown solid (110 mg, 61%).

¹H NMR (300 MHZ, DMSO-d6) δ=7.90 (d, J=1.83 Hz, 1H), 7.57 (d, J=8.9 Hz, 2H), 7.47 (s, 2H), 7.45 (dd, J=8.4, 1.9 Hz, 1H), 7.34 (d, J=8.5 Hz, 1H), 7.0 (d, J=8.9 Hz, 2H), 3.78 (s, 3H).

LC-MS: method X, RT=2.63 min (5 min run), MS (ESI) m/z: 257 (M+H)⁺

### Step 2:3-[[6-(4-methoxyphenyl)-1,3-benzothiazol-2-yl]carbamoyl]bicyclo[2.2.1]hept-5-ene-2-carboxylic acid

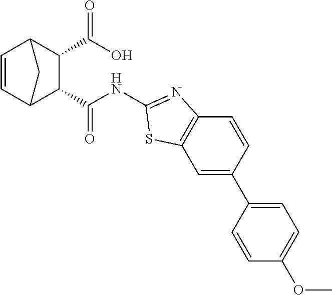 [SMILES: COc1ccc(-c2ccc3nc(NC(=O)[C@@H]4C5C=CC(C5)[C@@H]4C(=O)O)sc3c2)cc1]

To a solution of (1R,2S,6R,7S)-4-oxatricyclo[5.2.1.02,6]dec-8-ene-3,5-dione (70.4 mg, 0.43 mmol) in THF (3 mL) was added 6-(4-methoxyphenyl)-1,3-benzothiazol-2-amine (110 mg, 0.43 mmol). The mixture was stirred under reflux. After 3 h, the observed precipitate was filtrated, washed with ACN and THF and dried to afford the title compound as light beige solid (30 mg, 17%).

¹H NMR (300 MHZ, DMSO-d6) δ=12.27 (s, 1H), 11.85 (s, 1H), 8.17 (s, 1H), 7.75-7.61 (m, 4H), 7.03 (d, J=8.7 Hz, 2H), 6.16 (brs, 2H), 3.79 (s, 3H), 3.47 (dd, J=10.0, 3.1 Hz, 1H), 3.37 (dd, J=10.2, 3.1 Hz, 1H), 3.13 (s, 1H), 3.07 (s, 1H), 1.33 (dd, J=17.7, 8.2 Hz, 2H).

LC-MS: method X, RT=2.78 min (5 min run), MS (ESI) m/z: 421 (M+H)⁺; 419 (M−H)⁻

HRMS (ES+) calculated for C23H21N2O4S [M+H]⁺: 421.1222 found 421.1244

### Example 55:3-[[6-(4-chlorophenyl)-1,3-benzothiazol-2-yl]carbamoyl]bicyclo[2.2.1]hept-5-ene-2-carboxylic acid

### Step 1:6-(4-chlorophenyl)-1,3-benzothiazol-2-amine

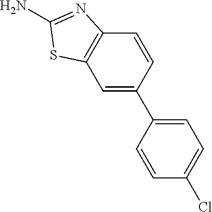 [SMILES: Nc1nc2ccc(-c3ccc(Cl)cc3)cc2s1]

In a sealed tube were introduced (4-chlorophenyl) boronic acid (164 mg, 1.05 mmool), 6-bromo-1,3-benzothiazol-2-amine (160 mg, 0.7 mmol), Pd(dppf)Cl₂(51.2 mg, 0.07 mmol), and K₂CO₃ (193 mg, 1.4 mmol). The mixture was purged with argon for 15 min, prior to the addition of a mixture of Dioxane/water (4 mL, 3:1). The solution was heated at 100° C. under microwave irradiation for 30 min. The reaction mixture was filtrated through cotton and the filtrate was evaporated under reduce pressure. The crude was dissolved in EtOAc, and washed twice with water. The organic layer was dried, filtered and concentrated under reduce pressure. The crude was resolubilized in ACN and the precipiate was filtered. The filtrate was evaporated and was resolubilized in DCM. The observed precipitate was filtrated and dried to afford the title compound as light beige solid (130 mg, 71%).

¹H NMR (300 MHZ, DMSO-d6) δ=7.99 (d, J=1.8 Hz, 1H), 7.67 (dd, J=8.7, 1.9 Hz, 2H), 7.60 (s, 2H), 7.50 (dd, J=8.5, 2.0 Hz, 1H), 7.47 (dd, J=8.7, 1.9 Hz, 2H), 7.37 (d, J=8.4 Hz, 1H).

LC-MS: method X, RT=2.77 min (5 min run), MS (ESI) m/z: 261 (M+H)⁺; 259 (M−H)⁻

### Step 2:3-[[6-(4-chlorophenyl)-1,3-benzothiazol-2-yl]carbamoyl]bicyclo[2.2.1]hept-5-ene-2-carboxylic acid

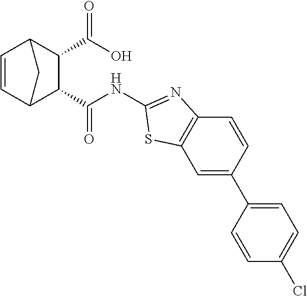 [SMILES: O=C(Nc1nc2ccc(-c3ccc(Cl)cc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (81.8 mg, 0.5 mmol) in THF (3 mL) was added 6-(4-chlorophenyl)-1,3-benzothiazol-2-amine (130 mg, 0.5 mmol). The mixture was stirred under reflux. After 3 h, the observed precipitate was filtrated, washed with ACN and THF and dried to afford the title compound as light beige solid (140 mg, 66%).

¹H NMR (300 MHZ, DMSO-d6) δ=12.39 (s, 1H), 11.91 (s, 1H), 8.28 (d, J=1.3 Hz, 1H), 7.80-7.67 (m, 4H), 7.51 (dt, J=9.4, 2.7 Hz, 2H), 6.19-6.11 (m, 2H), 3.52(dd, J=10.2, 3.2 Hz, 1H), 3.40 (dd, J=10.0, 3.4 Hz, 1H), 3.14 (s, 1H), 3.09 (s, 1H), 1.34 (m, 2H).

LC-MS: method X, RT=3.03 min (5 min run), MS (ESI) m/z: 425 (M+H)⁺; 423 (M−H)⁻

HRMS (ES+) calculated for C22H18N2O3ScL [M+H]⁺: 425.0727 found 425.0736

### Example 56:3-[[6-(3,4-dichlorophenyl)-1,3-benzothiazol-2-yl]carbamoyl]bicycle [2.2.1]hept-5-ene-2-carboxylic acid

### Step 1:6-(3,4-dichlorophenyl)-1,3-benzothiazol-2-amine

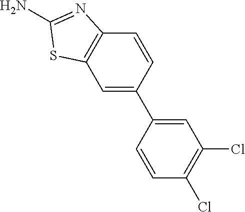 [SMILES: Nc1nc2ccc(-c3ccc(Cl)c(Cl)c3)cc2s1]

In a sealed tube were introduced (3,4-dichlorophenyl) boronic acid (200 mg, 1.05 mmol), 6-bromo-1,3-benzothiazol-2-amine (160 mg, 0.7 mmol), Pd(dppf)Cl₂(51.2 mg, 0.07 mmol), and K₂CO₃ (193 mg, 1.4 mmol). The mixture was purged with argon for 15 min, prior to the addition of a mixture of Dioxane/water (4 mL, 3:1). The solution was heated at 100° C. under microwave irradiation for 30 min. The reaction mixture was filtrated through cotton and a pad of celite and the filtrate was evaporated under reduce pressure. The crude was dissolved in EtOAc, and washed twice with water. The organic layer was dried, filtered and concentrated under reduce pressure. The crude was resolubilized in ACN. The observed precipitate was filtrated, washed with ACN and MeOH and dried to afford the title compound as beige solid (87 mg, 42%).

¹H NMR (300 MHZ, DMSO-d6) δ=8.06 (d, J=1.9 Hz, 1H), 7.92(s, 1H), 7.66 (m, 2H), 7.61 (brs, 2H), 7.56 (dd, J=8.4, 2.2 Hz, 1H), 7.38 (d, J=8.2 Hz, 1H).

LC-MS: method X, RT=3.15 min (5 min run), MS (ESI) m/z: 295 (M+H)⁺; 293 (M−H)⁻

### Step 2:3-[[6-(3,4-dichlorophenyl)-1,3-benzothiazol-2-yl]carbamoyl]bicycle [2.2.1]hept-5-ene-2-carboxylic acid

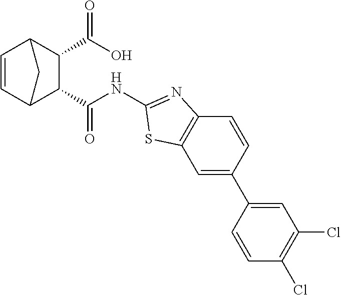 [SMILES: O=C(Nc1nc2ccc(-c3ccc(Cl)c(Cl)c3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (50.1 mg, 0.31 mmol) in THF (1.5 mL) was added 6-(3,4-dichlorophenyl)-1,3-benzothiazol-2-amine (90 mg, 0.31 mmol). The mixture was stirred under reflux overnight. Then, the observed precipitate was filtrated, washed with ACN and THF and dried to afford the title compound as white solid (82 mg, 58%).

¹H NMR (300 MHz, DMSO-d6) δ=12.35 (s, 1H), 11.87 (s, 1H), 8.34 (s, 1H), 8.01 (d, J=1.8 Hz, 1H), 7.65 (s, 2H), 7.74-7.68 (m, 2H), 6.16 (brs, 2H), 3.47 (dd, J=10.1, 3.9 Hz, 1H), 3.37 (dd, J=10.2, 3.2 Hz, 1H), 3.13 (s, 1H), 3.08 (s, 1H), 1.33 (dd, J=17.4, 8.2 Hz, 2H).

LC-MS: method X, RT=3.20 min (5 min run), MS (ESI) m/z: 459 (M+H)⁺; 457 (M−H)⁻

HRMS (ES+) calculated for C22H17N2O3SCI2 [M+H]⁺: 459.0337 found 459.0323

### Examples 57 and 58:3-[[6-[[2-(trifluoromethyl)phenyl]-1,3-benzothiazol-2-yl]carbamoyl]bicyclo[2.2.1]hept-5-ene-2-carboxylic acid/(1R,2S,6R,7S)-4-[6-12-(trifluoromethyl)phenyl]-1,3-benzothiazol-2-yl]-4-azatricyclo[5.2.1.02,6]dec-8-ene-3,5-dione

### Step 1:6-[2-(trifluoromethyl)phenyl]-1,3-benzothiazol-2-amine

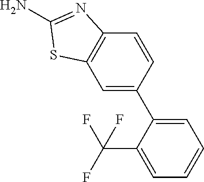 [SMILES: Nc1nc2ccc(-c3ccccc3C(F)(F)F)cc2s1]

In a sealed tube were introduced [2-(trifluoromethyl)phenyl]boronic acid (200 mg, 1.05 mmool), 6-bromo-1,3-benzothiazol-2-amine (160 mg, 0.7 mmol), Pd(dppf)Cl₂(51.2 mg, 0.07 mmol), and K₂CO₃ (193 mg, 1.4 mmol). The mixture was purged with argon for 15 min, prior to the addition of a mixture of Dioxane/water (4 mL, 3:1). The solution was heated at 100° C. under microwave irradiation for 30 min. The reaction mixture was evaporated under reduce pressure and purified by column chromatography (from 100% cyclohexane to 50/50 cyclohexane/AcOEt). A mixture of two products was afforded and purified by reverse column chromatography (from 90/10 water/MeOH to 100% MeOH) to give the title compound as white solid (55 mg, 27%).

LC-MS: method X, RT=4.63 min (10 min run), MS (ESI) m/z: 295 (M+H)⁺; 293 (M−H)⁻

### Step 2

### Example 57 3-[6-[[2-(trifluoromethyl)phenyl]-1,3-benzothiazol-2-yl]carbamoyl]bicyclo[2.2.1]hept-5-ene-2-carboxylic acid

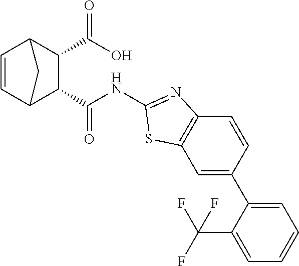 [SMILES: O=C(Nc1nc2ccc(-c3ccccc3C(F)(F)F)cc2s1)[C@@H]1C2C=CC(C2)[C@@H]1C(=O)O]

### Example 58 (1R,2S,6R,7S)-4-[6-[[2-(trifluoromethyl)phenyl]-1,3-benzothiazol-2-yl]-4-azatricyclo [5.2.1.02,6]dec-8-ene-3,5-dione

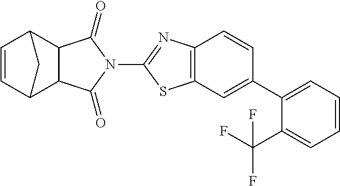 [SMILES: O=C1C2C3C=CC(C3)C2C(=O)N1c1nc2ccc(-c3ccccc3C(F)(F)F)cc2s1]

To a solution of (1R,2S,6R,7S)-4-oxatricyclo[5.2.1.02,6]dec-8-ene-3,5-dione (30.7 mg, 0.19 mmol) in THF (1 mL) was added 6-[[2-(trifluoromethyl)phenyl]-1,3-benzothiazol-2-amine (55.0 mg, 0.19 mmol). The mixture was stirred under reflux. After 6 h, the reaction mixture was evaporated and resolubilized in ACN. The observed precipitate was filtrated, washed with ACN and THF and dried. The precipitate was purified by reverse column chromatography (from 70/30 water/ACN to 100% ACN) to afford both title compounds as white solid (Example 57, 54 mg, 63%; Example 58, 14.0 mg, 17%).

### Example 57

¹H NMR (300 MHZ, DMSO-d6) δ=12.33 (s, 1H), 11.88 (s, 1H), 7.89 (s, 1H), 7.84 (d, J=7.9 Hz, 1H), 7.77-7.67 (m, 2H), 7.61 (t, J=7.5 Hz, 1H), 7.46 (d, J=7.5 Hz, 1H), 7.33 (d, J=7.9 Hz, 1H), 6.16 (brs, 2H), 3.48 (dd, J=10.0, 2.9 Hz, 1H), 3.38 (dd, J=10.0, 2.9 Hz, 1H), 3.13 (s, 1H), 3.08 (s, 1H), 1.40-1.25 (m, 2H).

LC-MS: method X, RT=4.90 min (10 min run), MS (ESI) m/z: 459 (M+H)⁺; 457 (M−H)⁻

HRMS (ES+) calculated for C23H18N2O3F3S [M+H]⁺: 459.0990 found 459.0989

### Example 58

¹H NMR (300 MHz, DMSO-d6) δ=8.10 (d, J=1.7 Hz, 1H) 8.04 (dd, J=8.4, 0.5 Hz, 1H), 7.86 (dd, J=7.6, 0.7 Hz, 1H), 7.75 (t, J=7.7 Hz, 1H), 7.65 (t, J=7.5 Hz, 1H), 7.51-7.43 (m, 2H), 6.22(t, J=1.8 Hz, 2H), 3.65 (dd, J=2.8, 1.5 Hz, 2H), 3.45-3.38 (m, 2H), 1.68-1.59 (m, 2H).

LC-MS: method X, RT=5.52 min (10 min run), MS (ESI) m/z: 441 (M+H)⁺

HRMS (ES+) calculated for C23H16N2O2F3S [M+H]⁺: 441.0888 found 441.0864

### Example 59: 3-[[6-[3-(trifluoromethyl)phenyl]-1,3-benzothiazol-2-yl]carbamoyl]bicyclo[2.2.1]hept-5-ene-2-carboxylic acid

### Step 1:6-[3-(trifluoromethyl)phenyl]-1,3-benzothiazol-2-amine

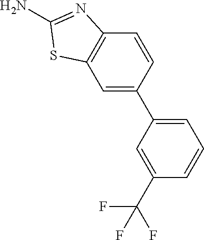 [SMILES: Nc1nc2ccc(-c3cccc(C(F)(F)F)c3)cc2s1]

In a sealed tube were introduced [3-(trifluoromethyl)phenyl]boronic acid (200 mg, 1.05 mmool), 6-bromo-1,3-benzothiazol-2-amine (160 mg, 0.7 mmol), Pd(dppf)Cl₂(51.2 mg, 0.07 mmol), and K₂CO₃ (193 mg, 1.4 mmol). The mixture was purged with argon for 15 min, prior to the addition of a mixture of Dioxane/water (4 mL, 3:1). The solution was heated at 100° C. under microwave irradiation for 30 min. The reaction mixture was evaporated under reduce pressure and purified by column chromatography (from 100% cyclohexane to 50/50 cyclohexane/AcOEt). A mixture of two products was afforded and was solubilized in DCM. The observed precipitate was filtrated to give the title compound as white solid (55 mg, 27%).

¹H NMR (300 MHZ, DMSO-d6) δ=8.10 (d, J=1.9 Hz, 1H), 8.00-7.92(m, 2H), 7.68-7.63 (m, 2H), 7.62-7.56 (m, 3H), 7.41 (d, J=8.4 Hz, 1H).

LC-MS: method X, RT=2.90 min (5 min run), MS (ESI) m/z: 295 (M+H)⁺; 293 (M−H)⁻

### Step 2: 3-[[6-[3-(trifluoromethyl)phenyl]-1,3-benzothiazol-2-yl]carbamoyl]bicyclo[2.2.1]hept-5-ene-2-carboxylic acid

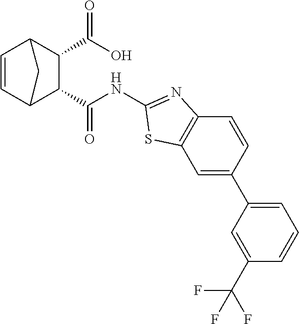 [SMILES: O=C(Nc1nc2ccc(-c3cccc(C(F)(F)F)c3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (30.7 mg, 0.19 mmol) in THF (1 mL) was added 6-[3-(trifluoromethyl)phenyl]-1,3-benzothiazol-2-amine (55.0 mg, 0.19 mmol). The mixture was stirred under reflux overnight. Then, the observed precipitate was washed with THF and ACN and dried to afford both title compounds as white solid (54 mg, 63%).

¹H NMR (300 MHZ, DMSO-d6) δ=12.36 (s, 1H), 11.89 (s, 1H), 8.38 (s, 1H), 8.09-7.93 (m, 2H), 7.89-7.76 (m, 2H), 7.64-7.63 (m, 2H), 6.16 (brs, 2H), 3.48 (dd, J=10.1, 3.2 Hz, 1H), 3.38 (dd, J=10.2, 3.2 Hz, 1H), 3.13 (s, 1H), 3.08 (s, 1H), 1.41-1.24 (m, 2H).

LC-MS: method X, RT=5.08 min (10 min run), MS (ESI) m/z: 459 (M+H)⁺; 457 (M−H)⁻

HRMS (ES+) calculated for C23H18N2O3SF3 [M+H]⁺: 459.0990 found 459.1003

### Example 60:3-[[6-[4-(trifluoromethyl)phenyl]-1,3-benzothiazol-2-yl]carbamoyl]bicyclo[2.2.1]hept-5-ene-2-carboxylic acid

### Step 1:6-[4-(trifluoromethyl)phenyl]-1,3-benzothiazol-2-amine

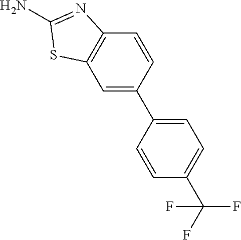 [SMILES: Nc1nc2ccc(-c3ccc(C(F)(F)F)cc3)cc2s1]

In a sealed tube were introduced [4-(trifluoromethyl)phenyl]boronic acid (200 mg, 1.05 mmool), 6-bromo-1,3-benzothiazol-2-amine (160 mg, 0.7 mmol), Pd(dppf)Cl₂(51.2 mg, 0.07 mmol), and K₂CO₃ (193 mg, 1.4 mmol). The mixture was purged with argon for 15 min, prior to the addition of a mixture of Dioxane/water (4 mL, 3:1). The solution was heated at 100° C. under microwave irradiation for 30 min. The reaction mixture was filtrated through cotton and evaporated under reduce pressure. The crude was purified by column chromatography (from 100% cyclohexane to 50/50 cyclohexane/AcOEt). The combined fractions were evaporated and resolubilized in DCM. The observed precipitate was filtrated to afford the title compound as white solid (132 mg, 64%).

¹H NMR (300 MHZ, DMSO-d6) δ=8.09 (d, J=1.8 Hz, 1H), 7.88 (d, J=8.1 Hz, 2H), 7.77 (d, J=8.4 Hz, 2H), 7.64 (s, 2H), 7.59 (dd, J=8.4, 1.8 Hz, 1H), 7.42(d, J=8.4 Hz, 1H).

LC-MS: method X, RT=2.97 min (5 min run), MS (ESI) m/z: 295 (M+H)⁺; 293 (M−H)⁻

### Step 2:3-[[6-[4-(trifluoromethyl)phenyl]-1,3-benzothiazol-2-yl]carbamoyl]bicyclo[2.2.1]hept-5-ene-2-carboxylic acid

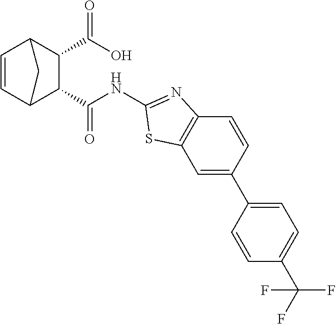 [SMILES: O=C(Nc1nc2ccc(-c3ccc(C(F)(F)F)cc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (45.7 mg, 0.28 mmol) in THF (3 mL) was added 6-[4-(trifluoromethyl)phenyl]-1,3-benzothiazol-2-amine (82.0 mg, 0.28 mmol). The mixture was stirred under reflux overnight. Then, the observed precipitate was washed with THF and ACN and dried to afford the title compound as beige solid (82 mg, 64%).

¹H NMR (300 MHZ, DMSO-d6) δ=12.35 (brs, 1H), 11.89 (brs, 1H), 8.36 (s 1H), 7.95 (d, J=8.2 Hz, 2H), 7.86-7.78 (m, 4H), 6.17 (m, 2H), 3.61 (dd, J=10.0, 3.1 Hz, 1H), 3.48 (dd, J=10.0, 3.2 Hz, 1H), 3.13 (s, 1H), 3.09 (s, 1H), 1.34 (dd, J=17.4, 8.4 Hz, 2H).

LC-MS: method X, RT=3.08 min (5 min run), MS (ESI) m/z: 459 (M+H)⁺; 457 (M−H)⁻

HRMS (ES+) calculated for C23H18N2O3SF3 [M+H]⁺: 459.0990 found 459.0992

### Example 61: (1R,2S,6R,7S)-4-[6-[4-(trifluoromethyl)phenyl]-1,3-benzothiazol-2-yl]-4-azatricyclo[5.2.1.02,6]dec-8-ene-3,5-dione

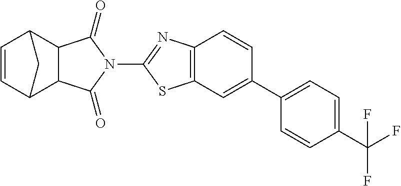 [SMILES: O=C1C2C3C=CC(C3)C2C(=O)N1c1nc2ccc(-c3ccc(C(F)(F)F)cc3)cc2s1]

To a solution of 3-[6-[[4-(trifluoromethyl)phenyl]-1,3-benzothiazol-2-yl]carbamoyl]bicyclo[2.2.1]hept-5-ene-2-carboxylic acid (example 55, 80 mg, 0.17 mmol) in AcOEt (1 mL) was added T3P (50% in AcOEt, 208 μL, 0.35 mmol) and the mixture was stirred at rt. After 5 h, the observed precipitate was removed by filtration. AcOEt was added and the organic phase was washed with water twice. The organic layer was dried, filtered and concentrated under reduce pressure. The crude was purified by column chromatography (from 100% cyclohexane to 50/50 cyclohexane/AcOEt) to afford the title compound as white solid (42 mg, 55%).

¹H NMR (300 MHZ, DMSO-d6) δ=8.54 (d, J=1.6 Hz, 1H), 8.10 (d, J=8.6 Hz, 1H), 7.99 (d, J=8.0 Hz, 2H), 7.91 (dd, J=8.9, 1.9 Hz, 1H), 7.86 (d, J=8.3 Hz, 2H), 6.23 (t, J=1.8 Hz, 2H), 3.65 (dd, J=2.8, 1.5 Hz, 2H), 3.44-3.37 (m, 2H), 1.69-1.58 (m, 2H).

LC-MS: method X, RT=5.72 min (10 min run), MS (ESI) m/z: 441 (M+H)⁺

HRMS (ES+) calculated for C23H16N2O2S [M+H]⁺: 441.0885 found 441.0883

### Example 62:3-[[6-[4-(trifluoromethoxy)phenyl]-1,3-benzothiazol-2-yl]carbamoyl]bicyclo[2.2.1]hept-5-ene-2-carboxylic acid

### Step 1:6-[4-(trifluoromethoxy)phenyl]-1,3-benzothiazol-2-amine

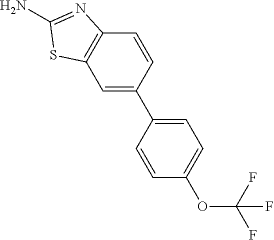 [SMILES: Nc1nc2ccc(-c3ccc(OC(F)(F)F)cc3)cc2s1]

In a sealed tube were introduced [4-(trifluoromethoxy)phenyl]boronic acid (200 mg, 0.48 mmol), 6-bromo-1,3-benzothiazol-2-amine (91.6 mg, 0.4 mmol), Pd(dppf)Cl₂(23.9 mg, 0.04 mmol), and K₂CO₃ (111 mg, 0.8 mmol). The mixture was purged with argon for 15 min, prior to the addition of a mixture of Dioxane/water (4 mL, 3:1). The solution was heated at 100° C. under microwave irradiation for 30 min. The reaction mixture was filtrated through cotton and evaporated under reduce pressure. The crude was purified by column chromatography (from 100% cyclohexane to 50/50 cyclohexane/AcOEt) to afford the title compound as beige solid (77 mg, 62%).

LC-MS: method X, RT=3.03 min (5 min run), MS (ESI) m/z: 311 (M+H)⁺; 309 (M−H)⁻

### Step 2:3-[[6-[4-(trifluoromethoxy)phenyl]-1,3-benzothiazol-2-yl]carbamoyl]bicyclo [2.2.1]hept-5-ene-2-carboxylic acid

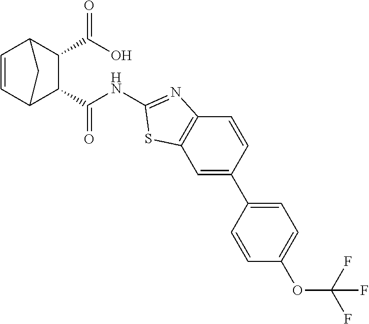 [SMILES: O=C(Nc1nc2ccc(-c3ccc(OC(F)(F)F)cc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (7.40 mg, 0.045 mmol) in THF (3 mL) was added 6-[4-(trifluoromethoxy)phenyl]-1,3-benzothiazol-2-amine (14.0 mg, 0.045 mmol). The mixture was stirred under reflux. After 5 h, the observed precipitate was washed with THF and ACN and dried. The precipitate was further purified by reverse column chromatography (C18, from 90/10 water/ACN to 100 ACN) to give the title compound as white solid (11 mg, 54%).

¹H NMR (300 MHZ, DMSO-d6) δ=8.24 (d, J=1.3 Hz, 1H), 7.83 (d, J=8.6 Hz, 2H), 7.74 (d, J=8.2 Hz, 1H), 7.69 (dd, J=8.3, 1.5 Hz, 1H), 7.44 (d, J=8.6 Hz, 2H), 6.15 (s, 2H), 3.41 (dd, J=10.0, 3.0 Hz, 1H), 3.31 (dd, J=10.1, 3.1 Hz, 1H), 3.14 (s, 1H), 3.09 (s, 1H), 1.37-1.21 (m, 2H).

LC-MS: method X, RT=3.12 min (5 min run), MS (ESI) m/z: 475 (M+H)⁺; 473 (M−H)⁻

HRMS (ES+) calculated for C23H18N2O4F3S [M+H]⁺: 475.0939 found 475.0947

### Example 63: (1R,2S,6R,7S)-4-[6-(4-hydroxyphenyl)-1,3-benzothiazol-2-yl]-4-azatricyclo[5.2.1.02,6]dec-8-ene-3,5-dione

### Step 1:4-(2-amino-1,3-benzothiazol-6-yl)phenol

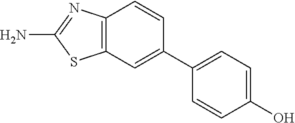 [SMILES: Nc1nc2ccc(-c3ccc(O)cc3)cc2s1]

In a sealed tube were introduced 4-(4,4,5,5-tetramethyl-1,3,2-dioxaborolan-2-yl)phenol (54.8 mg, 0.25 mmol), 6-bromo-1,3-benzothiazol-2-amine (57.0 mg, 0.25 mmol), Pd(dppf)Cl₂(36.4 mg, 0.05 mmol), and K₂CO₃ (68.8 mg, 0.5 mmol). The mixture was purged with argon for 15 min, prior to the addition of a mixture of Dioxane/water (4 mL, 3:1). The solution was heated at 100° C. under microwave irradiation for 1 hour. The reaction mixture was evaporated under reduce pressure. The crude was purified by column chromatography (from 100% cyclohexane to 50/50 cyclohexane/AcOEt) to afford the title compound as beige solid (30 mg, 50%).

LC-MS: method X, RT=2.02 min (5 min run), MS (ESI) m/z: 243 (M+H)⁺; 241 (M−H)⁻

### Step 2:(1R,2S,6R,7S)-4-[6-(4-hydroxyphenyl)-1,3-benzothiazol-2-yl]-4-azatricyclo[5.2.1.02,69dec-8-ene-3,5-dione

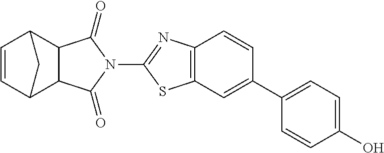 [SMILES: O=C1C2C3C=CC(C3)C2C(=O)N1c1nc2ccc(-c3ccc(O)cc3)cc2s1]

To a solution of (1R,2S,6R,7S)-4-oxatricyclo[5.2.1.02,6]dec-8-ene-3,5-dione (5.42 mg, 0.033 mmol) in THF (1 mL) was added 4-(2-amino-1,3-benzothiazol-6-yl)phenol (8.0 mg, 0.033 mmol). The mixture was stirred under reflux overnight. Then, the reaction mixture was evaporated under reduce pressure and ACN was added. The observed precipitate was washed with THF and ACN and dried to give the title compound as white solid (10 mg, 78%).

¹H NMR (300 MHZ, DMSO-d6) δ=9.62(s, 1H), 8.32(dd, J=2.0, 0.5 Hz, 1H), 8.00 (dd, J=8.7, 0.5 Hz, 1H), 7.76 (dd, J=8.7, 1.9 Hz, 1H), 7.63-7.54 (m, 2H), 6.91-6.84 (m, 2H), 6.24 (t, J=1.7 Hz, 2H), 3.64 (dd, J=2.9, 1.6 Hz, 2H), 3.43-3.37 (m, 2H), 1.64 (d, J=1.8 Hz, 2H).

LC-MS: method X, RT=2.65 min (5 min run), MS (ESI) m/z: 389 (M+H)⁺; 387 (M−H)⁻

HRMS (ES+) calculated for C22H17N2O3S [M+H]⁺: 389.0960 found 389.0970

### Example 64:3-[[6-(4-pyridyl)-1,3-benzothiazol-2-yl]carbamoyl]bicyclo[2.2.1]hept-5-ene-2-carboxylic acid

### Step 1:6-(4-pyridyl)-1,3-benzothiazol-2-amine

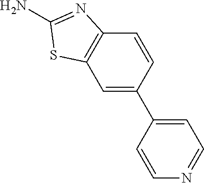 [SMILES: Nc1nc2ccc(-c3ccncc3)cc2s1]

In a sealed tube were introduced 4-pyridylboronic acid;hydrate (148 mg, 1.05 mmol), 6-bromo-1,3-benzothiazol-2-amine (160.0 mg, 0.7 mmol), Pd(dppf)Cl₂(49.1 mg, 0.07 mmol), and K₂CO₃ (193 mg, 1.40 mmol). The mixture was purged with argon for 15 min, prior to the addition of a mixture of Dioxane/water (4 mL, 3:1). The solution was heated at 120° C. under microwave irradiation for 1.5 hour. The reaction mixture was evaporated under reduce pressure. The crude was solubilized in MeOH, filtered through cotton and the filtrate was evaporated under reduce pressure. AcOEt was added and the organic phase was washed twice with water. The crude was purified by reverse column chromatography (C18, from 90/10 water/MeOH to 100% ACN) to afford the title compound as beige solid (18 mg, 11%).

¹H NMR (300 MHZ, DMSO-d6) δ=8.57 (d, J=6.2 Hz, 2H), 8.17 (d, J=2.0 Hz, 1H), 7.74-7.63 (m, 5H), 7.42(d, J=8.5 Hz, 1H).

LC-MS: method X, RT=1.43 min (5 min run), MS (ESI) m/z: 228 (M+H)⁺; 226 (M−H)⁻

### Step 2:3-[[6-(4-pyridyl)-1,3-benzothiazol-2-yl]carbamoyl]bicyclo[2.2.1]hept-5-ene-2-carboxylic acid

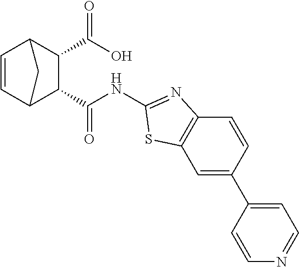 [SMILES: O=C(Nc1nc2ccc(-c3ccncc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (10.8 mg, 0.066 mmol) in THF (1.5 mL) was added 6-(4-pyridyl)-1,3-benzothiazol-2-amine (15.0 mg, 0.066 mmol). The mixture was stirred under reflux overnight. Then, the observed precipitate was washed with THF and ACN and dried to give the title compound as beige solid (23 mg, 89%).

¹H NMR (300 MHZ, DMSO-d6) δ=12.38 (s, 1H), 11.91 (s, 1H), 8.62(dd, J=4.5, 1.6 Hz, 2H), 8.45 (d, J=1.4 Hz, 1H), 7.86 (dd, J=8.6, 2.0 Hz, 1H), 7.80 (d, J=8.0 Hz, 1H), 7.77 (dd, J=4.7, 1.7 Hz, 2H), 6.21-6.12(m, 2H), 3.48 (dd, J=10.0, 3.0 Hz, 1H), 3.39 (dd, J=10.0, 3.1 Hz, 1H), 3.13 s, 1H), 3.08 (s, 1H), 1.34 (dd, J=17.7, 8.4 Hz, 2H).

LC-MS: method X, RT=2.10 min (5 min run), MS (ESI) m/z: 392(M+H)⁺; 390 (M−H)⁻

HRMS (ES+) calculated for C21H18N303S [M+H]⁺: 392.1069 found 392.1059

### Example 65: (1R,2S,6R,7S)-4-[6-(6-fluoro-3-pyridyl)-1,3-benzothiazol-2-yl]-4-azatricyclo[5.2.1.02.6]dec-8-ene-3,5-dione

### Step 1:6-(6-fluoro-3-pyridyl)-1,3-benzothiazol-2-amine

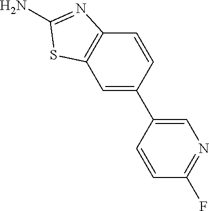 [SMILES: Nc1nc2ccc(-c3ccc(F)nc3)cc2s1]

In a sealed vial was introduced 6-bromo-1,3-benzothiazol-2-amine (100 mg, 0.436 mmol), 2-Fluoropyridine-5-boronic acid (80.0 mg, 0.567 mmol), Bis-(Triphenylphosphine) palladium (II) chloride (15.9 mg, 0.0218 mmol) and CsF (199 mg, 1.31 mmol) in a mixture of 1,4-dioxane (3.60 mL) and water (1.80 mL). The reaction mixture was purged under argon for 15 min and heated at 90° C. After 4 days, the reaction was cooled to room temperature and filtered on a pad of celite. DCM and water were added and the aqueous phase was extracted with DCM twice. The combined organic layers were washed with sat. solution of NaHCO₃ and then with brine. The organic layer was dried, filtered and concentrated under reduce pressure. The crude was purified by column chromatography (from 100% cyclohexane to 100% AcOEt) to afford the title compound as orange solid (55 mg, 51%).

¹H NMR (300 MHZ, DMSO-d6) δ=8.53 (d, J=2.5 Hz, 1H), 8.26 (td, J=8.3, 2.7 Hz, 1H), 8.05 (d, J=1.9 Hz, 1H), 7.62(s, 2H), 7.56 (dd, J=8.4, 2.0 Hz, 1H), 7.42(d, J=8.4 Hz, 1H), 7.26 (dd, J=8.6, 2.9 Hz, 1H).

LC-MS: method X, RT=2.20 min (5 min run), MS (ESI) m/z: 246 (M+H)⁺; 244 (M−H):

### Step 2:(1R,2S,6R,7S)-4-[6-(6-fluoro-3-pyridyl)-1,3-benzothiazol-2-yl]-4-azatricyclo[5.2.1.02,6]dec-8-ene-3,5-dione

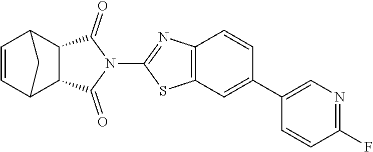 [SMILES: O=C1[C@@H]2C3C=CC(C3)[C@@H]2C(=O)N1c1nc2ccc(-c3ccc(F)nc3)cc2s1]

To a solution of (1R,2S,6R,7S)-4-oxatricyclo[5.2.1.02,6]dec-8-ene-3,5-dione (34.8 mg, 0.21 mmol) in THF (1.5 mL) was added 6-(6-fluoro-3-pyridyl)-1,3-benzothiazol-2-amine (52.0 mg, 0.21 mmol). The mixture was stirred under reflux. After 7 days, the mixture was evaporated under reduce pressure. The crude was purified by column chromatography (from 100% cyclohexane to 90/10 AcOEt, MeOH) to give the title compound as white solid (15 mg, 18%).

¹H NMR (300 MHZ, DMSO-d6) δ=8.65 (d, J=2.6 Hz, 1H), 8.52(d, J=1.5 Hz, 1H), 8.38 (td, J=8.2, 2.7 Hz, 1H), 8.11 (d, J=8.6 Hz, 1H), 7.90 (dd, J=8.6, 2.0 Hz, 1H), 7.34 (dd, J=8.6, 2.9 Hz, 1H), 6.24 (t, 2H), 3.66 (q, J=1.5 Hz, 2H), 3.41 (m, 2H), 1.65 (m, 2H).

LC-MS: method X, RT=11.38 min (30 min run), MS (ESI) m/z: 392(M+H)⁺

HRMS (ES+) calculated for C21H15N302FS [M+H]⁺: 392.0869 found 392.0872

### Example 66 and 67:3-[[6-[6-(trifluoromethyl)-3-pyridyl]-1,3-benzothiazol-2-yl]carbamoyl]bicyclo[2.2.1]hept-5-ene-2-carboxylic acid/(1R,2S,6R,7S)-4-[6-[[6(trifluoromethyl)-3-pyridyl]-1,3-benzothiazol-2-yl]-4-azatricyclo[5.2.1.02,6]dec-8-ene-3,5-dione

### Step 1:6-[6-(trifluoromethyl)-3-pyridyl]-1,3-benzothiazol-2-amine

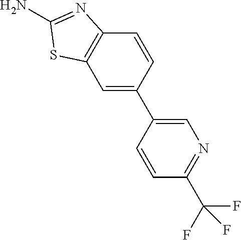 [SMILES: Nc1nc2ccc(-c3ccc(C(F)(F)F)nc3)cc2s1]

In a sealed vial was introduced 6-bromo-1,3-benzothiazol-2-amine (100 mg, 0.436 mmol), [6-(trifluoromethyl)-3-pyridyl]boronic acid (108 mg, 0.567 mmol), Bis(Triphenylphosphine) palladium (II) chloride (15.9 mg, 0.0218 mmol) and CsF (199 mg, 1.31 mmol) in a mixture of 1,4-dioxane (3.60 mL) and water (1.80 mL). The reaction mixture was purged under argon for 15 min and heated at 90° C. After 7 days, the reaction was cooled to room temperature and filtered on a pad of celite. DCM and water were added and the aqueous phase was extracted with DCM twice. The combined organic layers were washed with sat. solution of NaHCO₃ and then with brine. The organic layer was dried, filtered and concentrated under reduce pressure. The crude was purified by column chromatography (from 100% cyclohexane to 100% AcOEt) to afford the title compound as brown solid (78 mg, 61%).

¹H NMR (300 MHZ, DMSO-d6) δ=9.09 (d, J=2.3 Hz, 1H), 8.35 (dd, J=8.3, 2.2 Hz, 1H), 8.19 (d, J=1.8 Hz, 1H), 7.95 (d, J=8.3 Hz, 1H), 7.70 (s, 2H), 7.69 (dd, J=8.5, 2.0 Hz, 1H), 7.46 (d, J=8.4 Hz, 1H).

LC-MS: method X, RT=2.55 min (5 min run), MS (ESI) m/z: 294 (M−H)⁻ Step 2:

### Example 66 3-[[6-[6-(trifluoromethyl)-3-pyridyl]-1,3-benzothiazol-2-yl]carbamoyl]bicyclo [2.2.1]hept-5-ene-2-carboxylic acid

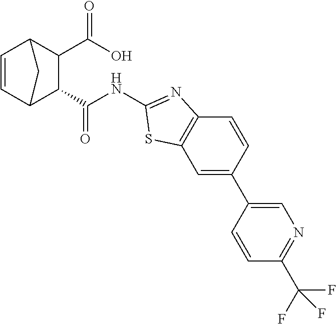 [SMILES: O=C(O)C1C2C=CC(C2)[C@H]1C(=O)Nc1nc2ccc(-c3ccc(C(F)(F)F)nc3)cc2s1]

### Example 67 (1R,2S,6R,7S)-4-[6-[6-(trifluoromethyl)-3-pyridyl]-1,3-benzothiazol-2-yl]-4-azatricyclo [5.2.1.02,6]dec-8-ene-3,5-dione

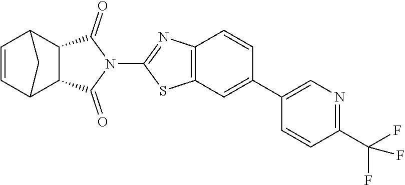 [SMILES: O=C1[C@@H]2C3C=CC(C3)[C@@H]2C(=O)N1c1nc2ccc(-c3ccc(C(F)(F)F)nc3)cc2s1]

To a solution of (1R,2S,6R,7S)-4-oxatricyclo[5.2.1.02,6]dec-8-ene-3,5-dione (37.2 mg, 0.23 mmol) in THF (1 mL) was added 6-[6-(trifluoromethyl)-3-pyridyl]-1,3-benzothiazol-2-amine (67.0 mg, 0.23 mmol). The mixture was stirred under reflux. After 48 h, the mixture was cooled to room temperature, and a mixture of ACN and THF was added. The observed precipitate was filtrated, washed with ACN and THF to afford the title acid compound as white solid (Example 66, 11 mg, 11%). The filtrate was evaporated and purified by column chromatography (from 80/20 cyclohexane/AcOEt to 90/10 AcOEt/MeOH) to give the title succinimide compound as white solid (Example 67, 14 mg, 14%).

### Example 66

¹H NMR (300 MHZ, DMSO-d6) δ=12.40 (s, 1H), 11.90 (s, 1H), 9.17 (d, J=1.9 Hz, 1H), 8.48 (d, J=1.5 Hz, 1H), 8.43 (dd, J=8.2, 2.1 Hz, 1H), 8.00 (d, J=8.3 Hz, 1H), 7.89 (dd, J=8.5, 1.8 Hz, 1H), 7.84 (d, J=8.4 Hz, 1H), 6.18 (m, 2H), 3.50 (dd, J=10.1, 3.1 Hz, 1H), 3.40 (dd, J=10.3, 3.5 Hz, 1H), 3.13 (d, J=15.8 Hz, 2H), 1.36 (m, 2H).

LC-MS: method X, RT=11.55 min (30 min run), MS (ESI) m/z: 460 (M+H)⁺; 458 (M−H)⁻

HRMS (ES+) calculated for C22H17N303F3S [M+H]⁺: 460.0943 found 460.0934

### Example 67

¹H NMR (300 MHZ, DMSO-d6) δ=9.20 (d, J=2.4 Hz, 1H), 8.65 (d, J=1.6 Hz, 1H), 8.47 (dd, J=8.3, 2.2 Hz, 1H), 8.16 (d, J=8.6 Hz, 1H), 8.05 (d, J=8.4 Hz, 1H), 8.01 (dd, J=8.6, 1.9 Hz, 1H), 6.25 (t, 2H), 3.67 (q, J=1.4 Hz, 2H), 3.42(m, 2H), 1.66 (m, 2H).

LC-MS: method X, RT=12.93 min (30 min run), MS (ESI) m/z: 442(M+H)⁺

HRMS (ES+) calculated for C22H15N302F3S [M+H]⁺: 442.0837 found 442.0845

### EXAMPLE 68: (1R,2S,6R,7S)-4-[6-(3-thienyl)-1,3-benzothiazol-2-yl]-4-azatricyclo[5.2.1.02,6]dec-8-ene-3,5-dione

### Step 1: tert-butyl N-(6-bromo-1,3-benzothiazol-2-yl)carbamate

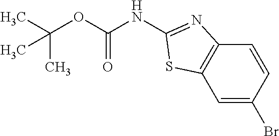 [SMILES: CC(C)(C)OC(=O)Nc1nc2ccc(Br)cc2s1]

To a solution of 6-bromo-1,3-benzothiazol-2-amine (500 mg, 2.18 mmol) in DCM (20 mL) at 0° C. was added DMAP (8 mg, 0.066 mmol) and BOC anhydride (714 mg, 3.27 mmol). The reaction was allowed to warm to room temperature and stirred overnight. After 16h, water was added to the reaction mixture and the aqueous phase was extracted 3 times with ethyl acetate. The combined organic phases were washed with water and brine, and then, dried, filtered and concentrated under reduce pressure. The crude was purified by column chromatography (from 100% cyclohexane to 70/30 cyclohexane/AcOEt) to afford the title compound as white solid (486.2 mg, 67.7% yield).

¹H NMR (300 MHZ, CDCl₃) δ=7.91 (d, J=1.9 Hz, 1H), 7.70 (d, J=8.6 Hz, 1H), 7.54-7.49 (dd, J=8.6, 1.9 Hz, 1H), 1.6 (s, 9H).

LC-MS: method X, RT=3.27 min (5 min run), MS (ESI) m/z: 275 (M−C(CH₃)₃)+; 329 (M−H)⁻

### Step 2: tert-butyl N-[6-(3-thienyl)-1,3-benzothiazol-2-yl]carbamate

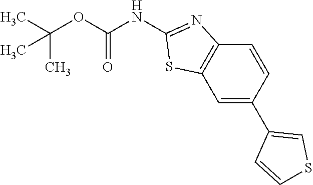 [SMILES: CC(C)(C)OC(=O)Nc1nc2ccc(-c3ccsc3)cc2s1]

In a sealed vial was introduced tert-butyl N-(6-bromo-1,3-benzothiazol-2-yl)carbamate (150 mg, 0.46 mmol), 3-thienylboronic acid (70.0 mg, 0.55 mmol), Bis(Triphenylphosphine) palladium (II) chloride (16.6 mg, 0.0228 mmol) and CsF (208 mg, 1.37 mmol) in a mixture of 1,4-dioxane (3.60 mL) and water (1.80 mL). The reaction mixture was purged under argon for 15 min and heated at 90° C. After 16 h, the reaction was cooled to room temperature and filtered on a pad of celite. DCM and water were added and the aqueous phase was extracted with DCM twice. The combined organic layers were washed with sat. solution of NaHCO₃ and then with brine. The organic layer was dried, filtered and concentrated under reduce pressure. The crude was purified by column chromatography (from 100% cyclohexane to 50/50 cyclohexane/AcOEt) to afford the title compound as yellow solid (74 mg, 49%).

¹H NMR (300 MHZ, CDCl₃) δ=7.99 (d, J=1.4 Hz, 1H), 7.87 (d, J=8.4 Hz, 1H), 7.66 (dd, J=8.4, 1.8 Hz, 1H), 7.49 (m, 1H), 7.45-7.42(m, 2H), 1.62(s, 9H).

LC-MS: method X, RT=3.32 min (5 min run), MS (ESI) m/z: 277 (M−C(CH₃)₃)+; 331 (M−H)⁻

### Step 3:6-(3-thienyl)-1,3-benzothiazol-2-amine

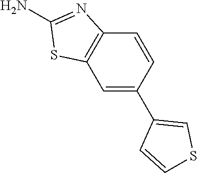 [SMILES: Nc1nc2ccc(-c3ccsc3)cc2s1]

To a solution of TFA (43.9 mg, 0.385 mmol) in DCM (5 mL) at 0° C. was added a solution of tert-butyl N-[6-(3-thienyl)-1,3-benzothiazol-2-yl]carbamate (64 mg, 0.193 mmol) in DCM (5 mL) dropwise. After 8 days, the solution was made alkaline by the addition of a saturated solution of NaHCO₃. The aqueous phase was extracted 3 times with DCM. The combined organic phases were dried, filtered and concentrated under reduce pressure. The crude was used for the next step without further purification.

¹H NMR (300 MHZ, CDCl₃) δ=8.02(d, J=1.7 Hz, 1H), 7.60 (dd, J=2.9, 1.3 Hz, 1H), 7.63-7.51 (m, 5H), 7.34 (d, J=8.4 Hz, 1H).

LC-MS: method X, RT=3.5 min (5 min run), MS (ESI) m/z: 233 (M+H)⁺

### Step 4:(1R,2S,6R,7S)-4-[6-(3-thienyl)-1,3-benzothiazol-2-yl]-4-azatricyclo[5.2.1.02,6]dec-8-ene-3,5-dione

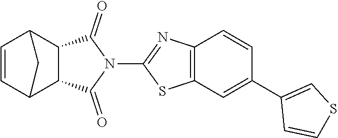 [SMILES: O=C1[C@@H]2C3C=CC(C3)[C@@H]2C(=O)N1c1nc2ccc(-c3ccsc3)cc2s1]

To a solution of (1R,2S,6R,7S)-4-oxatricyclo[5.2.1.02,6]dec-8-ene-3,5-dione (45.2 mg, 0.25 mmol) in THF (2 mL) was added 6-(3-thienyl)-1,3-benzothiazol-2-amine (58.3 mg, 0.25 mmol). The mixture was stirred under reflux. After 5 days, the reaction mixture was evaporated under reduce pressure. The crude was purified by column chromatography (from 100% cyclohexane to 100% AcOEt) to afford the title compound as white solid (39 mg, 41%).

¹H NMR (300 MHZ, DMSO-d6) δ=8.48 (d, J=1.8 Hz, 1H), 8.20-8.0 (d, J=8.5 Hz, 1H), 7.99-7.98 (dd, J=2.9, 1.4 Hz, 1H), 7.93-7.89 (dd, J=8.5, 1.8 Hz, 1H), 7.70-7.67 (dd, J=5.0, 2.9 Hz, 1H), 7.66-7.63 (dd, J=5.0, 1.4 Hz, 1H), 6.23 (d, J=1.8 Hz, 2H), 3.64-3.62(dd, J=2.9, 1.6 Hz, 2H), 3.39 (m, 2H), 1.63 (m, 2H).

LC-MS: method X, RT=12.90 min (30 min run), MS (ESI) m/z: 379 (M+H)⁺

HRMS (ES+) calculated for C20H15N2O2S2 [M+H]⁺: 379.0575 found 379.0580

### Examples 69 and 70:3-[[6-(3-furyl)-1,3-benzothiazol-2-yl]carbamoyl]bicyclo[2.2.1]hept-5-ene-2-carboxylic acid and (1R,2S,6R,7S)-4-[6-(3-furyl)-1,3-benzothiazol-2-yl]-4-azatricyclo[5.2.1.02,6]dec-8-ene-3,5-dione

### Step 1:6-(3-furyl)-1,3-benzothiazol-2-amine

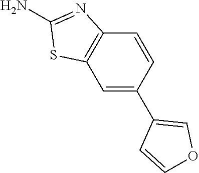 [SMILES: Nc1nc2ccc(-c3ccoc3)cc2s1]

In a sealed vial was introduced 6-bromo-1,3-benzothiazol-2-amine (100 mg, 0.436 mmol), 3-furylboronic acid (63.5 mg, 0.567 mmol), Bis(Triphenylphosphine) palladium (II) chloride (15.9 mg, 0.0218 mmol) and CsF (199 mg, 1.31 mmol) in a mixture of 1,4-dioxane (3.60 mL) and water (1.80 mL). The reaction mixture was purged under argon for 15 min and heated at 90° C. After 2 days, the reaction was cooled to room temperature and filtered on a pad of celite. DCM and water were added and the aqueous phase was extracted with DCM twice. The combined organic layers were washed with sat. solution of NaHCO₃ and then with brine. The organic layer was dried, filtered and concentrated under reduce pressure. The crude was purified by column chromatography (from 80/20 cyclohexane/AcOEt to 90/10 AcOEt/MeOH) to afford the title compound as brown solid (49 mg, 52%).

¹H NMR (300 MHZ, DMSO-d6) δ=8.11 (dd, J=1.5 Hz, 1H), 7.92(d, J=1.8 Hz, 1H), 7.71 (t, J=1.7 Hz, 1H), 7.49 (s, 2H), 7.46 (dd, J=8.3, 1.9 Hz, 1H), 7.32(d, J=8.4 Hz, 1H), 6.94 (d, J=1.9 Hz, 1H).

LC-MS: method X, RT=2.35 min (5 min run), MS (ESI) m/z: 217 (M+H)⁺; 215 (M−H)⁻ Step 2:

### Example 69 3-[[6-(3-furyl)-1,3-benzothiazol-2-yl]carbamoyl]bicyclo[2.2.1]hept-5-ene-2-carboxylic acid

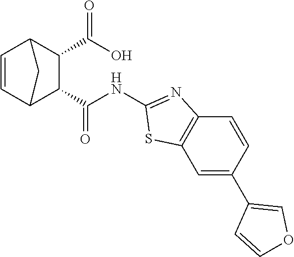 [SMILES: O=C(Nc1nc2ccc(-c3ccoc3)cc2s1)[C@@H]1C2C=CC(C2)[C@@H]1C(=O)O]

### Example 70 (1R,2S,6R,7S)-4-[6-(3-furyl)-1,3-benzothiazol-2-yl]-4-azatricyclo[5.2.1.02,6]dec-8-ene-3,5-dione

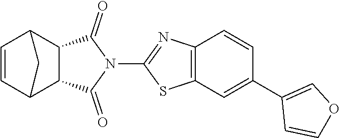 [SMILES: O=C1[C@@H]2C3C=CC(C3)[C@@H]2C(=O)N1c1nc2ccc(-c3ccoc3)cc2s1]

To a solution of (1R,2S,6R,7S)-4-oxatricyclo[5.2.1.02,6]dec-8-ene-3,5-dione (72.1 mg, 0.44 mmol) in THF (2 mL) was added 6-(3-furyl)-1,3-benzothiazol-2-amine (95.0 mg, 0.44 mmol). The mixture was stirred under reflux. After 9 h, the observed precipitate was filtrated, washed with ACN and THF and dried to afford the title acid compound as white solid (Example 69, 31 mg, 19%). The filtrate was evaporated and the crude was purified by column chromatography (from 100% cyclohexane to 90/10 AcOEt/MeOH) to give the title compound as white solid (Example 70, 31 mg, 20%).

### Example 69

¹H NMR (300 MHz, DMSO-d6) δ=12.27 (s, 1H), 11.87 (s, 1H), 8.22(t, J=1.2 Hz, 1H), 8.19 (d, J=1.3 Hz, 1H), 7.75 (t, J=1.7 Hz, 1H), 7.70 (d, J=7.7 Hz, 1H), 7.67 (d, 1H), 7.03 (dd, J=1.9, 0.8 Hz, 1H), 6.20 (m, 2H), 3.48 (dd, J=10.2, 3.1 Hz, 1H), 3.38 (dd, J=10.3, 3.5 Hz, 1H), 3.11 (d, J=14.9 Hz, 2H), 1.34 (q, 2H).

LC-MS: method X, RT=10.60 min (30 min run), MS (ESI) m/z: 381 (M+H)⁺; 379 (M−H)⁻

HRMS (ES+) calculated for C20H17N2O4S [M+H]⁺: 381.0909 found 381.0907

### Example 70

¹H NMR (300 MHz, DMSO-d6) δ=8.37 (d, J=1.6 Hz, 1H), 8.29 (dd, J=1.4, 0.9 Hz, 1H), 8.00 (d, J=8.5 Hz, 1H), 7.82(dd, J=8.6, 1.8 Hz, 1H), 7.79 (t, J=1.7 Hz, 1H), 7.05 (dd, J=1.9, 0.9 Hz, 1H), 6.23 (t, 2H), 3.64 (q, 2H), 3.40 (m, 2H), 1.64 (m, 2H).

LC-MS: method X, RT=12.10 min (30 min run), MS (ESI) m/z: 363 (M+H)⁺

HRMS (ES+) calculated for C20H15N2O3S [M+H]⁺: 363.0803 found 363.0809

### Example 71: (1R,2S,6R,7S)-4-[6-(4-prop-2-ynoxyphenyl)-1,3-benzothiazol-2-yl]-4-azatricyclo[5.2.1.02,6]dec-8-ene-3,5-dione

### Step 1:4-(2-amino-1,3-benzothiazol-6-yl)phenol

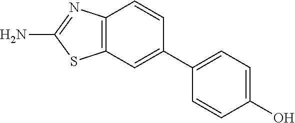 [SMILES: Nc1nc2ccc(-c3ccc(O)cc3)cc2s1]

In a sealed tube were introduced 4-(4,4,5,5-tetramethyl-1,3,2-dioxaborolan-2-yl)phenol (54.8 mg, 0.25 mmool), 6-bromo-1,3-benzothiazol-2-amine (57.0 mg, 0.25 mmol), Pd(dppf)Cl₂(36.4 mg, 0.05 mmol), and K₂CO₃ (68.8 mg, 0.5 mmol). The mixture was purged with argon for 15 min, prior to the addition of a mixture of 1,4-dioxane/water (4 mL, 3:1). The solution was heated at 100° C. under microwave irradiation for 1 h. The reaction mixture was filtrated through coton and the filtrate was evaporated under reduce pressure. The crude was purified by column chromatography (from 100% cyclohexane to 50/50 cyclohexnae/AcOEt) to afford the title compound as beige solid (30 mg, 50%).

LC-MS: method X, RT=2.02 min (5 min run), MS (ESI) m/z: 243 (M+H)⁺; 241 (M−H)⁻

### Step 2: (1R,2S,6R,7S)-4-[6-(4-hydroxyphenyl)-1,3-benzothiazol-2-yl]-4-azatricyclo[5.2.1.02,6]dec-8-ene-3,5-dione

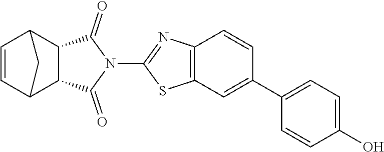 [SMILES: O=C1[C@@H]2C3C=CC(C3)[C@@H]2C(=O)N1c1nc2ccc(-c3ccc(O)cc3)cc2s1]

To a solution of (1R,2S,6R,7S)-4-oxatricyclo[5.2.1.02,6]dec-8-ene-3,5-dione (5.42 mg, 0.03 mmol) in THF (1 mL) was added 4-(2-amino-1,3-benzothiazol-6-yl)phenol (8.0 mg, 0.03 mmol). The mixture was stirred under reflux overnight. The reaction mixture was evaporated and ACN was added. The observed precipitate was filtrated, washed with ACN and THF and dried to afford the title compound as white solid (10 mg, 78%).

¹H NMR (300 MHZ, DMSO-d6) δ=9.62(s, 1H), 8.32(dd, J=2.0, 0.5 Hz, 1H), 8.00 (dd, J=8.7, 0.5 Hz, 1H), 7.76 (dd, J=8.7, 1.9 Hz, 1H), 7.63-7.54 (m, 2H), 6.91-6.84 (m, 2H), 6.24 (t, J=1.7 Hz, 2H), 3.64 (dd, J=2.9, 1.6 Hz, 2H), 3.43-3.37 (m, 2H), 1.64 (d, J=1.8 Hz, 2H).

LC-MS: method X, RT=2.65 min (5 min run), MS (ESI) m/z: 389 (M+H)⁺; 387 (M−H)⁻

HRMS (ES+) calculated for C22H17N2O3S [M+H]⁺: 389.0960 found 389.0970

### Step 3: (1R,2S,6R,7S)-4-[6-(4-prop-2-ynoxyphenyl)-1,3-benzothiazol-2-yl]-4-azatricyclo[5.2.1.02,6]dec-8-ene-3,5-dione

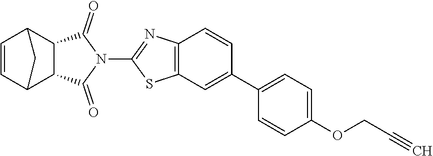 [SMILES: C#CCOc1ccc(-c2ccc3nc(N4C(=O)[C@@H]5C6C=CC(C6)[C@@H]5C4=O)sc3c2)cc1]

To a solution of 4-[6-(4-hydroxyphenyl)-1,3-benzothiazol-2-yl]-4-azatricyclo[5.2.1.02,6]dec-8-ene-3,5-dione (15 mg, 0.039 mmol) in acetone (1.5 mL) was added K₂CO₃ (18.7 mg, 0.077 mmol). The reaction mixture was stirred for 1 h 30 at room temperature prior to the addition of propargyl bromide (5.85 L, 0.077 mmol) dropwise. Then, the reaction was stirred at 50° C. overnight. The crude was evaporated under reduce pressure and purified by column chromatography (from 100% cyclohexane to 70/30 cyclohexane/AcOEt) to afford the title compound as white solid (4 mg, 24%).

¹H NMR (300 MHZ, DMSO-d6) δ=8.12(d, J=8.9 Hz, 1H), 8.01 (brs, 1H), 7.68 (dd, J=8.5, 2.4 Hz, 1H), 7.59 (d, J=8.6 Hz, 2H), 7.09 (d, J=8.9 Hz, 2H), 6.30 (t, J=1.6 Hz, 2H), 4.76 (d, J=2.4 Hz, 2H), 3.63-3.53 (m, 4H), 2.56 (t, J=2.5 Hz, 1H), 1.72-1.64 (m, 2H).

LC-MS: method X, RT=3.10 min (5 min run), MS (ESI) m/z: 427 (M+H)⁺

### Example 72: (1R,2S,6R,7S)-4-(6-ethynyl-1,3-benzothiazol-2-yl)-4-azatricyclo[5.2.1.02,6]dec-8-ene-3,5-dione

### Step 1:6-iodo-1,3-benzothiazol-2-amine

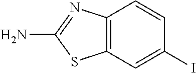 [SMILES: Nc1nc2ccc(I)cc2s1]

To a solution of 6-bromo-1,3-benzothiazol-2-amine (500 mg, 2.18 mmol), in 1,4-dioxane (5.0 mL) was added CuI (21 mg, 0.11 mmol, 5.0 mol %), sodium iodide (650 mg, 4.4 mmol), and (1R,2R)—N1,N2-dimethylcyclohexane-1,2-diamine (35 μL 0.218 mmol). The reaction mixture was heated at 110° C. for one week. Then, the reaction was evaporated and purified by reverse column chromatography (from 90/10 water/MeOH to 100% MeOH) to afford the title compound as white solid (430 mg).

LC-MS: method X, RT=2.50 min (5 min run), MS (ESI) m/z: 277 (M+H)⁺; 275 (M−H)⁻

### Step 2:6-(2-trimethylsilylethynyl)-1,3-benzothiazol-2-amine

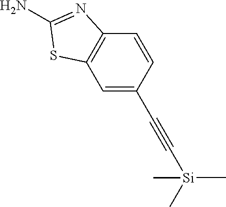 [SMILES: C[Si](C)(C)C#Cc1ccc2nc(N)sc2c1]

In a sealed tube was introduced 6-iodo-1,3-benzothiazol-2-amine (150 mg, 0.54 mmol), PdCl₂(PPh₃)₂(23 mg, 0.033 mmol), and CuI (20.6 mg, 0.108 mmol). The mixture was purged with argon for 15 min, prior to the addition of ACN (1 mL), TEA (1.28 mL, 9.20 mmol) and ethynyl(trimethyl) silane (0.101 mL, 0.806 mmol). The solution was heated at 100° C. under microwave irradiation for 45 min. Then, EtOAc and water were added and the aqueous layer was extracted twice with AcOEt. The combined organic layers were washed with brine, dried, filtered and concentrated under reduce pressure. The title compound was used without further purification and the yield was supposed to be quantitative (white solid, 134 mg, 100%).

LC-MS: method X, RT=3.08 min (5 min run), MS (ESI) m/z: 247 (M+H)⁺; 245 (M−H)⁻

### Step 3:(1R,2S,6R,7S)-4-[6-(2-trimethylsilylethynyl)-1,3-benzothiazol-2-yl]-4-azatricyclo[5.2.1.02,6]dec-8-ene-3,5-dione

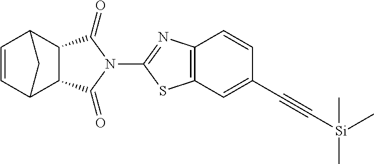 [SMILES: C[Si](C)(C)C#Cc1ccc2nc(N3C(=O)[C@@H]4C5C=CC(C5)[C@@H]4C3=O)sc2c1]

To a solution of (1R,2S,6R,7S)-4-oxatricyclo[5.2.1.02,6]dec-8-ene-3,5-dione (89.3 mg, 0.54 mmol) in THF (1 mL) was added 6-(2-trimethylsilylethynyl)-1,3-benzothiazol-2-amine (134 mg, 0.54 mmol). The mixture was stirred under reflux. After 48 h, the reaction mixture was evaporated and THF was added. The observed precipitate was filtrated, washed with ACN and THF and dried to afford the title compound as white solid (110 mg, 51%).

¹H NMR (300 MHZ, DMSO-d6) δ=8.29 (dd, J=1.7, 0.5 Hz, 1H), 7.95, (dd, J=8.5, 0.5 Hz, 1H), 7.557 (dd, J=8.5, 1.8 Hz, 1H), 6.21 (t, J=1.8 Hz, 2H), 3.64 (dd, J=2.9, 1.6 Hz, 2H), 3.43-3.37 (m, 2H), 1.68-1.58 (m, 2H), 0.26 (s, 9H).

LC-MS: method X, RT=6.02 min (10 min run), MS (ESI) m/z: 393 (M+H)⁺

### Step 4:(1R,2S,6R,7S)-4-(6-ethynyl-1,3-benzothiazol-2-yl)-4-azatricyclo[5.2.1.02,6]dec-8-ene-3,5-dione

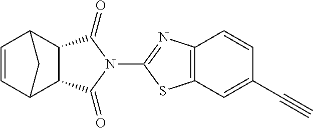 [SMILES: C#Cc1ccc2nc(N3C(=O)[C@@H]4C5C=CC(C5)[C@@H]4C3=O)sc2c1]

To a solution of (1R,2S,6R,7S)-4-[6-(2-trimethylsilylethynyl)-1,3-benzothiazol-2-yl]-4-azatricyclo[5.2.1.02,6]dec-8-ene-3,5-dione (80 mg, 0.20 mmol) in THF (2 mL) was added TBAF (64.3 mg, 0.20 mmol) and the reaction mixture was stirred overnight at rt. Then, AcOEt was added and was washed with water twice and brine. The organic layer was dried, filtered and concentrated under reduce pressure. The crude was purified by column chromatography (from 100% cyclohexane to 60/40 cyclohexane/AcOEt) to afford the title compound as white solid (20 mg, 31%).

¹H NMR (300 MHZ, CDCl₃) δ=8.04 (d, J=8.6 Hz, 1H), 8.02(d, J=1.8 Hz, 1H), 7.60 (dd, J=8.5, 1.7 Hz, 1H), 6.30 (t, J=1.7 Hz, 2H), 3.66-3.54 (m, 4H), 3.16 (s, 1H), 1.84 (dt, J=9.0, 4.6 Hz, 1H), 1.67 (d, J=9.0 Hz, 1H).

LC-MS: method X, RT=2.82 min (5 min run), MS (ESI) m/z: 321 (M+H)⁺

HRMS (ES+) calculated for C18H13N2O2S [M+H]⁺: 321.0698 found 321.0702

### Example 73: (1R,2S,6R,7S)-4-(6-prop-2-ynoxy-1,3-benzothiazol-2-yl)-4-azatricyclo[5.2.1.02,6]dec-8-ene-3,5-dione

### Step 1:(1R,2S,6R,7S)-4-(6-hydroxy-1,3-benzothiazol-2-yl)-4-azatricyclo[5.2.1.02,6]dec-8-ene-3,5-dione

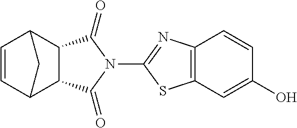 [SMILES: O=C1[C@@H]2C3C=CC(C3)[C@@H]2C(=O)N1c1nc2ccc(O)cc2s1]

To a solution of (1R,2S,6R,7S)-4-oxatricyclo[5.2.1.02,6]dec-8-ene-3,5-dione (500 mg, 3.05 mmol) in THF (1 mL) was added 2-amino-1,3-benzothiazol-6-ol (506 mg, 3.05 mmol) and the mixture was stirred under reflux for 4 h. Then, T3P (50% in AcOEt, 3.05 mmol) was added and the reaction was stirred overnight under reflux. The mixture was evaporated under reduce pressure and THF was added. The observed precipitate was filtrated, washed with THE, and dried to afford the title compound as white solid (750 mg, 79%).

¹H NMR (300 MHZ, DMSO-d6) δ=9.91 (s, 1H), 7.80 (d, J=8.9 Hz, 1H), 7.39 (d, J=2.4 Hz, 1H), 6.99 (dd, J=8.9, 2.5 Hz, 1H), 6.21 (t, J=1.9 Hz, 2H), 3.60 (dd, J=2.8, 1.5 Hz, 2H), 3.37 (m, 2H), 1.62(s, 2H).

LC-MS: method X, RT=3.47 min (10 min run), MS (ESI) m/z: 313 (M+H)⁺; 311 (M−H)⁻

### Step 2:(1R,2S,6R,7S)-4-(6-prop-2-ynoxy-1,3-benzothiazol-2-yl)-4-azatricyclo[5.2.1.02,6]dec-8-ene-3,5-dione

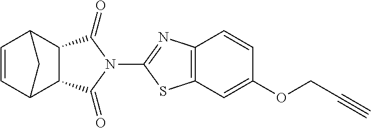 [SMILES: C#CCOc1ccc2nc(N3C(=O)[C@@H]4C5C=CC(C5)[C@@H]4C3=O)sc2c1]

To a solution of (1R,2S,6R,7S)-4-(6-hydroxy-1,3-benzothiazol-2-yl)-4-azatricyclo[5.2.1.02,6]dec-8-ene-3,5-dione (50 mg, 0.16 mmol) in acetone (1.5 mL) was added K₂CO₃ (77.4 mg, 0.56 mmol) and the mixture was stirred for 1 h 30 at room temperature. Then, a solution of propargyl bromide (24.3 μL, 0.32 mmol) in acetone (0.5 mL) was added and the reaction was stirred at 50° C. overnight. Then, the mixture was evaporated under reduce pressure and AcOEt was added. The organic layer was washed twice with water, dried, filtered and concentrated under reduce pressure. The crude was purified by column chromatography (from 100% cyclohexane to 60/40 cyclohexane/AcOEt) to afford the title compound as white solid (12 mg, 43%).

¹H NMR (300 MHZ, CDCl₃) δ=8.00 (d, J=9.1 Hz, 1H), 7.41 (d, J=2.5 Hz, 1H), 7.16 (dd, J=9.0, 2.6 Hz, 1H), 6.28 (t, J=1.8 Hz, 2H), 4.76 (d, J=2.4 Hz 2H), 3.61-3.51 (m, 4H), 2.55 (t, J=2.4 Hz, 1H), 1.82(dt, J=9.0, 1.8 Hz, 1H), 1.64 (d, J=9.0 Hz, 1H).

LC-MS: method X, RT=4.35 min (10 min run), MS (ESI) m/z: 351 (M+H)⁺

HRMS (ES+) calculated for C19H15N2O3S [M+H]⁺: 351.0803 found 351.0806

### Example 74: (1R,2S,3R,4S)-3-[(4-hydroxy-1,3-benzothiazol-2-yl)carbamoyl]bicyclo[2.2.1]hept-5-ene-2-carboxylic acid

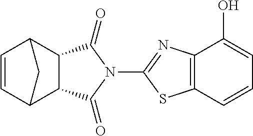 [SMILES: O=C1[C@@H]2C3C=CC(C3)[C@@H]2C(=O)N1c1nc2c(O)cccc2s1]

To a solution of (1R,2S,6R,7S)-4-oxatricyclo[5.2.1.02,6]dec-8-ene-3,5-dione (200 mg, 1.22 mmol) in THF (2 mL) was added 2-amino-1,3-benzothiazol-4-ol (202 mg, 1.22 mmol). The mixture was stirred under reflux overnight. Then, the reaction mixture was evaporated and the crude was purified by reverse column chromatography (from 90/10 water/ACN to 100% ACN) to afford the title compound as beige solid (126 mg, 33%).

¹H NMR (300 MHZ, DMSO-d6) δ=10.30 (s, 1H), 7.49 (dd, J=8.1, 1.0 Hz, 1H), 7.29 (t, J=8.1 Hz, 1H), 6.92(dd, J=7.9, 1.0 Hz, 1H), 6.24 (t, J=1.9 Hz, 2H), 3.64 (dd, J=2.9, 1.6 Hz, 2H), 3.41-3.38 (m, 2H), 1.64 (s, 2H).

LC-MS: method X, RT=4.18 min (10 min run), MS (ESI) m/z: 313 (M+H)⁺; 311 (M−H)⁻

HRMS (ES+) calculated for C16H13N2O3S [M+H]⁺: 313.0647 found 313.0648

### Example 75:3-[(4-prop-2-ynoxy-1,3-benzothiazol-2-yl)carbamoyl]bicyclo[2.2.1]hept-5-ene-2-carboxylic acid

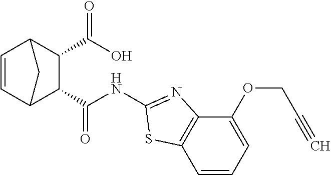 [SMILES: C#CCOc1cccc2sc(NC(=O)[C@@H]3C4C=CC(C4)[C@@H]3C(=O)O)nc12]

To a solution of (1R,2S,6R,7S)-4-(4-hydroxy-1,3-benzothiazol-2-yl)-4-azatricyclo[5.2.1.02,6]dec-8-ene-3,5-dione (100 mg, 0.32 mmol) in acetone (1.5 mL) was added K₂CO₃ (155 mg, 1.12 mmol) and the mixture was stirred for 1 h 30 at rt. Then, a solution of propargyl bromide (50 μL, 0.64 mmol) in acetone (0.5 mL) was added and the reaction was stirred at 50° C. overnight. Then, the mixture was evaporated under reduce pressure and DCM was added. The organic layer was washed an aqueous solution of 1N HCl and twice with brine. The organic layer was dried, filtered and concentrated under reduce pressure. The crude was purified by reverse column chromatography (from 90/10 water/ACN to 100% ACN) to afford the title compound as white solid (12 mg, 11%).

¹H NMR (300 MHZ, DMSO-d6) δ=12.43 (s, 1H), 11.83 (brs, 1H), 7.54 (dd, J=8.0, 0.9 Hz, 1H), 7.22(t, J=8.0 Hz, 1H), 7.04 (dd, J=8.0, 0.9 Hz, 1H), 6.16 (brs, 2H), 4.96 (d, J=2.4 Hz, 2H), 3.58 (t, J=2.5 Hz, 1H), 3.44 (dd, J=10.0, 3.1 Hz, 1H), 3.37 (dd, J=10.0, 3.2 Hz, 1H), 3.13 (brs, 1H), 3.08 (brs, 1H), 1.42-1.28 (m, 2H)

LC-MS: method X, RT=4.40 min (10 min run), MS (ESI) m/z: 369 (M+H)⁺; 367 (M−H)⁻

HRMS (ES+) calculated for C19H17N2O4S [M+H]⁺: 369.0909 found 369.0929

### Example 76: (1R,2S,6R,7S)-4-(4-prop-2-ynoxy-1,3-benzothiazol-2-yl)-4-azatricyclo[5.2.1.02,6]dec-8-ene-3,5-dione

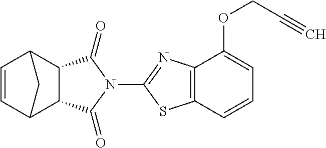 [SMILES: C#CCOc1cccc2sc(N3C(=O)[C@@H]4C5C=CC(C5)[C@@H]4C3=O)nc12]

To a solution of (1R,2S,6R,7S)-4-(4-hydroxy-1,3-benzothiazol-2-yl)-4-azatricyclo[5.2.1.02,6]dec-8-ene-3,5-dione (100 mg, 0.32 mmol) in acetone (1.5 mL) was added K₂CO₃ (155 mg, 1.12 mmol) and the mixture was stirred for 1 h 30 at rt. Then, a solution of propargyl bromide (50 μL, 0.64 mmol) in acetone (0.5 mL) was added and the reaction was stirred at 50° C. overnight. Then, the mixture was evaporated under reduce pressure and DCM was added. The organic layer was washed an aqueous solution of 1N HCl and twice with brine. The organic layer was dried, filtered and concentrated under reduce pressure. The crude was purified by reverse column chromatography (from 90/10 water/ACN to 100% ACN) to afford the title compound as white solid (12 mg, 11%).

¹H NMR (300 MHZ, DMSO-d6) δ=7.49 (dd, J=8.1, 1.0 Hz, 1H), 7.38 (t, J=8.1 Hz, 1H), 7.12(dd, J=8.0, 1.0 Hz, 1H), 6.29 (brs, 2H), 5.00 (d, J=2.3 Hz, 2H), 3.62-3.52(m, 4H), 2.53 (t, J=2.3 Hz, 1H), 1.82(d, J=8.9 Hz, 1H), 1.65 (d, J=8.8 Hz, 1H)

LC-MS: method X, RT=2.48 min (5 min run), MS (ESI) m/z: 351 (M+H)⁺

HRMS (ES+) calculated for C19H15N2O3S [M+H]⁺: 351.0803 found 351.0817

### Example 77:3-[[6-(2-phenylethynyl)-1,3-benzothiazol-2-yl]carbamoyl]bicyclo[2.2.1]hept-5-ene-2-carboxylic acid

### Step 1:6-iodo-1,3-benzothiazol-2-amine

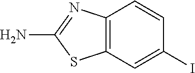 [SMILES: Nc1nc2ccc(I)cc2s1]

To a solution of 6-bromo-1,3-benzothiazol-2-amine (500 mg, 2.18 mmol), in 1,4-dioxane (5.0 mL) was added CuI (21 mg, 0.11 mmol, 5.0 mol %), sodium iodide (650 mg, 4.4 mmol), and (1R,2R)—N1,N2-dimethylcyclohexane-1,2-diamine (35 μL 0.218 mmol). The reaction mixture was heated at 110° C. for one week. Then, the reaction was evaporated and purified by reverse column chromatography (from 90/10 water/MeOH to 100% MeOH) to afford the title compound as white solid (430 mg).

LC-MS: method X, RT=2.50 min (5 min run), MS (ESI) m/z: 277 (M+H)⁺; 275 (M−H)⁻

### Step 2:6-(2-phenylethynyl)-1,3-benzothiazol-2-amine

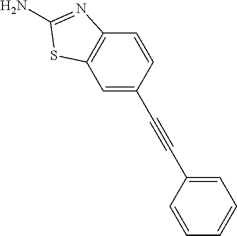 [SMILES: Nc1nc2ccc(C#Cc3ccccc3)cc2s1]

In a sealed tube was introduced 6-iodo-1,3-benzothiazol-2-amine (100 mg, 0.36 mmol), PdCl₂(PPh₃)₂(15.3 mg, 0.022 mmol), and CuI (13.8 mg, 0.07 mmol). The mixture was purged with argon for 15 min, prior to the addition of ACN (1 mL), TEA (0.858 mL, 6.16 mmol) and ethynylbenzene (0.060 mL, 0.54 mmol). The solution was heated at 100° C. under microwave irradiation for 30 min. Then, EtOAc and the organic layer was washed twice with water. The aqueous phase was extracted with EtOAc and the organic layer were combined, dried, filtered and concentrated under reduce pressure. The crude was purified by column chromatography (from 100% DCM to 90/10 DCM/MeOH). A second purification by reverse column chromatography (from 90/10 water/ACN to 100% ACN) was made to afford the title compound as light brown solid (73 mg, 81%).

¹H NMR (300 MHZ, DMSO-d6) δ=7.88 (d, J=1.30 Hz, 1H), 7.71 (s, 2H), 7.53 (m, 2H), 7.39 (m, 5H).

LC-MS: method X, RT=2.92 min (5 min run), MS (ESI) m/z: 251 (M+H)⁺; 249 (M−H)⁻

### Step 3:3-[[6-(2-phenylethynyl)-1,3-benzothiazol-2-yl]carbamoyl]bicyclo[2.2.1]hept-5-ene-2-carboxylic acid

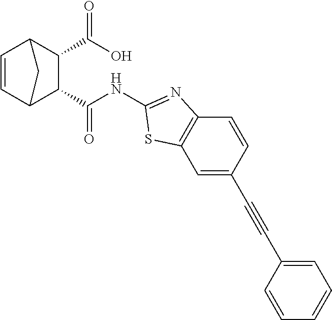 [SMILES: O=C(Nc1nc2ccc(C#Cc3ccccc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (47.9 mg, 0.29 mmol) in THF (3 mL) was added 6-(2-phenylethynyl)-1,3-benzothiazol-2-amine (73 mg, 0.29 mmol). The mixture was stirred under reflux overnight. The observed precipitate was filtrated, washed with ACN and dried. Then, the precipitate was solubilized in AcOEt, washed twice with HCl 1N and once with brine. The organic layer was evaporated under reduce pressure. ACN was added and the observed precipitate was filtrated, washed with ACN and dried to afford the title compound as white solid (17.8 mg, 14%).

¹H NMR (300 MHZ, DMSO-d6) δ=12.42(brs, 1H), 11.95 (brs, 1H), 8.18 (brs, 1H), 7.72(d, J=8.4 Hz, 1H), 7.58 (m, 3H), 7.44 (m, 3H), 6.17 (m, 2H), 3.48 (dd, J=9.7, 2.4 Hz, 1H), 3.40 (dd, J=9.6, 2.4 Hz, 1H), 3.14 (s, 1H), 3.10 (s, 1H), 1.35 (q, 2H).

LC-MS: method X, RT=3.03 min (5 min run), MS (ESI) m/z: 415 (M+H)⁺; 413 (M−H)⁻

HRMS (ES+) calculated for C24H19N2O3S [M+H]⁺: 415.1116 found 415.1129

### Example 78:3-[[6-(3,3-dimethylbut-1-ynyl)-1,3-benzothiazol-2-yl]carbamoyl]bicyclo [2.2.1]hept-5-ene-2-carboxylic acid

### Step 1: (HL3312) 6-(3,3-dimethylbut-1-ynyl)-1,3-benzothiazol-2-amine

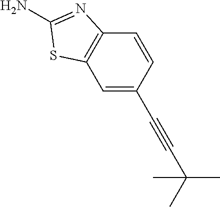 [SMILES: CC(C)(C)C#Cc1ccc2nc(N)sc2c1]

In a sealed tube was introduced 6-iodo-1,3-benzothiazol-2-amine (compound of step 1 of example 73, 100 mg, 0.36 mmol), PdCl₂(PPh₃)₂(15.3 mg, 0.022 mmol), and CuI (13.8 mg, 0.07 mmol). The mixture was purged with argon for 15 min, prior to the addition of ACN (1 mL), TEA (0.858 mL, 6.16 mmol) and 3,3-dimethylbut-1-yne (0.070 mL, 0.54 mmol). The solution was heated at 100° C. under microwave irradiation for 30 min. Then, EtOAc and the organic layer was washed twice with water. The aqueous phase was extracted with EtOAc and the organic layer were combined, dried, filtered and concentrated under reduce pressure. The crude was purified by column chromatography (from 100% DCM to 90/10 DCM/MeOH) to afford the title compound as light brown solid (36.6 mg, 44%).

¹H NMR (300 MHZ, DMSO-d6) δ=7.64 (d, J=1.74, 0.4 Hz, 1H), 7.42(d, J=8.47, 0.5 Hz, 1H), 7.35 (dd, J=8.31, 1.7 Hz, 1H), 1.35 (s, 9H).

LC-MS: method X, RT=2.97 min (5 min run), MS (ESI) m/z: 229 (M+H)⁺; 227 (M−H)⁻

### Step 2:3-[[6-(3,3-dimethylbut-1-ynyl)-1,3-benzothiazol-2-yl]carbamoyl]bicyclo[2.2.1]hept-5-ene-2-carboxylic acid

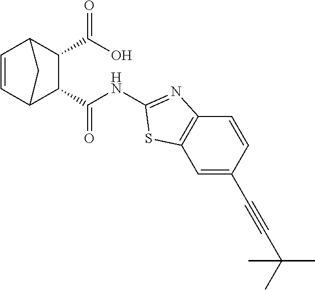 [SMILES: CC(C)(C)C#Cc1ccc2nc(NC(=O)[C@@H]3C4C=CC(C4)[C@@H]3C(=O)O)sc2c1]

To a solution of (1R,2S,6R,7S)-4-oxatricyclo[5.2.1.02,6]dec-8-ene-3,5-dione (26.1 mg, 0.16 mmol) in THF (3 mL) was added 6-(3,3-dimethylbut-1-ynyl)-1,3-benzothiazol-2-amine (37 mg, 0.16 mmol). The mixture was stirred under reflux overnight. Then ACN was added and the observed precipitate was filtrated, washed with ACN and dried to afford the title compound as white solid (22.3 mg, 36%).

¹H NMR (300 MHZ, DMSO-d6) δ=12.35 (brs, 1H), 11.87 (brs, 1H), 7.95 (d, J=1.2 Hz, 1H), 7.62(d, J=8.5 Hz, 1H), 7.36 (dd, J=8.4, 1.6 Hz, 1H), 6.16 (s, 2H), 3.47 (dd, J=10.1, 2.7 Hz, 1H), 3.38 (dd, J=10.1, 2.7 Hz, 1H), 3.13 (s, 1H), 3.08 (s, 1H), 1.30 (m, 11H).

LC-MS: method X, RT=3.08 min (5 min run), MS (ESI) m/z: 395 (M+H)⁺; 393 (M−H)⁻

HRMS (ES+) calculated for C22H23N2O3S [M+H]⁺: 395.1429 found 395.1451

### Example 79:3-[[6-(2-cyclopropylethynyl)-1,3-benzothiazol-2-yl]carbamoyl]bicyclo[2.2.1]hept-5-ene-2-carboxylic acid

### Step 1:6-(2-cyclopropylethynyl)-1,3-benzothiazol-2-amine

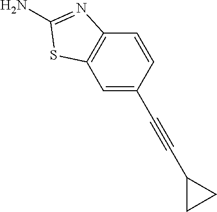 [SMILES: Nc1nc2ccc(C#CC3CC3)cc2s1]

In a sealed tube was introduced 6-iodo-1,3-benzothiazol-2-amine (compound of step 1 of example 73, 160 mg, 0.58 mmol), PdCl₂(PPh₃)₂(24.4 mg, 0.035 mmol), and CuI (22.1 mg, 0.12 mmol). The mixture was purged with argon for 15 min, prior to the addition of ACN (1 mL), TEA (1.7 mL, 12.2 mmol) and ethynylcyclopropane (0.142 mL, 1.68 mmol). The solution was heated at 100° C. under microwave irradiation for 30 min. Then, EtOAc and the organic layer was washed twice with water. The aqueous phase was extracted with EtOAc and the organic layer were combined, dried, filtered and concentrated under reduce pressure. The crude was purified by column chromatography (from 100% DCM to 90/10 DCM/MeOH). A second purification by reverse column chromatography (from 90/10 water/ACN to 100% ACN) was made to afford the title compound as light brown solid (33.2 mg, 26%).

LC-MS: method X, RT=2.62 min (5 min run), MS (ESI) m/z: 229 (M+H)⁺; 227 (M−H)⁻

### Step 2:3-[[6-(2-cyclopropylethynyl)-1,3-benzothiazol-2-yl]carbamoyl]bicyclo[2.2.1]hept-5-ene-2-carboxylic acid

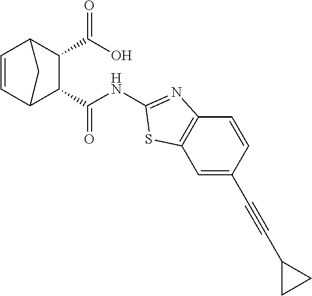 [SMILES: O=C(Nc1nc2ccc(C#CC3CC3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (25.4 mg, 0.16 mmol) in THF (3 mL) was added 6-(2-cyclopropylethynyl)-1,3-benzothiazol-2-amine (33.2 mg, 0.16 mmol). The mixture was stirred under reflux overnight. Then ACN was added and the observed precipitate was filtrated, washed with ACN and dried to afford the title compound as white solid (30 mg, 51%).

¹H NMR (300 MHZ, DMSO-d6) δ=12.34 (brs, 1H), 11.86 (brs, 1H), 7.96 (s, 1H), 7.62(d, J=8.3 Hz, 1H), 7.37 (dd, J=8.3, 1.2 Hz, 1H), 6.16 (s, 2H), 3.47 (dd, J=10.2, 2.8 Hz, 1H), 3.39 (dd, J=10.2, 3.2 Hz, 1H), 3.13 (s, 1H), 3.08 (s, 1H), 1.55 (m, 1H), 1.34 (q, J=7.8 Hz, 2H), 0.93 (m, 2H), 0.89 (m, 2H).

LC-MS: method X, RT=2.80 min (5 min run), MS (ESI) m/z: 379 (M+H)⁺; 377 (M−H)⁻

HRMS (ES+) calculated for C21H19N2O3S [M+H]⁺: 379.1116 found 379.1129

### Example 80:3-[(6-pyrimidin-5-yl-1,3-benzothiazol-2-yl)carbamoyl]bicyclo[2.2.1]hept-5-ene-2-carboxylic acid

### Step 1:6-(3-pyridyl)-1,3-benzothiazol-2-amine

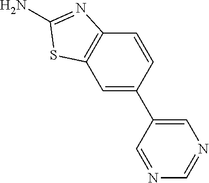 [SMILES: Nc1nc2ccc(-c3cncnc3)cc2s1]

In a sealed vial was introduced 6-bromo-1,3-benzothiazol-2-amine (300 mg, 1.31 mmol), 5-pyrimidinylboronic acid (243 mg, 1.96 mmol), Bis(Triphenylphosphine) palladium (II) chloride (47.9 mg, 0.066 mmol) and K₂CO₃ (362 mg, 2.62 mmol). The reaction mixture was purged under argon for 15 min prior to the addition of a mixture of 1,4-dioxane (6.90 mL) and water (1.80 mL). The mixture was submitted to irradiation for 60 min at 100° C. Then, the mixture was filtered on a pad of celite and the filtrate was evaporated under reduce pressure. The crude was purified by column chromatography (from 100% cyclohexane to 70/30 AcOEt/MeOH) to afford the title compound as brown solid (152.8 mg, 51%).

¹H NMR (300 MHZ, DMSO-d6) δ=9.12(m, 3H), 8.16 (d, J=1.7 Hz, 1H), 7.66 (m, 3H), 7.45 (d, J=8.4 Hz, 1H).

LC-MS: method X, RT=1.66 min (5 min run), MS (ESI) m/z: 229 (M+H)⁺; 227 (M−H)⁻

### Step 2:3-[(6-pyrimidin-5-yl-1,3-benzothiazol-2-yl)carbamoyl]bicyclo[2.2.1]hept-5-ene-2-carboxylic acid

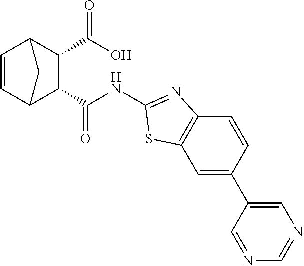 [SMILES: O=C(Nc1nc2ccc(-c3cncnc3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (105 mg, 0.64 mmol) in THF (3.5 mL) was added 6-pyrimidin-5-yl-1,3-benzothiazol-2-amine (146.0 mg, 0.64 mmol). The mixture was stirred under reflux. After 4 days, the reaction mixture was evaporated and purified by column chromatography (from 80/20 cyclohexane/AcOEt to90/10 AcOEt/MeOH) to give the title compound as white solid (17.2 mg, 7%).

¹H NMR (300 MHz, DMSO-d6) δ=12.37 (brs, 1H), 11.98 (brs, 1H), 9.21 (s, 2H), 9.17 (s, 1H), 8.44 (dd, J=1.8, 0.6 Hz, 1H), 7.89-7.85 (dd, J=8.5, 1.8 Hz, 1H), 7.84-7.81 (dd, J=8.5, 0.6 Hz, 1H), 6.17 (m, 2H), 3.51-3.46 (dd, J=10.0, 3.1 Hz, 1H), 3.41-3.37 (dd, J=10.0, 3.2 Hz, 1H), 3.15 (s, 1H), 3.09 (s, 1H), 3.34 (m, 2H).

LC-MS: method X, RT=9.00 min (30 min run), MS (ESI) m/z: 375 (M+H)⁺

HRMS (ES+) calculated for C20H17N403S [M+H]⁺: 393.1021 found 393.1032

### Example 81:3-[[6-(6-quinolyl)-1,3-benzothiazol-2-yl]carbamoyl]bicyclo[2.2.1]hept-5-ene-2-carboxylic acid

### Step 1:6-(6-quinolyl)-1,3-benzothiazol-2-amine

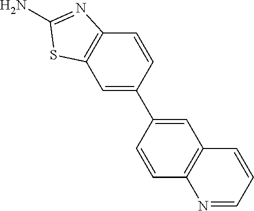 [SMILES: Nc1nc2ccc(-c3ccc4ncccc4c3)cc2s1]

In a sealed vial was introduced 6-bromo-1,3-benzothiazol-2-amine (100 mg, 0.436 mmol), 6-quinolylboronic acid (98.2 mg, 0.567 mmol), Bis(Triphenylphosphine) palladium (II) chloride (15.9 mg, 0.0218 mmol) and CsF (199 mg, 1.31 mmol) in a mixture of 1,4-dioxane (3.60 mL) and water (1.80 mL). The reaction mixture was purged under argon for 15 min and heated at 90° C. After 6 days, the reaction was cooled to room temperature and filtered on a pad of celite. DCM and water were added and the aqueous phase was extracted with DCM twice. The combined organic layers were washed with sat. solution of NaHCO₃ and then with brine. The organic layer was dried, filtered and concentrated under reduce pressure. The crude was purified by column chromatography (from 100% cyclohexane to 100% AcOEt) to afford the title compound as brown solid (29 mg, 24%).

¹H NMR (300 MHZ, DMSO-d6) δ=8.89 (dd, J=4.1, 1.7 Hz, 1H), 8.41 (dd, J=8.5, 1.6 Hz, 1H), 8.28 (d, J=2.0 Hz, 1H), 8.18 (d, J=2.0 Hz, 1H), 8.13 (dd, J=8.9, 2.1 Hz, 1H), 8.07 (d, J=8.9 Hz, 1H), 7.71 (dd, J=8.5, 2.0 Hz, 1H), 7.60 (s, 2H), 7.56 (q, J=4.1 Hz, 1H), 7.46 (d, J=8.3 Hz, 1H).

LC-MS: method X, RT=2.18 min (5 min run), MS (ESI) m/z: 278 (M+H)⁺

### Step 2:3-[[6-(6-quinolyl)-1,3-benzothiazol-2-yl]carbamoyl]bicyclo[2.2.1]hept-5-ene-2-carboxylic acid

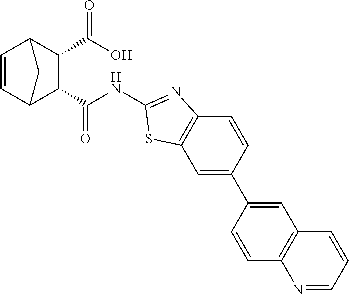 [SMILES: O=C(Nc1nc2ccc(-c3ccc4ncccc4c3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (38.5 mg, 0.24 mmol) in THF (3.5 mL) was added 6-(6-quinolyl)-1,3-benzothiazol-2-amine (65.0 mg, 0.24 mmol). The mixture was stirred under reflux. After 36 h, the observed precipitate was filtrated, washed with ACN and THF to afford the title acid compound as white solid (42.2 mg, 41%).

¹H NMR (300 MHZ, DMSO-d6) δ=12.37 (brs, 1H), 11.95 (brs, 1H), 8.91 (dd, J=4.2, 1.7 Hz, 1H), 8.47-8.36 (m, 3H), 8.21-8.17 (dd, J=8.9, 2.0 Hz, 1H), 8.10 (d, J=8.9 Hz, 1H), 7.91 (dd, J=8.5, 1.8 Hz, 1H), 7.84 (d, J=8.5 Hz, 1H), 7.57 (dd, J=8.3, 4.2 Hz, 1H), 6.19 (m, 2H), 3.50 (dd, J=10.0, 3.0 Hz, 1H), 3.40 (dd, J=10.0, 3.1 Hz, 1H), 3.16 (s, 1H), 3.10 (s, 1H), 1.36 (m, 2H).

LC-MS: method X, RT=2.43 min (5 min run), MS (ESI) m/z: 442(M+H)⁺; 440 (M−H);

HRMS (ES+) calculated for C25H2ON3O3S [M+H]⁺: 442.1225 found 442.1246

### Example 82: (1R,2S,6R,7S)-4-(6-anilino-1,3-benzothiazol-2-yl)-4-azatricyclo[5.2.1.02,6]dec-8-ene-3,5-dione

### Step 1: N6-phenyl-1,3-benzothiazole-2,6-diamine

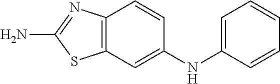 [SMILES: Nc1nc2ccc(Nc3ccccc3)cc2s1]

To a solution of N1-phenylbenzene-1,4-diamine (458 mg, 2.49 mmol) in acetic acid (5 mL) was added ammonium thiocyanate (946 mg, 12.4 mmol) and the reaction was stirred for 90 min. The mixture was cooled to 0° C. and a solution of Br₂(140 μL, 2.73 mmol) in acetic acid (2.5 mL) was added dropwise. The reaction was stirred for 1 h at 0° C. and then allowed to room temperature and stirred overnight. Then, the mixture was poored into cold water and a saturated solution of Na₂CO₃ was added till pH˜10. The aqueous phase was extracted with ethyl acetate (3 times). The combined organic phases were dried, filtered and concentrated under reduce pressure. The crude was purified by column chromatography (from 100% cyclohexane to 20/80 cyclohexane/ethyl acetate). The desired product was afforded as a brown solid (179 mg, 30%).

¹H NMR (300 MHZ, CDl₃OD) δ=7.38 (m, 2H), 6.98 (m, 2H), 6.88 (m, 2H), 6.76 (m, 2H).

LC-MS: method X, RT=2.48 min (5 min run), MS (ESI) m/z: 242(M+1H)⁺; 240 (M−1H)

### Step 2:(1R,2S,6R,7S)-4-(6-anilino-1,3-benzothiazol-2-yl)-4-azatricyclo[5.2.1.02,6]dec-8-ene-3,5-dione

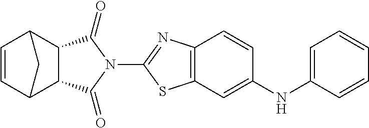 [SMILES: O=C1[C@@H]2C3C=CC(C3)[C@@H]2C(=O)N1c1nc2ccc(Nc3ccccc3)cc2s1]

To a solution of (1R,2S,6R,7S)-4-oxatricyclo[5.2.1.02,6]dec-8-ene-3,5-dione (68 mg, 0.41 mmol) in THF (2 mL) was added N6-phenyl-1,3-benzothiazole-2,6-diamine (100 mg, 0.41 mmol). The mixture was heated under reflux. After 6 h, the observed precipitate was filtered, washed with THF and ACN and dried to afford the title acid compound as white solid (The filtrate was purified by column chromatography (from 50/50 cyclohexane/ethyl acetate to 90/10 ethyl acetate/methanol). The desired product was afforded as white solid (25.3 mg, 16%).

¹H NMR (300 MHZ, CDCl₃) δ=7.45 (m, 2H), 7.16-7.04 (m, 6H), 6.26 (t, J=1.8 Hz, 2H), 6.01 (brs, 1H), 3.51 (m, 2H), 3.43 (m, 2H), 1.80 (dt, J=8.8, 1.6 Hz, 1H), 1.63 (d, J=8.8 Hz, 1H).

LC-MS: method X, RT=2.87 min (5 min run), MS (ESI) m/z: 388 (M+1H)⁺; 386 (M−1H)⁻

HRMS (ES+) calculated for C22H18N302S [M+H]+388.1120 found 388.1123

### Example 83: (1R,2S,6R,7S)-4-[6-(2-pyridylamino)-1,3-benzothiazol-2-yl]-4-azatricyclo[5.2.1.02,6]dec-8-ene-3,5-dione

### Step 1: tert-butyl N-(6-bromo-1,3-benzothiazol-2-yl)carbamate

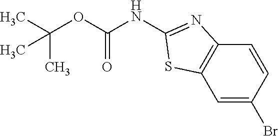 [SMILES: CC(C)(C)OC(=O)Nc1nc2ccc(Br)cc2s1]

To a solution of 6-bromo-1,3-benzothiazol-2-amine (500 mg, 2.18 mmol) in DCM (20 mL) at 0° C. was added DMAP (8 mg, 0.066 mmol) and BOC anhydride (714 mg, 3.27 mmol). The reaction was allowed to warm to room temperature and stirred overnight. After 16 h, water was added to the reaction mixture and the aqueous phase was extracted 3 times with ethyl acetate. The combined organic phases were washed with water and brine, and then, dried, filtered and concentrated under reduce pressure. The crude was purified by column chromatography (from 100% cyclohexane to 70/30 cyclohexane/AcOEt) to afford the title compound as white solid (486.2 mg, 67.7% yield).

¹H NMR (300 MHZ, CDCl₃) δ=7.91 (d, J=1.9 Hz, 1H), 7.70 (d, J=8.6 Hz, 1H), 7.54-7.49 (dd, J=8.6, 1.9 Hz, 1H), 1.6 (s, 9H).

LC-MS: method X, RT=3.27 min (5 min run), MS (ESI) m/z: 275 (M−C(CH₃)₃)+; 329 (M−H)⁻

### Step 2: tert-butyl N-[6-(2-pyridylamino)-1,3-benzothiazol-2-yl]carbamate

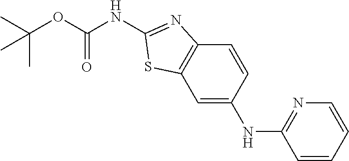 [SMILES: CC(C)(C)OC(=O)Nc1nc2ccc(Nc3ccccn3)cc2s1]

In a vial was introduced 1,4-dioxane degassed for 10 minutes prior to the addition of Pd(OAc) 2(40.9 mg, 0.18 mmol) and X-Phos (237 mg, 0.56 mmol). The mixture was stirred for 2 minutes at 110° C. followed by the addition of pyridin-2-amine (206 mg, 2.19 mmol) and tBuOK (614 mg, 5.47 mmol). The reaction mixture was stirred at 110° C. After 48 h, the reaction was cooled to room temperature and the solvent was evaporated. The crude was purified by column chromatography (from 80/20 cyclohexane/ethyl acetate to 90/10 ethyl acetate/methanol). The desired product was afforded as white solid (244.1 mg, 39%).

LC-MS: method X, RT=2.78 min (5 min run), MS (ESI) m/z: 343 (M+H)⁺; 341 (M−H)⁻

### Step 3: N6-(2-pyridyl)-1,3-benzothiazole-2,6-diamine

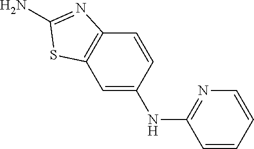 [SMILES: Nc1nc2ccc(Nc3ccccn3)cc2s1]

To a solution of DCM/TFA (5 mL/0.5 mL) was added tert-butyl N-[6-(2-pyridylamino)-1,3-benzothiazol-2-yl]carbamate (220 mg, 0.64 mmol) and the mixture was stirred at room temperature. Then, the solution was neutralized with a saturated solution of NaHCO₃. The aqueous phase was extracted with DCM (3 times). The combined organic phases were dried, filtered and concentrated under reduce pressure. The crude was purified by column chomatography (from 100% cyclohexane to 100% ethyl acetate). The desired product was afforded as white solid (90 mg, 58%).

¹H NMR (300 MHZ, CDCl₃) δ=8.88 (s, 1H), 8.14 (d, J=1.8 Hz, 1H), 8.10 (m, 1H), 7.50 (m, 1H), 7.29-7.21 (m, 4H), 6.76 (d, J=8.4 Hz, 1H), 6.67 (ddd, J=6.0, 5.0, 0.8 Hz, 1H).

### Step 4: 4-[6-(2-pyridylamino)-1,3-benzothiazol-2-yl]-4-azatricyclo[5.2.1.02,6]dec-8-ene-3,5-dione

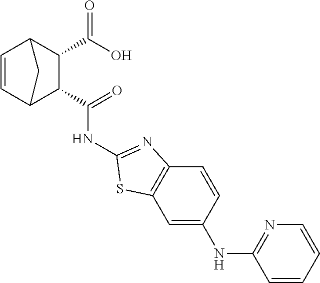 [SMILES: O=C(Nc1nc2ccc(Nc3ccccn3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (61.1 mg, 0.37 mmol) in THF (2 mL) was added N6-(2-pyridyl)-1,3-benzothiazole-2,6-diamine (90.2 mg, 0.37 mmol) and the mixture was heated under reflux overnight. Then, the solvent was evaporated under reduce pressure. The crude was purified by column chromatography (from 50/50 cyclohexane/ethyl acetate to 90/10 ethyl acetate/methanol) to afford the title compound as white solid (8.4 mg, 6%).

¹H NMR (300 MHZ, DMSO-d6) δ=12.12(brs, 1H), 11.85 (brs, 1H), 9.12(s, 1H), 8.42(d, J=2.0 Hz, 1H), 8.17 (m, 1H), 7.60-7.46 (m, 3H), 6.83 (d, J=8.4 Hz, 1H), 6.73 (m, 1H), 6.15 (m, 2H), 3.45 (dd, J=10.0, 3.3 Hz, 1H), 3.33 (m, 1H), 3.12(s, 1H), 3.07 (s, 1H), 1.33 (m, 2H).

LC-MS: method X, RT=2.20 min (5 min run), MS (ESI) m/z: 407 (M+H)⁺; 405 (M−H)⁻

HRMS (ES+) calculated for C21H19N403S [M+H]+407.1178 found 407.1171

### Example 84: 3-[[6-[(4-methyl-2-pyridyl)amino]-1,3-benzothiazol-2-yl]carbamoyl]bicyclo[2.2.1]hept-5-ene-2-carboxylic acid

### Step 2: N6-(4-methyl-2-pyridyl)-1,3-benzothiazole-2,6-diamine

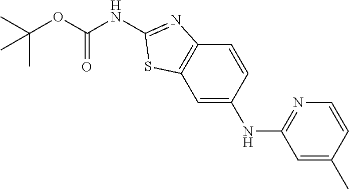 [SMILES: Cc1ccnc(Nc2ccc3nc(NC(=O)OC(C)(C)C)sc3c2)c1]

In a vial was introduced 1,4-dioxane degassed for 10 minutes prior to the addition of Pd(OAc) 2(40.9 mg, 0.18 mmol) and X-Phos (237 mg, 0.56 mmol). The mixture was stirred for 2 minutes at 110° C. followed by the addition of 4-methylpyridin-2-amine (237 mg, 2.19 mmol) and tBuOK (614 mg, 5.47 mmol). The reaction mixture was stirred at 110° C. After 48 h, the reaction was cooled to room temperature and the solvent was evaporated. The crude was purified by column chromatography (from 50/50 cyclohexane/ethyl acetate to 90/10 ethyl acetate/methanol). The desired product was afforded as white solid (136.9 mg, 21%).

LC-MS: method X, RT=2.73 min (5 min run), MS (ESI) m/z: 357 (M+H)⁺; 355 (M−H)⁻

### Step 3: N6-(4-methyl-2-pyridyl)-1,3-benzothiazole-2,6-diamine

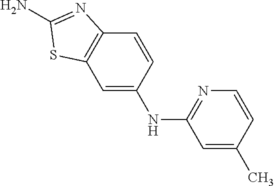 [SMILES: Cc1ccnc(Nc2ccc3nc(N)sc3c2)c1]

To a solution of DCM/TFA (5 mL/0.5 mL) was added tert-butyl N-[6-[(4-methyl-2-pyridyl)amino]-1,3-benzothiazol-2-yl]carbamate (120 mg, 0.34 mmol) and the mixture was stirred at room temperature. Then, the solution was neutralized with a saturated solution of NaHCO₃. The aqueous phase was extracted with DCM (3 times). The combined organic phases were dried, filtered and concentrated under reduce pressure. The crude was purified by column chomatography (from 100% cyclohexane to 100% ethyl acetate). The desired product was afforded as white solid (43.8 mg, 51%).

¹H NMR (300 MHZ, CDCl₃) δ=8.78 (s, 1H), 8.13 (d, J=1.8 Hz, 1H), 7.98 (d, J=5.2 Hz, 1H), 7.27 (dd, J=8.6, 2.0 Hz, 1H), 7.24 (s, 1H), 7.21 (s, 2H), 6.58 (s, 1H), 6.54 (d, J=5.2 Hz, 1H), 2.21 (s, 3H).

### Step 4: 3-[[6-[(4-methyl-2-pyridyl)amino]-1,3-benzothiazol-2-yl]carbamoyl]bicyclo[2.2.1]hept-5-ene-2-carboxylic acid

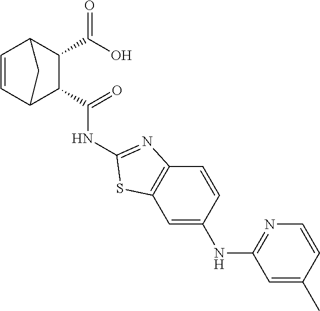 [SMILES: Cc1ccnc(Nc2ccc3nc(NC(=O)[C@@H]4C5C=CC(C5)[C@@H]4C(=O)O)sc3c2)c1]

To a solution of (1R,2S,6R,7S)-4-oxatricyclo[5.2.1.02,6]dec-8-ene-3,5-dione (26.1 mg, 0.16 mmol) in THF (3 mL) was added N6-(4-methyl-2-pyridyl)-1,3-benzothiazole-2,6-diamine (40.8 mg, 0.16 mmol) and the mixture was heated under reflux overnight. Then, the observed precipitate was filtrated, washed with a mixture ACN/THF and dried to afford the title acid compound as white solid (Example 84, 20.3 mg, 30%).

¹H NMR (300 MHZ, DMSO-d6) δ=12.10 (brs, 1H), 11.84 (brs, 1H), 9.01 (s, 1H), 8.41 (d, J=2.0 Hz, 1H), 8.03 (d, J=5.2 Hz, 1H), 7.58 (d, J=8.7 Hz, 1H), 7.47 (dd, J=8.8, 2.2 Hz, 1H), 6.64 (s, 1H), 6.59 (d, J=5.2 Hz, 1H), 6.15 (m, 2H), 3.46 (dd, J=10.2, 3.2 Hz, 1H), 3.35 (m, 1H), 3.12(s, 1H), 3.06 (s, 1H), 2.29 (s, 3H), 1.36 (d, J=8.4 Hz, 1H), 1.29 (d, J=8.4 Hz, 1H).

LC-MS: method X, RT=8.33 min (30 min run), MS (ESI) m/z: 421 (M+H)⁺; 419 (M−H)⁻

HRMS (ES+) calculated for C22H21N403S [M+H]⁺: 421.1334 found 421.1342

### Example 85:3-[[6-[(4-fluoro-2-pyridyl)amino]-1,3-benzothiazol-2-yl]carbamoyl]bicyclo[2.2.1]hept-5-ene-2-carboxylic acid

### Step 1 6-[(4-fluoro-2-pyridyl)amino]-1,3-benzothiazol-2,6-diamine

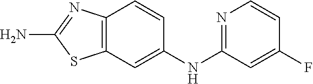 [SMILES: Nc1nc2ccc(Nc3cc(F)ccn3)cc2s1]

The tert-butyl-N-[6-bromo-1,3-benzothiazol-2-amino]carbamate (300.0 mg, 0.911 mmol), 4-fluoropyridin-2-amine (123.0 mg, 1.09 mmol), Pd₂dba₃ (26.2 mg, 0.0911 mmol), tBuONa (131.0 mg, 1.37 mmol) and tBuBrettPhos (44.2 mg, 0.0911 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The product was purified by flash chromatography (10/0 to 3/7 DCM/EA), yielding an off-white solid (159 mg, 67%).

¹H NMR (300 MHz, CD₃CN) δ=8.14 (dd, 1H, J=5.8 Hz, 9.4 Hz), 7.98 (d, 1H, J=2.2 Hz), 7.51 (s, 1H), 7.39 (d, 1H, J=8.6 Hz), 7.27 (dd, 1H, J=2.2 Hz, 8.6 Hz), 6.55 (dt, 1H, J=2.2 Hz, 8.0 Hz), 6.47 (dd, 1H, J=2.1 Hz, 11.8 Hz), 5.86 (s, 2H).

¹⁹F NMR (300 MHz, CD₃CN) δ=−105.93.

LC-MS: 1.63 mins (5 mins) MS (ESI) m/z: (M+H)+261.13; (M−H)⁻259.13

### Step 2 3-[[6-[(4-fluoro-2-pyridyl)amino]-1,3-benzothiazol-2-yl]carbamoyl]bicyclo[2.2.1]hept-5-ene-2-carboxylic acid

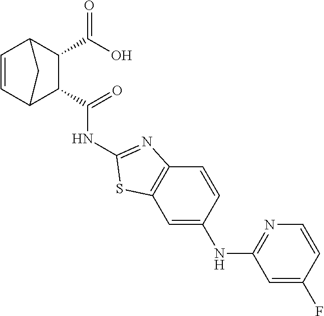 [SMILES: O=C(Nc1nc2ccc(Nc3cc(F)ccn3)cc2s1)[C@@H]1C2C=CC(C2)[C@@H]1C(=O)O]

To a solution of (1R,2S,6R,7S)-4-oxatricyclo[5.2.1.02,6]dec-8-ene-3,5-dione (95 mg, 0.365 mmol) in dry THF (0.6 mL) was added N6-(4-pyridyl)-1,3-benzothiazole-2,6-diamine (59.9 mg, 0.365 mmol). The mixture was stirred under reflux for 4 h, then cooled to rt, after which a white precipitate is observed. The precipitate was filtered then abundantly washed with THF and ACN, after which it was dried to afford the title compound as a white solid (42 mg, 28% yield).

¹H NMR (300 MHZ, DMSO-d6) δ=12.13 (s, 1H), 11.86 (s, 1H), 9.33 (s, 1H), 8.37 (d, 1H, J=2.0 Hz), 8.21 (dd, 1H, J=5.8 Hz, 9.7 Hz), 7.62(d, 1H, J=8.7 Hz), 7.47 (dd, 1H, J=2.1 Hz, 8.7 Hz), 6.67 (dt, 1H, J=2.2 Hz, 8.5 Hz), 6.59 (dd, 1H, J=2.1 Hz, 11.9 Hz), 6.17 (m, 2H), 3.48 (dd, 1H, J=3.1 Hz, 10.0 Hz), 3.36 (m, 1H), 3.11 (d, 2H, J=15.2 Hz), 1.35 (m, 2H).

¹⁹F NMR (300 MHz, DMSO-d6)·=−104.56.

LC-MS: method X, RT=9.01 min (30 min run), MS (ESI) m/z: (M+H)+425.29; (M−H)⁻ 423.24

HRMS (ES+) calculated for C21H18N4O3SF [M+H]⁺: 425.1084, found 425.1073.

### Example 86 and 87:3-[[6-[(5-fluoro-2-pyridyl)amino]-1,3-benzothiazol-2-yl]carbamoyl]bicyclo[2.2.1]hept-5-ene-2-carboxylic acid and 4-[6-[(5-fluoro-2-pyridyl)amino]-1,3-benzothiazol-2-yl]-4-azatricyclo[5.2.1.02,6]dec-8-ene-3,5-dione

### Step 1 tert-butyl N-[6-[[5-fluoro-2-pyridyl]amino]-1,3-benzothiazol-2-yl]carbamate

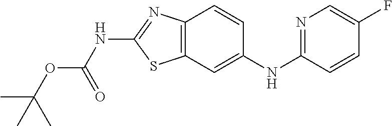 [SMILES: CC(C)(C)OC(=O)Nc1nc2ccc(Nc3ccc(F)cn3)cc2s1]

The tert-butyl-N-[6-bromo-1,3-benzothiazol-2-amino]carbamate (200.0 mg, 0.612 mmol), 5-fluoropyridin-2-amine (68.0 mg, 0.612 mmol), Pd₂dba₃ (27.9 mg, 0.0306 mmol), tBuONa (88.2 mg, 0.918 mmol) and tBuBrettPhos (29.7 mg, 0.0612 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The product was purified by flash chromatography (10/0 to 3/7 DCM/EA), yielding the title compound as an off-white solid (108 mg, 68%).

¹H NMR (300 MHz, MeOD) δ=8.27 (d, 1H, J=2.1 Hz), 8.03 (d, 1H, J=3.0 Hz), 7.57 (d, 1H, J=8.7 Hz), 7.45-7.35 (m, 2H), 6.84 (dd, 1H, J=3.5 Hz, 9.1 Hz).

¹⁹F NMR (300 MHz, MeOD) δ=−144.03.

LC-MS: 2.16 mins (5 min run) MS (ESI) m/z: (M+H)+361.19; (M−H)⁻359.14

### Step 2 N6-(5-fluoro-2-pyridyl)-1,3-benzothiazole-2,6-diamine

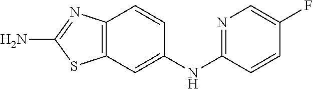 [SMILES: Nc1nc2ccc(Nc3ccc(F)cn3)cc2s1]

tert-butyl-N-[6-[(5-fluoro-2-pyridyl)amino]-1,3-benzothiazol-2-yl]carbamate (108 mg, 0.415 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a yellow oil (93 mg, 86% yield).

LC-MS: 1.07 mins (5 min run), MS (ESI) m/z: (M+H)+261.15.

### Step 3

### Example 86:3-[[6-[(5-fluoro-2-pyridyl)amino]-1,3-benzothiazol-2-yl]carbamoyl]bicyclo[2.2.1]hept-5-ene-2-carboxylic acid

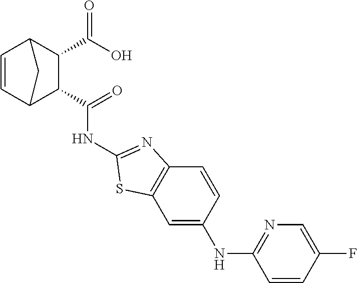 [SMILES: O=C(Nc1nc2ccc(Nc3ccc(F)cn3)cc2s1)[C@@H]1C2C=CC(C2)[C@@H]1C(=O)O]

### Example 87:4-[6-[(5-fluoro-2-pyridyl)amino]-1,3-benzothiazol-2-yl]-4-azatricyclo[5.2.1.02,6]dec-8-ene-3,5-dione

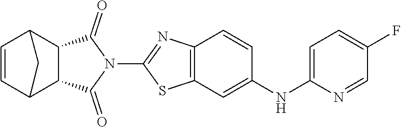 [SMILES: O=C1[C@@H]2C3C=CC(C3)[C@@H]2C(=O)N1c1nc2ccc(Nc3ccc(F)cn3)cc2s1]

To a solution of (1R,2S,6R,7S)-4-oxatricyclo[5.2.1.02,6]dec-8-ene-3,5-dione (70.6 mg, 0.430 mmol) in dry THF (1.0 mL) was added N6-(5-fluoro-2-pyridyl)-1,3-benzothiazole-2,6-diamine (112 mg, 0.430 mmol). The mixture was stirred under reflux for 4 h, then cooled to rt, after which a white precipitate is observed. The precipitate was filtered then abundantly washed with THF and ACN, after which it was dried to afford the title compound as a white solid (Example 86, 113 mg, 61% yield). The filtrate was then purified by flash chromatography (10/0 to 5/5 Cy/EA) to yield compound as an off-white solid (Example 87, 7 mg, 4% yield).

### Example 86

¹H NMR (300 MHz, DMSO-d6) δ=12.10 (s, 1H), 11.98 (s, 1H), 9.21 (s, 1H), 8.38 (d, 1H, J=2.1 Hz), 8.15 (d, 1H, J=3.03 Hz), 9.61-9.51 (m, 2H), 7.45 (dd, 1H, J=2.1 Hz, 8.8 Hz), 6.88 (dd, 1H, J=3.6 Hz, 9.2 Hz), 6.18-6.14 (m, 2H), 3.46-3.40 (m, 2H), 3.10 (d, 2H, J=15.8 Hz), 1.34 (q, 2H, J=8.3 Hz).

¹⁹F NMR (300 MHz, DMSO-d₆) δ=−141.8.

LC-MS: method X, RT=9.19 min (30 min run), MS (ESI) m/z: (M+H)+423.24; (M−H)⁻ 425.19

HRMS (ES+) calculated for C21H18N403SF [M+H]⁺: 425.1084, found 425.1084

### Example 87

¹H NMR (300 MHz, CDCl₃) δ=8.08 (dd, 2H, J=2.8 Hz, 8.9 Hz), 8.02(d, 1H, J=8.8 Hz), 7.41-7.30 (m, 2H), 7.05 (s, 1H), 6.87 (dd, 1H, J=3.1 Hz, 9.1 Hz), 6.31-6.29 (m, 2H), 3.62-3.54 (m, 4H), 1.83 (d, 1H, J=8.9 Hz), 1.67 (d, 1H, J=9.0 Hz).

¹⁹F NMR (300 MHz, CDCl₃) δ=−139.40.

LC-MS: method X, RT=11.07 min (30 min run), MS (ESI) m/z: (M+H)+407.35

HRMS (ES+) calculated for C21H16N4O2FS [M+H]⁺: 407.0978, found 407.0953.

### Example 88 and 89:3-[[6-[(6-fluoro-2-pyridyl)amino]-1,3-benzothiazol-2-yl]carbamoyl]bicyclo[2.2.1]hept-5-ene-2-carboxylic acid and 4-[6-[(6-fluoro-2-pyridyl)amino]-1,3-benzothiazol-2-yl]-4-azatricyclo[5.2.1.02,6]dec-8-ene-3,5-dione

### Step 1 tert-butyl N-[6-[[6-fluoro-2-pyridyl]amino]-1,3-benzothiazol-2-yl]carbamate

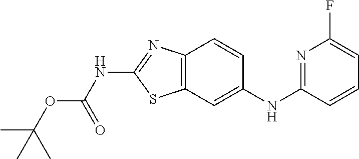 [SMILES: CC(C)(C)OC(=O)Nc1nc2ccc(Nc3cccc(F)n3)cc2s1]

The tert-butyl-N-[6-bromo-1,3-benzothiazol-2-amino]carbamate (200.0 mg, 0.608 mmol), 6-fluoropyridin-2-amine (68.1 mg, 0.608 mmol), Pd₂dba₃ (17.5 mg, 0.0304 mmol), tBuONa (87.6 mg, 0.911 mmol) and tBuBrettPhos (29.4 mg, 0.0608 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was triturated in a minimal amount of DCM, and a light yellow precipitate was obtained and filtered (119 mg, 54%).

¹H NMR (300 MHZ, MeOD) δ=7.99 (d, 1H, J=1.7 Hz), 7.65-7.56 (m, 1H), 7.34-7.25 (m, 2H), 6.60 (dd, 1H, J=2.0 Hz, 7.9 Hz), 6.26 (dd, 1H, J=2.3 Hz, 7.6 Hz).

¹⁹F NMR (300MHZ, MeOD) δ=−72.52.

LC-MS: 2.23 mins (5 min run), MS (ESI) m/z: (M+H)+361.19; (M−H)⁻359.09.

### Step 2 N6-(6-fluoro-2-pyridyl)-1,3-benzothiazole-2,6-diamine

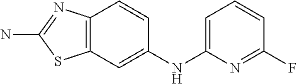 [SMILES: Nc1nc2ccc(Nc3cccc(F)n3)cc2s1]

tert-butyl-N-[6-[(6-fluoro-2-pyridyl)amino]-1,3-benzothiazol-2-yl]carbamate (119 mg, 0.330 mmol) was slurried in 0.7 mL DCM, to which was then added 0.3 mL TFA dropwise at rt.

The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a colorless oil (81 mg, 94% yield).

LC-MS: 1.73 mins (5 min run), MS (ESI) m/z: (M+H)+261.18; (M−H)⁻259.13.

### Step 3

### Example 88: 3-[[6-[(6-fluoro-2-pyridyl)amino]-1,3-benzothiazol-2-yl]carbamoyl]bicyclo[2.2.1]hept-5-ene-2-carboxylic acid

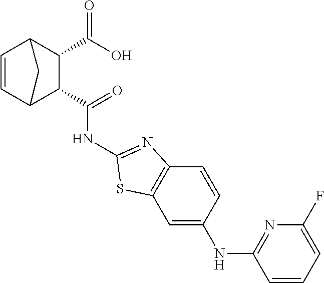 [SMILES: O=C(Nc1nc2ccc(Nc3cccc(F)n3)cc2s1)[C@@H]1C2C=CC(C2)[C@@H]1C(=O)O]

### Example 89:4-[6-[(6-fluoro-2-pyridyl)amino]-1,3-benzothiazol-2-yl]-4-azatricyclo[5.2.1.02,6]dec-8-ene-3,5-dione

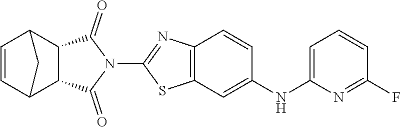 [SMILES: O=C1[C@@H]2C3C=CC(C3)[C@@H]2C(=O)N1c1nc2ccc(Nc3cccc(F)n3)cc2s1]

N6-[6-fluoro-2-pyridyl]-1,3-benzothiazole-2,6-diamine (81 mg, 0.311 mmol) and (1S,2R,6S,7R)-4-oxatricyclo[5.2.1.02,6]dec-8-ene-3,5-dione (56.1 mg, 0.311 mmol) were flushed with argon, then slurried in dry THF (1 mL), after which the mixture was heated at 70° C. After 4 h, the reaction was cooled to rt and a precipitate was observed. A first filtration was performed, the precipitate was washed with THF and acetonitrile. The title compound was obtained as a white solid (Example 88, 11 mg, 8% yield). The filtrate was dried, and the title compound was isolated by flash chromatography purification (9/1 to 5/5 Cy/EA) as a white solid (Example 89, 25 mg, 19% yield).

### Example 88

¹H NMR (300 MHz, DMSO-d₆) δ=12.27 (s, 1H), 11.73 (s, 1H), 9.44 (s, 1H), 8.22(d, 1H, J=2.1 Hz), 7.71 (q, 1H, J=8.8 Hz), 7.63 (d, 1H, J=8.7 Hz), 7.48 (dd, 1H, J=2.2 Hz, 8.8 Hz), 6.72(dd, 1H, J=2.1 Hz, 8.0 Hz), 6.40 (dd, 1H, J=2.4 Hz, 7.7 Hz), 6.19-6.14 (m, 2H), 3.46 (dd, 2H, J=3.1 Hz, 10.0 Hz), 3.11 (d, 2H, J=16.3 Hz), 1.34 (q, 2H, J=9.4 Hz).

¹⁹F NMR (300MHZ, DMSO-d₆) δ=−69.91.

LC-MS: method X, RT=9.60 mins (30 min run), MS (ESI) m/z: (M+H)+425.14; (M−H)⁻ 423.04

HRMS (ES+) calculated for C21H18N403SF [M+H]⁺: 425.1084, found 425.1086

### Example 89

¹H NMR (300 MHz, CDCl₃) δ=8.09 (d, 1H, J=2.0 Hz), 8.03 (d, 1H, J=8.8 Hz), 7.61 (dd, 1H, J=8.1 Hz, 16.1 Hz), 7.35 (dd, 1H, J=1.9 Hz, 8.7 Hz), 6.80 (s, 1H), 6.68 (dd, 1H, J=1.8 Hz, 8.0 Hz), 6.39 (dd, 1H, J=2.2 Hz, 7.8 Hz), 6.30 (t, 2H, J=1.6 Hz), 3.61-3.55 (m, 4H), 1.84 (d, 1H, J=8.7 Hz), 1.67 (d, 1H, J=8.8 Hz).

LC-MS: method X, RT=9.64 mins (30 min run), MS (ESI) m/z: 407.15 (M+H)⁺

HRMS (ES+) calculated for C21H16N402SF [M+H]⁺: 407.0978, found 407.0908.

### Example 90:4-[6-[[2-(trifluoromethyl)-4-pyridyl]amino]-1,3-benzothiazol-2-yl]-4-azatricyclo[5.2.1.02,6]dec-8-ene-3,5-dione

### Step 1 tert-butyl N-[6-[[2-(trifluoromethyl)-4-pyridyl]amino]-1,3-benzothiazol-2-yl]carbamate

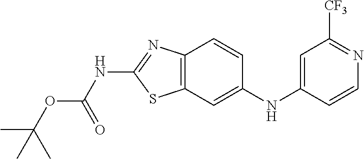 [SMILES: CC(C)(C)OC(=O)Nc1nc2ccc(Nc3ccnc(C(F)(F)F)c3)cc2s1]

The tert-butyl-N-[6-bromo-1,3-benzothiazol-2-amino]carbamate (200.0 mg, 0.873 mmol), 5-(trifluoromethyl)pyridin-4-amine (142.0 mg, 0.873 mmol), Pd₂dba₃ (25.1 mg, 0.043 mmol), tBuONa (126.0 mg, 1.31 mmol) and tBuBrettPhos (42.3 mg, 0.0873 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was triturated in a minimal amount of DCM, and a light yellow precipitate was obtained and filtered (220 mg, 61%).

¹H NMR (300 MHZ, MeOD) δ=8.23 (d, 1H, J=5.8 Hz), 7.75 (d, 1H, J=2.1 Hz), 7.71 (d, 1H, J=8.7 Hz), 7.31 (dd, 1H, J=2.2 Hz, 8.6 Hz), 7.19 (d, 1H, J=2.3 Hz), 7.05 (dd, 1H, J=2.3 Hz, 5.9 Hz).

¹⁹F NMR (300MHZ, MeOD) δ=−70.47.

LC-MS: 2.15 mins (5 min run), MS (ESI) m/z: 411.20 (M+H)⁺; 409.20 (M−H)⁻

### Step 2 N6-[[2-(trifluoromethyl)-4-pyridyl]-1,3-benzothiazole-2,6-diamine

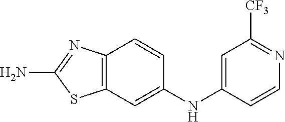 [SMILES: Nc1nc2ccc(Nc3ccnc(C(F)(F)F)c3)cc2s1]

tert-butyl N-[6-[(2-trifluoromethyl-4-pyridyl)amino]-1,3-benzothiazol-2-yl]carbamate (160 mg, 0.390 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a yellow oil (112 mg, 92% yield).

LC-MS: 1.66 mins (5 min run), MS (ESI) m/z: 311.08 (M+H)⁺; 309.03 (M−H)⁻

### Step 3

### Example 90:4-[6-[[2-(trifluoromethyl)-4-pyridyl]amino]-1,3-benzothiazol-2-yl]-4-azatricyclo[5.2.1.02,6]dec-8-ene-3,5-dione

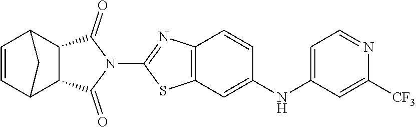 [SMILES: O=C1[C@@H]2C3C=CC(C3)[C@@H]2C(=O)N1c1nc2ccc(Nc3ccnc(C(F)(F)F)c3)cc2s1]

N6-[2-trifluoromethyl-4-pyridyl]-1,3-benzothiazole-2,6-diamine (112 mg, 0.361 mmol) and (1S,2R,6S,7R)-4-oxatricyclo[5.2.1.02,6]dec-8-ene-3,5-dione (59.3 mg, 0.361 mmol) were flushed with argon, then slurried in dry THF (1 mL), after which the mixture was heated at 70° C. After 4 h, the reaction was cooled to rt and a precipitate was observed. A first filtration was performed, the precipitate was washed with THF and acetonitrile. The title compound was obtained as a white solid (11 mg, 8% yield). The filtrate was dried, and the title compound was isolated by flash chromatography purification (9/1 to 5/5 Cy/EA) as a white solid (25 mg, 19% yield).

¹H NMR (300 MHz, CDCl₃) δ=8.39 (d, 1H, J=5.7 Hz), 8.09 (d, 1H, J=8.7 Hz), 7.72(d, 1H, J=1.9 Hz), 7.33 (dd, 1H, J=2.1 Hz, 8.7 Hz), 7.19 (d, 1H, J=2.1 Hz), 6.98 (dd, 1H, J=2.1 Hz, 5.7 Hz), 6.79 (s, 1H), 6.31-6.29 (m, 2H), 3.63-3.57 (m, 4H), 1.86 (d, 1H, J=9.0 Hz), 1.68 (d, 1H, J=8.9 Hz).

¹⁹F NMR (300 MHz, CDCl₃) δ=−68.74.

LC-MS: method X, RT=10.89 mins (30 min run), MS (ESI) m/z: 457.14 (M+H)⁺

HRMS (ES+) calculated for C22H16N402F3S [M+H]⁺: 457.0946, found 457.0945

### Example 91:3-[[6-[[5-(trifluoromethyl)-3-pyridyl]amino]-1,3-benzothiazol-2-yl]carbamoyl]bicyclo[2.2.1]hept-5-ene-2-carboxylic acid

### Step 1 tert-butyl N-[6-[[5-(trifluoromethyl)-3-pyridyl]amino]-1,3-benzothiazol-2-yl]carbamate

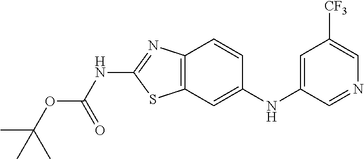 [SMILES: CC(C)(C)OC(=O)Nc1nc2ccc(Nc3cncc(C(F)(F)F)c3)cc2s1]

The tert-butyl-N-[6-bromo-1,3-benzothiazol-2-amino]carbamate (300.0 mg, 0.911 mmol), 5-(trifluoromethyl)pyridin-3-amine (177.0 mg, 1.09 mmol), Pd₂dba₃ (26.2 mg, 0.0456 mmol), tBuONa (131.0 mg, 1.37 mmol) and tBuBrettPhos (44.2 mg, 0.0911 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was triturated in a minimal amount of DCM, and a light yellow precipitate was obtained and filtered (251 mg, 67%).

¹H NMR (300 MHZ, MeOD) 0=8.47 (s, 1H), 8.22(s, 1H), 7.69 (m, 1H), 7.67 (d, 1H, J=4.9 Hz), 7.58 (m, 1H), 7.27 (dd, 1H, J=2.2 Hz, 8.7 Hz), 4.63 (s, 1H), 1.59 (s, 9H).

¹⁹F NMR (300 MHZ, MeOD) δ=−64.85.

LC-MS: 2.21 mins (5 mins), MS (ESI) m/z: 411.54 (M+H)⁺; 409.20 (M−H)⁻

### Step 2

### Example 91:3-[[6-[[5-(trifluoromethyl)-3-pyridyl]amino]-1,3-benzothiazol-2-yl]carbamoyl]bicyclo[2.2.1]hept-5-ene-2-carboxylic acid

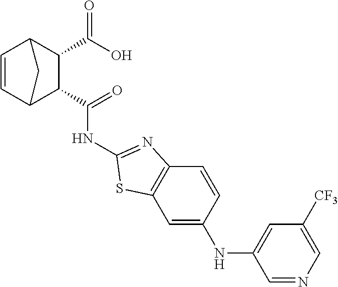 [SMILES: O=C(Nc1nc2ccc(Nc3cncc(C(F)(F)F)c3)cc2s1)[C@@H]1C2C=CC(C2)[C@@H]1C(=O)O]

tert-butyl-N-[6-[(5-trifluoromethyl-3-pyridyl)amino]-1,3-benzothiazol-2-yl]carbamate (200 mg, 0.365 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a yellow oil (117 mg, 76% yield). Without further purification, N-6-[[5-(trifluoromethyl)-3-pyridyl]amino]-1,3-benzothiazol-2,6-amine (117 mg, 0.361 mmol) and (1R,2S,6R,7S)-4-oxatricyclo[5.2.1.02,6]dec-8-ene-3,5-dione (59.3 mg, 0.361 mmol) were dissolved in 1 mL dry THF and heated at 70° C. After 5 h, the reaction mixture was cooled down and the resulting precipitate was filtered and washed several times with THF and ACN. The title product was obtained as a white solid (39 mg, 22% yield).

¹H NMR (300 MHz, DMSO-d₆) δ=12.20 (s, 1H), 11.94 (s, 1H), 8.82(s, 1H), 8.58 (d, 1H, J=2.2 Hz), 8.31 (s, 1H), 7.78 (d, 1H, J=2.1 Hz), 7.68 (d, 1H, J=8.6 Hz), 7.55 (t, 1H, J=2.2 Hz), 7.24 (dd, 1H, J=2.2 Hz, 8.7 Hz), 6.17 (t, 2H, J=1.5 Hz), 3.51-3.36 (m, 2H), 3.11 (d, 2H, J=14.7 Hz), 1.35 (q, 2H, J=8.1 Hz).

¹⁹F NMR (300 MHz, DMSO-d₆) δ=−61.70.

LC-MS: method X, RT=9.94 mins (30 min run), MS (ESI) m/z: (M+H)+473.30; (M−H)⁻ 475.15

HRMS (ES+) calculated for C22H18N403SF3 [M+H]⁺: 475.1062, found 475.1052

### Example 92:4-[6-[(2-methoxy-4-pyridyl)amino]-1,3-benzothiazol-2-yl]-4-azatricyclo[5.2.1.02,6]dec-8-ene-3,5-dione

### Step 1 tert-butyl N-[6-[[2-methoxy-4-pyridyl]amino]-1,3-benzothiazol-2-yl]carbamate

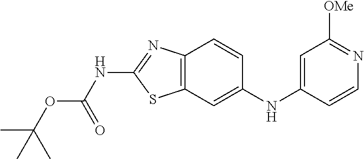 [SMILES: COc1cc(Nc2ccc3nc(NC(=O)OC(C)(C)C)sc3c2)ccn1]

The tert-butyl-N-[6-bromo-1,3-benzothiazol-2-amino]carbamate (300.0 mg, 0.911 mmol), 2-methoxypyridin-4-amine (113.0 mg, 0.911 mmol), Pd₂dba₃ (26.2 mg, 0.045 mmol), tBuONa (131.0 mg, 1.37 mmol) and tBuBrettPhos (44.2 mg, 0.0911 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was triturated in a minimal amount of DCM, and the title compound was obtained as a light yellow precipitate was obtained and filtered (247 mg, 72%).

¹H NMR (MeOD, 300 MHz) δ=7.77 (d, 1H, J=6.0 Hz), 7.68-7.61 (m, 2H), 7.26 (dd, 1H, J=2.1 Hz, 8.6 Hz), 6.57 (dd, 1H, J=1.9 Hz, 5.9 Hz), 6.28 (d, 1H, J=1.8 Hz), 3.84 (s, 3H), 1.59 (s, 9H).

LC-MS: 2.75 mins (5 min run), MS (ESI) m/z: 373.21 (M+H)⁺; 371.25 (M−H)⁻

### Step 2 N6-(2-methoxy-4-pyridyl)-1,3-benzothiazole-2,6-diamine

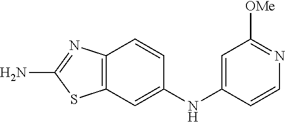 [SMILES: COc1cc(Nc2ccc3nc(N)sc3c2)ccn1]

tert-butyl N-[6-[(2-methoxy-4-pyridyl)amino]-1,3-benzothiazol-2-yl]carbamate (200 mg, 0.516 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The title product was obtained as a yellow oil (145 mg, 99% yield).

LC-MS: 1.94 mins (5 min run), MS (ESI) m/z: 272.87 (M+H)⁺; 271.13 (M−H)⁻

### Step 3 4-[6-[(2-methoxy-4-pyridyl)amino]-1,3-benzothiazol-2-yl]-4-azatricyclo[5.2.1.02,6]dec-8-ene-3,5-dione

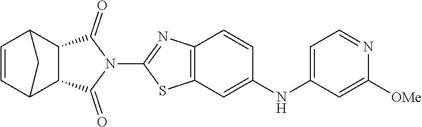 [SMILES: COc1cc(Nc2ccc3nc(N4C(=O)[C@@H]5C6C=CC(C6)[C@@H]5C4=O)sc3c2)ccn1]

To a solution of (1R,2S,6R,7S)-4-oxatricyclo[5.2.1.02,6]dec-8-ene-3,5-dione (33 mg, 0.202 mmol) in dry THF (1 mL) was added N6-(2-methoxy-4-pyridyl)-1,3-benzothiazole-2,6-diamine (55 mg, 0.202 mmol). The mixture was stirred under reflux overnight, then cooled to rt. DCM was then added, and the resulting precipitate was filtered off. The filtrate was then purified by flash chromatography 100/0 to 2/8 Cy/EA to yield title compound as an off-white solid (7 mg, 8% yield).

¹H NMR (300 MHz, CDCl₃) δ=8.03 (d, 1H, J=8.7 Hz), 7.91 (d, 1H, J=4.8 Hz), 7.71 (s, 1H), 7.33 (s, 1H), 7.03 (s, 1H), 6.58 (s, 1H), 6.34 (s, 1H), 6.31-6.26 (m, 2H), 3.92(s, 3H), 3.58 (d, 2H, J=6.4 Hz), 3.35 (s, 1H), 3.22(s, 1H), 1.83 (d, 1H, J=9.0 Hz), 1.67 (d, 1H, J=9.0 Hz).

LC-MS: method X, RT=11.31 mins (30 min run), MS (ESI) m/z: 419.24 (M+H)⁺

HRMS (ES+) calculated for C22H19N403S [M+H]⁺: 419.1178, found 419.1169.

### Example 93 and 94:3-[[6-[[5-(trifluoromethoxy)-2-pyridyl]amino]-1,3-benzothiazol-2-yl]carbamoyl]bicyclo[2.2.1]hept-5-ene-2-carboxylic acid and 4-[6-[[5(trifluoromethoxy)-2-pyridyl]amino]-1,3-benzothiazol-2-yl]-4-azatricyclo[5.2.1.02,6]dec-8-ene-3,5-dione

### Step 1 tert-butyl N-[6-[[5-(trifluoromethoxy)-2-pyridyl]amino]-1,3-benzothiazol-2-yl]carbamate

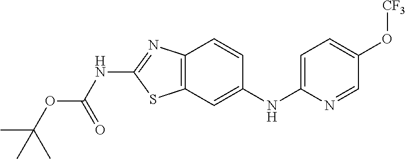 [SMILES: CC(C)(C)OC(=O)Nc1nc2ccc(Nc3ccc(OC(F)(F)F)cn3)cc2s1]

The tert-butyl-N-[6-bromo-1,3-benzothiazol-2-amino]carbamate (300.0 mg, 0.911 mmol), 5-trifluoromethoxypyridin-2-amine (102.0 mg, 0.911 mmol), Pd₂dba₃ (26.2 mg, 0.045 mmol), tBuONa (131.0 mg, 1.37 mmol) and tBuBrettPhos (44.2 mg, 0.0911 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was triturated in a minimal amount of DCM, and a light yellow precipitate was obtained and filtered, yielding title compound as a light yellow solid (230 mg, 59%).

¹H NMR (300 MHZ, DMSO-d₆) δ=8.15 (d, 1H, J=2.0 Hz), 7.88 (d, 1H, J=1.9 Hz), 7.74 (d, 1H, J=8.6 Hz), 7.57 (s, 1H), 7.46 (d, 1H, J=10.6 Hz), 7.32-7.29 (m, 1H), 6.86 (d, 1H, J=9.1 Hz), 1.61 (s, 9H).

¹⁹F NMR (300MHZ, DMSO-d₆)·=−59.47.

LC-MS: 2.41 mins (5 min run), MS (ESI) m/z: (M+H)+427.23; (M−H)⁻425.19

### Step 2 N6-[[5-(trifluoromethoxy)-2-pyridyl]-1,3-benzothiazole-2,6-diamine

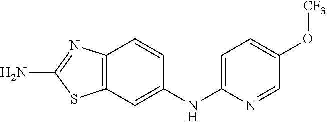 [SMILES: Nc1nc2ccc(Nc3ccc(OC(F)(F)F)cn3)cc2s1]

tert-butyl N-[6-[(5-trifluoromethoxy-2-pyridyl)amino]-1,3-benzothiazol-2-yl]carbamate (220 mg, 0.516 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The title compound was obtained as a white solid (164 mg, 97% yield).

LC-MS: 1.94 mins (5 min run), MS (ESI) m/z: (M+H)+327.07; (M−H)⁻325.07

### Step 3 Example 93:3-[6-[[5-(trifluoromethoxy)-2-pyridyl]amino]-1,3-benzothiazol-2-yl]carbamoyl]bicyclo[2.2.1]hept-5-ene-2-carboxylic acid

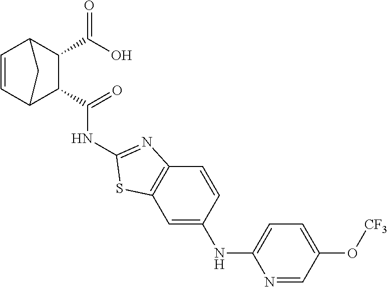 [SMILES: O=C(Nc1nc2ccc(Nc3ccc(OC(F)(F)F)cn3)cc2s1)[C@@H]1C2C=CC(C2)[C@@H]1C(=O)O]

### Example 94:4-[6-[[5-(trifluoromethoxy)-2-pyridyl]amino]-1,3-benzothiazol-2-yl]-4-azatricyclo[5.2.1.02,6]dec-8-ene-3,5-dione

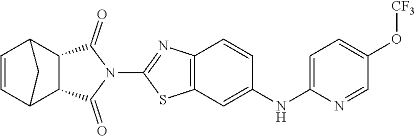 [SMILES: O=C1[C@@H]2C3C=CC(C3)[C@@H]2C(=O)N1c1nc2ccc(Nc3ccc(OC(F)(F)F)cn3)cc2s1]

N6-[[5-(trifluoromethoxy)-2-pyridyl]-1,3-benzothiazole-2,6-diamine (164 mg, 0.503 mmol) and (1S,2R,6S,7R)-4-oxatricyclo[5.2.1.02,6]dec-8-ene-3,5-dione (82.5 mg, 0.503 mmol) were flushed with argon, then slurried in dry THF (1 mL), after which the mixture was heated at 70° C. and the reaction was followed by LC-MS. After 2h30 mins, the reaction was cooled to rt and an excess of THF was added. A first filtration was performed, the precipitate was washed with THF and acetonitrile. The filtrate was concentrated, a second precipitate is observed and filtered. The title compound was obtained as a white solid (Example 93, 157 mg, 63% yield). The filtrate is dried, and the title compound was isolated by flash chromatography purification (9/1 to 5/5 Cy/EA) as a white solid (Example 94, 8 mg, 3% yield).

### Example 93

¹H NMR (300 MHz, DMSO-d₆) δ=12.12(s, 1H), 11.88 (s, 1H), 9.44 (1H), 8.40-8.24 (m, 2H), 7.64-7.50 (m, 2H), 6.93 (d, 1H), 6.17 (s, 2H), 3.46-3.09 (m, 4H), 1.35 (m, 2H).

¹⁹F NMR (300 MHz, DMSO-d₆) δ=−58.31.

LC-MS: method X, RT=11.45 min (30 min run), MS (ESI) m/z: (M+H)+491.09; (M−H)⁻ 488.99

HRMS: (ES+) calculated for C22H18F3N404S [M+H]⁺: 491.1001 found 491.0996.

### Example 94

¹H NMR (300 MHz, DMSO-d₆) δ=9.66 (s, 1H), 8.62(d, 1H, J=2.1 Hz), 8.27 (d, 1H, J=2.8 Hz), 7.91 (d, 1H, J=8.9 Hz), 7.71 (dd, 1H, J=2.0 Hz, 9.1 Hz), 7.62(dd, 1H, J=2.2 Hz, 8.9 Hz), 6.99 (d, 1H, J=9.1 Hz), 6.23 (t, 2H, J=1.8 Hz), 3.63 (dd, 2H, J=1.5 Hz, 2.8 Hz), 3.39 (m, 1H), 1.64 (m, 2H).

¹⁹F NMR (300 MHz, DMSO-d₆) δ=−58.26.

LC-MS: method X, RT=13.28 min (30 min run), MS (ESI) m/z: 473.20 (M+H)⁺; 471.20 (M−H)−

HRMS: (ES+) calculated for C22H16F3N403S [M+H]⁺: 473.0895 found 473.0900.

### Example 95 and 96:4-[[6-[[5-(trifluoromethyl)-2-pyridyl]amino]-1,3-benzothiazol-2-yl]-4-azatricyclo[5.2.1.02,6]dec-8-ene-3,5-dione and 3-[[6-[[5-(trifluoromethyl)-2-pyridyl]amino]-1,3-benzothiazol-2-yl]carbamoyl]bicyclo[2.2.1]hept-5-ene-2-carboxylic acid

### Step 1 tert-butyl-N-[6-[[5-(trifluoromethyl)-2-pyridyl]amino]-1,3-benzothiazol-2-yl]carbamate

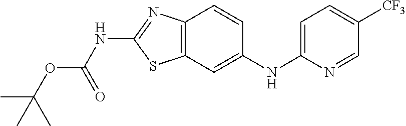 [SMILES: CC(C)(C)OC(=O)Nc1nc2ccc(Nc3ccc(C(F)(F)F)cn3)cc2s1]

The tert-butyl-N-[6-bromo-1,3-benzothiazol-2-amino]carbamate (200.0 mg, 0.873 mmol), 5-trifluoromethylpyridin-2-amine (141.0 mg, 0.873 mmol), Pd₂dba₃ (25.0 mg, 0.0435 mmol), tBuONa (125.0 mg, 1.30 mmol) and tBuBrettPhos (42.3 mg, 0.0873 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was purified by flash chromatography (100/0 to 80/20 Cy/EA), the isolated fractions were then triturated in a minimal amount of DCM and the corresponding precipitate was filtered and washed with DCM and Et₂O to give the title compound as an off-white solid (171 mg, 63% yield).

¹H NMR (300 MHz, CDCl₃) δ=8.60 (d, 1H, J=2.4 Hz), 8.36 (s, 1H), 7.84 (d, 1H, J=8.6 Hz), 7.61 (d, 1H, J=2.1 Hz), 7.53 (s, 1H), 7.24 (dd, 1H, J=2.0 Hz, 8.7 Hz), 6.38 (s, 1H), 1.61 (s, 9H).

¹⁹F NMR (300MHZ, CDCl₃)·=−63.21.

LC-MS: 2.18 mins (5 min run), MS (ESI) m/z: (M+H)+411.24; (M−H)⁻409.24.

### Step 2 N-[6-[[5-(trifluoromethyl)-2-pyridyl]amino]-1,3-benzothiazol-2,6-diamine

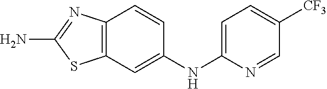 [SMILES: Nc1nc2ccc(Nc3ccc(C(F)(F)F)cn3)cc2s1]

tert-butyl N-[6-[(5-trifluoromethyl-2-pyridyl)amino]-1,3-benzothiazol-2-yl]carbamate (171 mg, 0.417 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a white solid (114 mg, 88% yield).

LC-MS: 1.73 mins (5 min run), MS (ESI) m/z: (M+H)+311.13; (M−H)⁻308.93.

### Step 3

### Example 95:4-[[6-[[5-(trifluoromethyl)-2-pyridyl]amino]-1,3-benzothiazol-2-yl]-4-azatricyclo[5.2.1.02,6]dec-8-ene-3,5-dione

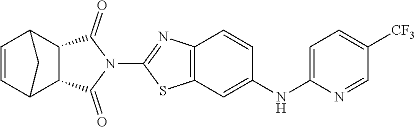 [SMILES: O=C1[C@@H]2C3C=CC(C3)[C@@H]2C(=O)N1c1nc2ccc(Nc3ccc(C(F)(F)F)cn3)cc2s1]

### Example 96:3-[[6-[[5-(trifluoromethyl)-2-pyridyl]amino]-1,3-benzothiazol-2-yl]carbamoyl]bicyclo[2.2.1]hept-5-ene-2-carboxylic acid

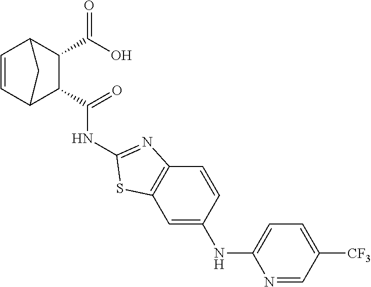 [SMILES: O=C(Nc1nc2ccc(Nc3ccc(C(F)(F)F)cn3)cc2s1)[C@@H]1C2C=CC(C2)[C@@H]1C(=O)O]

N6-[6-(trifluoromethyl)-2-pyridyl]-1,3-benzothiazole-2,6-diamine (114 mg, 0.367 mmol) and the (1S,2R,6S,7R)-4-oxatricyclo[5.2.1.02,6]dec-8-ene-3,5-dione (60.3 mg, 0.367 mmol) were flushed with argon, then slurried in dry THF (1 mL), after which the system was heated at 70° C. After 6 h, the reaction was cooled to rt and a small amount of ACN was added to induce precipitation. The precipitate was filtered and washed with THF and ACN, producing title compound as a white solid (Example 96, 41 mg, 23%). The filtrate was concentrated, after which a flash chromatography purification (100/0 to 3/7 Cy/EA) led to the title compound as an off-white solid (Example 95, 14 mg, 8%).

### Example 95

¹H NMR (300 MHz, CDCl₃)·=9.26 (d, 1H, J=2.0 Hz), 8.50 (dd, 1H, J=2.4 Hz, 8.3 Hz), 7.98 (d, 1H, J=8.7 Hz), 7.66 (dd, 1H, J=1.9 Hz, 8.7 Hz), 7.33 (d, 1H, J=8.7 Hz), 6.36 (t, 2H, J=1.7 Hz), 3.58-3.51 (m, 4H), 1.84 (d, 1H, J=8.8 Hz), 1.66 (d, 1H, J=8.8 Hz).

LC-MS: method X, RT=12.49 min (30 min run), MS (ESI) m/z: 452.02(M+H)⁺

HRMS: (ES+) calculated for C22H16F3N4O2S [M+H]⁺: 457.0953 found 457.0946.

### Example 96

¹H NMR (300 MHz, DMSO-d₆) δ=12.20 (s, 2H), 9.71 (s, 1H), 8.52(s, 1H), 8.41 (d, 1H, J=1.9 Hz), 7.86 (dd, 1H, J=2.3 Hz, 8.8 Hz), 7.66 (d, 1H, J=8.7 Hz), 7.53 (dd, 1H, J=2.0 Hz, 8.8 Hz), 6.95 (d, 1H, J=8.9 Hz), 6.19-6.16 (m, 2H), 3.50-3.35 (m, 2H), 3.11 (d, 2H, J=16.0 Hz), 1.34 (q, 2H, J=8.3 Hz).

¹⁹F NMR (300 MHz, DMSO-d₆) δ=−60.11.

LC-MS: method X, RT=11.18 min (30 min run), MS (ESI) m/z: 473.10 (M−H)⁻

HRMS: (ES+) calculated for C22H18F3N403S [M+H]⁺: 475.1052 found 475.1024.

### Example 97:4-[6-[[6-(trifluoromethyl)-2-pyridyl]amino]-1,3-benzothiazol-2-yl]-4-azatricyclo[5.2.1.02,6]dec-8-ene-3,5-dione

### Step 1 tert-butyl N-[6-[[6-(trifluoromethyl)-2-pyridyl]amino]-1,3-benzothiazol-2-yl]carbamate

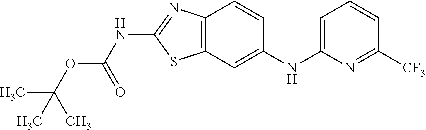 [SMILES: CC(C)(C)OC(=O)Nc1nc2ccc(Nc3cccc(C(F)(F)F)n3)cc2s1]

The tert-butyl-N-[6-bromo-1,3-benzothiazol-2-amino]carbamate (200.0 mg, 0.608 mmol), 6-trifluoromethylpyridin-2-amine (98.5 mg, 0.608 mmol), Pd₂dba₃ (27.8 mg, 0.0304 mmol), tBuONa (87.6 mg, 0.911 mmol) and tBuBrettPhos (29.4 mg, 0.0608 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was purified by flash chromatography (100/0 to 80/20 Cy/EA) to give the title compound as an off-white solid (128 mg, 51% yield).

¹H NMR (300 MHZ, CDCl₃) δ=8.10 (s, 1H), 7.68-7.57 (m, 2H), 7.34 (dd, 1H, J=2.2 Hz, 8.7 Hz), 7.07 (d, 1H, J=7.3 Hz), 6.94-6.89 (m, 1H), 1.56 (s, 9H).

¹⁹F NMR (300 MHz, CDCl₃) δ=−69.27.

LC-MS: 2.32 min (5 min run) MS (ESI) m/z: (M+H)+411.25 (M−H)⁻409.20.

### Step 2 N-[6-[[6-(trifluoromethyl)-2-pyridyl]amino]-1,3-benzothiazol-2,6-amine

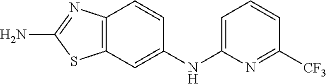 [SMILES: Nc1nc2ccc(Nc3cccc(C(F)(F)F)n3)cc2s1]

tert-butyl N-[6-[(6-trifluoromethyl-2-pyridyl)amino]-1,3-benzothiazol-2-yl]carbamate (100 mg, 0.312 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a white solid (90 mg, 93% yield).

LC-MS: 1.93 min (5 min run) MS (ESI) m/z: (M+H)+311.13 (M−H)⁻309.03.

### Step 3 4-[6-[[6-(trifluoromethyl)-2-pyridyl]amino]-1,3-benzothiazol-2-yl]-4-azatricyclo[5.2.1.02,6]dec-8-ene-3,5-dione

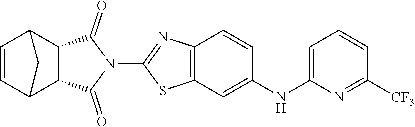 [SMILES: O=C1[C@@H]2C3C=CC(C3)[C@@H]2C(=O)N1c1nc2ccc(Nc3cccc(C(F)(F)F)n3)cc2s1]

N6-[6-(trifluoromethyl)-2-pyridyl]-1,3-benzothiazole-2,6-diamine (90 mg, 0.290 mmol) and the (1S,2R,6S,7R)-4-oxatricyclo[5.2.1.02,6]dec-8-ene-3,5-dione (47.6 mg, 0.290 mmol) were flushed with argon, then slurried in dry THF (1 mL), after which the system was heated at 70° C. After 2h30 mins, the reaction was cooled to rt and an excess of THF was added. A first filtration was performed, the precipitate was washed with THF and acetonitrile, and the filtrate was concentrated, which after a flash chromatography purification (9/1 to 5/5 Cy/EA) led to the title compound as a white solid (19 mg, 14%).

¹H NMR (300 MHz, CDCl₃) δ=8.19 (d, 1H, J=2.1 Hz), 8.00 (d, 1H, J=8.8 Hz), 7.65 (t, 1H, J=7.9 Hz), 7.34 (dd, 1H, J=2.2 Hz, 8.8 Hz), 7.14 (d, 1H, J=7.4 Hz), 7.07 (s, 1H), 6.98 (d, 1H, J=8.5 Hz), 6.30-6.29 (m, 2H), 3.60-3.54 (m, 4H), 1.84 (d, 1H, J=8.9 Hz), 1.66 (d, 1H, J=8.9 Hz).

¹⁹F NMR (300 MHz, CDCl₃) δ=−69.09.

LC-MS: method X, RT=12.98 min (30 min run), MS (ESI) m/z: (M+H)+457.06; (M−H)⁻ 455.06

HRMS: (ES+) calculated for C22H16F3N4O2S [M+H]⁺: 457.0946 found 457.0958.

### Example 98 and 99:4-[6-[(2-fluoro-4-pyridyl)amino]-1,3-benzothiazol-2-yl]-4-azatricyclo[5.2.1.02,6]dec-8-ene-3,5-dione and 3-[[6-[(2-fluoro-4-pyridyl)amino]-1,3-benzothiazol-2-yl]carbamoyl]bicyclo[2.2.1]hept-5-ene-2-carboxylic acid

### Step 1 tert-butyl N-[6-[(2-fluoro-4-pyridyl)amino]-1,3-benzothiazol-2-yl]carbamate

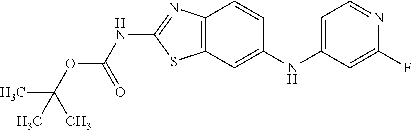 [SMILES: CC(C)(C)OC(=O)Nc1nc2ccc(Nc3ccnc(F)c3)cc2s1]

The tert-butyl-N-[6-bromo-1,3-benzothiazol-2-amino]carbamate (300.0 mg, 0.911 mmol), 2-fluoropyridin-4-amine (102.0 mg, 0.911 mmol), Pd₂dba₃ (26.2 mg, 0.045 mmol), tBuONa (131.0 mg, 1.37 mmol) and tBuBrettPhos (44.2 mg, 0.0911 mmol) were flushed with argon and slurried in previously degassed dioxane (2 mL, dry), then heated at 110° C. After 1 h, the reaction mixture was cooled to rt, treated with an NaHCO₃5% aqueous solution then extracted with EA, the organic phases were washed several times with water and brine, then dried over MgSO₄. The crude product was triturated in a minimal amount of DCM, and a white precipitate was obtained and filtered (117 mg, 35%).

¹H NMR (300 MHz, CDCl₃) δ=7.79 (d, 1H, J=6.0 Hz), 7.73 (d, 1H, J=2.0 Hz), 7.69 (d, 1H, J=8.6 Hz), 7.29 (dd, 1H, J=2.2 Hz, 8.6 Hz), 6.77 (dt, 1H, J=1.7 Hz, 4.2 Hz), 6.42(d, 1H, J=1.2 Hz).

¹⁹F NMR (300 MHz, CDCl₃) δ=−73.86.

LC-MS: 2.01 min (5 min run) MS (ESI) m/z: (M+H)+361.24 (M−H)⁻359.09.

### Step 2 N6-(2-fluoro-4-pyridyl)-1,3-benzothiazole-2,6-diamine

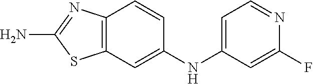 [SMILES: Nc1nc2ccc(Nc3ccnc(F)c3)cc2s1]

tert-butyl N-[6-[(5-fluoro-3-pyridyl)amino]-1,3-benzothiazol-2-yl]carbamate (200 mg, 0.555 mmol) was slurried in 0.7 mL DCM, to which was then added 0.3 mL TFA dropwise at rt. The reaction mixture was stirred for 3 h at rt, after which a saturated Na₂CO₃ aqueous solution was added and the product was extracted with EA. The organic layers collected were washed several times with water and brine, then dried over MgSO₄. The product was obtained as a white solid (72 mg, 85%).

LC-MS: 1.49 min (5 min run) MS (ESI) m/z: (M+H)+261.13 (M−H)⁻259.18.

### Step 3

### Example 98:4-[6-[(2-fluoro-4-pyridyl)amino]-1,3-benzothiazol-2-yl]-4-azatricyclo[5.2.1.02,6]dec-8-ene-3,5-dione

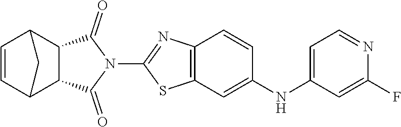 [SMILES: O=C1[C@@H]2C3C=CC(C3)[C@@H]2C(=O)N1c1nc2ccc(Nc3ccnc(F)c3)cc2s1]

### Example 99:3-[[6-[(2-fluoro-4-pyridyl)amino]-1,3-benzothiazol-2-yl]carbamoyl]bicyclo[2.2.1]hept-5-ene-2-carboxylic acid

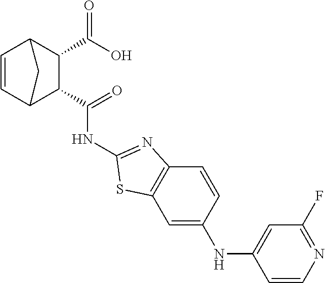 [SMILES: O=C(Nc1nc2ccc(Nc3ccnc(F)c3)cc2s1)[C@@H]1C2C=CC(C2)[C@@H]1C(=O)O]

In a two-necked flask fitted with a condenser, N6-(2-fluoro-4-pyridyl)-1,3-benzothiazole-2,6-diamine (75.5 mg, 0.290 mmol) and the (1S,2R,6S,7R)-4-oxatricyclo[5.2.1.02,6]dec-8-ene-3,5-dione (47.6 mg, 0.290 mmol) were flushed with argon, then slurried in dry THF (1 mL), after which the system was heated at 70° C. and the reaction was followed by LC-MS. After 2.5h, the reaction was cooled to rt and an excess of THF was added. A first filtration was performed, the precipitate was washed with THF and acetonitrile. The title compound was recovered as a white solid (Example 99, 45.0 mg, 38% yield). The filtrate was concentrated, which after a flash chromatography purification (10/0 to 5/5 Cy/EA) led to the title compound as a white solid (Example 98, 21 mg, 17% yield).

### Example 98

¹H NMR (300 MHz, CDCl₃) δ=8.02(d, 1H, J=8.7 Hz), 7.92(d, 1H, J=5.8 Hz), 7.67 (d, 1H, J=2.1 Hz), 7.30 (d, 1H, J=1.3 Hz), 6.70 (s, 1H), 6.68 (t, 1H, J=1.8 Hz), 6.40 (d, 1H, J=1.9 Hz), 6.29 (t, 2H, J=1.5 Hz), 3.62-3.56 (m, 4H), 1.84 (d, 1H, J=8.9 Hz), 1.67 (d, 1H, J=8.9 Hz)

¹⁹F NMR (300 MHz, CDCl₃) δ=−68.99.

LC-MS: method X, RT=9.52 min (30 min run), MS (ESI) m/z: (M+H)+405.15; (M−H)⁻ 407.20

HRMS: (ES+) calculated for C21H16FN4O2S [M+H]⁺: 407.0968 found 407.0978.

### Example 99

¹H NMR (300 MHz, DMSO-d₆) δ=12.22(s, 1H), 12.01 (s, 1H), 9.19 (s, 1H), 7.83 (s, 2H), 7.69 (d, 1H, J=8.2 Hz), 7.26 (d, 1H, J=7.7 Hz), 6.78 (s, 1H), 6.43 (s, 1H), 6.17 (s, 2H), 3.46-3.42(m, 2H), 3.11 (d, 2H, J=13.4 Hz), 1.35 (d, 2H, J=8.5 Hz).

¹⁹F NMR (300 MHz, DMSO-d₆) δ=−69.99.

LC-MS: method X, RT=8.19 min (30 min run), MS (ESI) m/z: (M+H)+425.19; (M−H)⁻ 423.24.

HRMS: (ES+) calculated for C21H18FN403S [M+H]⁺: 425.1084 found 425.1096.

### Example 100:3-[[6-[4-(1-piperidyl) anilino]-1,3-benzothiazol-2-yl]carbamoyl]bicyclo[2.2.1]hept-5-ene-2-carboxylic acid

### Step 1 tert-butyl N-[6-[4-(1-piperidyl)phenyl]-1,3-benzothiazol-2-yl]carbamate

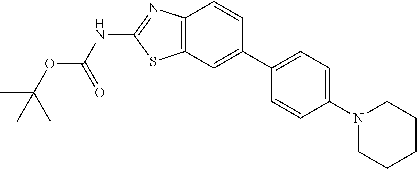 [SMILES: CC(C)(C)OC(=O)Nc1nc2ccc(-c3ccc(N4CCCCC4)cc3)cc2s1]

The tert-butyl-N-[6-bromo-1,3-benzothiazol-2-amino]carbamate (200.0 mg, 0.607 mmol), 1-[4-(4,4,5,5-tetramethyl-1,3,2-dioxaborolan-2-yl)phenyl]piperidine (209.2 mg, 0.728 mmol), Pd(dppf)Cl₂(88.7 mg, 0.121 mmol) and K₃PO₄ (193.3 mg, 0.910 mmol), were flushed with argon and slurried in previously degassed mixture of 1,4-dioxane and water (4 mL, 4/1 vol/vol mixture), then heated at 110° C. After 7 h, the reaction mixture was cooled to rt, treated with an NaHCO₃5% aqueous solution then extracted with EA, the organic phases were washed several times with water and brine, then dried over MgSO₄. The product was purified by flash chromatography (100/0 to 9/1 Cy/EA), and the isolated fractions were then precipitated from a DCM/Pentane solvent mixture (50 mg, 20% yield).

¹H NMR (300 MHz, CDCl₃) δ=10.79 (s, 1H), 7.96 (d, 1H, J=1.7 Hz), 7.92(dd, 1H, J=3.4 Hz, 8.4 Hz), 7.62(dd, 1H, J=1.7 Hz, 8.4 Hz), 7.58 (d, 2H, J=9.1 Hz), 7.10 (m, 2H), 3.27 (t, 4H, J=4.8 Hz), 1.80 (m, 4H), 1.63 (m, 11H).

LC-MS: 2.74 min (5 min run) MS (ESI) m/z: (M+H)+410.30.

### Step 2 N-[6-[4-(1-piperidyl)phenyl]-1,3-benzothiazol-2,6-diamine

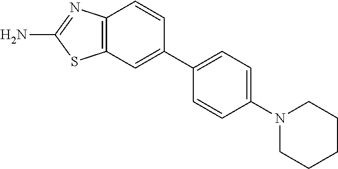 [SMILES: Nc1nc2ccc(-c3ccc(N4CCCCC4)cc3)cc2s1]

tert-butyl N-[6-[4-(1-piperidyl)phenyl]-1,3-benzothiazol-2-yl]carbamate (72 mg, 0.176 mmol) was dissolved in 1.0 mL DCM, then TFA was slowly added (0.5 mL) and the reaction was stirred at rt. The compound was then treated with a saturated solution of Na₂CO₃ and extracted with EA. The organic layers were washed with water and brine, then dried over MgSO₄. Trituration in DCM led to the clean product as a white solid (51 mg, 93%).

LC-MS: 2.22 min (5 min run) MS (ESI) m/z: (M+H)+310.18.

### Step 3 3-[6-[4-(1-piperidyl) anilino]-1,3-benzothiazol-2-yl]carbamoyl]bicyclo[2.2.1]hept-5-ene-2-carboxylic acid

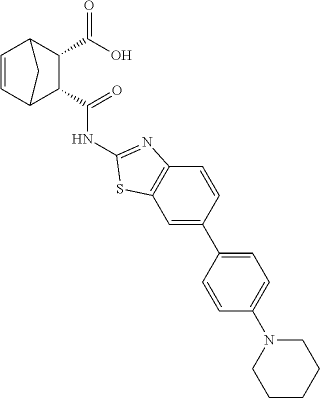 [SMILES: O=C(Nc1nc2ccc(-c3ccc(N4CCCCC4)cc3)cc2s1)[C@@H]1C2C=CC(C2)[C@@H]1C(=O)O]

N-[6-[4-(1-piperidyl)phenyl]-1,3-benzothiazol-2,6-diamine (51 mg, 0.165 mmol) and (1S,2R,6S,7R)-4-oxatricyclo[5.2.1.02,6]dec-8-ene-3,5-dione (27.1 mg, 0.165 mmol) were flushed with argon, then slurried in dry THF (1 mL), after which the system was heated at 70° C. After overnight, the reaction was cooled to rt, and a white precipitate started to form. A first filtration was performed, the precipitate was washed with THF and acetonitrile, and the title compound was obtained as a white solid (37 mg, 47% yield).

¹H NMR (300 MHz, DMSO-d₆) δ=12.24 (s, 1H), 11.94 (s, 1H), 8.15 (d, 1H, J=1.4 Hz), 7.73-7.63 (m, 2H), 7.58 (d, 1H, J=8.8 Hz), 7.01 (d, 2H, J=8.9 Hz), 6.17 (s, 2H), 3.22-3.08 (m, 6H), 1.69-1.53 (m, 6H), 1.35 (q, 2H, J=9.1 Hz).

LC-MS: method X, RT=13.02 min (30 min run), MS (ESI) m/z: (M+H)+474.20; (M−H)⁻ 472.20

HRMS: (ES+) calculated for C27H28N303S [M+H]⁺: 474.1851 found 474.1834.

### Example 101: tert-butyl 4-[4-[2-[(1R,2S,6R,7S)-3,5-dioxo-4-azatricyclo[5.2.1.02,6]dec-8-en-4-yl]-1,3-benzothiazol-6-yl]phenyl]piperazine-1-carboxylate

### Step 1 tert-butyl 4-[4-(2-amino-1,3-benzothiazol-6-yl)phenyl]piperazine-1-carboxylate

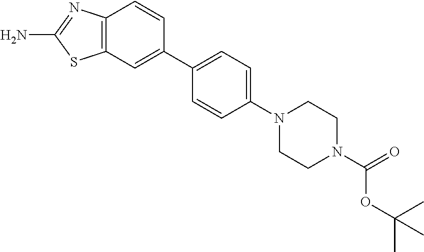 [SMILES: CC(C)(C)OC(=O)N1CCN(c2ccc(-c3ccc4nc(N)sc4c3)cc2)CC1]

6-bromo-1,3-benzothiazol-2-amine (200.0 mg, 0.873 mmol), tert-butyl 4-[4-(4,4,5,5-tetramethyl-1,3,2-dioxaborolan-2-yl)phenyl]piperazine-1-carboxylate (407.2 mg, 1.05 mmol), Pd(dppf)Cl₂(128.0 mg, 0.175 mmol) and K₃PO₄ (278.0 mg, 1.31 mmol), were flushed with argon and slurried in previously degassed mixture of 1,4-dioxane and water (4 mL, 4/1 vol/vol mixture), then heated at 110° C. After 7 h, the reaction mixture was cooled to rt, treated with an NaHCO₃5% aqueous solution then extracted with EA, the organic phases were washed several times with water and brine, then dried over MgSO₄. The product was purified by flash chromatography (100/0 to 3/7 DCM/EA), the title compound was obtained as a white solid (86 mg, 24% yield).

¹H NMR (300 MHz, CDCl₃) δ=7.76 (s, 1H), 7.63-7.54 (m, 2H), 7.51 (d, 2H, J=8.7 Hz), 7.01 (d, 2H, J=8.8 Hz), 3.63 (t, 4H, J=4.9 Hz), 3.22(t, 4H, J=5.2 Hz), 1.51 (s, 9H).

LC-MS: 2.21 min (5 min run) MS (ESI) m/z: (M+H)+411.25, (M−H)⁻409.35.

### tert-butyl 4-[4-[2-[(1R,2S,6R,7S)-3,5-dioxo-4-azatricyclo[5.2.1.02,6]dec-8-en-4-yl]-1,3-benzothiazol-6-yl]phenyl]piperazine-1-carboxylate

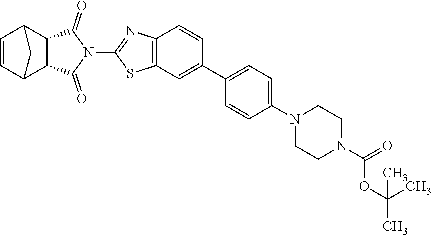 [SMILES: CC(C)(C)OC(=O)N1CCN(c2ccc(-c3ccc4nc(N5C(=O)[C@@H]6C7C=CC(C7)[C@@H]6C5=O)sc4c3)cc2)CC1]

tert-butyl 4-[4-(2-amino-1,3-benzothiazol-6-yl)phenyl]piperazine-1-carboxylate (60 mg, 0.146 mmol) and (1S,2R,6S,7R)-4-oxatricyclo[5.2.1.02,6]dec-8-ene-3,5-dione (24.0 mg, 0.146 mmol) were flushed with argon, then slurried in dry THF (1 mL), after which the system was heated at 70° C. After overnight, the reaction was cooled to rt, and a white precipitate started to form. A first filtration was performed, the precipitate was washed with THF and acetonitrile, and the title compound was obtained as a white solid (31 mg, 38% yield).

¹H NMR (300 MHz, CDCl₃) δ=8.12(d, 1H, J=8.5 Hz), 8.02(d, 1H, J=1.6 Hz), 7.71 (dd, 1H, J=1.8 Hz, 8.5 Hz), 7.60 (d, 2H, J=8.7 Hz), 7.13-7.04 (m, 2H), 6.31 (t, 2H, J=1.6 Hz), 3.70-3.55 (m, 8H), 3.24 (t, 4H, J=4.8 Hz), 1.84 (d, 1H, J=9.0 Hz), 1.67 (d, 1H, J=9.0 Hz), 1.52(s, 9H).

LC-MS: method X, RT=15.42 min (30 min run), MS (ESI) m/z: (M+H)+557.15.

HRMS: (ES+) calculated for C31H32N404S [M+H]⁺: 557.2223, found 557.2261.

### Example 102:4-[6-(4-morpholinophenyl)-1,3-benzothiazol-2-yl]-4-azatricyclo[5.2.1.02,6]dec-8-ene-3,5-dione

### Step 1 tert-butyl N-[6-(4-morpholinophenyl)-1,3-benzothiazol-2-yl]carbamate

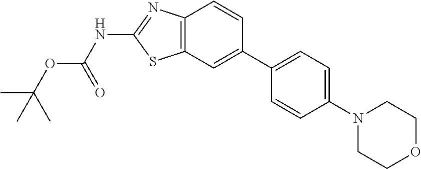 [SMILES: CC(C)(C)OC(=O)Nc1nc2ccc(-c3ccc(N4CCOCC4)cc3)cc2s1]

The tert-butyl-N-[6-bromo-1,3-benzothiazol-2-amino]carbamate (100.0 mg, 0.365 mmol), 1-[4-(4,4,5,5-tetramethyl-4-[4-(4,4,5,5-tetramethyl-1,3,2- dioxaborolan-2-yl)phenyl]morpholine (105 mg, 0.365 mmol), Pd(dppf)Cl₂(44.5 mg, 0.0608 mmol) and K₃PO₄ (96.8 mg, 0.456 mmol), were flushed with argon and slurried in previously degassed mixture of 1,4-dioxane and water (4 mL, 4/1 vol/vol mixture), then heated at 110° C. After 7 h, the reaction mixture was cooled to rt, treated with an NaHCO₃5% aqueous solution then extracted with EA, the organic phases were washed several times with water and brine, then dried over MgSO₄. The product was purified by flash chromatography (100/0 to 9/1 Cy/EA), to produce the title compound as an off-white solid (91 mg, 72% yield).

¹H NMR (300 MHz, CDCl₃) δ=7.96 (d, 1H, J=1.6 Hz), 7.82(d, 1H, J=8.4 Hz), 7.65-7.59 (m, 3H), 7.12(d, 2H, J=8.8 Hz), 3.97 (t, 4H, J=4.5 Hz), 3.28 (t, 4H, J=4.7 Hz), 1.61 (s, 9H).

LC-MS: 2.33 min (5 min run) MS (ESI) m/z: (M+H)+412.15.

### Step 2 6-(4-morpholinophenyl)-1,3-benzothiazol-2-amine

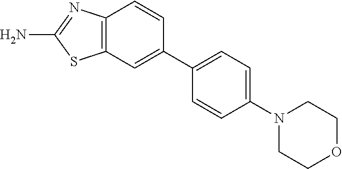 [SMILES: Nc1nc2ccc(-c3ccc(N4CCOCC4)cc3)cc2s1]

tert-butyl N-[6-(4-morpholinophenyl)-1,3-benzothiazol-2-yl]carbamate (91 mg, 0.221 mmol) was dissolved in 1.0 mL DCM, then TFA was slowly added (0.5 mL) and the reaction was stirred at rt. The compound was then treated with a saturated solution of Na₂CO₃ and extracted with EA. The organic layers were washed with water and brine, then dried over MgSO₄. The product was obtained as a white solid (66 mg, 95%).

LC-MS: 1.81 min (5 min run) MS (ESI) m/z: (M+H)+311.98.

### Step 3 4-[6-(4-morpholinophenyl)-1,3-benzothiazol-2-yl]-4-azatricyclo[5.2.1.02,6]dec-8-ene-3,5-dione

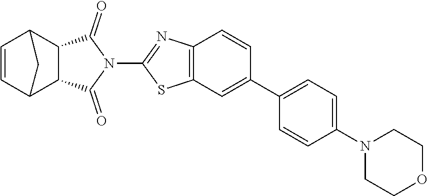 [SMILES: O=C1[C@@H]2C3C=CC(C3)[C@@H]2C(=O)N1c1nc2ccc(-c3ccc(N4CCOCC4)cc3)cc2s1]

6-(4-morpholinophenyl)-1,3-benzothiazol-2-amine (55 mg, 0.177 mmol) and (1S,2R,6S,7R)-4-oxatricyclo[5.2.1.02,6]dec-8-ene-3,5-dione (29.0 mg, 0.177 mmol) were flushed with argon, then slurried in dry THF (1 mL), after which the system was heated at 70° C. After overnight, the reaction was cooled to rt, and a white precipitate started to form. A first filtration was performed, the precipitate was washed with THF and acetonitrile, and the title compound was obtained as a white solid (19 mg, 23% yield).

¹H NMR (300 MHz, CDCl₃) δ=8.12(d, 1H, J=8.5 Hz), 8.02(d, 1H, J=1.5 Hz), 7.71 (dd, 1H, J=1.7 Hz, 8.5 Hz), 7.60 (d, 2H, J=8.3 Hz), 6.31 (t, 2H, J=1.7 Hz), 3.92(t, 4H, J=4.7 Hz), 3.60-3.56 (m, 4H), 3.26 (t, 4H, J=4.9 Hz), 1.85 (d, 1H, J=8.9 Hz), 1.67 (d, 1H, J=8.9 Hz).

LC-MS: method X, RT=12.34 min (30 min run), MS (ESI) m/z: (M+H)+458.16.

HRMS: (ES+) calculated for C26H24N303S [M+H]⁺: 458.1538, found 458.1530.

### Part B: Activity of the Compounds According to the Invention

### Material and Methods

### 1. Construction of Mycobacterial Recombinant Strains.

Strain M. tuberculosis H37Rv_gfp. A recombinant strain of M. tuberculosis H37Rv expressing the green fluorescent protein (H37Rv_GFP) was obtained by transformation of the integrative plasmid pNIP48 (Abadie et al., 2005; Cremer et al., 2002). In this plasmid derived from the Ms6 mycobacteriophage, the GFP gene was cloned under the strong mycobacterial promoter pBlaF and the GFP is constitutively expressed. This plasmid also contained a hygromycin resistance gene.

Strain M. tuberculosis H37Rv_DdnL49P_gfp. The M. tuberculosis strain H37Rv_DdnL49P_gfp is a derivative of the strain H37Rv_gfp that was selected on pretomanid-containing agar plates (2 μg/ml). This strain carries a Leu49Pro mutation in Ddn.

### 2. Growth and Preparation of the Fluorescent Mycobacteria

Bacterial stocks (1 ml) kept at −80° C. were used to inoculate 25 ml of Middlebrook 7H9 medium supplemented with oleic acid-albumin-dextrose-catalase (OADC, Difco, Sparks MD, USA) with 50 mg·mL⁻¹ hygromycin (Invitrogen, Carlsbad, CA USA) in 125 ml culture flasks (Dutscher 29674). Flasks were incubated at 37° C. without shaking for 7 days. Cultures were then diluted with fresh culture medium to reach an OD600 of 0.2 and were cultivated 3 days at 37° C. without shaking.

### 3. Microplates Preparation

Pretomanid and delamanid were dissolved in DMSO at 10 mg·mL⁻¹ and aliquots were stored frozen at −20° C. Test-compounds were resuspended in DMSO at a final concentration of 10 mM and stored at −20° C. Pretomanid, delamanid and test-compounds were transferred to a 384-well low dead volume polypropylene plate (Corning, no. 3672) and used to prepare assay plates. Ten 3-fold serial dilutions of compounds (50 to 0.02 μM) were performed into black Greiner 384-well clear bottom polystyrene plates (Greiner, no. 781091) using an Echo 550 liquid Handler (Labcyte). DMSO volume were compensated so that the concentration across all wells is equal (0.5%). Controls in the assay plate include DMSO at 0.5% (negative control) and Rif at 0.1 μg·mL⁻¹ (positive control).

Pretomanid, delamanid or DMSO were then transferred to the 384-well plates, using Echo. The final concentration of pretomanid and delamanid were 20 ng·mL⁻¹ or 4 ng·mL⁻¹ respectively for assays involving H37Rv_gfp, and were 5 μg·mL⁻¹ for pretomanid in the assay involving H37Rv_DdnL49P_gfp. The final amount of DMSO in the assay plate was 0.6 and 0.55% v/v for the assays involving H37Rv_gfp and H37Rv_DdnL49P_gfp, respectively.

Cultures were diluted to an OD600 nm of 0.02 (using fresh culture medium with no added Hygromycin) and 50 μL were transferred to each assay plate. Assay plates were incubated at 37° C. for 5 days. Fluorescent signal was acquired on a Victor 3 multilabel plate reader (Perkin Elmer), using excitation=485 nm/emission=535 nm. EC50 H37Rv_gfp and EC50 H37Rv_DdnL49P_gfp were obtained from this assay.

### Results

Biological Activities in Combination with Pretomanid:

### The Results are Shown in the Table 1 Below:

TABLE 1

| | EC50 | EC50 |
|---|---|---|
| | H37Rv_gfp | H37Rv_DdnL49P_gfp |
| Example | (μM) | (μM) |
| 1 | +++ | ++ |
| 1a | ++++ | ++++ |
| 1b | ++ | + |
| 2 | ++++ | ++ |
| 3 | +++ | +++ |
| 4 | + | |
| 5 | +++ | |
| 6 | +++ | |
| 7 | ++++ | ++++ |
| 8 | ++++ | +++ |
| 9 | ++ | ++ |
| 10 | + | + |
| 11 | ++++ | +++ |
| 12 | ++++ | ++++ |
| 13 | ++++ | +++ |
| 14 | ++++ | |
| 15 | ++++ | +++ |
| 16 | ++++ | +++ |
| 17 | ++++ | ++++ |
| 18 | ++++ | ++++ |
| 19 | ++++ | +++ |
| 20 | ++++ | ++++ |
| 21 | ++++ | ++++ |
| 22 | +++ | +++ |
| 23 | +++ | +++ |
| 24 | ++ | ++ |
| 25 | +++ | +++ |
| 26 | +++ | +++ |
| 27 | ++++ | ++++ |
| 28 | ++++ | ++++ |
| 29 | ++++ | ++++ |
| 30 | ++++ | ++++ |
| 31 | +++ | ++ |
| 32 | ++++ | ++++ |
| 34 | +++ | +++ |
| 35 | +++ | +++ |
| 36 | ++++ | ++++ |
| 37 | +++ | +++ |
| 38 | +++ | ++ |
| 40 | +++ | ++ |
| 41 | +++ | +++ |
| 42 | +++ | +++ |
| 43 | ++++ | +++ |
| 44 | ++++ | +++ |
| 45 | +++ | ++ |
| 46 | +++ | |
| 47 | ++++ | |
| 48 | +++ | |
| 49 | +++ | +++ |
| 50 | + | |
| 51 | ++++ | +++ |
| 52 | ++++ | +++ |
| 53 | ++++ | ++++ |
| 54 | ++++ | ++++ |
| 55 | ++++ | ++++ |
| 56 | ++++ | +++ |
| 57 | +++ | + |
| 58 | ++ | + |
| 59 | ++++ | +++ |
| 60 | ++++ | ++++ |
| 61 | ++++ | ++++ |
| 62 | ++++ | ++++ |
| 63 | ++ | ++ |
| 64 | +++ | ++ |
| 65 | +++ | +++ |
| 66 | ++++ | +++ |
| 67 | +++ | +++ |
| 68 | +++ | +++ |
| 69 | ++++ | ++++ |
| 70 | +++ | +++ |
| 71 | ++++ | ++++ |
| 72 | +++ | +++ |
| 73 | ++++ | ++++ |
| 74 | ++++ | +++ |
| 75 | ++++ | +++ |
| 76 | + | |
| 77 | ++++ | ++++ |
| 78 | ++++ | ++++ |
| 79 | ++++ | ++++ |
| 80 | + | + |
| 81 | +++ | +++ |
| 82 | | |
| 83 | +++ | ++ |
| 84 | +++ | +++ |
| 85 | ++++ | ++++ |
| 86 | ++++ | ++++ |
| 87 | ++++ | ++++ |
| 88 | ++++ | ++++ |
| 89 | +++ | +++ |
| 90 | ++++ | ++++ |
| 91 | +++ | ++ |
| 92 | +++ | +++ |
| 93 | ++++ | ++++ |
| 95 | ++++ | ++++ |
| 96 | +++ | ++++ |
| 97 | ++++ | ++++ |
| 98 | ++ | |
| 99 | ++ | +++ |
| 100 | ++++ | ++++ |

EC50 (μM) < 1.0 μM = ++++
EC50 (μM) > 1.0 μM and EC50 (μM) < 5.0 μM = +++
EC50 (μM) > 5.0 μM and EC50 (μM) < 10.0 μM = ++
EC50 (μM) > 10.0 μM and EC50 (μM) < 20.0 μM = +

In particular, each of examples 1a, 7, 12, 20, 21, 27, 28, 30, 32, 36, 53, 54, 55, 60, 61, 62, 69, 71, 73, 77, 78, 79 were found to have an average EC50 H37Rv_gfp of <500 nM and an average EC50 H37Rv_DdnL49P_gfp of <1 μM.

Specific exemplary compounds are detailed below.

Example 1a was found to have an average EC50 H37Rv_gfp of 0.25 and an average EC50 H37Rv_DdnL49P_gfp of 0.72.

Example 7 was found to have an average EC50 H37Rv_gfp of 0.15 and an average EC50 H37Rv_DdnL49P_gfp of 1.0.

Example 21 was found to have an average EC50 H37Rv_gfp of 0.27 and an average EC50 H37Rv_DdnL49P_gfp of 0.4.

Example 27 was found to have an average EC50 H37Rv_gfp of 0.11 and an average EC50 H37Rv_DdnL49P_gfp of 0.23.

Example 28 was found to have an average EC50 H37Rv_gfp of 0.13 and an average EC50 H37Rv_DdnL49P_gfp of 0.21.

Example 32 was found to have an average EC50 H37Rv_gfp of 0.32 and an average EC50 H37Rv_DdnL49P_gfp of 0.32.

Example 36 was found to have an average EC50 H37Rv_gfp of 0.22 and an average EC50 H37Rv_DdnL49P_gfp of 0.49.

Example 54 was found to have an average EC50 H37Rv_gfp of 0.11 and an average EC50 H37Rv_DdnL49P_gfp of 0.69.

Example 60 was found to have an average EC50 H37Rv_gfp of 0.05 and an average EC50 H37Rv_DdnL49P_gfp of 0.48.

Example 78 was found to have an average EC50 H37Rv_gfp of 0.13 and an average EC50 H37Rv_DdnL49P_gfp of 0.22.

Biological Activities in Combination with Delamanid:

The results are shown in the Table 2 below:

TABLE 2

| | EC50 |
|---|---|
| | H37Rv_gfp |
| Example | (μM) |
| 1 | 0.31 |
| 2 | 0.42 |
| 18 | 0.16 |
| 27 | 0.15 |

1. A compound of formula (I): 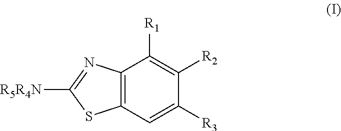
Wherein:
R₁ is chosen from
—H;
—OH;
linear or branched -(C₁-C₆)alkyl; and
linear or branched -(C₁-C₆)alcoxy(C₂-C₆)alkynyl;
R₂ is chosen from:
—H;
linear or branched -(C₁-C₆)alkyl;
linear or branched -(C₁-C₆)alkoxy; and
an halogen atom;
R₃ is chosen from:
—H;
an halogen atom;
linear or branched -(C₁-C₆)alkyl optionally substituted by one or more halogen atoms;
linear or branched -(C₁-C₆)alkoxy optionally substituted by one or more halogen atoms;
linear or branched -(C₂-C₅)alkynyl;
linear or branched -(C₁-C₆)alkoxy-(C₂-C₆)alkynyl;
—OR;
—NHR;
-(C₂-C₅) cycloalkyl:
linear or branched -(C₂-C₆)alkynyl-(C₆-C₁₀)aryl;
linear or branched -(C₁-C₆)alkyl-(C₆-C₁₀)aryl;
-(C₆-C₁₀)aryl optionally substituted by one or more —OH, halogen atoms, linear or branched (C₁-C₆)alkyl optionally substituted by one or more halogen atoms, linear or branched (C₁-C₆)alkoxy optionally substituted by one or more halogen atoms, —NRaRb;
an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom;linear or branched -(C₂-C₆)alkynyl-(C₂-C₆) cycloalkyl;
linear or branched -(C₆-C₁₀)aryl-(C₁-C₆)alkoxy-(C₂-C₆)alkynyl;
—NO₂;
linear or branched —SO₂(C₁-C₃)alkyl;
—CN;
—COO—(C₁-C₃)alkyl;
R is chosen from:
—H;
linear or branched -(C₁-C₆)alkyl optionally substituted by one or more halogen atoms;
-(C₆-C₁₀)aryl, said -(C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atom; linear or branched -(C₁-C₆)alkoxy optionally substituted by one or more halogen atom; —CN;
Ra and Rb, identical or different, are chosen from:
—H;
linear or branched -(C₁-C₆)alkyl, wherein one of the carbon atoms of the alkyl group can be substituted by an heteroatom;
or Ra and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo;
Rc and Rd, identical or different, are chosen from:
—H; and
linear or branched -(C₁-C₆)alkyl;
or R₂ and R₃ are linked together to form —O—(C₁-C₆)alkyl-O— group;
R₄ is H and R₅ is: 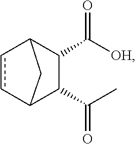
or R₄ and R₅, together with the nitrogen atom to which they are linked
form: 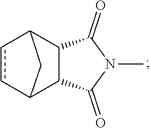
or its pharmaceutically acceptable salts or optical isomers, racemates, diastereoisomers, enantiomers or tautomers;
with the exclusion of the following compounds:
3-[(1,3-benzothiazol-2-yl)carbamoyl]bicyclo[2.2.1]hept-5-ene-2-carboxylic acid;
3-[(6-methyl-1,3-benzothiazol-2-yl)carbamoyl]bicyclo[2.2.1]hept-5-ene-2-carboxylic acid;
3-[(4-methyl-1,3-benzothiazol-2-yl)carbamoyl]bicyclo[2.2.1]hept-5-ene-2-carboxylic acid;
3-[(5,6-dimethyl-1,3-benzothiazol-2-yl)carbamoyl]bicyclo[2.2.1]hept-5-ene-2-carboxylic acid;
3-[(6-bromo-1,3-benzothiazol-2-yl)carbamoyl]bicyclo[2.2.1]hept-5-ene-2-carboxylic acid;
3-[(6-chloro-1,3-benzothiazol-2-yl)carbamoyl]bicyclo[2.2.1]hept-5-ene-2-carboxylic acid;
3-[(4,6-dimethyl-1,3-benzothiazol-2-yl)carbamoyl]bicyclo[2.2.1]hept-5-ene-2-carboxylic acid;
3-[(6-ethoxy-1,3-benzothiazol-2-yl)carbamoyl]bicyclo[2.2.1]hept-5-ene-2-carboxylic acid;
3-[(6-nitro-1,3-benzothiazol-2-yl)carbamoyl]bicyclo[2.2.1]hept-5-ene-2-carboxylic acid;
3-[(6-methylsulfonyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
2-[(6-ethyl-1,3-benzothiazol-2-yl)carbamoyl]bicyclo[2.2.1]hept-5-ene-3-carboxylic acid;
2-[(6-fluoro-1,3-benzothiazol-2-yl)carbamoyl]bicyclo[2.2.1]hept-5-ene-3-carboxylic acid;
2-[(5-ethoxy-1,3-benzothiazol-2-yl)carbamoyl]bicyclo[2.2.1]hept-5-ene-3-carboxylic acid;
(2-(benzo[d]thiazol-2-yl)-3a,4,7,7a-tetrahydro-1H-4,7-methanoisoindole-1,3 (2H)-dione
2-(5-chloro-6-methylbenzo[d]thiazol-2-yl)-3a,4,7,7a-tetrahydro-1H-4,7-methanoisoindole-1,3 (2H)-dione
2-(4-methylbenzo[d]thiazol-2-yl)-3a,4,7,7a-tetrahydro-1H-4,7-methanoisoindole-1,3 (2H)-dione
2-(6-bromobenzo[d]thiazol-2-yl)-3a,4,7,7a-tetrahydro-1H-4,7-methanoisoindole-1,3 (2H)-dione
2-(6-methoxybenzo[d]thiazol-2-yl)-3a,4,7,7a-tetrahydro-1H-4,7-methanoisoindole-1,3 (2H)-dione;
Composé décrit dans D3=ChemCats 2008; 3-[(6-methoxy-1,3-benzothiazol-2-yl)carbamoyl]norbornane-2-carboxylic acid
4-(6-methoxy-1,3-benzothiazol-2-yl)-4-azatricyclo[5.2.1.02,6]dec-8-ene-3,5-dione;
4-(5-chloro-6-methyl-1,3-benzothiazol-2-yl)-4-azatricyclo[5.2.1.02,6]dec-8-ene-3,5-dione;
4-(4-methyl-1,3-benzothiazol-2-yl)-4-azatricyclo[5.2.1.02,6]dec-8-ene-3,5-dione;
4-(6-bromo-1,3-benzothiazol-2-yl)-4-azatricyclo[5.2.1.02,6]dec-8-ene-3,5-dione;
4-(4-methyl-1,3-benzothiazol-2-yl)-4-azatricyclo[5.2.1.02,6]decane-3,5-dione.

2. A compound of formula (I) according to claim 1, wherein said compound is chosen from:
3-[(6-methyl-1,3-benzothiazol-2-yl)carbamoyl]norbornane-2-carboxylic acid;
(1S,2S,6R,7R)-4-(6-methyl-1,3-benzothiazol-2-yl) 4-azatricyclo[5.2.1.02,6]decane-3,5-dione;
3-[(5-methoxy-1,3-benzothiazol-2-yl)carbamoyl]bicyclo[2.2.1]hept-5-ene-2-carboxylic acid;
3-[(6-methoxy-1,3-benzothiazol-2-yl)carbamoyl]bicyclo[2.2.1]hept-5-ene-2-carboxylic acid;
3-[(5,6-dimethoxy-1,3-benzothiazol-2-yl)carbamoyl]bicyclo[2.2.1]hept-5-ene-2-carboxylic acid;
3-(6,7-dihydro-[1,4]dioxino[2,3-f][1,3]benzothiazol-2-ylcarbamoyl) bicyclo[2.2.1]hept-5-ene-2-carboxylic acid;
3-[(6-ethyl-1,3-benzothiazol-2-yl)carbamoyl]bicyclo[2.2.1]hept-5-ene-2-carboxylic acid;
3-[(6-tert-butyl-1,3-benzothiazol-2-yl)carbamoyl]bicyclo[2.2.1]hept-5-ene-2-carboxylic acid;
3-[(4,5-dimethyl-1,3-benzothiazol-2-yl)carbamoyl]bicyclo[2.2.1]hept-5-ene-2-carboxylic acid;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
3-[(6-benzyl-1,3-benzothiazol-2-yl)carbamoyl]bicyclo[2.2.1]hept-5-ene-2-carboxylic acid;
3-[[6-(4-fluorophenoxy)-1,3-benzothiazol-2-yl]carbamoyl]bicyclo[2.2.1]hept-5-ene-2-carboxylic acid;
3-[6-[[4-(trifluoromethyl)phenoxy]-1,3-benzothiazol-2-yl]carbamoyl]bicyclo[2.2.1]hept-5-ene-2-carboxylic acid;
1 (1R,2S,6R,7S)-4-[6-[4-(trifluoromethyl)phenoxy]-1,3-benzothiazol-2-yl]-4-azatricyclo[5.2.1.02,6]dec-8-ene-3,5-dione;
3-[[6-(4-pyridyloxy)-1,3-benzothiazol-2-yl]carbamoyl]bicyclo[2.2.1]hept-5-ene-2-carboxylic acid;
3-[[6-[[3-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[3-(trifluoromethyl)-2-pyridyl]oxy]-1,3-benzothiazol-2-yl]-4-azatricyclo[5.2.1.02,6]dec-8-ene-3,5-dione;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[4-(trifluoromethyl)-2-pyridyl]oxy]-1,3-benzothiazol-2-yl]-4-azatricyclo[5.2.1.02,6]dec-8-ene-3,5-dione;
3-[[6-[(4-methyl-2-pyridyl)oxy]-1,3-benzothiazol-2-yl]carbamoyl]bicyclo [2.2.1]hept-5-ene-2-carboxylic acid;
2-[[2-[(1R,2S,6R,7S)-3,5-dioxo-4-azatricyclo[5.2.1.02,6]dec-8-en-4-yl]-1,3-benzothiazol-6-yl]oxy]pyridine-4-carbonitrile;
3-[[6-[[2-(trifluoromethyl)-4-pyridyl]oxy]-1,3-benzothiazol-2-yl]carbamoyl]bicyclo[2.2.1]hept-5-ene-2-carboxylic acid;
(1R,2S,6R,7S)-4-[6-[[6-(trifluoromethyl)-2-pyridyl]oxy]-1,3-benzothiazol-2-yl]-4-azatricyclo[5.2.1.02,6]dec-8-ene-3,5-dione;
3-[[6-[[5-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[5-(trifluoromethyl)-2-pyridyl]oxy]-1,3-benzothiazol-2-yl]-4-azatricyclo[5.2.1.02,6]dec-8-ene-3,5-dione;
3-[[6-[(5-bromo-2-pyridyl)oxy]-1,3-benzothiazol-2-yl]carbamoyl]bicyclo [2.2.1]hept-5-ene-2-carboxylic acid;
3-[(6-cyano-1,3-benzothiazol-2-yl)carbamoyl]bicyclo[2.2.1]hept-5-ene-2-carboxylic acid;
3-[(6-methoxycarbonyl-1,3-benzothiazol-2-yl)carbamoyl]bicyclo[2.2.1]hept-5-ene-2-carboxylic acid;
3-[(6-fluoro-1,3-benzothiazol-2-yl)carbamoyl]bicyclo[2.2.1]hept-5-ene-2-carboxylic acid;
3-[[6-(trifluoromethyl)-1,3-benzothiazol-2-yl]carbamoyl]bicyclo[2.2.1]hept-5-ene-2-carboxylic acid;
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[(5-bromo-1,3-benzothiazol-2-yl)carbamoyl]bicyclo[2.2.1]hept-5-ene-2-carboxylic acid;
3-[(6-iodo-1,3-benzothiazol-2-yl)carbamoyl]bicyclo[2.2.1]hept-5-ene-2-carboxylic acid;
3-[(6-hydroxy-1,3-benzothiazol-2-yl)carbamoyl]bicyclo[2.2.1]hept-5-ene-2-carboxylic acid;
3-[(6-phenyl-1,3-benzothiazol-2-yl)carbamoyl]-7-oxabicyclo[2.2.1]hept-5-ene-2-carboxylic acid;
3-[(6-cyclohexyl-1,3-benzothiazol-2-yl)carbamoyl]bicyclo[2.2.1]hept-5-ene-2-carboxylic acid;
3-[[6-(p-tolyl)-1,3-benzothiazol-2-yl]carbamoyl]bicyclo[2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[[6-(4-chlorophenyl)-1,3-benzothiazol-2-yl]carbamoyl]bicyclo[2.2.1]hept-5-ene-2-carboxylic acid;
3-[[6-(3,4-dichlorophenyl)-1,3-benzothiazol-2-yl]carbamoyl]bicycle [2.2.1]hept-5-ene-2-carboxylic acid;
3-[[6-[[2-(trifluoromethyl)phenyl]-1,3-benzothiazol-2-yl]carbamoyl]bicyclo[2.2.1]hept-5-ene-2-carboxylic acid;
(1R,2S,6R,7S)-4-[6-[[2-(trifluoromethyl)phenyl]-1,3-benzothiazol-2-yl]-4-azatricyclo[5.2.1.02,6]dec-8-ene-3,5-dione;
3-[[6-[3-(trifluoromethyl)phenyl]-1,3-benzothiazol-2-yl]carbamoyl]bicyclo[2.2.1]hept-5-ene-2-carboxylic acid;
3-[6-[[4-(trifluoromethyl)phenyl]-1,3-benzothiazol-2-yl]carbamoyl]bicyclo[2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[6-[[4-(trifluoromethoxy)phenyl]-1,3-benzothiazol-2-yl]carbamoyl]bicyclo[2.2.1]hept-5-ene-2-carboxylic acid;
(1R,2S,6R,7S)-4-[6-(4-hydroxyphenyl)-1,3-benzothiazol-2-yl]-4-azatricyclo[5.2.1.02,6]dec-8-ene-3,5-dione;
3-[[6-(4-pyridyl)-1,3-benzothiazol-2-yl]carbamoyl]bicyclo[2.2.1]hept-5-ene-2-carboxylic acid;
(1R,2S,6R,7S)-4-[6-(6-fluoro-3-pyridyl)-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo[2.2.1]hept-5-ene-2-carboxylic acid;
(1R,2S,6R,7S)-4-[6-[[6-(trifluoromethyl)-3-pyridyl]-1,3-benzothiazol-2-yl]-4-azatricyclo[5.2.1.02,6]dec-8-ene-3,5-dione;
3-[(6-pyrimidin-5-yl-1,3-benzothiazol-2-yl)carbamoyl]bicyclo[2.2.1]hept-5-ene-2-carboxylic acid
3-[[6-(6-quinolyl)-1,3-benzothiazol-2-yl]carbamoyl]bicyclo[2.2.1]hept-5-ene-2-carboxylic acid
4-[6-(3-thienyl)-1,3-benzothiazol-2-yl]-4-azatricyclo[5.2.1.02,6]dec-8-ene-3,5-dione
3-[[6-(3-furyl)-1,3-benzothiazol-2-yl]carbamoyl]bicyclo[2.2.1]hept-5-ene-2-carboxylic acid;
(1R,2S,6R,7S)-4-[6-(3-furyl)-1,3-benzothiazol-2-yl]-4-azatricyclo[5.2.1.02,6]dec-8-ene-3,5-dione;
4-[6-(4-prop-2-ynoxyphenyl)-1,3-benzothiazol-2-yl]-4-azatricyclo[5.2.1.02,6]dec-8-ene-3,5-dione;
(1R,2S,6R,7S)-4-(6-ethynyl-1,3-benzothiazol-2-yl)-4-azatricyclo[5.2.1.02,6]dec-8-ene-3,5-dione;
1-(1R,2S,6R,7S)-4-(6-prop-2-ynoxy-1,3-benzothiazol-2-yl)-4-azatricyclo[5.2.1.02,6]dec-8-ene-3,5-dione;
(1R,2S,3R,4S)-3-[(4-hydroxy-1,3-benzothiazol-2-yl)carbamoyl]bicyclo[2.2.1]hept-5-ene-2-carboxylic acid;
3-[(4-prop-2-ynoxy-1,3-benzothiazol-2-yl)carbamoyl]bicyclo[2.2.1]hept-5-ene-2-carboxylic acid;
(1R,2S,6R,7S)-4-(4-prop-2-ynoxy-1,3-benzothiazol-2-yl)-4-azatricyclo[5.2.1.02,6]dec-8-ene-3,5-dione;
3-[[6-(2-phenylethynyl)-1,3-benzothiazol-2-yl]carbamoyl]bicyclo[2.2.1]hept-5-ene-2-carboxylic acid;
3-[[6-(3,3-dimethylbut-1-ynyl)-1,3-benzothiazol-2-yl]carbamoyl]bicyclo [2.2.1]hept-5-ene-2-carboxylic acid;
3-[[6-(2-cyclopropylethynyl)-1,3-benzothiazol-2-yl]carbamoyl]bicyclo[2.2.1]hept-5-ene-2-carboxylic acid;
[2-hydroxy-1,1-bis(hydroxymethyl)ethyl]ammonium;(1R,2S,3R,4S)-3-[(6-phenoxy-1,3-benzothiazol-2-yl)carbamoyl]bicyclo[2.2.1]hept-5-ene-2-carboxylate;
(1R,2S,6R,7S)-4-[[6-[(4-methyl-2-pyridyl)oxy]-1,3-benzothiazol-2-yl]-4-azatricyclo[5.2.1.02,6]dec-8-ene-3,5-dione;
1 (1R,2S,6R,7S)-4-[6-[(2-chloro-4-pyridyl)oxy]-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo[2.2.1]hept-5-ene-2-carboxylic acid;
(1R,2S,6R,7S)-4-(6-anilino-1,3-benzothiazol-2-yl)-4-azatricyclo[5.2.1.02,6]dec-8-ene-3,5-dione;
4-[6-(2-pyridylamino)-1,3-benzothiazol-2-yl]-4-azatricyclo[5.2.1.02,6]dec-8-ene-3,5-dione; and
3-[[6-[(4-methyl-2-pyridyl)amino]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2 yl]carbamoyl]bicyclo[2.2.1]hept-5-ene-2-carboxylic acid;
3-[[6-[(5-fluoro-2-pyridyl)amino]-1,3-benzothiazol-2 yl]carbamoyl]bicyclo[2.2.1]hept-5-ene-2-carboxylic acid;
4-[6-[(5-fluoro-2-pyridyl)amino]-1,3-benzothiazol-2-yl]-4-azatricyclo[5.2.1.02,6]dec-8-ene-3,5-dione;
3-[[6-[(6-fluoro-2-pyridyl)amino]-1,3-benzothiazol-2-yl]carbamoyl]bicyclo[2.2.1]hept-5-ene-2-carboxylic acid;
4-[6-[(6-fluoro-2-pyridyl)amino]-1,3-benzothiazol-2-yl]-4-azatricyclo[5.2.1.02,6]dec-8-ene-3,5-dione;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethyl)-3-pyridyl]amino]-1,3-benzothiazol-2-yl]carbamoyl]bicyclo[2.2.1]hept-5-ene-2-carboxylic acid;
4-[6-[(2-methoxy-4-pyridyl)amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[6-[[5-(trifluoromethoxy)-2-pyridyl]amino]-1,3-benzothiazol-2-yl]-4-azatricyclo[5.2.1.02,6]dec-8-ene-3,5-dione;
4-[[6-[[5-(trifluoromethyl)-2-pyridyl]amino]-1,3-benzothiazol-2-yl]-4-azatricyclo[5.2.1.02,6]dec-8-ene-3,5-dione;
3-[[6-[[5-(trifluoromethyl)-2-pyridyl]amino]-1,3-benzothiazol-2-yl]carbamoyl]bicyclo[2.2.1]hept-5-ene-2-carboxylic acid;
4-[6-[[6-(trifluoromethyl)-2-pyridyl]amino]-1,3-benzothiazol-2-yl]-4-azatricyclo[5.2.1.02,6]dec-8-ene-3,5-dione;
4-[6-[(2-fluoro-4-pyridyl)amino]-1,3-benzothiazol-2-yl]-4-azatricyclo[5.2.1.02,6]dec-8-ene-3,5-dione;
3-[[6-[(2-fluoro-4-pyridyl)amino]-1,3-benzothiazol-2-yl]carbamoyl]bicyclo[2.2.1]hept-5-ene-2-carboxylic acid;
3-[[6-[4-(1-piperidyl) anilino]-1,3-benzothiazol-2-yl]carbamoyl]bicyclo[2.2.1]hept-5-ene-2-carboxylic acid;
tert-butyl4-[4-[2-[(1R,2S,6R,7S)-3,5-dioxo-4-azatricyclo[5.2.1.02,6]dec-8-en-4-yl]-1,3-benzothiazol-6-yl]phenyl]piperazine-1-carboxylate;
4-[6-(4-morpholinophenyl)-1,3-benzothiazol-2-yl]-4-azatricyclo[5.2.1.02,6]dec-8-ene-3,5-dione;
or its pharmaceutically acceptable salts or optical isomers, racemates, diastereoisomers, enantiomers or tautomers.

3. A compound of formula (I) according to claim 1, wherein:
R₁ is H;
R₂ is H;
R₃ is chosen from:
linear or branched -(C₂-C₆)alkynyl;
linear or branched -(C₁-C₆)alkoxy substituted by one or more halogen atoms;
—OR;
—NHR;
-(C₆-C₁₀)aryl optionally substituted by one or more linear or branched -(C₁-C₆)alkyl optionally substituted by one or more halogen atoms, linear or branched -(C₁-C₆)alkoxy optionally substituted by one or more halogen atoms, NRaRb; and
R is chosen from:
linear or branched -(C₁-C₆)alkyl substituted by one or more halogen atoms;
-(C₆-C₁₀)aryl optionally substituted by one or more linear or branched (C₁-C₆)alkyl optionally substituted by one or more halogen atoms;
an heteroaryl comprising 3 to 10 members having at least one heteroatom chosen from O, N, or S; said heteroaryl being optionally substituted by one or more linear or branched -(C₁-C₆)alkyl optionally substituted by one or more halogen atoms;
Ra and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo; and
Rc and Rd, identical or different, are chosen from:
—H; and
linear or branched -(C₁-C₆)alkyl.

4. A compound of formula (I) according to claim 1, wherein:
R₁ is H;
R₂ is H;
R₃ is chosen from:
trifluoromethoxy;
3,3-dimethylbut-1-ynyl;
—OR;
—NHR;
phenyl optionally substituted by one or more trifluoromethyl, methoxy trifluroromethoxy and/or piperidyl; and
R is chosen from:
linear or branched (C₁-C₃)alkyl substituted by one or more halogen atoms, preferably one or more fluor atoms;
phenyl optionally substituted by one or more methyl and/or trifluoromethyl;
a heteroaryl comprising 6 members and having at least one nitrogen atom; said heteroaryl being optionally substituted by one or more trifluoromethyl, trifluoromethoxy and/or fluorine; for example a pyridyl substituted by one or more trifluoromethyl, trifluoromethoxy and/or fluorine.

5. A compound of formula (I) according to claim 1, wherein said compound is chosen from:
3-[[6-(trifluoromethoxy)-1,3-benzothiazol-2-yl]carbamoyl]bicyclo[2.2.1]hept-5-ene-2-carboxylic acid;
1 (1R,2S,6R,7S)-4-[6-(trifluoromethoxy)-1,3-benzothiazol-2-yl]-4-azatricyclo[5.2.1.02,6]dec-8-ene-3,5-dione;
3-[[6-(3,3-dimethylbut-1-ynyl)-1,3-benzothiazol-2-yl]carbamoyl]bicyclo [2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[6-[[4-(trifluoromethoxy)phenyl]-1,3-benzothiazol-2-yl]carbamoyl]bicyclo[2.2.1]hept-5-ene-2-carboxylic acid;
3-[6-[[4-(trifluoromethyl)phenyl]-1,3-benzothiazol-2-yl]carbamoyl]bicyclo[2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2-yl]carbamoyl]bicyclo[2.2.1]hept-5-ene-2-carboxylic acid;
3-[[6-[(5-fluoro-2-pyridyl)amino]-1,3-benzothiazol-2-yl]carbamoyl]bicyclo[2.2.1]hept-5-ene-2-carboxylic acid;
3-[[6-[(6-fluoro-2-pyridyl)amino]-1,3-benzothiazol-2-yl]carbamoyl]bicyclo[2.2.1]hept-5-ene-2-carboxylic acid;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[[6-[[5-(trifluoromethyl)-2-pyridyl]amino]-1,3-benzothiazol-2-yl]-4-azatricyclo[5.2.1.02,6]dec-8-ene-3,5-dione;
4-[6-[[6-(trifluoromethyl)-2-pyridyl]amino]-1,3-benzothiazol-2-yl]-4-azatricyclo[5.2.1.02,6]dec-8-ene-3,5-dione;
3-[[6-[4-(1-piperidyl) anilino]-1,3-benzothiazol-2-yl]carbamoyl]bicyclo[2.2.1]hept-5-ene-2-carboxylic acid;
or its pharmaceutically acceptable salts or optical isomers, racemates, diastereoisomers, enantiomers or tautomers.

6. A pharmaceutical composition comprising compounds of formula (I) as defined in claim 1, and a pharmaceutically acceptable excipient.

7. A pharmaceutical composition according to claim 5, further comprising at least one other anti-mycobacterial agent.

8. A method of prevention and/or treatment of a subject in need thereof, said treatment comprising administering to said subject a therapeutically effective amount of a compound of formula (I): 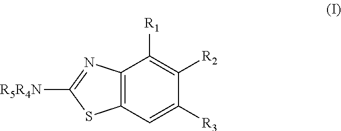
Wherein:
R₁ is chosen from
—H;
—OH;
linear or branched -(C₁-C₆)alkyl; and
linear or branched -(C₁-C₆)alcoxy(C₂-C₆)alkynyl;
R₂ is chosen from:
—H;
linear or branched -(C₁-C₆)alkyl;
linear or branched -(C₁-C₆)alkox;, and
an halogen atom;
R₃ is chosen from:
—H;
an halogen atom;
linear or branched -(C₁-C₆)alkyl optionally substituted by one or more halogen atoms;
linear or branched -(C₁-C₆)alkoxy optionally substituted by one or more halogen atoms;
linear or branched -(C₂-C₅)alkynyl;
linear or branched -(C₁-C₆)alkoxy-(C₂-C₆)alkynyl;
—OR;
—NHR;
-(C₂-C₅) cycloalkyl;
linear or branched -(C₂-C₆)alkynyl-(C₆-C₁₀)aryl;
linear or branched -(C₁-C₆)alkyl-(C₆-C₁₀)aryl;
-(C₆-C₁₀)aryl optionally substituted by one or more —OH, halogen atoms, linear or branched (C₁-C₆)alkyl optionally substituted by one or more halogen atom, one or more linear or branched (C₁-C₆)alkoxy optionally substituted by one or more halogen atoms, NRaRb;
an heteroaryl comprising 3 to 10 members and having at least one heteroatom chosen from O, N, or S; said heteroaryl being optionally substituted by one or more halogen atoms, linear or branched -(C₁-C₆)alkyl optionally substituted by one or more halogen atoms; linear or branched -(C₂-C₆)alkynyl-(C₂-C₆) cycloalkyl;
linear or branched -(C₆-C₁₀)aryl-(C₁-C₆)alkoxy-(C₂-C₆)alkynyl;
—NO₂;
linear or branched —SO₂(C₁-C₃)alkyl;
—CN;
—COO—(C₁-C₃)alkyl;
R is chosen from:
—H;
linear or branched -(C₁-C₆)alkyl optionally substituted by one or more halogen atoms;
-(C₆-C₁₀)aryl, said (C₆-C₁₀)aryl being optionally substituted by one or more halogen atoms, —OH, linear or branched -(C₁-C₆)alkyl optionally substituted by one or more halogen atoms, -(C₁-C₆)alkoxy optionally substituted by one or more halogen atoms;
an heteroaryl comprising 3 to 10 members having at least one heteroatom chosen from O, N, or S; said heteroaryl being optionally substituted by one or more halogen atoms, linear or branched -(C₁-C₆)alkyl optionally substituted by one or more halogen atoms; linear or branched -(C₁-C₆)alkoxy optionally substituted by one or more halogen atom-CN;
Ra and Rb, identical or different, are chosen from:
—H;
linear or branched -(C₁-C₆)alkyl, wherein one of the carbon atoms of the alkyl group can be substituted by an heteroatom;
or Rₐ and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo;
Rc and Rd, identical or different, are chosen from:
—H; and
linear or branched -(C₁-C₆)alkyl;
or R₂ and R₃ are linked together to form —O—(C₁-C₆)alkyl-O— group;
R₄ is H and R₅ is: 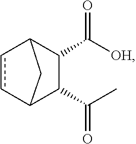
or R₄ and R₅, together with the nitrogen atom to which they are linked form: 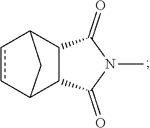
or its pharmaceutically acceptable salts or optical isomers, racemates, diastereoisomers, enantiomers or tautomers;
with the exclusion of the following compound:
3-[(6-methylsulfonyl-1,3-benzothiazol-2-yl)carbamoyl]bicyclo[2.2.1]hept-5-ene-2-carboxylic acid.

9. The method according to claim 8, wherein said compound of formula (I) is chosen from:
3-[(1,3-benzothiazol-2-yl)carbamoyl]bicyclo[2.2.1]hept-5-ene-2-carboxylic acid;
3-[(6-methyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
3-[(6-methyl-1,3-benzothiazol-2-yl)carbamoyl]norbornane-2-carboxylic acid;
1 (1S,2S,6R,7R)-4-(6-methyl-1,3-benzothiazol-2-yl) 4-azatricyclo[5.2.1.02,6]decane-3,5-dione;
3-[(5-methoxy-1,3-benzothiazol-2-yl)carbamoyl]bicyclo[2.2.1]hept-5-ene-2-carboxylic acid;
3-[(6-methoxy-1,3-benzothiazol-2-yl)carbamoyl]bicyclo[2.2.1]hept-5-ene-2-carboxylic acid;
3-[(6-ethoxy-1,3-benzothiazol-2-yl)carbamoyl]bicyclo[2.2.1]hept-5-ene-2-carboxylic acid;
(1R,2S,6R,7S)-4-(6-ethoxy-1,3-benzothiazol-2-yl)-4-azatricyclo[5.2.1.02,6]dec-8-ene-3,5-dione;
3-[(5,6-dimethoxy-1,3-benzothiazol-2-yl)carbamoyl]bicyclo[2.2.1]hept-5-ene-2-carboxylic acid;
3-(6,7-dihydro-[1,4]dioxino[2,3-f][1,3]benzothiazol-2-ylcarbamoyl) bicyclo[2.2.1]hept-5-ene-2-carboxylic acid;
3-[(6-ethyl-1,3-benzothiazol-2-yl)carbamoyl]bicyclo[2.2.1]hept-5-ene-2-carboxylic acid;
3-[(6-tert-butyl-1,3-benzothiazol-2-yl)carbamoyl]bicyclo[2.2.1]hept-5-ene-2-carboxylic acid;
3-[(4,6-dimethyl-1,3-benzothiazol-2-yl)carbamoyl]bicyclo[2.2.1]hept-5-ene-2-carboxylic acid;
3-[(5,6-dimethyl-1,3-benzothiazol-2-yl)carbamoyl]bicyclo[2.2.1]hept-5-ene-2-carboxylic acid;
3-[(4,5-dimethyl-1,3-benzothiazol-2-yl)carbamoyl]bicyclo[2.2.1]hept-5-ene-2-carboxylic acid;
3-[(4-methyl-1,3-benzothiazol-2-yl)carbamoyl]bicyclo[2.2.1]hept-5-ene-2-carboxylic acid;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
3-[(6-benzyl-1,3-benzothiazol-2-yl)carbamoyl]bicyclo[2.2.1]hept-5-ene-2-carboxylic acid;
3-[[6-(4-fluorophenoxy)-1,3-benzothiazol-2-yl]carbamoyl]bicyclo[2.2.1]hept-5-ene-2-carboxylic acid;
3-[6-[[4-(trifluoromethyl)phenoxy]-1,3-benzothiazol-2-yl]carbamoyl]bicyclo[2.2.1]hept-5-ene-2-carboxylic acid;
1 (1R,2S,6R,7S)-4-[6-[4-(trifluoromethyl)phenoxy]-1,3-benzothiazol-2-yl]-4-azatricyclo[5.2.1.02,6]dec-8-ene-3,5-dione;
3-[[6-(4-pyridyloxy)-1,3-benzothiazol-2-yl]carbamoyl]bicyclo[2.2.1]hept-5-ene-2-carboxylic acid;
3-[[6-[[3-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[3-(trifluoromethyl)-2-pyridyl]oxy]-1,3-benzothiazol-2-yl]-4-azatricyclo[5.2.1.02,6]dec-8-ene-3,5-dione;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[4-(trifluoromethyl)-2-pyridyl]oxy]-1,3-benzothiazol-2-yl]-4-azatricyclo[5.2.1.02,6]dec-8-ene-3,5-dione;
3-[[6-[(4-methyl-2-pyridyl)oxy]-1,3-benzothiazol-2-yl]carbamoyl]bicyclo [2.2.1]hept-5-ene-2-carboxylic acid;
2-[[2-[(1R,2S,6R,7S)-3,5-dioxo-4-azatricyclo[5.2.1.02,6]dec-8-en-4-yl]-1,3-benzothiazol-6-yl]oxy]pyridine-4-carbonitrile;
3-[[6-[[2-(trifluoromethyl)-4-pyridyl]oxy]-1,3-benzothiazol-2-yl]carbamoyl]bicyclo[2.2.1]hept-5-ene-2-carboxylic acid;
(1R,2S,6R,7S)-4-[6-[[6-(trifluoromethyl)-2-pyridyl]oxy]-1,3-benzothiazol-2-yl]-4-azatricyclo[5.2.1.02,6]dec-8-ene-3,5-dione;
3-[[6-[[5-(trifluoromethyl)-2-pyridyl]oxy]-1,3-benzothiazol-2-yl]carbamoyl]bicyclo[2.2.1]hept-5-ene-2-carboxylic acid;
(1R,2S,6R,7S)-4-[6-[[5-(trifluoromethyl)-2-pyridyl]oxy]-1,3-benzothiazol-2-yl]-4-azatricyclo[5.2.1.02,6]dec-8-ene-3,5-dione;
3-[[6-[(5-bromo-2-pyridyl)oxy]-1,3-benzothiazol-2-yl]carbamoyl]bicyclo [2.2.1]hept-5-ene-2-carboxylic acid;
3-[(6-nitro-1,3-benzothiazol-2-yl)carbamoyl]bicyclo[2.2.1]hept-5-ene-2-carboxylic acid;
3-[(6-methylsulfonyl-1,3-benzothiazol-2-yl)carbamoyl]bicyclo[2.2.1]hept-5-ene-2-carboxylic acid;
3-[(6-cyano-1,3-benzothiazol-2-yl)carbamoyl]bicyclo[2.2.1]hept-5-ene-2-carboxylic acid;
3-[(6-methoxycarbonyl-1,3-benzothiazol-2-yl)carbamoyl]bicyclo[2.2.1]hept-5-ene-2-carboxylic acid;
3-[(6-fluoro-1,3-benzothiazol-2-yl)carbamoyl]bicyclo[2.2.1]hept-5-ene-2-carboxylic acid;
3-[[6-(trifluoromethyl)-1,3-benzothiazol-2-yl]carbamoyl]bicyclo[2.2.1]hept-5-ene-2-carboxylic acid;
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[(6-chloro-1,3-benzothiazol-2-yl)carbamoyl]bicyclo[2.2.1]hept-5-ene-2-carboxylic acid;
3-[(6-bromo-1,3-benzothiazol-2-yl)carbamoyl]bicyclo[2.2.1]hept-5-ene-2-carboxylic acid;
3-[(5-bromo-1,3-benzothiazol-2-yl)carbamoyl]bicyclo[2.2.1]hept-5-ene-2-carboxylic acid;
3-[(6-iodo-1,3-benzothiazol-2-yl)carbamoyl]bicyclo[2.2.1]hept-5-ene-2-carboxylic acid;
3-[(6-hydroxy-1,3-benzothiazol-2-yl)carbamoyl]bicyclo[2.2.1]hept-5-ene-2-carboxylic acid;
3-[(6-phenyl-1,3-benzothiazol-2-yl)carbamoyl]-7-oxabicyclo[2.2.1]hept-5-ene-2-carboxylic acid;
3-[(6-cyclohexyl-1,3-benzothiazol-2-yl)carbamoyl]bicyclo[2.2.1]hept-5-ene-2-carboxylic acid;
3-[[6-(p-tolyl)-1,3-benzothiazol-2-yl]carbamoyl]bicyclo[2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[[6-(4-chlorophenyl)-1,3-benzothiazol-2-yl]carbamoyl]bicyclo[2.2.1]hept-5-ene-2-carboxylic acid;
3-[[6-(3,4-dichlorophenyl)-1,3-benzothiazol-2-yl]carbamoyl]bicycle [2.2.1]hept-5-ene-2-carboxylic acid;
3-[[6-[[2-(trifluoromethyl)phenyl]-1,3-benzothiazol-2-yl]carbamoyl]bicyclo[2.2.1]hept-5-ene-2-carboxylic acid;
(1R,2S,6R,7S)-4-[6-[[2-(trifluoromethyl)phenyl]-1,3-benzothiazol-2-yl]-4-azatricyclo[5.2.1.02,6]dec-8-ene-3,5-dione;
3-[[6-[3-(trifluoromethyl)phenyl]-1,3-benzothiazol-2-yl]carbamoyl]bicyclo[2.2.1]hept-5-ene-2-carboxylic acid;
3-[6-[[4-(trifluoromethyl)phenyl]-1,3-benzothiazol-2-yl]carbamoyl]bicyclo[2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[6-[[4-(trifluoromethoxy)phenyl]-1,3-benzothiazol-2-yl]carbamoyl]bicyclo[2.2.1]hept-5-ene-2-carboxylic acid;
(1R,2S,6R,7S)-4-[6-(4-hydroxyphenyl)-1,3-benzothiazol-2-yl]-4-azatricyclo[5.2.1.02,6]dec-8-ene-3,5-dione;
3-[[6-(4-pyridyl)-1,3-benzothiazol-2-yl]carbamoyl]bicyclo[2.2.1]hept-5-ene-2-carboxylic acid;
(1R,2S,6R,7S)-4-[6-(6-fluoro-3-pyridyl)-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo[2.2.1]hept-5-ene-2-carboxylic acid;
(1R,2S,6R,7S)-4-[6-[[6-(trifluoromethyl)-3-pyridyl]-1,3-benzothiazol-2-yl]-4-azatricyclo[5.2.1.02,6]dec-8-ene-3,5-dione;
3-[(6-pyrimidin-5-yl-1,3-benzothiazol-2-yl)carbamoyl]bicyclo[2.2.1]hept-5-ene-2-carboxylic acid;
3-[[6-(6-quinolyl)-1,3-benzothiazol-2-yl]carbamoyl]bicyclo[2.2.1]hept-5-ene-2-carboxylic acid;
4-[6-(3-thienyl)-1,3-benzothiazol-2-yl]-4-azatricyclo[5.2.1.02,6]dec-8-ene-3,5-dione;
3-[[6-(3-furyl)-1,3-benzothiazol-2-yl]carbamoyl]bicyclo[2.2.1]hept-5-ene-2-carboxylic acid;
(1R,2S,6R,7S)-4-[6-(3-furyl)-1,3-benzothiazol-2-yl]-4-azatricyclo[5.2.1.02,6]dec-8-ene-3,5-dione;
4-[6-(4-prop-2-ynoxyphenyl)-1,3-benzothiazol-2-yl]-4-azatricyclo[5.2.1.02,6]dec-8-ene-3,5-dione;
(1R,2S,6R,7S)-4-(6-ethynyl-1,3-benzothiazol-2-yl)-4-azatricyclo[5.2.1.02,6]dec-8-ene-3,5-dione;
(1R,2S,6R,7S)-4-(6-prop-2-ynoxy-1,3-benzothiazol-2-yl)-4-azatricyclo[5.2.1.02,6]dec-8-ene-3,5-dione;
(1R,2S,3R,4S)-3-[(4-hydroxy-1,3-benzothiazol-2-yl)carbamoyl]bicyclo[2.2.1]hept-5-ene-2-carboxylic acid;
3-[(4-prop-2-ynoxy-1,3-benzothiazol-2-yl)carbamoyl]bicyclo[2.2.1]hept-5-ene-2-carboxylic acid;
(1R,2S,6R,7S)-4-(4-prop-2-ynoxy-1,3-benzothiazol-2-yl)-4-azatricyclo[5.2.1.02,6]dec-8-ene-3,5-dione;
3-[[6-(2-phenylethynyl)-1,3-benzothiazol-2-yl]carbamoyl]bicyclo[2.2.1]hept-5-ene-2-carboxylic acid;
3-[[6-(3,3-dimethylbut-1-ynyl)-1,3-benzothiazol-2-yl]carbamoyl]bicyclo [2.2.1]hept-5-ene-2-carboxylic acid;
3-[[6-(2-cyclopropylethynyl)-1,3-benzothiazol-2-yl]carbamoyl]bicyclo[2.2.1]hept-5-ene-2-carboxylic acid;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
(1R,2S,6R,7S)-4-[[6-[(4-methyl-2-pyridyl)oxy]-1,3-benzothiazol-2-yl]-4-azatricyclo[5.2.1.02,6]dec-8-ene-3,5-dione;
1 (1R,2S,6R,7S)-4-[6-[(2-chloro-4-pyridyl)oxy]-1,3-benzothiazol-2-yl]-4-azatricyclo[5.2.1.02,6]dec-8-ene-3,5-dione;
3-[[6-[6-(trifluoromethyl)-3-pyridyl]-1,3-benzothiazol-2-yl]carbamoyl]bicyclo[2.2.1]hept-5-ene-2-carboxylic acid;
(1R,2S,6R,7S)-4-(6-anilino-1,3-benzothiazol-2-yl)-4-azatricyclo[5.2.1.02,6]dec-8-ene-3,5-dione;
4-[6-(2-pyridylamino)-1,3-benzothiazol-2-yl]-4-azatricyclo[5.2.1.02,6]dec-8-ene-3,5-dione; and
3-[[6-[(4-methyl-2-pyridyl)amino]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2 yl]carbamoyl]bicyclo[2.2.1]hept-5-ene-2-carboxylic acid;
3-[[6-[(5-fluoro-2-pyridyl)amino]-1,3-benzothiazol-2 yl]carbamoyl]bicyclo[2.2.1]hept-5-ene-2-carboxylic acid;
4-[6-[(5-fluoro-2-pyridyl)amino]-1,3-benzothiazol-2-yl]-4-azatricyclo[5.2.1.02,6]dec-8-ene-3,5-dione;
3-[[6-[(6-fluoro-2-pyridyl)amino]-1,3-benzothiazol-2-yl]carbamoyl]bicyclo[2.2.1]hept-5-ene-2-carboxylic acid;
4-[6-[(6-fluoro-2-pyridyl)amino]-1,3-benzothiazol-2-yl]-4-azatricyclo[5.2.1.02,6]dec-8-ene-3,5-dione;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethyl)-3-pyridyl]amino]-1,3-benzothiazol-2-yl]carbamoyl]bicyclo[2.2.1]hept-5-ene-2-carboxylic acid;
4-[6-[(2-methoxy-4-pyridyl)amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[6-[[5-(trifluoromethoxy)-2-pyridyl]amino]-1,3-benzothiazol-2-yl]-4-azatricyclo[5.2.1.02,6]dec-8-ene-3,5-dione;
4-[[6-[[5-(trifluoromethyl)-2-pyridyl]amino]-1,3-benzothiazol-2-yl]-4-azatricyclo[5.2.1.02,6]dec-8-ene-3,5-dione;
3-[[6-[[5-(trifluoromethyl)-2-pyridyl]amino]-1,3-benzothiazol-2-yl]carbamoyl]bicyclo[2.2.1]hept-5-ene-2-carboxylic acid;
4-[6-[[6-(trifluoromethyl)-2-pyridyl]amino]-1,3-benzothiazol-2-yl]-4-azatricyclo[5.2.1.02,6]dec-8-ene-3,5-dione;
4-[6-[(2-fluoro-4-pyridyl)amino]-1,3-benzothiazol-2-yl]-4-azatricyclo[5.2.1.02,6]dec-8-ene-3,5-dione;
3-[[6-[(2-fluoro-4-pyridyl)amino]-1,3-benzothiazol-2-yl]carbamoyl]bicyclo[2.2.1]hept-5-ene-2-carboxylic acid;
3-[[6-[4-(1-piperidyl) anilino]-1,3-benzothiazol-2-yl]carbamoyl]bicyclo[2.2.1]hept-5-ene-2-carboxylic acid;
tert-butyl 4-[4-[2-[(1R,2S,6R,7S)-3,5-dioxo-4-azatricyclo[5.2.1.02,6]dec-8-en-4-yl]-1,3-benzothiazol-6-yl]phenyl]piperazine-1-carboxylate;
4-[6-(4-morpholinophenyl)-1,3-benzothiazol-2-yl]-4-azatricyclo[5.2.1.02,6]dec-8-ene-3,5-dione;
or its pharmaceutically acceptable salts or optical isomers, racemates, diastereoisomers, enantiomers or tautomers.

10. The method according to claim 8, wherein said compound of formula (I) is characterized in that:
R₁ is H;
R₂ is H;
R₃ is chosen from:
linear or branched -(C₁-C₆)alkyl optionally substituted by one or more halogen atoms;
linear or branched -(C₁-C₆)alkoxy optionally substituted by one or more halogen atoms;
linear or branched (C₂-C₆)alkynyl;
—OR;
—NHR;
-(C₆-C₁₀)aryl optionally substituted by one or more linear or branched (C₁-C₆)alkyl optionally substituted by one or more halogen atoms, linear or branched (C₁-C₆)alkoxy optionally substituted by one or more halogen atoms, NRaRb; and
R is chosen from:
linear or branched -(C₁-C₆)alkyl optionally substituted by one or more halogen atoms;
-(C₆-C₁₀)aryl optionally substituted by one or more linear or branched -(C₁-C₆)alkyl optionally substituted by one or more halogen atoms;
an heteroaryl comprising 3 to 10 members having at least one heteroatom chosen from O, N, or S; said heteroaryl being optionally substituted by one or more linear or branched -(C₁-C₆)alkyl optionally substituted by one or more halogen atoms;
Ra and Rb are linked together to form an heterocycloalkyl comprising 3 to 10 members; said heterocycloalkyl being optionally substituted by one or more linear or branched -(C₁-C₆)alkyl, —COO—(C₁-C₃)alkyl, —NRcRd; oxo; and
Rc and Rd, identical or different, are chosen from:
—H; and
linear or branched -(C₁-C₆)alkyl.

11. The method according to claim 8, wherein said compound of formula (I) is characterized in that:
R₁ is H;
R₂ is H;
R₃ is chosen from:
linear or branched -(C₁-C₃)alkyl, preferably methyl;
trifluorometoxhy;
ethoxy;
3,3-dimethylbut-1-ynyl;
—OR,
—NHR;
phenyl optionally substituted by one or more trifluoromethyl, methoxy trifluroromethoxy and/or piperidyl; and
R is chosen from:
linear or branched -(C₁-C₃)alkyl optionally substituted by one or more halogen atoms, preferably one or more fluor atoms;
phenyl optionally substituted by one or more methyl and/or trifluoromethyl;
a heteroaryl comprising 6 members and having at least one nitrogen atom; said heteroaryl being optionally substituted by one or more trifluromethyl, trifluoromethoxy and/or fluorine; for example a pyridyl substituted by one or more trifluoromethyl, trifluoromethoxy and/or fluorine.

12. The method according to claim 8, wherein said compound of formula (I) is chosen from:
3-[(6-ethoxy-1,3-benzothiazol-2-yl)carbamoyl]bicyclo[2.2.1]hept-5-ene-2-carboxylic acid;
(1R,2S,6R,7S)-4-(6-ethoxy-1,3-benzothiazol-2-yl)-4-azatricyclo[5.2.1.02,6]dec-8-ene-3,5-dione;
3-[[6-(trifluoromethoxy)-1,3-benzothiazol-2-yl]carbamoyl]bicyclo[2.2.1]hept-5-ene-2-carboxylic acid;
(1R,2S,6R,7S)-4-[6-(trifluoromethoxy)-1,3-benzothiazol-2-yl]-4-azatricyclo[5.2.1.02,6]dec-8-ene-3,5-dione;
3-[[6-(3,3-dimethylbut-1-ynyl)-1,3-benzothiazol-2-yl]carbamoyl]bicyclo [2.2.1]hept-5-ene-2-carboxylic acid;
3-[[6-(4-methoxyphenyl)-1,3-benzothiazol-2-yl]carbamoyl]bicyclo[2.2.1]hept-5-ene-2-carboxylic acid;
3-[6-[[4-(trifluoromethoxy)phenyl]-1,3-benzothiazol-2-yl]carbamoyl]bicyclo[2.2.1]hept-5-ene-2-carboxylic acid;
3-[[6-[4-(trifluoromethyl)phenyl]-1,3-benzothiazol-2-yl]carbamoyl]bicyclo[2.2.1]hept-5-ene-2-carboxylic acid;
(1R,2S,6R,7S)-4-[6-[4-(trifluoromethyl)phenyl]-1,3-benzothiazol-2-yl]-4-azatricyclo[5.2.1.02,6]dec-8-ene-3,5-dione;
3-[(6-methyl-1,3-benzothiazol-2-yl)carbamoyl]bicyclo[2.2.1]hept-5-ene-2-carboxylic acid;
4-(6-methyl-1,3-benzothiazol-2-yl)-4-azatricyclo[5.2.1.02,6]dec-8-ene-3,5-dione;
3-[(6-phenoxy-1,3-benzothiazol-2-yl)carbamoyl]bicyclo[2.2.1]hept-5-ene-2-carboxylic acid;
(1R,2S,6R,7S)-4-(6-phenoxy-1,3-benzothiazol-2-yl)-4-azatricyclo[5.2.1.02,6]dec-8-ene-3,5-dione;
[2-hydroxy-1,1-bis(hydroxymethyl)ethyl]ammonium; (1R,2S,3R,4S)-3-[(6-phenoxy-1,3-benzothiazol-2-yl)carbamoyl]bicyclo[2.2.1]hept-5-ene-2-carboxylate;
3-[[6-[[4-(trifluoromethyl)-2-pyridyl]oxy]-1,3-benzothiazol-2-yl]carbamoyl]bicyclo[2.2.1]hept-5-ene-2-carboxylic acid;
3-[[6-[(4-fluoro-2-pyridyl)amino]-1,3-benzothiazol-2-yl]carbamoyl]bicyclo[2.2.1]hept-5-ene-2-carboxylic acid;
3-[[6-[(5-fluoro-2-pyridyl)amino]-1,3-benzothiazol-2-yl]carbamoyl]bicyclo[2.2.1]hept-5-ene-2-carboxylic acid;
3-[[6-[(6-fluoro-2-pyridyl)amino]-1,3-benzothiazol-2-yl]carbamoyl]bicyclo[2.2.1]hept-5-ene-2-carboxylic acid;
4-[6-[[2-(trifluoromethyl)-4-pyridyl]amino]-1,3-benzothiazol-2-yl]-4-azatricyclo[5.2.1.02,6]dec-8-ene-3,5-dione;
3-[[6-[[5-(trifluoromethoxy)-2-pyridyl]amino]-1,3-benzothiazol-2-yl]carbamoyl]bicyclo[2.2.1]hept-5-ene-2-carboxylic acid;
4-[[6-[[5-(trifluoromethyl)-2-pyridyl]amino]-1,3-benzothiazol-2-yl]-4-azatricyclo[5.2.1.02,6]dec-8-ene-3,5-dione;
4-[6-[[6-(trifluoromethyl)-2-pyridyl]amino]-1,3-benzothiazol-2-yl]-4-azatricyclo[5.2.1.02,6]dec-8-ene-3,5-dione;
3-[[6-[4-(1-piperidyl) anilino]-1,3-benzothiazol-2-yl]carbamoyl]bicyclo[2.2.1]hept-5-ene-2-carboxylic acid;
or its pharmaceutically acceptable salts or optical isomers, racemates, diastereoisomers, enantiomers or tautomers.

13. The method according to claim 8 any of claims 8 to 12, in the prevention and/or treatment of a mycobacterial infection or in the treatment of a disease caused by infection with a mycobacterium.

14. The method according to claim 13, wherein the mycobacterial infection is a Mycobacterium tuberculosis infection.

15. The method according to claim 8, for the prevention and/or treatment of tuberculosis.

16. The method according to claim 8, wherein said compound is administered used in combination with at least one other anti-mycobacterial agent.

17. A combination of (a) a compound of Formula (I) as defined in claim 1 and (b) at least one other anti-mycobacterial agent.

18. A pharmaceutical composition according to claim 7, wherein said at least one other anti-mycobacterial agent is an anti-tuberculosis agent.

19. A pharmaceutical composition according to claim 18 wherein the anti-tuberculosis agent is selected from nitroimidazole compounds such as pretomanid and delamanid.

20. The method according to claim 16 wherein said at least one other anti-mycobacterial agent is an anti-tuberculosis agent.

21. The method according to claim 20 wherein the anti-tuberculosis agent is selected from nitroimidazole compounds such as pretomanid and delamanid.

22. The combination of claim 17 wherein said at least one other anti-mycobacterial agent is an anti-tuberculosis agent.

23. The method according to claim 22 wherein the anti-tuberculosis agent is selected from nitroimidazole compounds such as pretomanid and delamanid.
